SEQUENCE LISTING (39 sequences)

SEQ ID NO: 1 - protein, 5 aa, synthetic construct
NYWMS

SEQ ID NO: 2 - protein, 17 aa, synthetic construct
DIHGNRGFNYHASWAKG

SEQ ID NO: 3 - protein, 9 aa, synthetic construct
ADDSGSHDI

SEQ ID NO: 4 - protein, 12 aa, synthetic construct
QSSQSVYSAKLS

SEQ ID NO: 5 - protein, 7 aa, synthetic construct
YGSTLAS

SEQ ID NO: 6 - protein, 12 aa, synthetic construct
QGTFYGPDWYFA

SEQ ID NO: 7 - protein, 116 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 8 - protein, 111 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 9 - protein, 446 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 446 aa, synthetic construct
QQQLVESGGRLVTPGGSLTLTCTASGFSLNNYWMSWVRQAPGKGLEWIGDIHGNRGFNYHASWAKGRFTVSRTSTTVDLRMTSLTTEDTAIYFCARADDSGSHDIWGPGTLVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPMVTCVVVDVSEDDPDVQISWFVNNVEVLTAQTQTHREDYNSTLRVVSALPIQHQDWMSGKEFKCKVNNKALPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 14 - protein, 218 aa, synthetic construct
ELDLTQTPSPVSAAVGDTVTINCQSSQSVYSAKLSWYQQKPGQPPKLLIYYGSTLASGVPSRFKGSGSGTQFSLTISDVQCADAATYYCQGTYYGPDWYFAFGGGTEVVVKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 245 aa, synthetic construct
ELDLTQTPASVSAAVGGTVTINCQSSQSPYSNEWLSWYQQKPGQAPKVLISRISTLASGVSSRFKGSGSGTQFTLTISDLECGDAATYFCAGGYNFGLFPFGGGTELEILSSGGGGSGGGGGGSSRSSQSVKESEGRLVTPGGSLTLTCTVSGIDLSRDWMNWVRQAPGEGLEWIGAIGRSGDTYYATWAKGRFTISKTSSRTVTLTVTDLQRSDTATYFCARIPYFGWNNGDIWGPGTLVTISS

SEQ ID NO: 16 - protein, 247 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 17 - protein, 247 aa, synthetic construct
EVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSGGGGSGGGGSGGGGSGGGGSDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIK

SEQ ID NO: 18 - protein, 242 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSS

SEQ ID NO: 19 - protein, 501 aa, synthetic construct
MALPVTALLLPLALLLHAARPDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDYKDDDDK

SEQ ID NO: 20 - protein, 21 aa, synthetic construct
MALPVTALLLPLALLLHAARP

SEQ ID NO: 21 - protein, 15 aa, synthetic construct
GGGGSGGGGSGGGGS

SEQ ID NO: 22 - protein, 20 aa, synthetic construct
GGGGSGGGGSGGGGSGGGGS

SEQ ID NO: 23 - protein, 62 aa, synthetic construct
ALSNSIMYFSHFVPVFLPAKPTTTPAPRPPTPAPTIASQPLSLRPEASRPAAGGAVHTRGLD

SEQ ID NO: 24 - protein, 70 aa, synthetic construct
KPFWVLVVVGGVLACYSLLVTVAFIIFWVRSKRSRLLHSDYMNMTPRRPGPTRKHYQPYAPPRDFAAYRS

SEQ ID NO: 25 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 21 aa, synthetic construct
GSGEGRGSLLTCGDVEENPGP

SEQ ID NO: 27 - protein, 367 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 28 - protein, 895 aa, synthetic construct
MALPVTALLLPLALLLHAARPDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYKAKPLSQEESTLIERATATINSIPISEDYSVASAALSSDGRIFTGVNVYHFTGGPCAELVVLGTAAAAAAGNLTCIVAIGNENRGILSPCGRCRQVLLDLHPGIKAIVKDSDGQPTAVGIRELLPSGYVWEG

SEQ ID NO: 29 - protein, 18 aa, synthetic construct
GSTGSGSKPGSGEGSTKG

SEQ ID NO: 30 - protein, 49 aa, synthetic construct
KPTTTPAPRPPTPAPTIASQPLSLRPEASRPAAGGAVHTRGLDFASDKP

SEQ ID NO: 31 - protein, 24 aa, synthetic construct
IYIWAPLAGTCGVLLLSLVITLYC

SEQ ID NO: 32 - protein, 154 aa, synthetic construct
KRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 8 aa, synthetic construct
DYKDDDDK

SEQ ID NO: 34 - protein, 493 aa, synthetic construct
MALPVTALLLPLALLLHAARPDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 472 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSKPTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 505 aa, synthetic construct
MALPVTALLLPLALLLHAARPDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 484 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 507 aa, synthetic construct
MALPVTALLLPLALLLHAARPDIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 486 aa, synthetic construct
DIQMTQSPSSLSASVGDRVTITCQSSQSVYSAKLSWYQQKPGKAPKLLIYYGSTLASGVPSRFSGSGSGTQFTLTISSLQPEDFATYYCQGTFYGPDWYFAFGGGTKVEIKGGGGSGGGGSGGGGSEVQLVESGGGLVQPGGSLRLSCAASGFTFSNYWMSWVRQAPGKGLEWVGDIHGNRGFNYHASWAKGRFTVSRSKNTLYLQMNSLRAEDTAVYYCAKADDSGSHDIWGQGTLVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2026/0325580 A1
(43) Pub. Date: Oct. 22, 2026

(54) CYTOTOXICITY TARGETING CHIMERAS FOR CAR-T AND CAR-NK CELLS

(71) Applicant: GLAXOSMITHKLINE INTELLECTUAL PROPERTY DEVELOPMENT LIMITED, Brentford, Middlesex (GB)

(72) Inventors: Adolfo Alfonso, Collegeville, PA (US); Craig Leach, Collegeville, PA (US); Christina Ng Di Marco, Collegeville, PA (US); Matthew Robert Sender, Collegeville, PA (US); Brandon James Turunen, Boston, MA (US)

(21) Appl. No.: 19/474,925

(22) PCT Filed: Apr. 19, 2024
(86) PCT No.: PCT/EP2024/060800

§ 371 Date: Oct. 13, 2025

Related U.S. Application Data
(60) Provisional application No. 63/460,435, filed on Apr. 19, 2023.

(51) Int. Cl. A61K 35/17; A61K 40/11; A61K 40/15; A61K 40/31; A61K 40/40; A61K 47/54; A61P 35/00; C07K 16/44

(52) U.S. Cl. CPC A61K 35/17; A61K 40/11; A61K 40/15; A61K 40/31; A61K 40/429; A61K 47/555; A61P 35/00; C07K 16/44; A61K 2239/13; A61K 2239/21; A61K 2239/22; C07K 2317/565; C07K 2317/622

(57) ABSTRACT

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antigen binding domain. The present disclosure also relates to agents capable of binding to a receptor on a surface of a pathogenic cell and inducing the depletion or killing of the pathogenic cell by an immunomodulatory cell expressing the antigen binding domain in a subject for use in the treatment of cancer, inflammatory diseases, autoimmune diseases, viral infection, or bacterial infection.

### CROSS-REFERENCE TO RELATED APPLICATIONS

This application claims the benefit of and priority to U.S. Provisional Patent Application Ser. No. 63/460,435, filed Apr. 19, 2023, the contents of which are hereby incorporated by reference in their entirety.

### REFERENCE TO AN ELECTRONIC SEQUENCE LISTING

This application contains a sequence listing which has been submitted electronically in ST.26 format and is hereby incorporated by reference in its entirety (said ST.26 copy, created on Apr. 16, 2024, is named “209279_seqlist.xml” and is 42,890 bytes in size).

### FIELD OF THE DISCLOSURE

The present disclosure relates to heterobifunctional molecules, referred to as cytotoxicity targeting chimeras (CyTaCs) or antibody recruiting molecules (ARMs) that are able to simultaneously bind a target cell-surface protein as well as an exogenous antigen binding domain. The present disclosure also relates to agents capable of binding to a receptor on a surface of a pathogenic cell and inducing depletion or killing of the pathogenic cell by an immunomodulatory cell expressing the antigen binding domain in a subject for use in the treatment of cancer, inflammatory diseases, autoimmune diseases, viral infection, or bacterial infection.

### BACKGROUND

Cell-surface proteins and their ligands play key roles in a range of inflammatory, infectious, and autoimmune diseases as well as tumor initiation, growth and metastasis. Antibody-based therapeutics have promising properties as drug candidates for these indications due to their selectivity for pathogenic cell-surface targets and their ability to direct immune surveillance to target-expressing tissues or cells to induce depletion of the pathogenic cells. Examples of such depletion mechanisms include antibody-dependent cellular.cytotoxicity (ADCC), antibody-dependent cellular phagocytosis (ADCP), and complement-dependent cytotoxicity (CDC). However, antibody-based therapeutics often suffer from a lack of bioavailability, high cost, thermal instability, and difficult manufacturing due to their size, complexity, and peptide based structures. Conversely, small molecule therapeutics often provide affordability, stability, and the convenience of oral dosing, but may suffer from poor selectivity and off-target effects, while also lacking the immune control of therapeutic antibodies.

Adoptive cell transfer (ACT) therapeutics, especially with T-cells or NK-cells transduced with Chimeric Antigen Receptors (CARs), has also shown promise in a range of inflammatory, infectious, and autoimmune diseases as well as tumor initiation, growth and metastasis. However, ACT therapeutics often suffer from high cost, instability, safety concerns, and difficult manufacturing due to their complexity. Conversely, small molecule therapeutics often provide affordability, stability, and the convenience of oral dosing, but may suffer from poor selectivity and off-target effects, while also lacking the immune control of cell-based therapeutics.

Accordingly, a need exists for improved therapeutic approaches that target pathogenic cells for use in the treatment of disease. Such compositions and related methods are provided in the present disclosure.

### SUMMARY

In one aspect, the present disclosure provides a chimeric antigen receptor comprising an extracellular ligand binding domain; a transmembrane domain; and an intracellular signalling domain; wherein the extracellular ligand binding domain comprises an anti-cotinine antibody or antigen-binding fragment thereof. In a further aspect, the anti-cotinine antibody or antigen-binding fragment thereof comprises a heavy chain CDR1, CDR2, and CDR3 having SEQ ID NO: 1, SEQ ID NO: 2, and SEQ ID NO: 3, respectively, and a light chain CDR1, CDR2, and CDR3 having SEQ ID NO: 4, SEQ ID NO: 5, and SEQ ID NO: 6, respectively

In a further aspect, the present disclosure provides a polynucleotide encoding the chimeric antigen receptor. In a further aspect, the present disclosure provides an expression vector comprising the polynucleotide. In a further aspect, the present disclosure provides a cell comprising the polynucleotide. In a further aspect, the present disclosure provides a cell comprising the expression vector. In a further aspect, the cell is an immunomodulatory cell. In a further aspect, the immunomodulatory cell is a T-cell. In a further aspect, the immunomodulatory cell is a natural killer (NK) cell.

In a further aspect, the present disclosure provides a combination comprising a modified immunomodulatory cell as described herein and a heterobifunctional molecule, referred to as a cytoxicity targeting chimera (CyTaC) or an antibody recruiting molecule (ARM). In a further aspect, the ARM comprises a moiety that binds a target cell-surface protein on a cell and a moiety that binds an exogenous antibody. In a further aspect, the ARM comprises a divalent linker that links the target-binding moiety to the antibody-binding moiety. In a further aspect, the exogenous antibody is an anti-cotinine antibody, or antigen-binding fragment thereof. In a further aspect, the ARM comprises a moiety that binds a target cell-surface protein covalently linked to a cotinine moiety or a cotinine derivative moiety.

In a further aspect, the ARM is a compound of Formula (I):

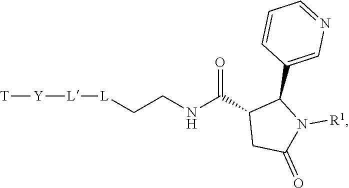

or a pharmaceutically acceptable salt thereof,
wherein:
T is a target binding moiety;
R¹ is C₁₋₄ alkyl or C₃₋₆ cycloalkyl;
L′ is a bond,

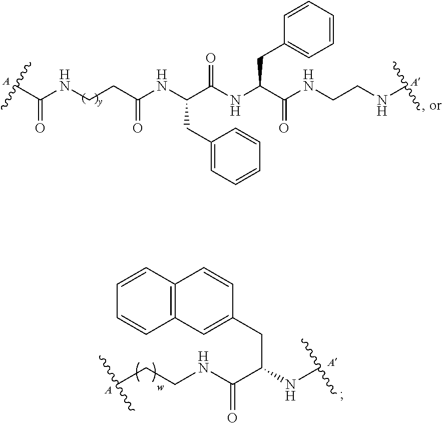

y is an integer of 1 to 9;
w is an integer of 0 to 5;
Y is a bond or a divalent spacer moiety of one to twelve atoms in length; and
L is a divalent linker as described herein;
wherein each

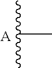

represents a covalent bond to the Y group of Formula (I), or when Y is a bond, a covalent bond to the T group of Formula (I), and each

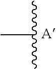

represents a covalent bond to the L group of Formula (I).

In one aspect, the present disclosure provides a method of treating and/or preventing a disease or disorder in a patient in need thereof, comprising: administering to the patient a therapeutically effective amount of a combination comprising a modified immunomodulatory cell as disclosed herein and a heterobifunctional molecule as disclosed herein.

In one aspect, the present disclosure provides a method of increasing cell killing of target-expressing cells comprising: contacting the cells with an effective amount of a combination comprising a modified immunomodulatory cell as disclosed herein and a heterobifunctional molecule as disclosed herein.

In one aspect, the present disclosure provides a method of depleting target-expressing cells comprising: contacting the cells with an effective amount of a combination comprising a modified immunomodulatory cell as disclosed herein and a heterobifunctional molecule as disclosed herein.

In one aspect, the present disclosure provides a combination comprising a modified immunomodulatory cell as disclosed herein and a heterobifunctional molecule as disclosed herein for use in therapy.

In one aspect, the present disclosure provides a combination comprising a modified immunomodulatory cell as disclosed herein and a heterobifunctional molecule as disclosed herein for use in the treatment of a disease or disorder.

In one aspect, the present disclosure provides use of a combination comprising a modified immunomodulatory cell as disclosed herein and a heterobifunctional molecule as disclosed herein in the manufacture of a medicament for the treatment of a disease or disorder.

In one aspect, the present disclosure provides a combination comprising a modified immunomodulatory cell as disclosed herein and a heterobifunctional molecule as disclosed herein for use in increasing cell killing of target-expressing cells, wherein a target-binding moiety of the molecule binds the target expressed on the cells.

In one aspect, the present disclosure provides use of a combination comprising a modified immunomodulatory cell as disclosed herein and a heterobifunctional molecule as disclosed herein in the manufacture of a medicament for increasing cell killing of target-expressing cells, wherein a target-binding moiety of the molecule binds the target expressed on the cells.

In one aspect, the present disclosure provides a combination comprising a modified immunomodulatory cell as disclosed herein and a heterobifunctional molecule as disclosed herein for use in depleting target-expressing cells, wherein a target-binding moiety of the molecule binds the target expressed on the cells.

In one aspect, the present disclosure provides use of a combination comprising a modified immunomodulatory cell as disclosed herein and a heterobifunctional molecule as disclosed herein in the manufacture of a medicament for depleting target-expressing cells, wherein a target-binding moiety of the molecule binds the target expressed on the cells.

### BRIEF DESCRIPTION OF THE FIGURES

FIG. 1: Schematic representation of cytotoxicity targeting chimeras (CyTaCs) technology compared to current antibody technology, wherein the platform reagent can be a platform CAR-T cell or platform CAR-NK cell according to embodiments disclosed herein.

FIG. 2A: Graph showing evaluation of CAR-NK cell killing of PSMA⁺ and αvβ6⁺ target cells mediated by PSMA targeting CyTaC (compound 1) and αvβ6 targeting CyTaC (compound 5) via CellTiter-Glo® cell viability assay as described in Example 5 (method 1).

FIG. 2B: Graph showing % cell killing of αVβ6⁺ target cells treated with anti-cotinine CAR-NK cells and PSMA targeting CyTaC (compound 1) or αvβ6 targeting CyTaC (compound 5) via CellTiter-Glo® cell viability assay as described in Example 5 (method 1).

FIG. 2C: Graph showing IFNγ levels in supernatant of target cells (PSMA⁺, αvβ6⁺, FOLR⁺, or FAP⁺ cells) treated with 1 nM PSMA targeting CyTaC (compound 1), αvβ6 targeting CyTaC (compound 5), FOLR targeting CyTaC (compound 6), or FAP targeting CyTaC (compound 7) and anti-cotinine CAR-NK cells (5:1 ratio of effector cells:target cells) as described in Example 5 (method 1).

FIG. 3: Real-time cell analysis (RTCA) impedance (xCELLigence®) data trace showing cell killing of LNCap (PSMA⁺) cells treated with 100 nM PSMA-targeting CyTaC (compound 1) and anti-cotinine CAR-T cells (10:1 ratio of effector cells:target cells) at 24 hour time point, determined as described in Example 5 (method 2).

FIG. 4: Graph showing cell killing of LNCap-GPP (PSMA+) target cells by anti-cotinine CAR-T cells mediated by PSMA targeting CyTaC (compound 1) visualized by live cell analysis (Incucyte®) as described in Example 5 (method 3).

FIG. 5: Graph showing specific lysis of CCR2 expressing cells by anti-cotinine CAR-NK cells mediated by CCR2 targeting CyTaC as measured by calcein-AM release (as described in Example 6).

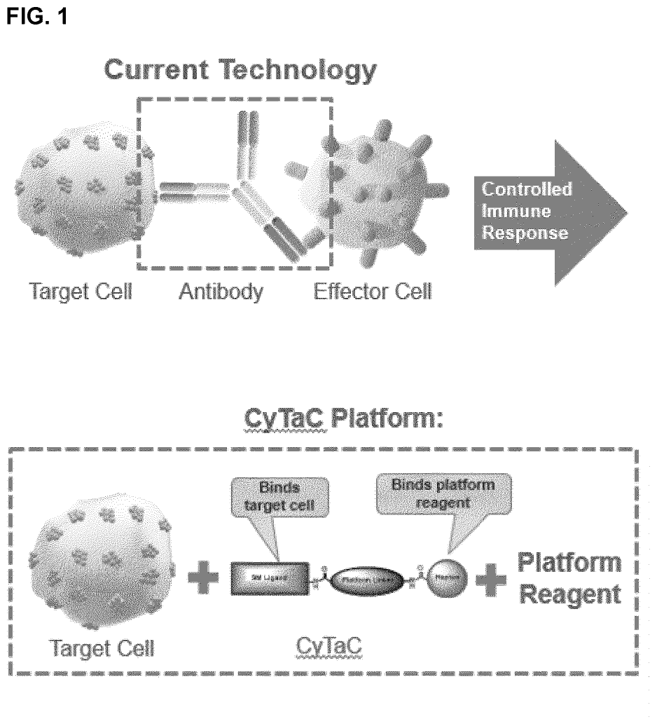

### DETAILED DESCRIPTION

### Definitions

As used herein and in the claims, the singular forms “a” and “the” include plural reference unless the context clearly dictates otherwise.

As used herein and in the claims, the term “comprising” encompasses “including” or “consisting” e.g. a composition “comprising” X may consist exclusively of X or may include something additional, e.g., X+Y.

The term “consisting essentially of” limits the scope of the feature to the specified materials or steps and those that do not materially affect the basic characteristic(s) of the claimed feature.

The term “consisting of” excludes the presence of any additional component(s).

The term “pathogenic cells” includes a cell subset that causes or is capable of causing disease. Examples of pathogenic cells include, but are not limited to, pathogenic immune cells, cancer or tumor cells, and stromal cells. A pathogenic cell can also be a pathogenic agent capable of causing an infection, such as a virus or a bacterial cell.

The term “pathogenic immune cells” includes a particular immune cell subset that causes or is capable of causing disease. These cellular subsets are resident cells or are recruited to particular locations and secrete cytokines, chemokines and other mediators and contribute to the persistence and progression of disease such as cancer in the case of a tumor microenvironment or chronic inflammation of the lung in the case of asthma. Examples of pathogenic immune cells include, but are not limited to myeloid-derived suppressor cells (MDSCs), T regulatory cells (Tregs), neutrophils, macrophages, B regulatory cells (Bregs), CD8 regulatory cells, (CD8regs), and exhausted T cells.

The term “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients therefor.

The terms “effective amount” and “therapeutically effective amount” refer to an amount of a compound, or antibody, or antigen-binding portion thereof, according to the invention, which when administered to a patient in need thereof, is sufficient to effect treatment for disease-states, conditions, or disorders for which the compounds have utility. Such an amount would be sufficient to elicit the biological or medical response of a tissue system, or patient that is sought by a researcher or clinician. The amount of a compound according to the invention which constitutes a therapeutically effective amount will vary depending on such factors as the compound and its biological activity, the composition used for administration, the time of administration, the route of administration, the rate of excretion of the compound, the duration of the treatment, the type of disease-state or disorder being treated and its severity, drugs used in combination with or coincidentally with the compounds of the invention, and the age, body weight, general health, sex and diet of the patient. Such a therapeutically effective amount can be determined routinely by one of ordinary skill in the art having regard to their own knowledge, the state of the art, and this disclosure.

The term “alkyl” represents a saturated, linear or branched hydrocarbon moiety having the specified number of carbon atoms. The term “C₁₋₃ alkyl” refers to an unsubstituted alkyl moiety containing 1, 2 or 3 carbon atoms; exemplary alkyls include methyl, ethyl and propyl.

The term “alkylene” represents a saturated, linear or branched hydrocarbon moiety having the specified number of carbon atoms, with two points of attachment. The two points of attachment can be from the same or different carbon atoms. The term “C₁₋₃ alkylene” refers to an unsubstituted alkylene moiety containing 1, 2 or 3 carbon atoms with two points of attachment; exemplary C₁₋₃ alkylene groups include methylene, ethylene and propylene.

The term “alkenyl” represents an unsaturated, linear or branched hydrocarbon moiety having the specified number of carbon atoms. The term “C₂₋₆ alkenyl” refers to an unsubstituted alkenyl moiety containing 2, 3, 4, 5, or 6 carbon atoms; exemplary alkenyls include propenyl, butenyl, pentenyl and hexenyl.

The term “alkenylene” represents an unsaturated, linear or branched hydrocarbon moiety having the specified number of carbon atoms, with two points of attachment. The two points of attachment can be from the same or different carbon atoms. The term “C₂₋₆ alkenylene” refers to an unsubstituted alkenylene moiety containing 2, 3, 4, 5, or 6 carbon atoms with two points of attachment; exemplary C₂₋₆ alkenylene groups include propenylene, butenylene, pentenylene and hexenylene.

The term “cycloalkyl” represents a saturated cyclic hydrocarbon moiety having the specified number of carbon atoms. The term “C₃₋₆ cycloalkyl” refers to an unsubstituted cycloalkyl moiety containing 3, 4, 5 or 6 carbon atoms; exemplary cycloalkyls include cyclopropyl, cyclobutyl, cyclopentyl, and cyclohexyl.

The term “cycloalkylene” represents a saturated cyclic hydrocarbon moiety having the specified number of carbon atoms, with two points of attachment. The two points of attachment can be from the same or different carbon atoms. The term “C₄₋₆ cycloalkylene” refers to an unsubstituted cycloalkylene moiety containing 4, 5, or 6 carbon atoms with two points of attachment. Exemplary cycloalkylene groups include cyclobutane-1,3-diyl, cyclopentane-1,3-diyl, cyclohexane-1,3-diyl, or cyclohexane-1,4-diyl.

The term “cycloalkenylene” represents an unsaturated cyclic hydrocarbon moiety having the specified number of carbon atoms, with two points of attachment. The two points of attachment can be from the same or different carbon atoms. The term “C₃₋₆ cycloalkenylene” refers to an unsubstituted cycloalkenylene moiety containing 3, 4, 5, or 6 carbon atoms with two points of attachment.

The term “heterocycloalkylene” refers to a saturated cyclic hydrocarbon moiety containing 1 or 2 heteroatoms independently selected from oxygen, sulphur or nitrogen atoms, with two points of attachment. The two points of attachment can be from the same or different carbon atoms. The term “3- to 6-membered heterocycloalkylene” refers to a 3- to 6-membered saturated cyclic moiety containing 2, 3, 4 or 5 carbon atoms in addition to 1 or 2 oxygen, sulphur or nitrogen atoms, with two points of attachment. Suitably, the 3- to 6-membered heterocycloalkylene group contains 1 oxygen or nitrogen atom. Suitably such group contains 3 carbon atoms and 1 oxygen or nitrogen atom, such as azetidindiyl or oxetandiyl. Suitably such group contains 4 or 5 carbon atoms and 1 oxygen or nitrogen atom, such as tetrahydrofurandiyl, tetrahydropyrandiyl, pyrrolidindiyl or piperidindiyl.

The term “bridged bicyclic cycloalkylene” refers to a saturated bicyclic hydrocarbon moiety having at least one bridge, with two points of attachment. A “bridge” is an unbranched chain of atoms or an atom or a valence bond connecting two bridgeheads, where a “bridgehead” is any skeletal atom of the ring system which is bonded to three or more skeletal atoms (excluding hydrogen). The two points of attachment can be from the same or different carbon atoms. The term “C₇₋₉ bridged bicyclic cycloalkylene” refers to an unsubstituted bridged bicyclic cycloalkylene moiety containing 7, 8, or 9 carbon atoms with two points of attachment.

The term “arylene” refers to a monocyclic or bicyclic ring system wherein at least one ring in the system is aromatic, with two points of attachment. Exemplary arylene groups include phenylene, biphenylene, naphthylene, and anthracylene.

The term “heteroarylene” refers to a monocyclic or bicyclic ring system wherein at least one ring in the system is aromatic, and having, in addition to carbon atoms, from one to five heteroatoms independently selected from oxygen, sulphur or nitrogen atoms, with two points of attachment. The term “5- to 6-membered heteroarylene” refers to a 5- to 6-membered cyclic aromatic moiety containing 2, 3, 4 or 5 carbon atoms in addition to 1, 2, or 3 heteroatoms independently selected from oxygen, sulphur or nitrogen atoms, with two points of attachment.

The skilled artisan will appreciate that salts, including pharmaceutically acceptable salts, of the compounds according to Formula (I) may be prepared. Indeed, in certain embodiments of the invention, salts including pharmaceutically-acceptable salts of the compounds according to Formula (I) may be preferred over the respective free or unsalted compound. Accordingly, the invention is further directed to salts, including pharmaceutically-acceptable salts, of the compounds according to Formula (I). The invention is further directed to free or unsalted compounds of Formula (I).

The salts, including pharmaceutically acceptable salts, of the compounds of the invention are readily prepared by those of skill in the art.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, trifluoroacetate, undecanoate, undecylenate, and valerate.

Representative pharmaceutically acceptable base addition salts include, but are not limited to, aluminium, 2-amino-2-(hydroxymethyl)-1,3-propanediol (TRIS, tromethamine), arginine, benethamine (N-benzylphenethylamine), benzathine (N,N′-dibenzylethylenediamine), b/s-(2-hydroxyethyl)amine, bismuth, calcium, chloroprocaine, choline, clemizole (1-p chlorobenzyl-2-pyrrolidine-1′-ylmethylbenzimidazole), cyclohexylamine, dibenzylethylenediamine, diethylamine, diethyltriamine, dimethylamine, dimethylethanolamine, dopamine, ethanolamine, ethylenediamine, L-histidine, iron, isoquinoline, lepidine, lithium, lysine, magnesium, meglumine (N-methylglucamine), piperazine, piperidine, potassium, procaine, quinine, quinoline, sodium, strontium, t-butylamine, and zinc.

The compounds according to Formula (I) may contain one or more asymmetric centers (also referred to as a chiral center) and may, therefore, exist as individual enantiomers, diastereomers, or other stereoisomeric forms, or as mixtures thereof. Chiral centers, such as chiral carbon atoms, may be present in a substituent such as an alkyl group. Where the stereochemistry of a chiral center present in a compound of Formula (I), or in any chemical structure illustrated herein, if not specified the structure is intended to encompass all individual stereoisomers and all mixtures thereof. Thus, compounds according to Formula (I) containing one or more chiral centers may be used as racemic mixtures, enantiomerically enriched mixtures, or as enantiomerically pure individual stereoisomers.

A mixture of stereoisomers in which the relative configuration of all of the stereocenters is known may be depicted using the symbol “&” together with an index number (e.g., “&1”). For example, a group of two stereogenic centers labeled with the symbol “&1” represents a mixture of two possible stereoisomers in which the two stereogenic centers have a relative configuration as depicted.

Divalent groups are groups having two points of attachment. For all divalent groups, unless otherwise specified, the orientation of the group is implied by the direction in which the formula or structure of the group is written.

Unless defined otherwise, all technical and scientific terms used herein have the same meaning as commonly understood by one of ordinary skill in the art to which this invention belongs. Although any compositions and methods similar or equivalent to those described herein can be used in the practice or testing of the methods of the disclosure, exemplary compositions and methods are described herein. Any of the aspects and embodiments of the disclosure described herein may also be combined. For example, the subject matter of any dependent or independent claim disclosed herein may be multiply combined (e.g., one or more recitations from each dependent claim may be combined into a single claim based on the independent claim on which they depend).

Ranges provided herein include all values within a particular range described and values about an endpoint for a particular range.

Concentrations described herein are determined at ambient temperature and pressure. This may be, for example, the temperature and pressure at room temperature or in a particular portion of a process stream. Preferably, concentrations are determined at a standard state of 25° C. and 1 bar of pressure.

### Chimeric Antigen Receptors (CARs)

The present disclosure provides genetically engineered receptors that redirect immune effector cells toward cancer cells expressing an epitope as described herein. These genetically engineered receptors, referred to herein as chimeric antigen receptors (CARs), are molecules that combine antibody-based specificity for a desired antigen/epitope with a T cell receptor-activating intracellular domain to generate a chimeric protein that exhibits a specific cellular immune activity. The term “chimeric” describes being composed of parts of different proteins or DNAs from different origins.

In one aspect, the present disclosure provides a chimeric antigen receptor comprising: an extracellular ligand binding domain; a transmembrane domain; and an intracellular signalling domain. In one embodiment of the disclosure, the extracellular ligand binding domain comprises an anti-cotinine antibody or antigen-binding fragment thereof comprising a heavy chain CDR1 having SEQ ID NO: 1, a heavy chain CDR2 having SEQ ID NO: 2, a heavy chain CDR3 having SEQ ID NO: 3, a light chain CDR1 having SEQ ID NO: 4, a light chain CDR2 having SEQ ID NO: 5, and a light chain CDR3 having SEQ ID NO: 6.

Engagement of the antigen binding domain of the CAR with the target antigen on the surface of a target cell results in clustering of the CAR and delivers an activation stimulus to the CAR-containing cell. The main characteristic of CARs is their ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors.

In various embodiments, a CAR comprises an extracellular binding domain that comprises an antigen binding domain (e.g., an anti-cotinine antibody or antigen-binding fragment thereof); a transmembrane domain; one or more co-stimulatory signalling domains; and one or more intracellular signalling domains.

The term “chimeric antigen receptor” (“CAR”) as used herein, refers to an engineered receptor comprising an extracellular antigen binding domain (usually derived from a monoclonal antibody, or fragment thereof, e.g., a VH domain in the form of a single-domain antibody (sdAb) or a VH domain and a VL domain in the form of a scFv), and optionally a spacer region, a transmembrane region, and one or more intracellular effector domains. In particular embodiments, the CAR further comprises a hinge region between the antigen binding domain and the intracellular signalling domain. The CAR may also comprise hinge domains or spacer domains between any of the extracellular binding domain, the transmembrane domain, the co-stimulatory domains and/or the intracellular signalling domains. CARs have also been referred to as chimeric T cell receptors or chimeric immunoreceptors (CIRs). CARs are genetically introduced into hematopoietic cells, such as T cells, to redirect T cell specificity for a desired cell-surface antigen, resulting in a CAR-T therapeutic. The term “spacer domain” as used herein, refers to an oligo- or polypeptide that functions to link the transmembrane domain to the extracellular antigen/target binding domain. This region may also be referred to as a “hinge domain” or “stalk domain.” The size of the spacer can be varied depending on the position of the target epitope in order to have optimal function upon CAR:target/antigen binding. In some instances, without wishing to be bound by any theories, optimal function may be achieved by maintaining a set distance (e.g., 14 nm) upon CAR:target/antigen binding.

### Extracellular Binding Domain

The present disclosure provides CARs comprising an extracellular binding domain that comprises an antibody, or antigen-binding fragment thereof, that binds to a cotinine moiety. As used herein, the term “anti-cotinine antibody or antigen-binding fragment thereof” refers to an antibody, or antigen binding fragment thereof that binds to a cotinine moiety. Cotinine has the following structure:

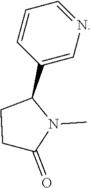

As used herein, the term “cotinine moiety” refers to cotinine or an analog of cotinine. Compounds of Formula (I) described herein comprise a cotinine moiety linked via a linker to a target-binding moiety, such as a PSMA-binding moiety, a CCR2-binding moiety, or a CCR8-binding moiety. In one embodiment, the cotinine moiety has the following structure:

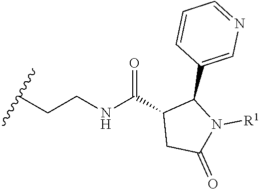

wherein R¹ is C₁₋₄ alkyl or C₃₋₆ cycloalkyl. In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

The term “antibody” is used herein in the broadest sense to refer to molecules with an immunoglobulin-like domain (for example IgG, IgM, IgA, IgD or IgE) and includes monoclonal, recombinant, polyclonal, chimeric, human, humanised, multispecific antibodies, including bispecific antibodies, and heteroconjugate antibodies; a single variable domain (e.g., a domain antibody (DAB)), antigen binding antibody fragments, Fab, F(ab′)2, Fv, disulphide linked Fv, single chain Fv, disulphide-linked scFv, diabodies, TANDABS, etc. and modified versions of any of the foregoing (for a summary of alternative “antibody” formats see Holliger and Hudson, Nature Biotechnology, 2005, 23(9): 1126-1136).

The term, full, whole or Intact antibody, used interchangeably herein, refers to a heterotetrameric glycoprotein with an approximate molecular weight of 150,000 daltons. An intact antibody is composed of two identical heavy chains (HCs) and two identical light chains (LCs) linked by covalent disulphide bonds. This H2L2 structure folds to form three functional domains comprising two antigen-binding fragments, known as ‘Fab’ fragments, and a ‘Fc’ crystallisable fragment. The Fab fragment is composed of the variable domain at the amino-terminus, variable heavy (VH) or variable light (VL), and the constant domain at the carboxyl terminus, CH1 (heavy) and CL (light). The Fc fragment is composed of two domains formed by dimerization of paired CH2 and CH3 regions. The Fc may elicit effector functions by binding to receptors on immune cells or by binding C1q, the first component of the classical complement pathway. The five classes of antibodies IgM, IgA, IgG, IgE and IgD are defined by distinct heavy chain amino acid sequences, which are called μ, α, γ, ε and δ respectively, each heavy chain can pair with either a K or λ light chain. The majority of antibodies in the serum belong to the IgG class, there are four isotypes of human IgG (IgG1, IgG2, IgG3 and IgG4), the sequences of which differ mainly in their hinge region.

“CDRs” are defined as the complementarity determining region amino acid sequences of an antibody or antigen binding fragment thereof. These are the hypervariable regions of immunoglobulin heavy and light chains. There are three heavy chain and three light chain CDRs (or CDR regions) in the variable portion of an immunoglobulin. Thus, “CDRs” as used herein refers to all three heavy chain CDRs, all three light chain CDRs, all heavy and light chain CDRs, or at least two CDRs.

Throughout this specification, amino acid residues in variable domain sequences and variable domain regions within full-length antigen binding sequences, e.g. within an antibody heavy chain sequence or antibody light chain sequence, are numbered according to the Kabat numbering convention. Similarly, the terms “CDR”, “CDRL1”, “CDRL2”, “CDRL3”, “CDRH1”, “CDRH2”, “CDRH3” used in the Examples follow the Kabat numbering convention. For further information, see Kabat et al., Sequences of Proteins of Immunological Interest, 4^{th} Ed., U.S. Department of Health and Human Services, National Institutes of Health (1987).

It will be apparent to those skilled in the art that there are alternative numbering conventions for amino acid residues in variable domain sequences and full-length antibody sequences. There are also alternative numbering conventions for CDR sequences, for example those set out in Chothia et al., Nature, 1989, 342: 877-883. The structure and protein folding of the antigen binding protein may mean that other residues are considered part of the CDR sequence and would be understood to be so by a skilled person.

Other numbering conventions for CDR sequences available to a skilled person include “AbM” (University of Bath) and “contact” (University College London) methods.

Table 1 below represents one definition using each numbering convention for each CDR or binding unit. It should be noted that some of the CDR definitions may vary depending on the individual publication used.

TABLE 1

| | Kabat CDR | Chothia CDR | AbM CDR | Contact CDR |
|---|---|---|---|---|
| H1 | 31-35/35A/35B | 26-32/33/34 | 26-35/35A/35B | 30-35/35A/35B |
| H2 | 50-65 | 52-56 | 50-58 | 47-58 |
| H3 | 95-102 | 95-102 | 95-102 | 93-101 |
| L1 | 24-34 | 24-34 | 24-34 | 30-36 |
| L2 | 50-56 | 50-56 | 50-56 | 46-55 |
| L3 | 89-97 | 89-97 | 89-97 | 89-96 |

In a further embodiment, the anti-cotinine antibody or antigen binding fragment thereof is humanized.

In a further embodiment, the anti-cotinine antibody, or antigen binding fragment thereof, comprises a heavy chain variable region CDR1 having SEQ ID NO: 1, a heavy chain variable region CDR2 having SEQ ID NO: 2, a heavy chain variable region CDR3 having SEQ ID NO: 3, a light chain variable region CDR1 having SEQ ID NO: 4, a light chain variable region CDR2 having SEQ ID NO: 5, and a light chain variable region CDR3 having SEQ ID NO: 6. In a further embodiment, the anti-cotinine antibody has a heavy chain and a light chain, the heavy chain comprising a CDR1 having SEQ ID NO: 1, a CDR2 having SEQ ID NO: 2, and a CDR3 having SEQ ID NO: 3, and the light chain comprising a CDR1 having SEQ ID NO: 4, a CDR2 having SEQ ID NO: 5, and a CDR3 having SEQ ID NO: 6.

In a further embodiment, the anti-cotinine antibody, or antigen binding fragment thereof, comprises a heavy chain variable region (VH) having SEQ ID NO: 7, a light chain variable region (VL) having SEQ ID NO: 8.

In another embodiment, the anti-cotinine antibody or antigen-binding fragment thereof is a single chain variable fragment (scFv). In another embodiment, the scFv comprises a heavy chain variable region CDR1 having SEQ ID NO: 1, a heavy chain variable region CDR2 having SEQ ID NO: 2, a heavy chain variable region CDR3 having SEQ ID NO: 3, a light chain variable region CDR1 having SEQ ID NO: 4, a light chain variable region CDR2 having SEQ ID NO: 5, and a light chain variable region CDR3 having SEQ ID NO: 6. In another embodiment, the scFv comprises a heavy chain variable region (VH) and a light chain variable region (VL). The VH and VL can be directly fused to each other, e.g., via a peptide bond, or the VH and the VL can be joined by a linker, e.g., via a peptide linker. In some embodiments, the VL is located at the N-terminus of the VH. In other embodiments, the VH is located at the N-terminus of the VL. In another embodiment, the VL is located at the N-terminus of the VH, and the VL and VH are joined by peptide linker. In another embodiment, the VH comprises the sequence set forth in SEQ ID NO: 7 and the VL comprises the sequence set forth in SEQ ID NO: 8. In another embodiment, the scFv comprises the sequence set forth in SEQ ID NO: 15. In another embodiment, the scFv comprises the sequence set forth in SEQ ID NO: 16. In another embodiment, the scFv comprises the sequence set forth in SEQ ID NO: 17. In another embodiment, the scFv comprises the sequence set forth in SEQ ID NO: 18.

### Polypeptide Linkers

In certain embodiments, the CARs comprise linker residues between the various domains, e.g., between VH and VL domains, added for appropriate spacing and conformation of the molecule. In particular embodiments the linker is a variable region linking sequence. A “variable region linking sequence” is an amino acid sequence that connects the VH and VL domains and provides a spacer function compatible with interaction of the two sub-binding domains so that the resulting polypeptide retains a specific binding affinity to the same target molecule as an antibody that comprises the same light and heavy chain variable regions. In particular embodiments, a linker separates one or more heavy or light chain variable domains, hinge domains, transmembrane domains, co-stimulatory domains and/or intracellular signalling domains. CARs can comprise one, two, three, four, five or more linkers. In particular embodiments, the length of a linker is about 1 to about 25 amino acids, about 5 to about 20 amino acids, about 10 to about 20 amino acids or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25 or more amino acids long.

Illustrative examples of linkers include glycine polymers (G)ₙ; glycine-serine polymers (G₁₋₅S₁₋₅)ₙ, where n is an integer of at least one, two, three, four or five; glycine-alanine polymers; alanine-serine polymers; and other flexible linkers known in the art. In some embodiments, the linker is a glycine-serine polymer (G₁₋₅S₁₋₅)ₙ, wherein n is an integer of at least one, two, three, four, or five. In other embodiments, the linker is a glycine-serine polymer (GGGGS)ₙ, wherein n is an integer of at least one, two, three, four, or five. An exemplary linker is a glycine-serine polymer as shown in SEQ ID NO: 21 or SEQ ID NO: 22. Another exemplary linker is the Whitlow linker as shown in SEQ ID NO: 29.

In some embodiments, the anti-cotinine antibody or antigen-binding fragment thereof is a single chain variable fragment (scFv) comprising a heavy chain variable region (VH) and a light chain variable region (VL), wherein the VH and VL are joined by a linker comprising (GGGGS)ₙ, wherein n is an integer of at least one, two, three, four, or five. In another embodiment, the VH comprises the sequence set forth in SEQ ID NO: 7 and the VL comprises the sequence set forth in SEQ ID NO: 8, wherein the VH and VL are joined by a linker comprising (GGGGS)ₙ, wherein n is an integer of at least one, two, three, four, or five.

### Spacer Domain

In particular embodiments, the extracellular domain, i.e., binding domain of the CAR is followed by one or more “spacer domains” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 domains of IgG1, IgG4 or IgD.

### Hinge Domain

The binding domain of a CAR is generally followed by one or more “hinge domains,” which plays a role in positioning the antigen binding domain away from the effector cell surface to enable proper cell/cell contact, antigen binding and activation. A CAR generally comprises one or more hinge domains between the binding domain and the transmembrane domain 1. The hinge domain may be derived either from a natural, synthetic, semi-synthetic or recombinant source. The hinge domain can include the amino acid sequence of a naturally occurring immunoglobulin hinge region or an altered immunoglobulin hinge region.

Illustrative hinge domains suitable for use in the CARs described herein include the hinge region derived from the extracellular regions of type I membrane proteins such as CD8a, and CD4, which may be wild-type hinge regions from these molecules or may be altered. In a particular embodiment, the hinge domain is derived from or comprises a CD8a hinge region. In another embodiment, the hinge domain is a modified CD8a hinge domain comprising SEQ ID NO: 23. In another embodiment, the hinge domain derived from CD8a comprises SEQ ID NO: 30.

### Transmembrane Domain

In particular embodiments, a CAR further comprises a transmembrane domain. The “transmembrane domain” I is the portion of the CAR that fuses the extracellular binding portion and co-stimulatory domain/intracellular signalling domain and anchors the CAR to the plasma membrane of the immune effector cell, e.g., by traversing the cell membrane. The TM domain may be derived either from a natural, synthetic, semi-synthetic or recombinant source. The TM domain may be derived from (e.g., comprise) at least the transmembrane region(s) of alpha or beta chain of the T-cell receptor, CD3δ, CD3ε, CD3γ, CD3ζ, CD4, CD5, CD8α, CD9, CD16, CD22, CD27, CD28, CD33, CD37, CD45, CD64, CD80, CD86, CD134, CD137 (4-1BB), CD152, CD154, CD278 (ICOS) and PD1. In a particular embodiment, the TM domain is synthetic and predominantly comprises hydrophobic residues such as leucine and valine.

In one embodiment, the CARs comprise a TM domain derived from CD8α. In another embodiment, a CAR comprises a TM domain derived from CD8a and a short oligo- or polypeptide linker, preferably between 1, 2, 3, 4, 5, 6, 7, 8, 9 or 10 amino acids in length that links the TM domain and the co-stimulatory/intracellular signalling domain of the CAR. A glycine-serine based linker provides a particularly suitable linker. An exemplary TM domain derived from CD8a is shown in SEQ ID NO: 31.

In one embodiment, the CARs comprise a TM domain derived from CD28. In another embodiment, a CAR comprises a TM domain derived from CD28 and a short oligo- or polypeptide linker, preferably between 1, 2, 3, 4, 5, 6, 7, 8, 9 or 10 amino acids in length that links the TM domain and the co-stimulatory/intracellular signalling domain of the CAR. A glycine-serine based linker provides a particularly suitable linker. An exemplary TM domain derived from CD28 is shown in SEQ ID NO: 24.

### Intracellular Signalling Domains

In particular embodiments, a CAR further comprises an intracellular signalling domain. An “intracellular signalling domain” (also referred to as “intracellular effector domain” or “signalling domain”) refers to the part of a CAR that participates in transducing the message of effective binding of the extracellular domain (e.g., anti-cotinine CAR binding) to a target antigen (e.g., cotinine or a derivative thereof) into the interior of the immune effector cell to elicit effector cell function. The intracellular signalling domain is responsible for the activation of at least one of the normal effector functions of the immune cell in which the CAR is expressed, e.g., activation, cytokine production, proliferation and/or cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or helper activity including the secretion of a cytokine. Thus, the term “intracellular signalling domain” refers to the portion of a protein which transduces the effector function signal and that directs the cell to perform a specialized function. While usually the entire intracellular signalling domain can be employed, in many cases it is not necessary to use the entire domain. To the extent that a truncated portion of an intracellular signalling domain is used, such truncated portion may be used in place of the entire domain as long as it transduces the effector function signal.

The term intracellular signalling domain is meant to include any truncated portion of the intracellular signalling domain sufficient to transduce effector function signal.

It is known that signals generated through the T cell receptor (TCR) alone are insufficient for full activation of the T cell and that a secondary or co-stimulatory signal is also required. Thus, T cell activation can be said to be mediated by two distinct classes of signalling domains: intracellular signalling domains that initiate antigen-dependent primary activation through the TCR (e.g., a TCR/CD3 complex) and co-stimulatory signalling domains that act in an antigen-independent manner to provide a secondary or co-stimulatory signal. In some embodiments, a CAR comprises at least one “co-stimulatory signalling domain” and at least one “intracellular signalling domain.”

Intracellular signalling domains regulate primary activation of the TCR complex either in a stimulatory way or in an inhibitory way. Intracellular signalling domains that act in a stimulatory manner may contain signalling motifs which are known as immunoreceptor tyrosine-based activation motifs or ITAMs.

Illustrative examples of ITAM containing intracellular signalling domains that are suitable for use in particular embodiments of CARs described herein include those derived from FcRγ, FcRβ, CD3γ, CD3ε, CD3δ, CD3ζ, CD22, CD66d, CD79a, and CD79b. In one embodiment, the one or more intracellular signalling domain is CD3ζ. An exemplary CD3ζ intracellular signalling domain is shown in SEQ ID NO: 25. In particular preferred embodiments, a CAR comprises a CD3ζ intracellular signalling domain and one or more co-stimulatory signalling domains. The intracellular signalling and co-stimulatory signalling domains may be linked in any order in tandem to the carboxyl terminus of the transmembrane domain.

### Co-Stimulatory Domains

In particular embodiments, a CAR further comprises one or more co-stimulatory signalling domains to enhance the efficacy and expansion of T cells expressing CARs. As used herein, the term “co-stimulatory signalling domain” or “co-stimulatory domain” refers to an intracellular signalling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include CARD11, CD2, CD7, CD27, CD28, CD30, CD40, CD54 (ICAM), CD83, CD134 (OX40), CD137 (4-1EE), CD278 (ICOS), DAP10, LAT, NKD2C, SLP76, TLR1, TLR2, TLR3, TLR4, TLR5, TLR6, TLR7, TLR8, TLR9, TLR10, TRIM and ZAP70.

In one embodiment, a CAR comprises one or more co-stimulatory signalling domains selected from the group consisting of CD28, CD134 (OX40) and CD137 (4-1EE). In a further embodiment, the one or more co-stimulatory domain is CD137 (4-1EE). In another embodiment, a CAR comprises a CD137 (4-1BE) co-stimulatory signalling domain and a CD3ζ intracellular signalling domain. In some embodiments, a CAR comprises a CD137 (4-1EE) co-stimulatory signalling domain and a CD3ζ intracellular signalling domain, wherein the CD3ζ intracellular signalling domain comprises SEQ ID NO: 25. In some embodiments, a CAR comprises a CD137 (4-1EE) co-stimulatory signalling domain and a CD3ζ intracellular signalling domain comprising SEQ ID NO: 32.

In certain embodiments, a CAR further comprises a leader sequence. In particular embodiments, the leader sequence is a CD8 leader sequence. An exemplary CD8 leader sequence is set forth in SEQ ID NO: 20.

In a further embodiment, the CAR comprises the sequence of SEQ ID NO: 19. In a further embodiment, the CAR comprises the sequence of SEQ ID NO: 34. In a further embodiment, the CAR comprises the sequence of SEQ ID NO: 35. In a further embodiment, the CAR comprises the sequence of SEQ ID NO: 36. In a further embodiment, the CAR comprises the sequence of SEQ ID NO: 37. In a further embodiment, the CAR comprises the sequence of SEQ ID NO: 28. In a further embodiment, the CAR comprises the sequence of SEQ ID NO: 38. In a further embodiment, the CAR comprises the sequence of SEQ ID NO: 39.

### Polypeptide

Various polypeptides are contemplated herein, including, but not limited to, CAR polypeptides and fragments thereof, cells and compositions comprising the same, antibodies and vectors that express polypeptides. In preferred embodiments, a polypeptide comprising one or more CARs is provided. In particular embodiments, the CAR is a cotinine binding CAR.

“Polypeptide,” “polypeptide fragment,” “peptide,” and “protein” are used interchangeably, unless specified to the contrary, and according to conventional meaning, i.e., as a sequence of amino acids. Polypeptides may be synthesized or recombinantly produced. Polypeptides are not limited to a specific length, e.g., they may comprise a full length protein sequence or a fragment of a full length protein, and may include post-translational modifications of the polypeptide, for example, glycosylations, acetylations, phosphorylations and the like, as well as other modifications known in the art, both naturally occurring and non-naturally occurring. In various embodiments, the CAR polypeptides comprise a signal (or leader) sequence at the N-terminal end of the protein, which co-translationally or posttranslationally directs transfer of the protein. Illustrative examples of suitable signal sequences useful in CARs contemplated herein include, but are not limited to the IgG1 heavy chain signal polypeptide, a CD8α signal polypeptide, or a human GM-CSF receptor alpha signal polypeptide. Polypeptides can be prepared using any of a variety of well-known recombinant and/or synthetic techniques. Polypeptides contemplated herein specifically encompass the CARs of the present disclosure, or sequences that have deletions from, additions to, and/or substitutions of one or more amino acids of a CAR as contemplated herein.

An “isolated peptide” or an “isolated polypeptide” and the like, as used herein, refer to in vitro isolation and/or purification of a peptide or polypeptide molecule from a cellular environment, and from association with other components of the cell, i.e., it is not significantly associated with in vivo substances. Similarly, an “isolated cell” refers to a cell that has been obtained from an in vivo tissue or organ and is substantially free of extracellular matrix.

Polypeptides include “polypeptide variants”. Polypeptide variants may differ from a naturally occurring polypeptide in one or more substitutions, deletions, additions and/or insertions. Such variants may be naturally occurring or may be synthetically generated, for example, by modifying one or more of the above polypeptide sequences.

It will be appreciated that polypeptides comprising CARs, such as a CAR comprising the amino acid sequence of SEQ ID NO: 19 as provided herein may comprise further and/or additional polypeptide sequences or elements. Such additional elements include, but are not limited to, ablation or control elements which may be used to either control expression of the polypeptide sequence in a cell or to target a polypeptide-containing cell. Elements or polypeptide sequences which control expression may comprise an internal ribosome entry site (IRES), translation start sequences and/or cleavage sites which allow for the separation of elements of the polypeptide sequence after translation.

Thus, in one embodiment, the polypeptide contemplated herein further comprises an ablation element. As used herein, an “ablation element” refers to a polypeptide sequence and/or protein expressed on the surface of a cell and which may be used to target or detect said cell (also known as “elimination markers”). For example, the ablation element may be a polypeptide sequence of a cell surface protein which comprises an extracellular epitope or binding region for an antibody or antigen binding fragment thereof. Thus, in one embodiment, the ablation element is a cell surface protein which is targeted for antibody-dependent cellular cytotoxicity (ADCC) and/or complement-dependent cytotoxicity (CDC) using an antibody or antigen binding fragment thereof specific for the cell surface protein. By utilising such mechanisms, it will therefore be appreciated that cells expressing the polypeptide contemplated herein may be specifically labelled/detected and may be specifically selected or isolated from, for example, a mixed population of transduced and untransduced cells. Furthermore, cells expressing a polypeptide comprising an ablation element may be specifically and selectively eliminated, such as eliminated/removed from the circulation of a treated subject.

Examples of suitable ablation elements include, but are not limited to, truncated human EGFR polypeptide (huEGFRt) and CD20, which may be recognised by cetuximab and rituximab, respectively (Wang et al., Blood, 2011; 118(5): 1255-1263, Paszkiewicz et al., J Clin Invest, 2016; 126(11):4262-4272, Vogler et al., Mol Ther J Am Soc Gene Ther, 2010; 18:1330-8, Griffioen et al., Haematologica, 2009; 94:1316-20 and Philip et al., Blood, 2014; 124:1277-87). Another example of a suitable ablation element is a short polypeptide epitope tag incorporated into the extracellular domain of the CAR (a so called “E-tag”) to which anti-epitope tag CARs may then be generated (Koristka et al., Cancer Immunol Immunother CII, 2019; 68:1401-15).

Thus, in one embodiment, the ablation element is selected from the group consisting of: truncated human EGFR polypeptide (huEGFRt) and CD20. In a particular embodiment, the ablation element is CD20.

In some embodiments, the ablation element is cleaved from the CAR polypeptide sequence. Thus, in one embodiment, the polypeptide contemplated herein comprises a cleavage site, such as a P2A cleavage site.

In certain embodiments, a polypeptide comprises the sequence set forth in SEQ ID NO: 19. In a further embodiment, a polypeptide comprises the sequence of SEQ ID NO: 34. In a further embodiment, a polypeptide comprises the sequence of SEQ ID NO: 35. In a further embodiment, a polypeptide comprises the sequence of SEQ ID NO: 36. In a further embodiment, a polypeptide comprises the sequence of SEQ ID NO: 37. In a further embodiment, a polypeptide comprises the sequence of SEQ ID NO: 28. In a further embodiment, a polypeptide comprises the sequence of SEQ ID NO: 38. In a further embodiment, a polypeptide comprises the sequence of SEQ ID NO: 39.

### Polynucleotide

In another aspect, a polynucleotide encoding one or more CARs as described herein is provided. As used herein, the terms “polynucleotide” or “nucleic acid” refer to messenger RNA (mRNA), RNA, genomic RNA (gRNA), plus strand RNA (RNA(+)), minus strand RNA (RNA(−)), genomic DNA (gDNA), complementary DNA (cDNA) or recombinant DNA. Polynucleotides include single and double stranded polynucleotides.

In various illustrative embodiments, polynucleotides include expression vectors, viral vectors, and transfer plasmids, and compositions and cells comprising the same. In various illustrative embodiments, polynucleotides encode a CAR or polypeptide contemplated herein, including, but not limited to a CAR having the sequence of SEQ ID NO: 19 or a polynucleotide sequence encoding SEQ ID NO: 19.

As used herein, “isolated polynucleotide” refers to a polynucleotide that has been purified from the sequences which flank it in a naturally-occurring state, e.g., a DNA fragment that has been removed from the sequences that are normally adjacent to the fragment. An “isolated polynucleotide” also refers to a complementary DNA (cDNA), a recombinant DNA, or other polynucleotide that does not exist in nature and that has been made by the hand of man.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

### Vectors

In another aspect, the present invention provides vectors which comprise a polynucleotide encoding one or more CARs and/or polypeptides as described herein.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell or may include sequences sufficient to allow integration into host cell DNA. Useful vectors include, for example, plasmids (e.g., DNA plasmids or RNA plasmids), transposons, cosmids, bacterial artificial chromosomes and viral vectors. Useful viral vectors include, e.g., replication defective retroviruses and lentiviruses.

In particular embodiments, the vectors are expression vectors. Expression vectors may be used to produce CARs and polypeptides contemplated herein. In addition, expression vectors may include additional components which allow for the production of viral vectors, which in turn comprise a polynucleotide contemplated herein. Viral vectors may be used for delivery of the polynucleotides contemplated herein to a subject or a subject's cells. Examples of expression vectors include, but are not limited to, plasmids, autonomously replicating sequences and transposable elements. Additional exemplary vectors include, without limitation, plasmids, phagemids, cosmids, transposons, artificial chromosomes such as yeast artificial chromosome (YAC), bacterial artificial chromosome (BAC), or PI-derived artificial chromosome (PAC), bacteriophages such as lambda phage or MI 3 phage, and animal viruses.

Additional examples of expression vectors are pClneo vectors (Promega) for expression in mammalian cells; pLenti4N5-DEST™ pLenti6N5-DEST™ and pLenti6.2/V5-GW/lacZ (Invitrogen)) for lentivirus-mediated gene transfer and expression in mammalian cells. In particular embodiments, the coding sequences of the CARs and polypeptides disclosed herein can be ligated into such expression vectors for the expression of the CARs and/or polypeptides in mammalian cells.

In particular embodiments, the expression vectors provided herein are BACs which comprise a polynucleotide as described herein. In particular embodiments, the BACs additionally comprise one or more polynucleotides encoding for proteins necessary to allow the production of a viral vector when expressed in a producer or packaging cell line. By way of example, PCT applications WO2017/089307 and WO2017/089308 describe expression vectors used to produce retroviral vectors, in particular lentiviral vectors. In a particular embodiment, the expression vectors described in WO2017/089307 and WO2017/089308, comprising a polynucleotide as described herein are provided.

The “control elements” or “regulatory sequences” present in an expression vector are those non-translated regions of the vector-origin of replication, selection cassettes, promoters, enhancers, translation initiation signals (Shine Dalgarno sequence or Kozak sequence), introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

### Vectors for Delivery

Also provided are vectors for delivery of the polynucleotides described herein to a subject and/or subject's cells. Examples of such vectors include, but are not limited to, plasmids, autonomously replicating sequences, transposable elements, phagemids, cosmids, artificial chromosomes such as yeast artificial chromosome (YAC), bacterial artificial chromosome (BAC), or PI-derived artificial chromosome (PAC), bacteriophages such as lambda phage or MI 3 phage, and viral vectors.

Examples of categories of animal viruses useful as viral vectors include, without limitation, retrovirus (including lentivirus), adenovirus, adeno-associated virus (AAV), herpesvirus (e.g., herpes simplex virus), poxvirus, baculovirus, papillomavirus, and papovavirus (e.g., SV40). These vectors are referred to herein as “viral vectors”.

As the skilled person will appreciate, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Retroviruses are a common tool for gene delivery (Miller, 2000, Nature. 357: 455-460). In particular embodiments, a retrovirus is used to deliver a polynucleotide encoding a CAR as described herein to a cell. As used herein, the term “retrovirus” refers to an RNA virus that reverse transcribes its genomic RNA into a linear double-stranded DNA copy and subsequently covalently integrates its genomic DNA into a host genome. Once the virus is integrated into the host genome, it is referred to as a “provirus”. The provirus serves as a template for RNA polymerase II and directs the expression of RNA molecules which encode the structural proteins and enzymes needed to produce new viral particles.

Illustrative retroviruses suitable for use in particular embodiments, include, but are not limited to: Moloney murine leukaemia virus (M-MuLV), Moloney murine sarcoma virus (MoMSV), Harvey murine sarcoma virus (HaMuSV), murine mammary tumour virus (MuMTV), gibbon ape leukaemia virus (GaLV), feline leukaemia virus (FLV), spumavirus, Friend murine leukaemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV); the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

Retroviral vectors and more particularly lentiviral vectors may be used in practicing particular embodiments. Accordingly, the term “retrovirus” or “retroviral vector” as used herein is meant to include “lentivirus” and “lentiviral vectors” respectively.

Viral particles will typically include various viral components and sometimes also host cell components in addition to nucleic acid(s).

The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus. The term “hybrid vector” refers to a vector, LTR or other nucleic acid containing both retroviral, e.g., lentiviral, sequences and non-lentiviral viral sequences. In one embodiment, a hybrid vector refers to a vector or transfer plasmid comprising retroviral e.g., lentiviral, sequences for reverse transcription, replication, integration and/or packaging.

In particular embodiments, the terms “lentiviral vector” and “lentiviral expression vector” may be used to refer to lentiviral transfer plasmids and/or infectious lentiviral particles. Where reference is made herein to elements such as cloning sites, promoters, regulatory elements, heterologous nucleic acids, etc., it is to be understood that the sequences of these elements are present in RNA form in the lentiviral particles and are present in DNA form in the DNA plasmids.

At each end of the provirus are structures called “long terminal repeats” or “LTRs”. The term “long terminal repeat (LTR)” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals and sequences needed for replication and integration of the viral genome. The viral LTR is divided into three regions called U3, R and U5. The U3 region contains the enhancer and promoter elements. The U5 region is the sequence between the primer binding site and the R region and contains the polyadenylation sequence. The R (repeat) region is flanked by the U3 and U5 regions. The LTR comprises U3, R, and U5 regions and appears at both the 5′ and 3′ ends of the viral genome. Adjacent to the 5′ LTR are sequences necessary for reverse transcription of the genome (the tRNA primer binding site) and for efficient packaging of viral RNA into particles (the Psi site).

As used herein, the term “packaging signal” or “packaging sequence” refers to sequences located within the retroviral genome which are required for insertion of the viral RNA into the viral capsid or particle, see e.g., Clever et al., J Virol. 1995; 69(4): 2101-9. Several retroviral vectors use the minimal packaging signal (also referred to as the psi [W] sequence) needed for encapsidation of the viral genome. Thus, as used herein, the terms “packaging sequence”, “packaging signal”, “psi” and the symbol “W” are used in reference to the non-coding sequence required for encapsidation of retroviral RNA strands during viral particle formation.

In various embodiments, vectors comprise modified 5′ LTR and/or 3′ LTRs. Either or both of the LTRs may comprise one or more modifications including, but not limited to, one or more deletions, insertions or substitutions. Modifications of the 3′ LTR are often made to improve the safety of lentiviral or retroviral systems by rendering viruses replication defective.

As used herein, the term “replication-defective” refers to virus that is not capable of complete, effective replication such that infective virions are not produced (e.g., replication-defective lentiviral progeny). The term “replication-competent” refers to wildtype virus or mutant virus that is capable of replication, such that viral replication of the virus is capable of producing infective virions (e.g., replication-competent lentiviral progeny).

“Self-inactivating” (SIN) vectors refers to replication-defective vectors, e.g., retroviral or lentiviral vectors, in which the right (3′) LTR enhancer-promoter region, known as the U3 region, has been modified (e.g., by deletion or substitution) to prevent viral transcription beyond the first round of viral replication. This is because the right (3′) LTR U3 region is used as a template for the left (5′) LTR U3 region during viral replication and, thus, the viral transcript cannot be made without the U3 enhancer-promoter. In a further embodiment, the 3′ LTR is modified such that the U5 region is replaced, for example, with an ideal poly(A) sequence. It should be noted that modifications to the LTRs such as modifications to the 3′ LTR, the 5′ LTR, or both 3′ and 5′ LTRs, are also included.

An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukaemia virus (MoMLV), Rous sarcoma virus (RSV) and herpes simplex virus (HSV) (thymidine kinase) promoters. Typical promoters are able to drive high levels of transcription in a Tat-independent manner. This replacement reduces the possibility of recombination to generate replication-competent virus because there is no complete U3 sequence in the virus production system. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

According to certain embodiments, most or all of the viral vector backbone sequences are derived from a lentivirus, e.g., HIV-l. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (Science. 1996; 272(5259): 263-7; Proc Natl Acad Sci USA. 1996; 93(21): 11382-8; Curr Opin Biotechnol. 1998; 9(5): 457-63); Zufferey al., Nat Biotechnol. 1997; 15(9): 871-5; Dull et al., J Virol. 1998; 72(11): 8463-71; U.S. Pat. Nos. 6,013,516; and 5,994,136, many of which may be adapted to produce a viral vector or transfer plasmid.

In various embodiments, vectors comprise a promoter operably linked to a polynucleotide encoding a CAR or polypeptide as described herein. In particular embodiments, the vector is a non-integrating vector, including but not limited to, an episomal vector or a vector that is maintained extrachromosomally. As used herein, the term “episomal” refers to a vector that is able to replicate without integration into chromosomal DNA of a host and without gradual loss from a dividing host cell also meaning that said vector replicates extrachromosomally or episomally.

In some embodiments, a vector described herein is a viral vector.

In some embodiments, a viral vector described herein is a retroviral vector.

In some embodiments, a retroviral vector described herein is a lentiviral vector.

In some embodiments, a retroviral vector as described herein is selected from the group consisting of: human immunodeficiency virus I (HIV-1); human immunodeficiency virus 2 (HIV-2), visna-maedi virus (VMV) virus; caprine arthritis-encephalitis virus (CAEV); equine infectious anemia virus (EIAV); feline immunodeficiency virus (FIV); bovine immune deficiency virus (BIV); and simian immunodeficiency virus.

### Control Elements

In particular embodiments, vectors, which include but are not limited to expression vectors and viral vectors, will include exogenous, endogenous or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked” refers to a juxtaposition wherein the components described are in a relationship permitting them to function in their intended manner. In one embodiment, the term refers to a functional linkage between a nucleic acid expression control sequence (such as a promoter and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer or promoter/enhancer that continually or continuously allows for transcription of an operably linked sequence. A constitutive expression control sequence may be a “ubiquitous” promoter, enhancer or promoter/enhancer that allows expression in a wide variety of cell and tissue types or a “cell-specific”, “cell type-specific”, “cell lineage-specific” or “tissue-specific” promoter, enhancer or promoter/enhancer that allows expression in a restricted variety of cell and tissue types, respectively. Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukaemia virus (MoN4LV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), beta-kinesin (betaKIN), the human ROSA 26 locus (Irions et al., Nature Biotechnology 25, 1477-1482 (2007)), a Ubiquitin C promoter (UBC), a phosphoglycerate kinase-1 (PGK) promoter, a cytomegalovirus enhancer/chicken beta-actin (CAG) promoter, a beta-actin promoter and a myeloproliferative sarcoma virus enhancer, negative control region deleted, d1587rev primer binding site substituted (MND) promoter (Challita et al., J Virol. 69(2), 748-55 (1995)).

In one embodiment, a vector comprises a CMV promoter.

### Vector Production

In particular embodiments, a cell (e.g., an immune effector cell) is transduced with a retroviral vector, e.g., a lentiviral vector, encoding a CAR. For example, an immune effector cell is transduced with a vector encoding a CAR as described herein. These transduced cells can elicit a CAR-mediated cytotoxic response. A “host cell” includes cells electroporated, transfected, infected or transduced in vivo, ex vivo or in vitro with a vector or a polynucleotide. Host cells may include packaging cells, producer cells and cells transduced with viral vectors.

In particular embodiments, host cells transduced with viral vectors are administered to a subject in need of therapy. In certain embodiments, the term “target cell” is used interchangeably with host cell and refers to transfected, infected or transduced cells of a desired cell type. In one embodiment, the target cell is a T cell. In another embodiment, the target cell is an NK cell.

Large scale viral vector production is often necessary to achieve a suitable viral titre. Viral particles may be produced by transfecting a transfer vector into a packaging cell line that comprises viral structural and/or accessory genes, e.g., gag, POI, env, tat, rev, vif, vpr, vpu, vpx, or nef genes or other retroviral genes.

As used herein, the term “packaging vector” refers to an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. Methods for transfection, transduction or infection are well known by those of skill in the art. In particular embodiments, a retroviral/lentiviral transfer vector is introduced into a packaging cell line, via transfection, transduction or infection, to generate a producer cell or cell line. In particular embodiments, packaging vectors are introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

As used herein, the term “packaging cell lines” is used in reference to cell lines that do not contain a packaging signal but do stably or transiently express viral structural proteins and replication enzymes (e.g., gag, pol and env) which are necessary for the correct packaging of viral particles. Any suitable cell line can be employed to prepare packaging cells. Generally, the cells are mammalian cells. In a particular embodiment, the cells used to produce the packaging cell line are human cells. Suitable cell lines which can be used include, for example, CHO cells, BHK cells, NOCK cells, C3H IOT1/2 cells, FLY cells, Psi2 cells, BOSC 23 cells, PA317 cells, WEHI cells, COS cells, BSC 1 cells, BSC 40 cells, BMT 10 cells, VERO cells, W 138 cells, MRC5 cells, A549 cells, HT1080 cells, HEK293 cells, HEK293T cells, B-50 cells, 3T3 cells, NIH3T3 cells, HepG2 cells, Saos-2 cells, Huh7 cells, HeLa cells, W 163 cells, 211 cells and 21 IA cells.

As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Producer cell line includes those cell lines described in, e.g., WO2017/089307 and WO2017/089308, which comprise all of the elements which are necessary for the production of a retroviral vector, in a single locus in the host cell genome.

Methods of preparing viral stock solutions are known in the art and are illustrated by, e.g., Y. Soneoka et al., (1995) Nucl. Acids Res. 23:628-633 and N. R. Landau et al., (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Viral envelope proteins (env) determine the range of host cells which can ultimately be infected and transformed by recombinant retroviruses generated from the cell lines. In the case of lentiviruses, such as HIV-1, HIV-2, SIV, FIV and EIV, the env proteins include gp41 and gp120.

The terms “pseudotype” or “pseudotyping” as used herein, refer to a virus whose viral envelope proteins have been substituted with those of another virus possessing preferable characteristics. For example, HIV can be pseudotyped with vesicular stomatitis virus G protein (VSV-G) envelope proteins, which allows HIV to infect a wider range of cells because HIV envelope proteins (encoded by the env gene) normally target the virus to CD4+ presenting cells. In a preferred embodiment, lentiviral envelope proteins are pseudotyped with VSV-G. In one embodiment, packaging cells produce a recombinant retrovirus, e.g., lentivirus, pseudotyped with the VSV-G envelope glycoprotein.

In other embodiments, viral vectors may be pseudotyped with an envelope protein from either another retrovirus or an unrelated virus. The skilled person will appreciate that the viral vectors described herein may be pseudotyped with any suitable envelope protein.

The delivery of a gene(s) or other polynucleotide sequence using a retroviral or lentiviral vector by means of viral infection rather than by transfection is referred to as “transduction”. In one embodiment, retroviral vectors are transduced into a cell through infection and provirus integration. In certain embodiments, a target cell, e.g., a T cell or NK cell, is “transduced” if it comprises a gene or other polynucleotide sequence delivered to the cell by infection using a viral or retroviral vector. In particular embodiments, a transduced cell comprises one or more genes or other polynucleotide sequences delivered by a retroviral or lentiviral vector in its cellular genome.

### Immune Effector Cell

In another aspect, provided is an immune effector cell (also referred to as an immunomodulatory cell) comprising a CAR, polypeptide, polynucleotide, and/or vector as described herein. In various embodiments, cells genetically modified to express the CARs contemplated herein, for use in the treatment of cancer are provided. As used herein, the term “genetically engineered” or “genetically modified” refers to the addition of extra genetic material in the form of DNA or RNA into the total genetic material in a cell. The terms “genetically modified cells”, “modified cells” and “redirected cells” are used interchangeably. As used herein, the term “gene therapy” refers to the introduction of extra genetic material in the form of DNA or RNA into the total genetic material in a cell that restores, corrects, or modifies expression of a gene, or for the purpose of expressing a therapeutic polypeptide, e.g., a CAR.

In particular embodiments, the CARs contemplated herein are introduced and expressed in immune effector cells so as to redirect specificity of the immune effector cell to a target antigen of interest, e.g., cotinine or a derivative thereof.

An “immune effector cell” is any cell of the immune system that has one or more effector functions (e.g., cytotoxic cell killing activity, secretion of cytokines, induction of ADCC and/or CDC). The illustrative immune effector cells contemplated herein are T lymphocytes, in particular cytotoxic T cells (CTLs; CD8+ T cells), tumour infiltrating lymphocytes (TILs) and helper T cells (HTLs; CD4+ T cells). In one embodiment, immune effector cells include natural killer (NK) cells. In one embodiment, immune effector cells include natural killer T cells. In another embodiment, immune effector cells include macrophages. Immune effector cells can be autologous/autogeneic (“self” or non-autologous (“nonself”), e.g., allogeneic, syngeneic or xenogeneic).

“Autologous” as used herein, refers to cells from the same subject.

“Allogeneic” as used herein, refers to cells of the same species that differ genetically to the cell in comparison.

“Syngeneic” as used herein, refers to cells of a different subject that are genetically identical to the cell in comparison.

“Xenogeneic” as used herein, refers to cells of a different species to the cell in comparison. In preferred embodiments, the cells, e.g., immune effector cells, are allogeneic.

Illustrative immune effector cells used with the CARs contemplated herein include T lymphocytes. The terms “T cell” or “T lymphocyte” are art-recognized and are intended to include thymocytes, immature T lymphocytes, mature T lymphocytes, resting T lymphocytes or activated T lymphocytes. A T cell can be a T helper (Th) cell, for example a T helper I (Th1) or a T helper 2 (Th2) cell. The T cell can be a helper T cell (HTL; CD4+ T cell), a cytotoxic T cell (CTL; CD8+ T cell), CD4+ CD8+ T cell, CD4− CD8− T cell or any other subset of T cells. Other illustrative populations of T cells suitable for use in particular embodiments include naive T cells and memory T cells.

In some embodiments, the immune effector cell is selected from the group consisting of: a T lymphocyte, a natural killer T lymphocyte (NKT) cell, a macrophage, and a natural killer (NK) cell.

In one embodiment, the immune effector cell is a cytotoxic T lymphocyte (CD8+).

In one embodiment, the immune effector cell is a natural killer (NK) cell.

As would be understood by the skilled person, other cells may also be used as immune effector cells with the CARs as described herein. In particular, immune effector cells also include NK cells, NKT cells, neutrophils and macrophages. Immune effector cells also include progenitors of effector cells wherein such progenitor cells can be induced to differentiate into an immune effector cell in vivo or in vitro. Thus, in particular embodiments, immune effector cell includes progenitors of immune effectors cells such as hematopoietic stem cells (HSCs) contained within the CD34 population of cells derived from cord blood, bone marrow or mobilized peripheral blood which upon administration in a subject differentiate into mature immune effector cells, or which can be induced in vitro to differentiate into mature immune effector cells.

As used herein, immune effector cells genetically engineered to contain, e.g., a cotinine specific CAR may be referred to as “antigen-specific redirected immune effector cells” or “AG-specific redirected immune effector cells”.

Methods for making or generating the immune effector cells which express the CARs described herein are provided in particular embodiments. In various embodiments, such methods comprise introducing into an immune effector cell a polynucleotide and/or vector as described herein. In one embodiment, the method comprises transfecting or transducing immune effector cells isolated from an individual such that the immune effector cells express one or more CARs contemplated herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before and/or after being genetically modified (i.e., transduced or transfected to express a CAR contemplated herein). Thus, in certain embodiments, the immune effector cells may be stimulated and induced to proliferate by contacting the cell with antibodies or antigen binding fragments that bind CD3 and/or antibodies or antigen binding fragments that bind to CD28; thereby generating a population of immune effector cells. In further embodiments, the method of generating immune effector cells contemplated herein comprises stimulating the immune effector cell and inducing the cell to proliferate by contacting the cell with antibodies or antigen binding fragments that bind CD3 and antibodies or antigen binding fragments that bind to CD28; thereby generating a population of immune effector cells.

In particular embodiments, prior to in vitro manipulation or genetic modification of the immune effector cells described herein, the immune effector cells are obtained from a subject. In particular embodiments, the CAR-modified immune effector cells comprise T cells.

In particular embodiments, peripheral blood mononuclear cells (PBMCs) may be directly genetically modified to express CARs using methods contemplated herein. In certain embodiments, after isolation of PBMCs, T lymphocytes are further isolated and in certain embodiments, both cytotoxic and helper T lymphocytes can be sorted into naive, memory and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the CARs contemplated herein (e.g., transduced with a viral vector comprising a nucleic acid encoding a CAR) and then are activated and expanded in vitro. In various embodiments, T cells can be activated and expanded before or after genetic modification to express a CAR.

In particular embodiments, a population of modified immune effector cells for the treatment of cancer comprises a CAR as disclosed herein. For example, a population of modified immune effector cells are prepared from peripheral blood mononuclear cells (PBMCs) obtained from a patient diagnosed with cancer (autologous donors). The PBMCs form a heterogeneous population of T lymphocytes that can be CD4+, CD8+, or CD4+ and CD8+.

The PBMCs also can include other cytotoxic lymphocytes such as NK cells or NKT cells. A vector carrying the coding sequence of a CAR described herein can be introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR protein expressing T cells in addition to cell activation using anti-CD3 antibodies and or anti-CD28 antibodies and IL-2 or any other methods known in the art as described elsewhere herein.

Standard procedures are used for cryopreservation of T cells expressing the CAR protein for storage and/or preparation for use in a human subject.

In a further embodiment, a mixture of, e.g., one, two, three, four, five or more, different vectors can be used in genetically modifying a donor population of immune effector cells wherein each vector encodes a different chimeric antigen receptor protein as contemplated herein. The resulting modified immune effector cells forms a mixed population of modified cells, with a proportion of the modified cells expressing more than one different CAR proteins.

In a further embodiment, the disclosure provides a modified immunomodulatory cell, wherein the modified immunomodulatory cell expresses the chimeric antigen receptor as disclosed herein. In a further embodiment, the modified immunomodulatory cell is a T cell. In a further embodiment, the modified immunomodulatory cell is an NK cell.

### Heterobifunctional Molecules

In another embodiment, the disclosure provides a combination comprising a modified immunomodulatory cell as disclosed herein and a heterobivalent molecule comprising cotinine or a derivative thereof covalently linked to a target binding moiety. In another embodiment, the combination comprises a modified immunomodulatory cell as disclosed herein and a heterobifunctional molecule comprising a moiety that binds a target cell-surface protein covalently linked to a cotinine moiety or a cotinine derivative moiety.

In another embodiment, the heterobifunctional molecule is a compound of Formula (I):

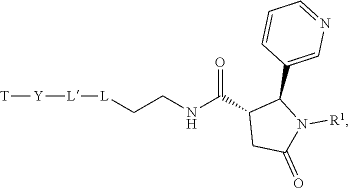

or a pharmaceutically acceptable salt thereof,
wherein:
T is a target binding moiety;
R¹ is C₁₋₄ alkyl or C₃₋₆ cycloalkyl;
L′ is a bond

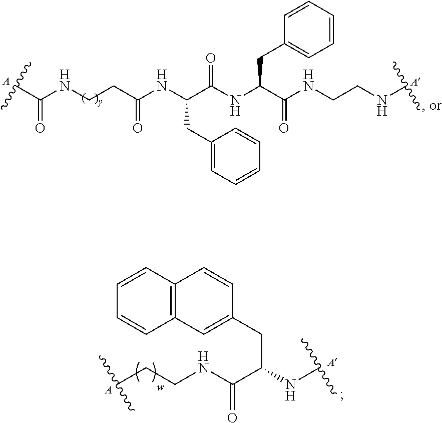

y is an integer of 1 to 9;
w is an integer of 0 to 5;
Y is a bond or a divalent spacer moiety of one to twelve atoms in length; and
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), (L-p), (L-q), (L-r), or (L-s);
wherein each

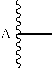

represents a covalent bond to the Y group of Formula (I), or when Y is a bond, a covalent bond to the T group of Formula (I), and each

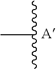

represents a covalent bond to the L group of Formula (I).

In another embodiment, L is a divalent linker of Formula (L-a):

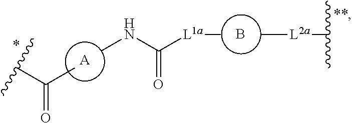

or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

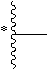

represents a covalent bond to the L′ group of Formula (I), or when L′ is a bond, a covalent bond to the Y group of Formula (I), or when both L′ and Y are a bond, a covalent bond to the T group of Formula (I), and

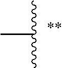

represents a covalent bond to the methylene group of Formula (I).

In another embodiment, Ring A and Ring B of Formula (L-a) are each independently

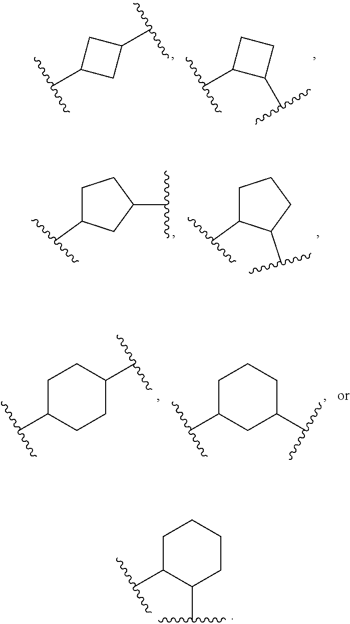

In another embodiment, L is a divalent linker of Formula (L-a-i):

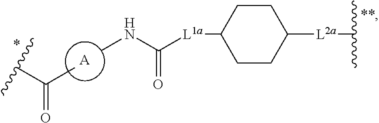

or a stereoisomer thereof,
wherein:
Ring A is C₄₋₆ cycloalkylene;
L^{1a} is C₃₋₅ linear alkylene, wherein 1 or 2 methylene units are replaced with —O— or —NR^{a}—;
wherein

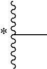

represents a covalent bond to the L′ group of Formula (I), or when L′ is a bond, a covalent bond to the Y group of Formula (I), or when both L′ and Y are a bond, a covalent bond to the T group of Formula (I), and

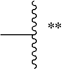

represents a covalent bond to the methylene group of Formula (I).

In another embodiment, Ring A of Formula (L-a-i) is

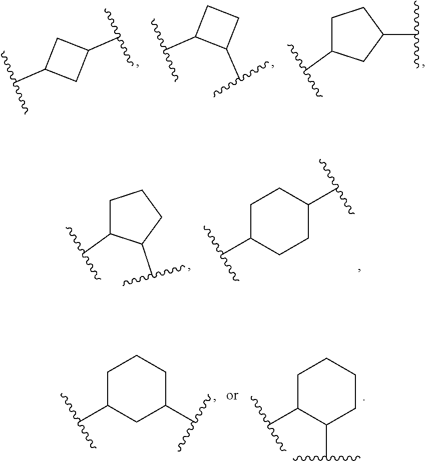

In another embodiment, L is a divalent linker of Formula (L-a-ii):

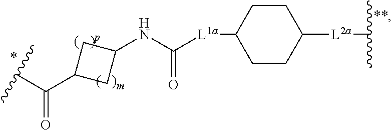

or a stereoisomer thereof,
wherein:
L^{1a} is C₃₋₅ linear alkylene, wherein 1 or 2 methylene units are replaced with —O— or —NR^{a}—;
each R^{a} is independently hydrogen or C₁₋₃ alkyl;
L^{2a} is —O—, —NHC(O)—, or —CH₂—O—;
p is 1 or 2; and
m is 1 or 2;
wherein

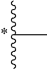

represents a covalent bond to the L′ group of Formula (I), or when L′ is a bond, a covalent bond to the Y group of Formula (I), or when both L′ and Y are a bond, a covalent bond to the T group of Formula (I), and

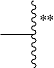

represents a covalent bond to the methylene group of Formula (I).

In another embodiment, L^{1a} of Formula (L-a), (L-a-i), or (L-a-ii) is selected from

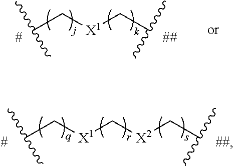

wherein:
j is 1, 2, 3, or 4;
k is 0, 1, 2, or 3;
the sum of j and k is 2, 3, or 4;
q is 1 or 2;
r is 1 or 2;
s is 0 or 1;
the sum of q, r, and s is 2 or 3;
X¹ and X² are independently —O— or NR^{a}; and
each R^{a} is independently hydrogen or C₁₋₃ alkyl;
wherein

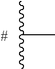

represents a covalent bond to the C(O) group of Formula (L-a), (L-a-i), or (L-a-ii), and

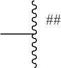

represents a covalent bond to Ring B of Formula (L-a) or to the cyclohexylene group of Formula (L-a-i) or (L-a-ii).

In another embodiment, L^{1a} of Formula (L-a), (L-a-i), or (L-a-ii) is selected from —(CH₂)₂O—, —(CH₂)₃O—, —(CH₂)₄O—, —(CH₂)₂OCH₂—, —(CH₂)₃OCH₂—, —(CH₂)₂O(CH₂)₂—, —CH₂OCH₂—, —CH₂O(CH₂)₂—, —CH₂O(CH₂)₃—, —CH₂OCH₂O—, or —CH₂OCH₂OCH₂—. In another embodiment, L^{1a} of Formula (L-a), (L-a-i), or (L-a-ii) is selected from —(CH₂)₂O—, —(CH₂)₃O—, —(CH₂)₂OCH₂—, or —(CH₂)₃OCH₂—. In another embodiment, L^{1a} of Formula (L-a), (L-a-i), or (L-a-ii) is selected from —(CH₂)₂NR^{a}—, —(CH₂)₃NR^{a}—, —(CH₂)₄NR^{a}—, —(CH₂)₂NR^{a}CH₂—, —(CH₂)₃NR^{a}CH₂—, —(CH₂)₂NR^{a}(CH₂)₂—, —CH₂NR^{a}CH₂—, —CH₂NR^{a}(CH₂)₂—, —CH₂NR^{a}(CH₂)₃—, —CH₂NR^{a}CH₂NR^{a}—, or —CH₂NR^{a}CH₂NR^{a}CH₂—, wherein each R^{a} is independently hydrogen or C₁₋₃ alkyl. In another embodiment, L^{1a} of Formula (L-a), (L-a-i), or (L-a-ii) is selected from —(CH₂)₂NR^{a}—, —(CH₂)₃NR^{a}—, —(CH₂)₂NR^{a}CH₂—, or —(CH₂)₃NR^{a}CH₂—, wherein R^{a} is hydrogen or C₁₋₃ alkyl. In another embodiment, L^{1a} of Formula (L-a), (L-a-i), or (L-a-ii) is selected from —(CH₂)₂NH—, —(CH₂)₃NH—, —(CH₂)₄NH—, —(CH₂)₂NHCH₂—, —(CH₂)₃NHCH₂—, —(CH₂)₂NH(CH₂)₂—, —CH₂NHCH₂—, —CH₂NH(CH₂)₂—, —CH₂NH(CH₂)₃—, —CH₂NHCH₂NH—, or —CH₂NHCH₂NHCH₂—. In another embodiment, L^{1a} of Formula (L-a), (L-a-i), or (L-a-ii) is selected from —(CH₂)₂NH—, —(CH₂)₃NH—, —(CH₂)₂NHCH₂—, or —(CH₂)₃NHCH₂—. In another embodiment, L^{1a} of Formula (L-a), (L-a-i), or (L-a-ii) is selected from —CH₂OCH₂NR^{a}—, —CH₂NR^{a}CH₂O—, —CH₂OCH₂NR^{a}CH₂—, —CH₂NR^{a}CH₂OCH₂—, wherein R^{a} is independently hydrogen or C₁₋₃ alkyl. In another embodiment, L^{1a} of Formula (L-a), (L-a-i), or (L-a-ii) is selected from —CH₂OCH₂NH—, —CH₂NHCH₂O—, —CH₂OCH₂NHCH₂—, —CH₂NHCH₂OCH₂—.

In another embodiment L is a divalent linker of Formula (L-a-iii):

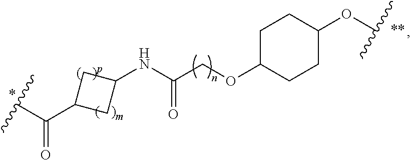

or a stereoisomer thereof,
wherein:
p is 1 or 2;
m is 1 or 2; and
n is 1, 2, or 3;
wherein

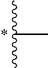

represents a covalent bond to the L′ group of Formula (I), or when L′ is a bond, a covalent bond to the Y group of Formula (I), or when both L′ and Y are a bond, a covalent bond to the T group of Formula (I), and

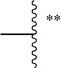

represents a covalent bond to the methylene group of Formula (I).

In another embodiment, L is a divalent linker of Formula (L-a) selected from the group consisting of:

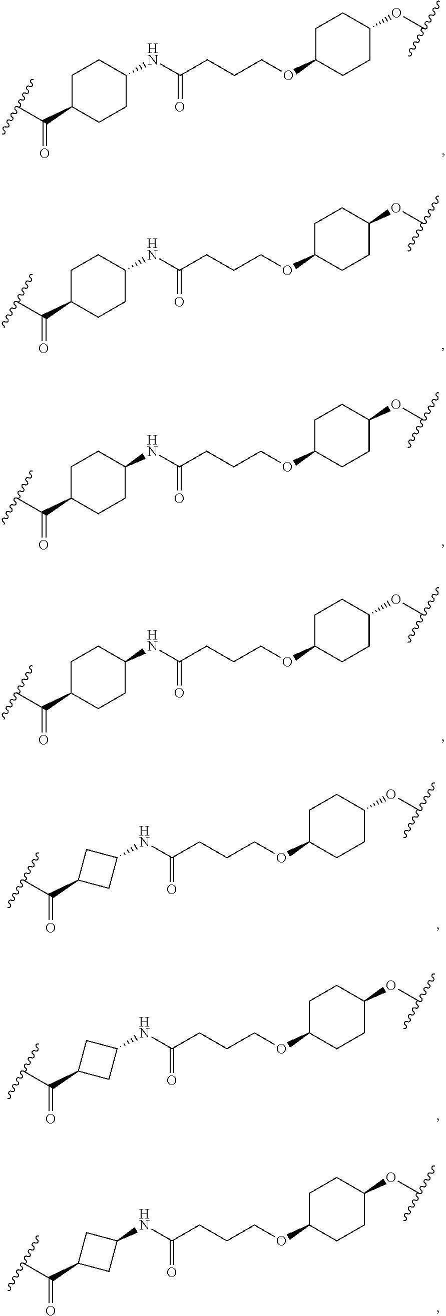

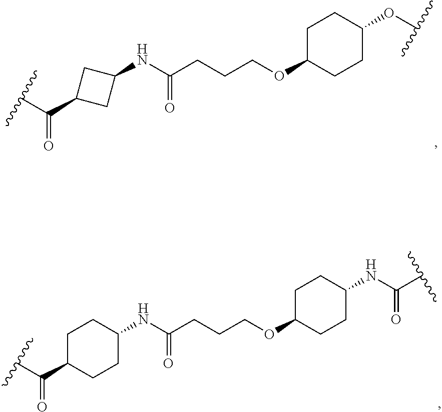 [SMILES: *O[C@H]1CC[C@H](OCCCC(=O)N[C@H]2C[C@@H](C(*)=O)C2)CC1 ; *C(=O)N[C@H]1CC[C@H](OCCCC(=O)N[C@H]2CC[C@H](C(*)=O)CC2)CC1]

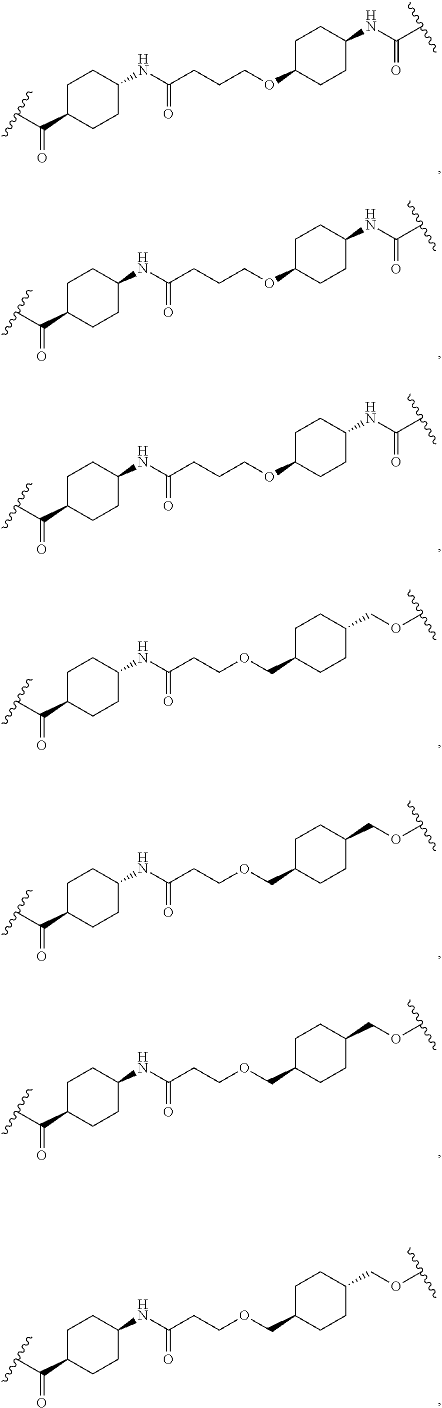

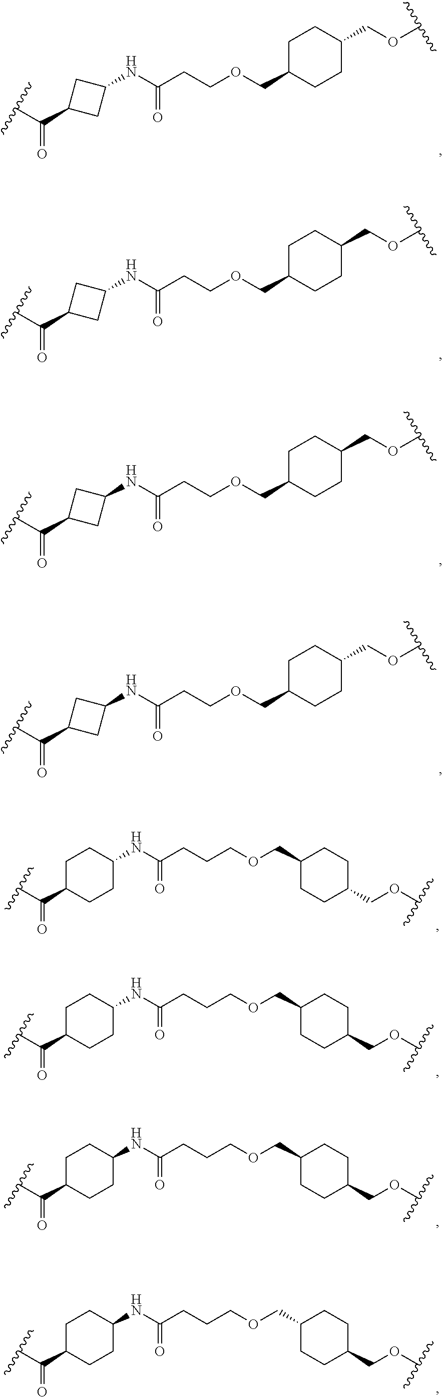

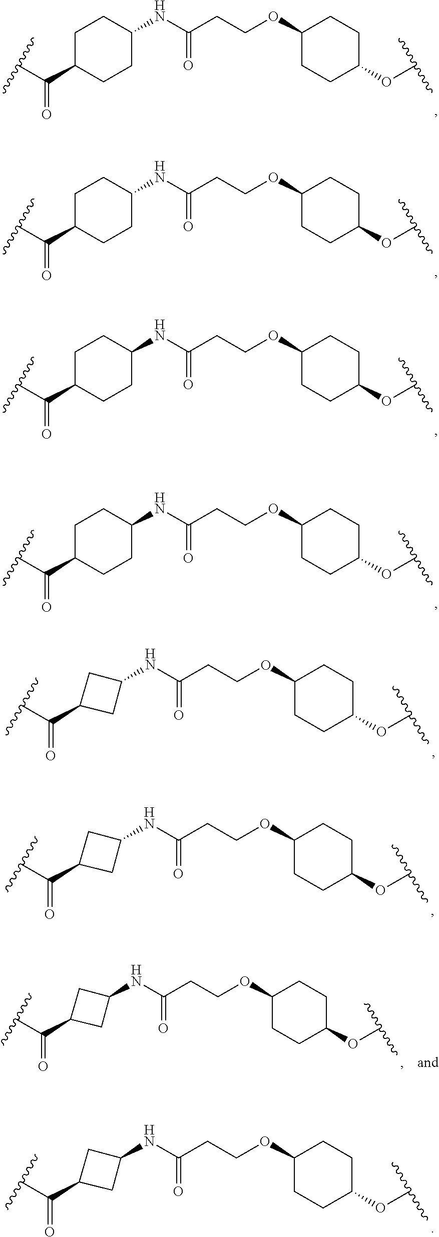

In another embodiment, L is a divalent linker of Formula (L-b):

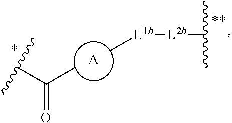

or a stereoisomer thereof,
wherein:
Ring A is C₄₋₆ cycloalkylene or C₇-9 bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is

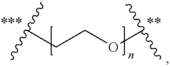

wherein n is 1, 2, 3, or 4, and

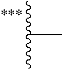

represents a covalent bond to L^{1b}; and
each R^{1b} is independently hydrogen or C₁₋₃ alkyl;
wherein

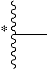

represents a covalent bond to the L′ group of Formula (I), or when L′ is a bond, a covalent bond to the Y group of Formula (I), or when both L′ and Y are a bond, a covalent bond to the T group of Formula (I), and

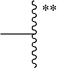

represents a covalent bond to the methylene group of Formula (I).

In another embodiment, Ring A of Formula (L-b) is

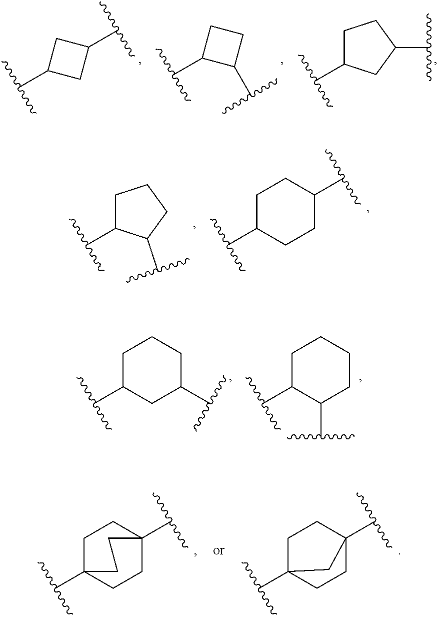

In another embodiment, L is a divalent linker of Formula (L-b-i):

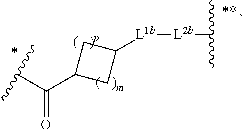

or a stereoisomer thereof,
wherein:
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}, —C(O)NR^{1b}—, or —NR^{1b}C(O)—; or
L^{2b} is

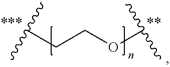

wherein n is 1, 2, 3, or 4, and

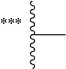

represents a covalent bond to L^{1b}; each R^{1b} is independently hydrogen or C₁₋₃ alkyl;
p is 1 or 2; and
m is 1 or 2;
wherein

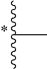

represents a covalent bond to the L′ group of Formula (I), or when L′ is a bond, a covalent bond to the Y group of Formula (I), or when both L′ and Y are a bond, a covalent bond to the T group of Formula (I), and

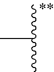

represents a covalent bond to the methylene group of Formula (I).

In another embodiment, L^{2b} of Formula (L-b) or (L-b-i) is selected from

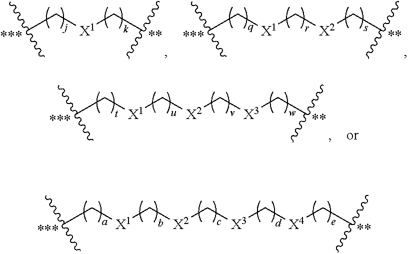

wherein:
j is 1, 2, 3, 4, 5, 6, 7, 8, 9, or 10;
k is 1, 2, 3, 4, 5, 6, 7, 8, 9, or 10;
the sum of j and k is 5, 6, 7, 8, 9, 10, or 11;
q is 1, 2, 3,4,5,6,7,8, or 9;
r is 1, 2, 3, 4, 5, 6, 7, 8, or 9;
s is 0, 1, 2, 3, 4, 5, 6, 7, or 8;
the sum of q, r, and s is 4, 5, 6, 7, 8, 9, or 10;
t is 1, 2, 3, 4, 5, 6, or 7;
u is 1, 2, 3, 4, 5, 6, or 7;
v is 1, 2, 3, 4, 5, 6, or 7;
w is 0, 1, 2, 3, 4, 5, or 6;
the sum of t, u, v, and w is 3, 4, 5, 6, 7, 8, or 9;
a is 1, 2, 3, 4, or 5;
b is 1, 2, 3, 4, or 5;
cis 1, 2, 3, 4, or 5;
d is 1, 2, 3, 4, or 5;
e is 0, 1, 2, 3, or 4;
the sum of a, b, c, d, and e is 4, 5, 6, 7, or 8;
X¹, X², X³, and X⁴ are independently —O—, —NR^{1b}—, —C(O)NR^{1b}—, or —NR^{1b}C(O)—; and
each R^{1b} is independently hydrogen or C₁₋₃ alkyl;
wherein

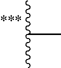

represents a covalent bond to L^{1b} of Formula (L-b) or (L-b-i), and

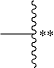

represents a covalent bond to the methylene group of Formula (I).

In another embodiment, L is a divalent linker of Formula (L-b) selected from the group consisting of:

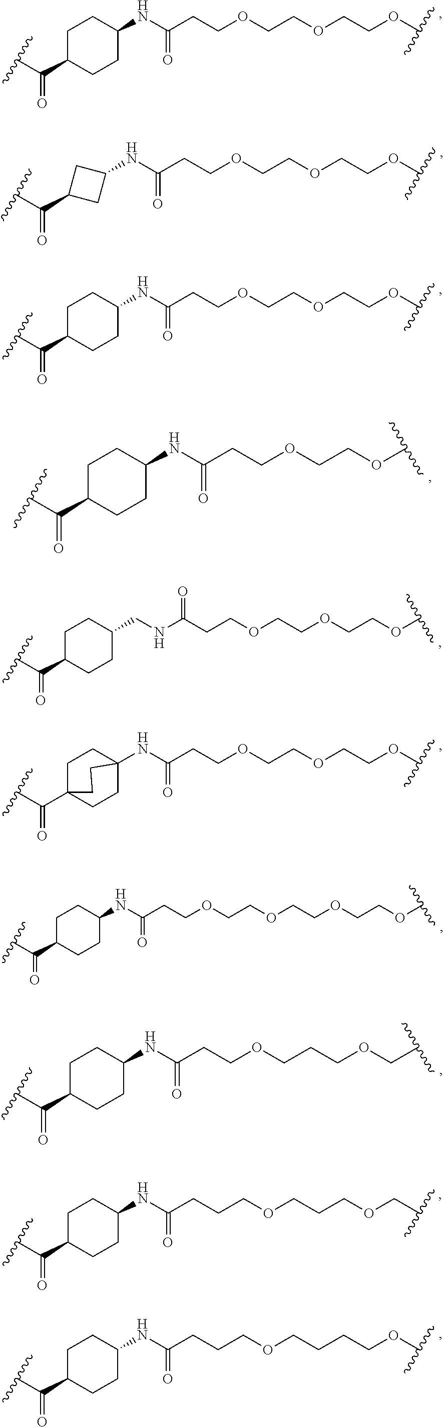

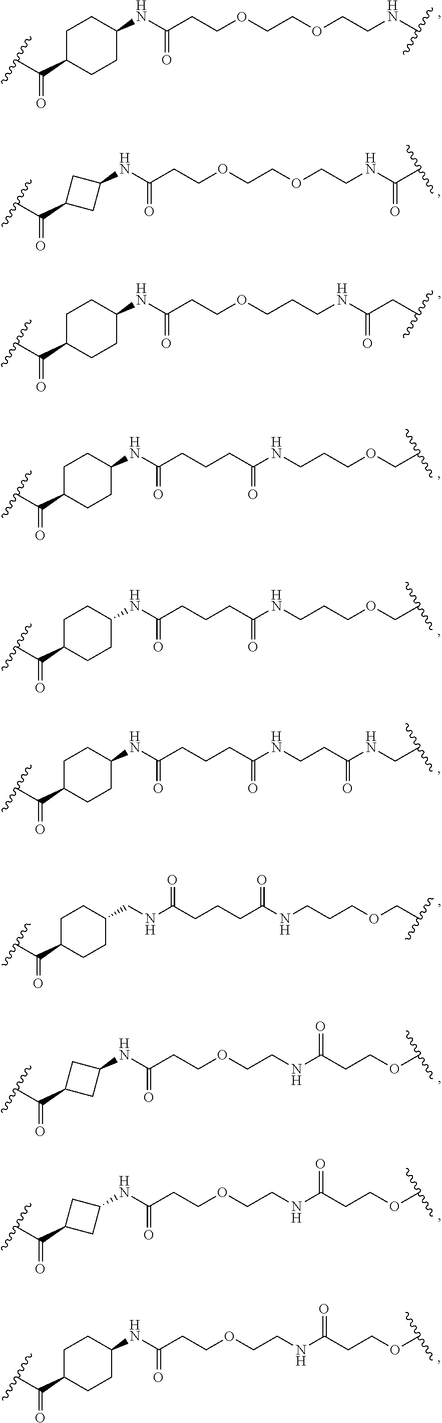

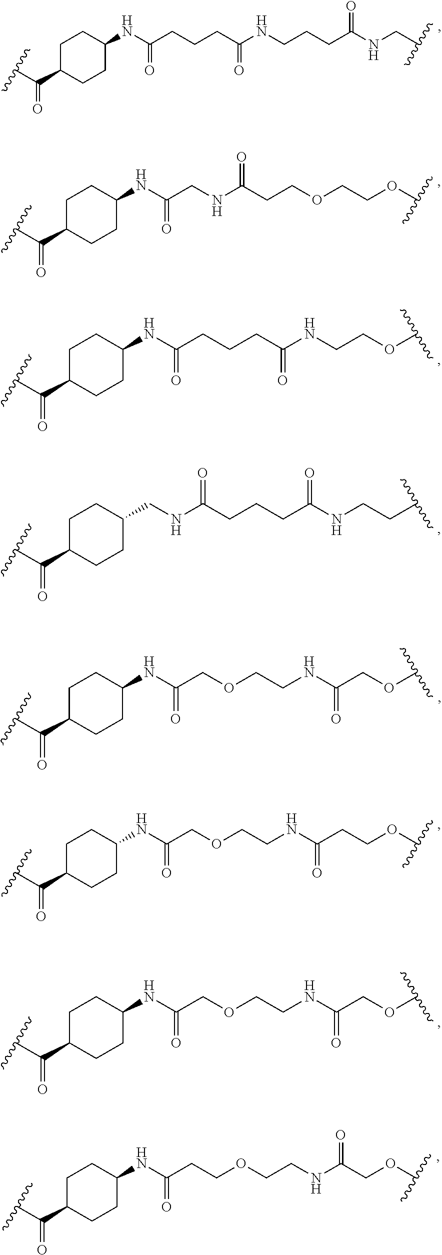

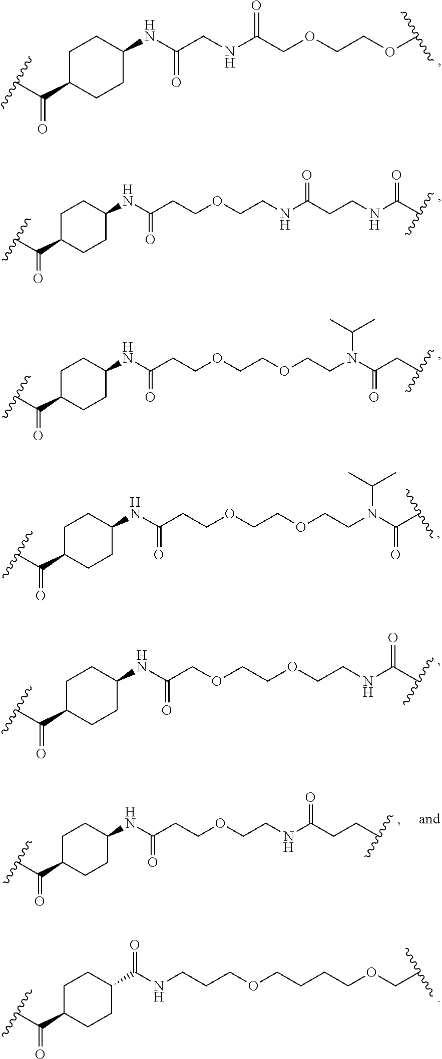

In another embodiment, L is a divalent linker of Formula (L-c):

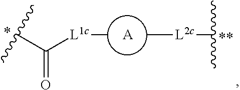

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇-9 bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—;
wherein

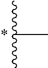

represents a covalent bond to the L′ group of Formula (I), or when L′ is a bond, a covalent bond to the Y group of Formula (I), or when both L′ and Y are a bond, a covalent bond to the T group of Formula (I), and

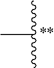

represents a covalent bond to the methylene group of Formula (I).

In another embodiment, Ring A of Formula (L-c) is

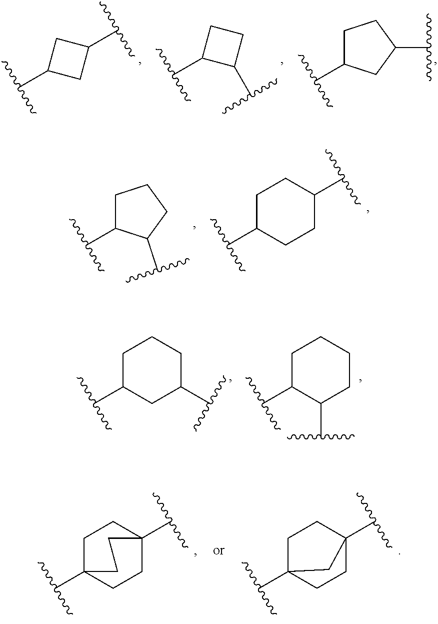

In another embodiment, L is a divalent linker of Formula (L-c-i):

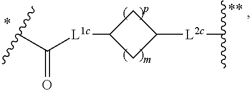

or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
L^{2c} is —O— or a saturated C₂₋₁₀ linear alkylene, wherein 1, 2, or 3 methylene units are replaced with —O—, —NH—, —NHC(O)—, or —C(O)NH—;
p is 1 or 2; and
m is 1 or 2;
wherein

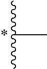

represents a covalent bond to the L′ group of Formula (I), or when L′ is a bond, a covalent bond to the Y group of Formula (I), or when both L′ and Y are a bond, a covalent bond to the T group of Formula (I), and

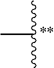

represents a covalent bond to the methylene group of Formula (I).

In another embodiment, L^{1c} of Formula (L-c) or (L-c-i) is selected from

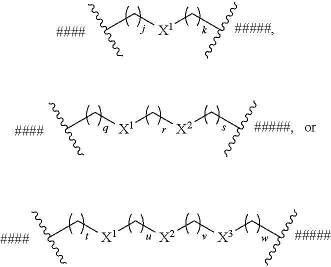

wherein:
j is 1, 2, 3, 4, 5, 6, 7, 8, or 9;
k is 0, 1, 2, 3, 4, 5, 6, 7, or 8;
the sum of j and k is 1, 2, 3, 4, 5, 6, 7, 8, or 9;
q is 1, 2, 3, 4, 5, 6, or 7;
r is 1, 2, 3, 4, 5, 6, or 7;
s is 0, 1, 2, 3, 4, 5, or 6;
the sum of q, r, and s is 2, 3, 4, 5, 6, 7, or 8;
t is 1, 2, 3, 4, or 5;
u is 1, 2, 3, 4, or 5;
v is 1, 2, 3, 4, or 5;
w is 0, 1, 2, 3, or 4;
the sum of t, u, v, and w is 3, 4, 5, 6, or 7; and
X¹, X² and X³ are independently —O—, —NH—, —NHC(O)—, or —C(O)NH—;
wherein

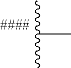

represents a covalent bond to the C(O) group of Formula (L-c) or (L-c-i), and

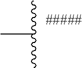

represents a covalent bond to the ring of Formula (L-c) or (L-c-i).

In another embodiment, L^{2c} of Formula (L-c) or (L-c-i) is selected from

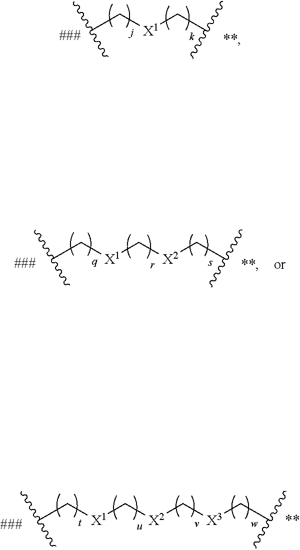

wherein:
j is 0, 1, 2, 3, 4, 5, 6, 7, 8, or 9;
k is 0, 1, 2, 3, 4, 5, 6, 7, 8, or 9;
the sum of j and k is 1, 2, 3, 4, 5, 6, 7, 8, or 9;
q is 0, 2, 3, 4, 5, 6, or 7;
r is 1, 2, 3, 4, 5, 6, 7, or 8;
s is 0, 1, 2, 3, 4, 5, 6, or 7;
the sum of q, r, and s is 1, 2, 3, 4, 5, 6, 7, or 8;
t is 0, 1, 2, 3, 4, or 5;
u is 1, 2, 3, 4, 5, or 6;
v is 1, 2, 3, 4, 5, or 6;
w is 0, 1, 2, 3, 4, or 5;
the sum of t, u, v, and w is 2, 3, 4, 5, 6, or 7; and
X¹, X² and X³ are independently —O—, —NH—, —NHC(O)—, or —C(O)NH—;
wherein

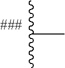

represents a covalent bond to the ring of Formula (L-c) or (L-c-i), and

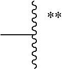

represents a covalent bond to the methylene group of Formula (I).

In another embodiment, L is a divalent linker of Formula (L-c) selected from the group consisting of:

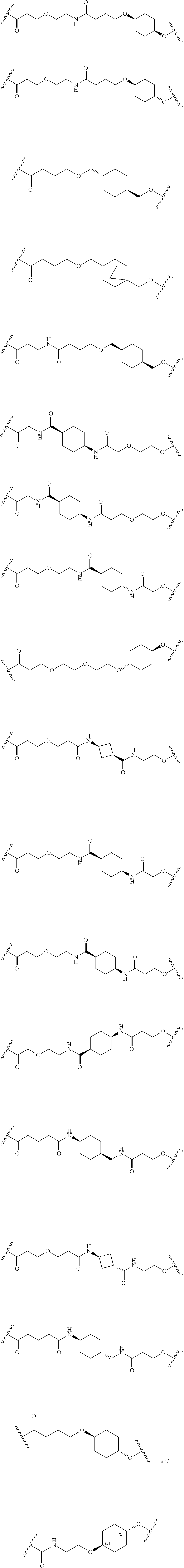

In another embodiment, L is a divalent linker of Formula (L-d):

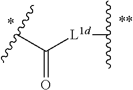

wherein:
L^{1d} is C₁₂₋₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—;
wherein

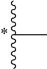

represents a covalent bond to the L′ group of Formula (I), or when L′ is a bond, a covalent bond to the Y group of Formula (I), or when both L′ and Y are a bond, a covalent bond to the T group of Formula (I), and

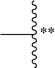

represents a covalent bond to the methylene group of Formula (I). In another embodiment, L^{1d} is a C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, or C₃₁ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—. In another embodiment, L^{1d} is C₁₂₋₂₂ linear alkylene, for example, C₁₂, C₁₃, C₁₄, C₁₅, C₁₆, C₁₇, C₁₈, C₁₉, C₂₀, C₂₁, or C₂₂, wherein 1, 2, 3, 4, or 5 methylene units are replaced with —NH—, —O—, —C(O)NH—, —NHC(O)—, or —NHC(O)—NH—.

In another embodiment, L^{1d} of Formula (L-d) is selected from

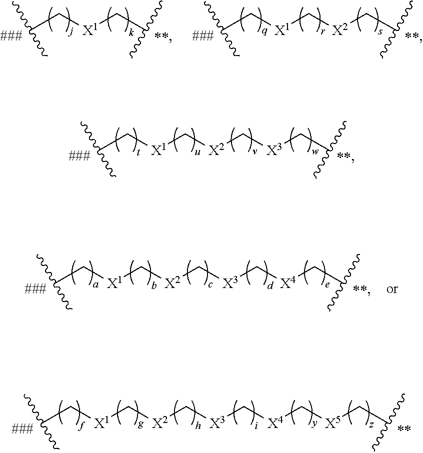

wherein:
j is 1, 2, 3, 4, 5, 6, 7, 8, 9, 10, 11, 12, 13, 14, 15, 16, 17, 18, 19, or 20;
k is 1, 2, 3, 4, 5, 6, 7, 8, 9, 10, 11, 12, 13, 14, 15, 16, 17, 18, 19, or 20;
the sum of j and k is 11, 12, 13, 14, 15, 16, 17, 18, 19, 20, or 21;
q is 1, 2, 3, 4, 5, 6, 7, 8, 9, 10, 11, 12, 13, 14, 15, 16, 17, 18, or 19;
r is 1, 2, 3, 4, 5, 6, 7, 8, 9, 10, 11, 12, 13, 14, 15, 16, 17, 18, or 19;
s is 0, 1, 2, 3, 4, 5, 6, 7, 8, 9, 10, 11, 12, 13, 14, 15, 16, 17, or 18;
the sum of q, r, and s is 10, 11, 12, 13, 14, 15, 16, 17, 18, 19, or 20;
t is 1, 2, 3, 4, 5, 6, 7, 8, 9, 10, 11, 12, 13, 14, 15, 16, or 17;
u is 1, 2, 3, 4, 5, 6, 7, 8, 9, 10, 11, 12, 13, 14, 15, 16, or 17;
v is 1, 2, 3, 4, 5, 6, 7, 8, 9, 10, 11, 12, 13, 14, 15, 16, or 17;
w is 0, 1, 2, 3, 4, 5, 6, 7, 8, 9, 10, 11, 12, 13, 14, 15, or 16;
the sum of t, u, v, and w is 9, 10, 11, 12, 13, 14, 15, 16, 17, 18, or 19;
a is 1, 2, 3, 4, 5, 6, 7, 8, 9, 10, 11, 12, 13, 14, or 15;
b is 1, 2, 3, 4, 5, 6, 7, 8, 9, 10, 11, 12, 13, 14, or 15;
c is 1, 2, 3, 4, 5, 6, 7, 8, 9, 10, 11, 12, 13, 14, or 15;
d is 1, 2, 3, 4, 5, 6, 7, 8, 9, 10, 11, 12, 13, 14, or 15;
e is 0, 1, 2, 3, 4, 5, 6, 7, 8, 9, 10, 11, 12, 13, or 14;
the sum of a, b, c, d, and e is 8, 9, 10, 11, 12, 13, 14, 15, 16, 17, or 18;
f is 1, 2, 3, 4, 5, 6, 7, 8, 9, 10, 11, 12, or 13;
g is 1, 2, 3, 4, 5, 6, 7, 8, 9, 10, 11, 12, or 13;
h is 1, 2, 3, 4, 5, 6, 7, 8, 9, 10, 11, 12, or 13;
i is 1, 2, 3, 4, 5, 6, 7, 8, 9, 10, 11, 12, or 13;
y is 1, 2, 3, 4, 5, 6, 7, 8, 9, 10, 11, 12, or 13;
z is 0, 1, 2, 3, 4, 5, 6, 7, 8, 9, 10, 11, or 12;
the sum of f, g, h, i, y, and z is 7, 8, 9, 10, 11, 12, 13, 14, 15, 16, or 17; and
X¹, X², X³, X⁴, and X⁵ are independently —NH—, —O—, —C(O)NH—, —NHC(O)—, or —NHC(O)—NH—;
wherein

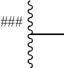

represents a covalent bond to the C(O) group of Formula (L-d), and

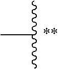

represents a covalent bond to the methylene group of Formula (I).

In another embodiment, L^{1d} of Formula (L-d) is

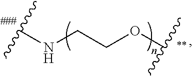

wherein n is 4, 5, 6, 7, 8, 9 or 10;
wherein

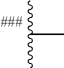

represents a covalent bond to the C(O) group of Formula (L-d), and

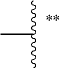

represents a covalent bond to the methylene group of Formula (I).

In another embodiment, L is a divalent linker of Formula (L-d) selected from the group consisting of:

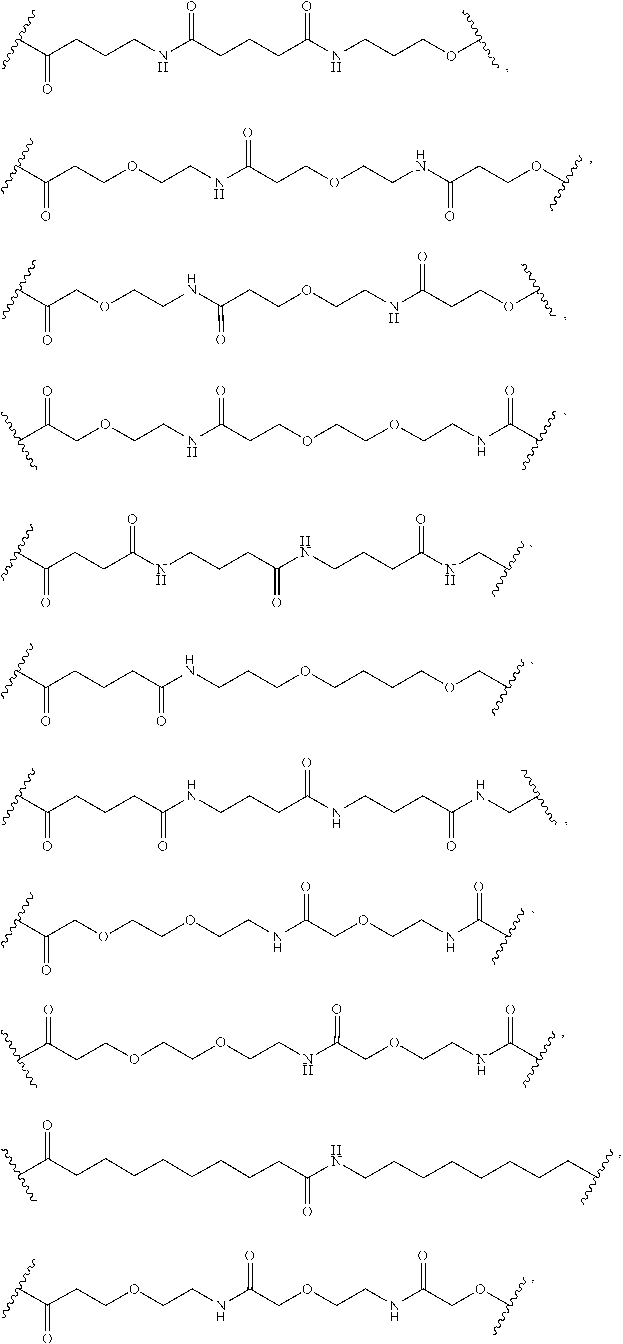

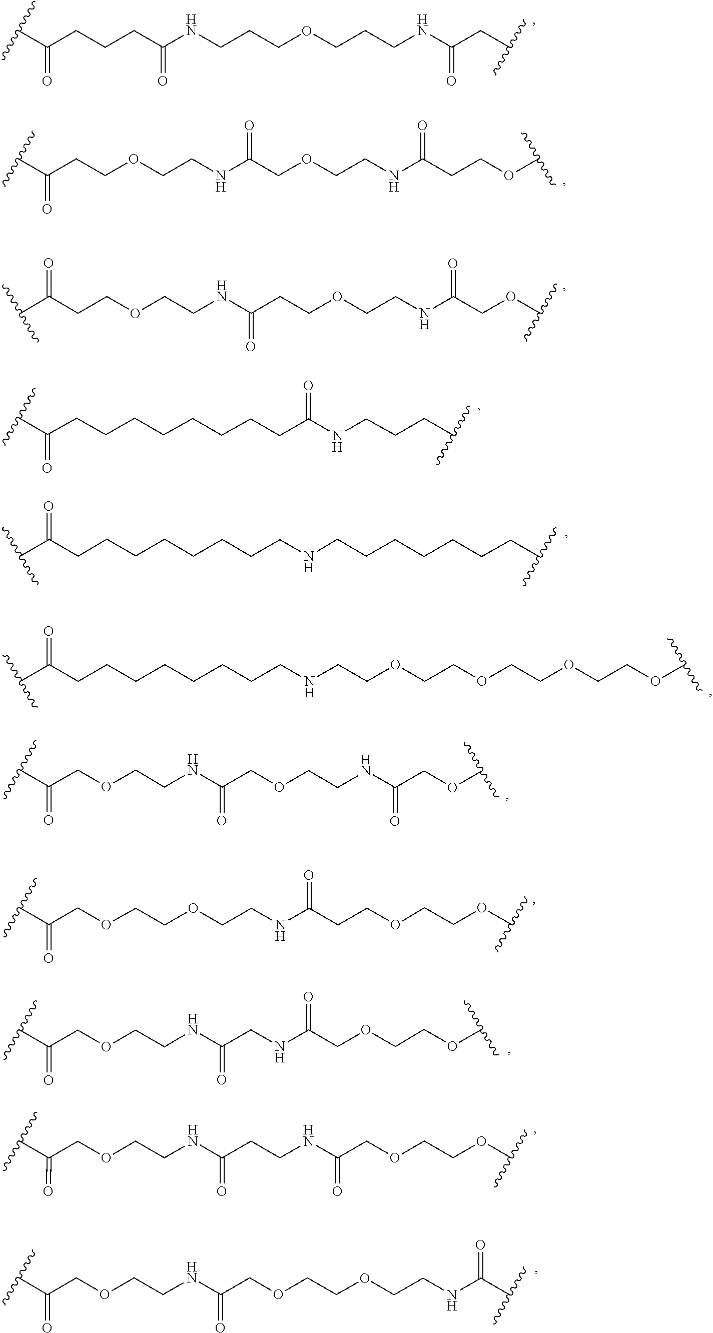

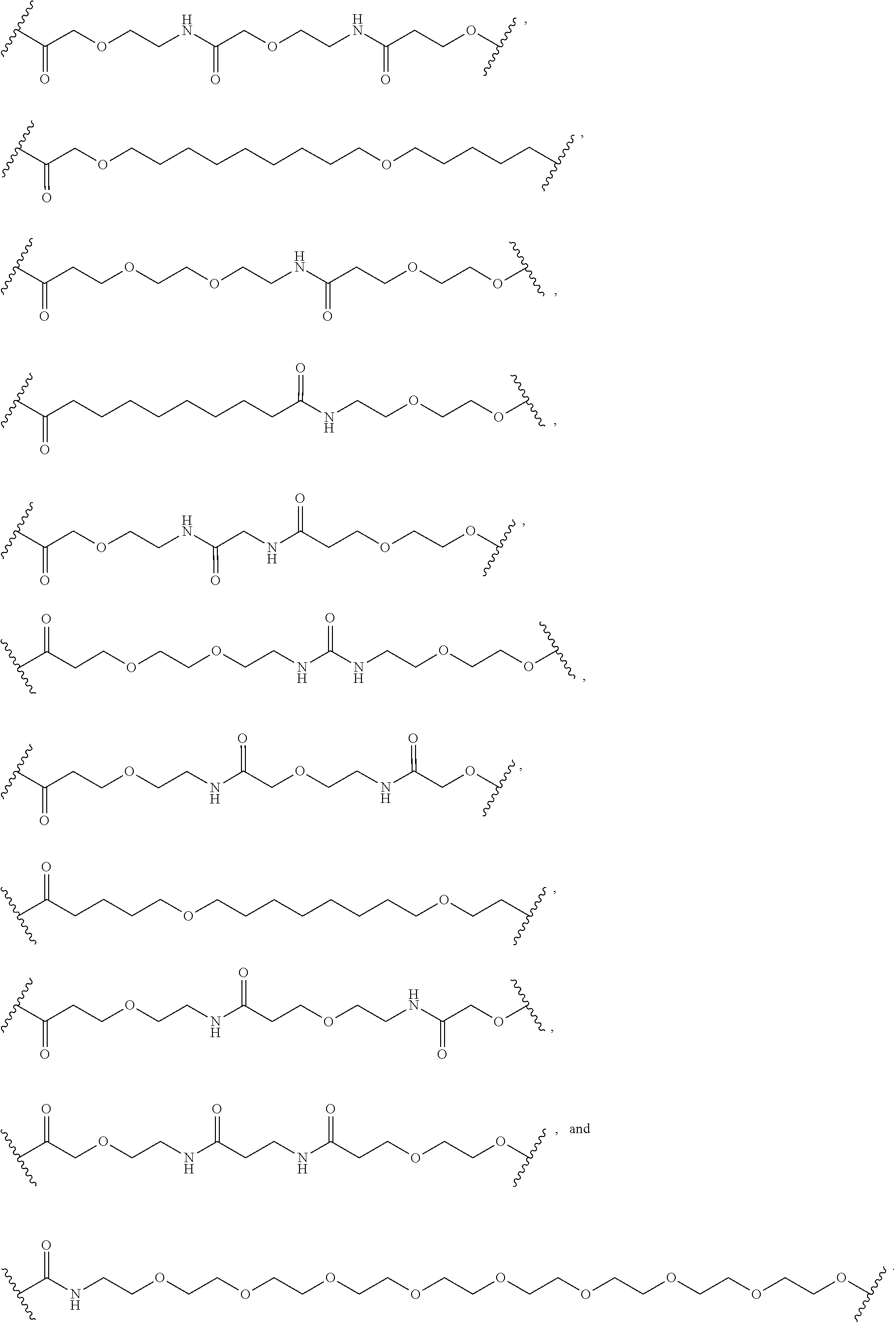

In another embodiment, L is a divalent linker of Formula (L-e):

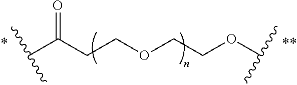

wherein:
n is an integer of 3 to 50;
wherein

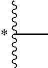

represents a covalent bond to the L′ group of Formula (I), or when L′ is a bond, a covalent bond to the Y group of Formula (I), or when both L′ and Y are a bond, a covalent bond to the T group of Formula (I), and

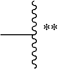

represents a covalent bond to the methylene group of Formula (I).

In another embodiment, n of Formula (L-e) is 3 to 25, 3 to 10, 3 to 8, 3 to 7, 3 to 5, or 3 to 4. In another embodiment, n of Formula (L-e) is 5 to 22, 7 to 15, or 9 to 13. In another embodiment, n of Formula (L-e) is 3, 4, 5, 7, 8, 11, 22, or 50.

In another embodiment, n of Formula (L-e) is 12 to 50, 15 to 30, 17 to 25, 18 to 24, 18 to 20, 20 to 22, or 22 to 24. In another embodiment, n of Formula (L-e) is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50. In another embodiment, n of Formula (L-e) is 19 or 23.

In another embodiment, L is a divalent linker of Formula (L-f):

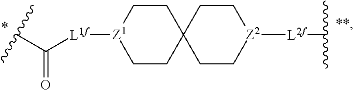

or a stereoisomer thereof,
wherein:
L^{1f} is a bond; C₁₋₆ linear alkylene, wherein 0, 1, or 2 methylene units are replaced with —O—, —NH—, or —C(O)—; or —(C₃₋₆ cycloalkylene)-NHC(O)—;
L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH;
wherein

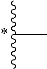

represents a covalent bond to the L′ group of Formula (I), or when L′ is a bond, a covalent bond to the Y group of Formula (I), or when both L′ and Y are a bond, a covalent bond to the T group of Formula (I), and

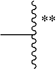

represents a covalent bond to the methylene group of Formula (I).

In another embodiment, L^{1f} of Formula (L-f) is selected from

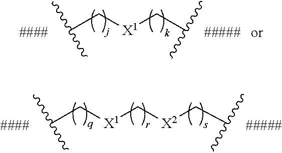

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2;
the sum of q, r, and s is 2, 3, or 4; and
X¹ and X² are independently —O—, —NH—, or —C(O)—; or —(C₃₋₆ cycloalkylene)-NHC(O)—;
wherein

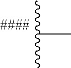

represents a covalent bond to the C(O) group of Formula (L-f), and

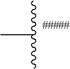

represents a covalent bond to the ring of Formula (L-f).

In another embodiment, L^{2f} of Formula (L-f) is selected from

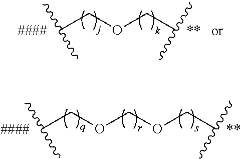

wherein:
j is 1, 2, 3, 4, or 5;
k is 0, 1, 2, 3, or 4;
the sum of j and k is 1, 2, 3, 4, or 5;
q is 1, 2, or 3;
r is 1, 2, or 3;
s is 0, 1, 2; and
the sum of q, r, and s is 2, 3, or 4;
wherein

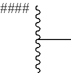

represents a covalent bond to the ring of Formula (L-f), and

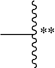

represents a covalent bond to the methylene group of Formula (I).

In another embodiment, L is a divalent linker of Formula (L-f) selected from the group consisting of:

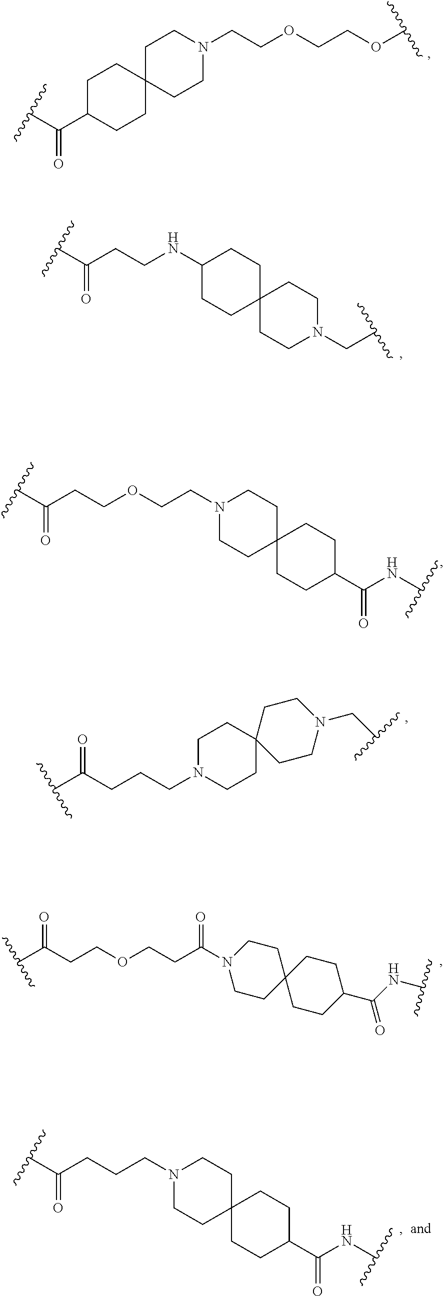 [SMILES: *OCCOCCN1CCC2(CCC(C(*)=O)CC2)CC1 ; *CN1CCC2(CCC(NCCC(*)=O)CC2)CC1 ; *NC(=O)C1CCC2(CC1)CCN(CCOCCC(*)=O)CC2 ; *CN1CCC2(CC1)CCN(CCCC(*)=O)CC2 ; *NC(=O)C1CCC2(CC1)CCN(C(=O)CCOCCC(*)=O)CC2 ; *NC(=O)C1CCC2(CC1)CCN(CCCC(*)=O)CC2]

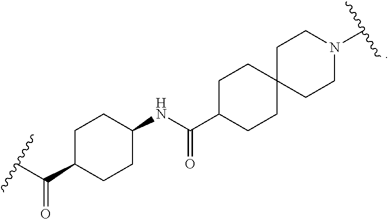 [SMILES: *C(=O)[C@H]1CC[C@@H](NC(=O)C2CCC3(CC2)CCN(*)CC3)CC1]

In another embodiment, L is a divalent linker of Formula (L-g):

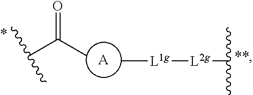

wherein:
Ring A is a 5 to 6 membered heteroarylene having 1 or 2 nitrogen ring atoms;
L^{1g} is a bond, —CH₂—, —NH—, or —O—; and
L^{2g} is

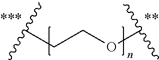

wherein n is 1, 2, 3, 4, or 5, and

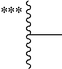

represents a covalent bond to L^{1g};
wherein

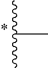

represents a covalent bond to the L′ group of Formula (I), or when L′ is a bond, a covalent bond to the Y group of Formula (I), or when both L′ and Y are a bond, a covalent bond to the T group of Formula (I),

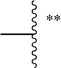

represents a covalent bond to the methylene group of Formula (I).

In another embodiment, L is a divalent linker of Formula (L-g-i):

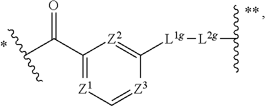

wherein:
L^{1g} is a bond, —CH₂—, —NH—, or —O—;
L^{2g} is

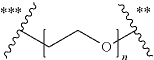

wherein n is 1, 2, 3, 4, or 5, and

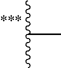

represents a covalent bond to L^{1g};
Z¹, Z², and Z³ are each independently selected from N or CH, provided that one or two of Z¹, Z², and Z³ is N;
wherein

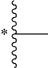

represents a covalent bond to the L′ group of Formula (I), or when L′ is a bond, a covalent bond to the Y group of Formula (I), or when both L′ and Y are a bond, a covalent bond to the T group of Formula (I), and

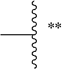

represents a covalent bond to the methylene group of Formula (I).

In another embodiment, L is a divalent linker of Formula (L-g) selected from the group consisting of:

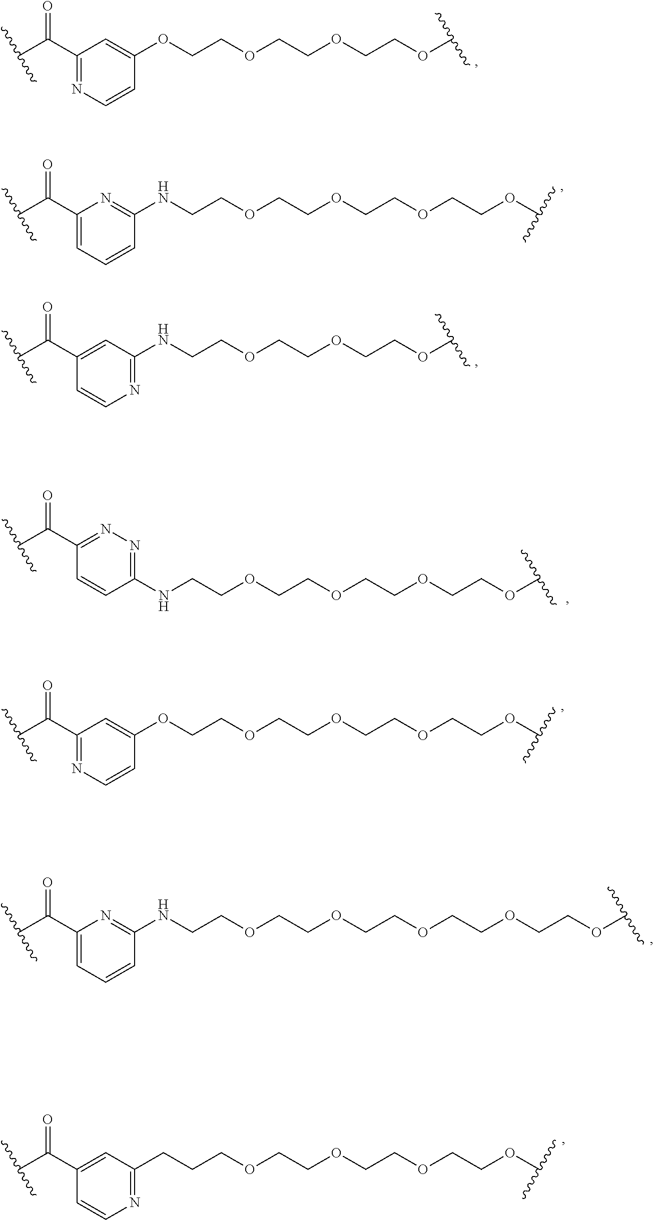

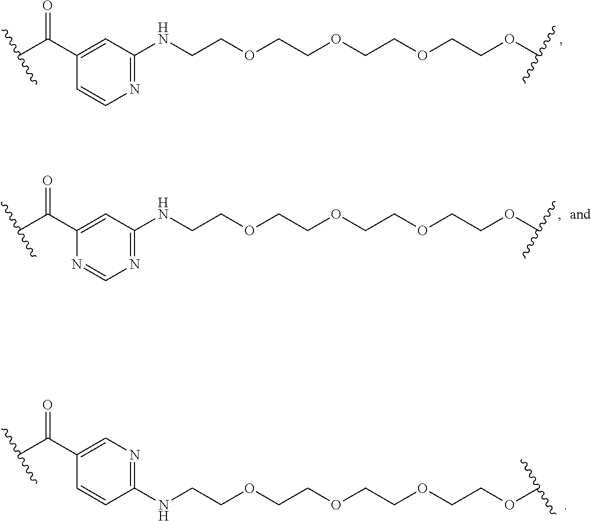 [SMILES: *OCCOCCOCCOCCNc1cc(C(*)=O)ccn1 ; *OCCOCCOCCOCCNc1cc(C(*)=O)ncn1 ; *OCCOCCOCCOCCNc1ccc(C(*)=O)cn1]

In another embodiment, L is a divalent linker of Formula (L-h):

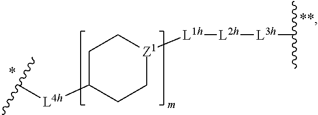

or a stereoisomer thereof,
wherein:
each Z¹ is independently N or CH;
L^{1h} is a bond, —C(O)—, —C(O)—NH—, or —NHC(O)—;
L² h is C₂₋₁₀ linear alkylene or

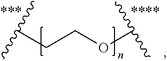

wherein n is 1, 2, 3, or 4, and

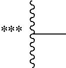

represents a covalent bond to L^{1h} and

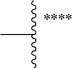

represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3;
wherein

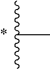

represents a covalent bond to the L′ group of Formula (I), or when L′ is a bond, a covalent bond to the Y group of Formula (I), or when both L′ and Y are a bond, a covalent bond to the T group of Formula (I), and

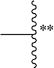

represents a covalent bond to the methylene group of Formula (I).

In another embodiment, L is a divalent linker of Formula (L-h) selected from the group consisting of:

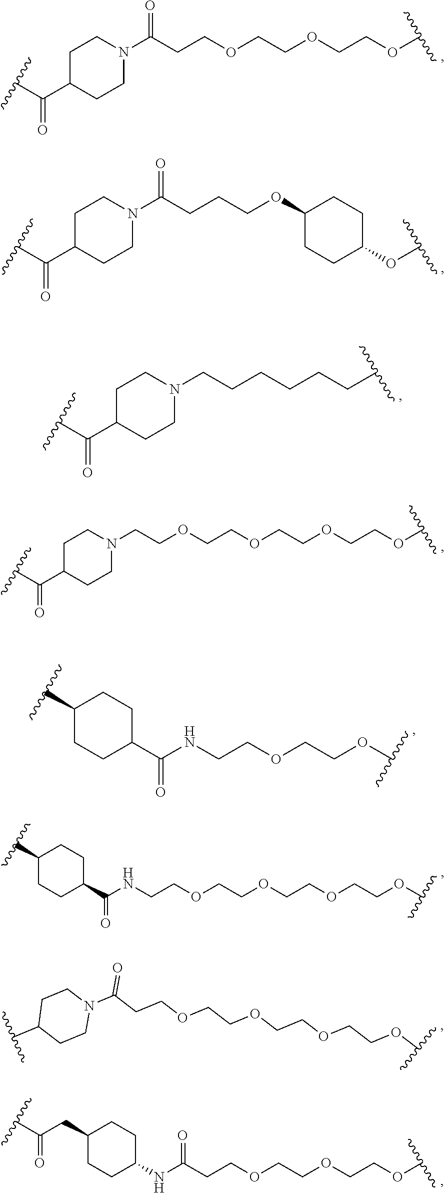

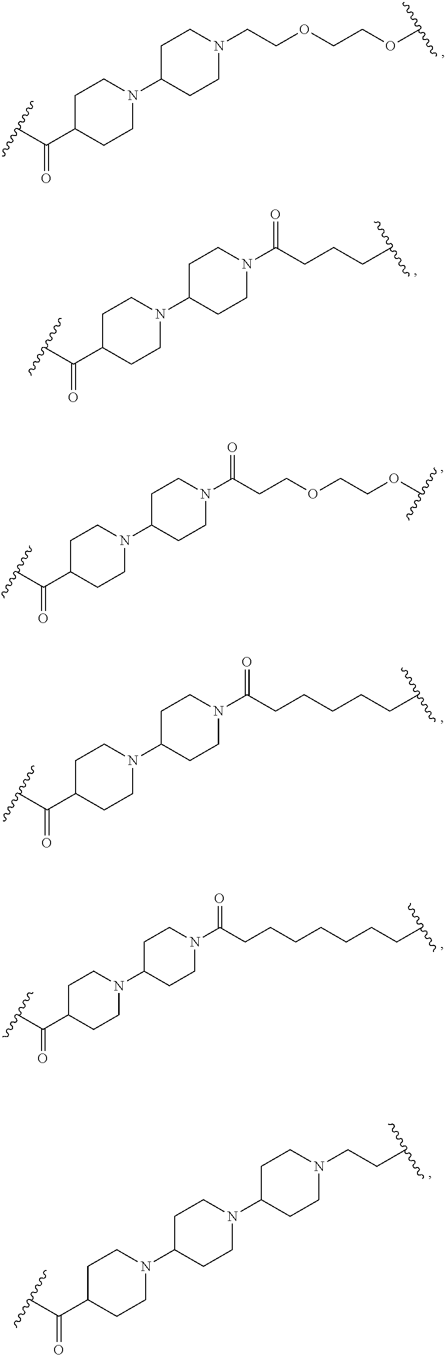 [SMILES: *OCCOCCN1CCC(N2CCC(C(*)=O)CC2)CC1 ; *CCCC(=O)N1CCC(N2CCC(C(*)=O)CC2)CC1 ; *OCCOCCC(=O)N1CCC(N2CCC(C(*)=O)CC2)CC1 ; *CCCCCC(=O)N1CCC(N2CCC(C(*)=O)CC2)CC1 ; *CCCCCCCC(=O)N1CCC(N2CCC(C(*)=O)CC2)CC1 ; *CCN1CCC(N2CCC(N3CCC(C(*)=O)CC3)CC2)CC1]

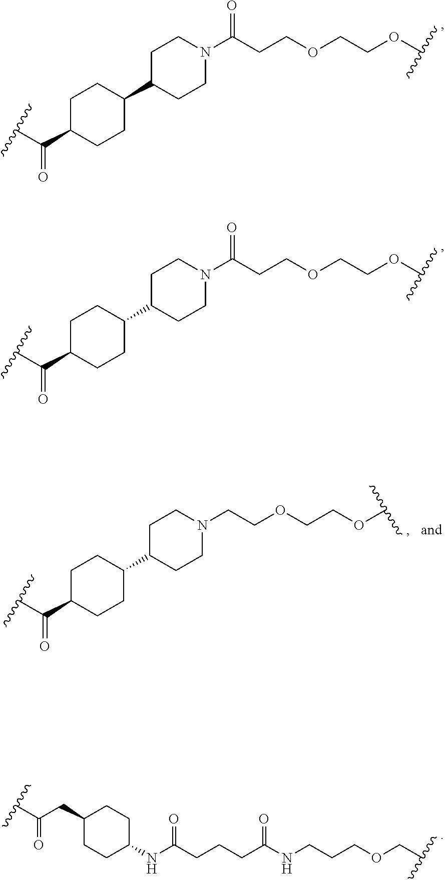 [SMILES: *OCCOCCC(=O)N1CCC([C@H]2CC[C@@H](C(*)=O)CC2)CC1 ; *OCCOCCC(=O)N1CCC([C@H]2CC[C@H](C(*)=O)CC2)CC1 ; *OCCOCCN1CCC([C@H]2CC[C@H](C(*)=O)CC2)CC1 ; *COCCCNC(=O)CCCC(=O)N[C@H]1CC[C@H](CC(*)=O)CC1]

In another embodiment, L is a divalent linker of Formula (L-i):

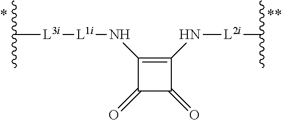

wherein:
L¹ⁱ is a bond, C₁₋₁₂ linear alkylene, or

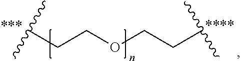

wherein n is 1, 2, 3, 4, or 5, and

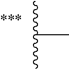

represents a covalent bond to L³ⁱ and

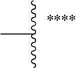

represents a covalent bond to NH;
L²ⁱ is a bond, C₁₋₁₂ linear alkylene, or

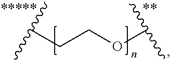

wherein n is 1, 2, 3, 4, or 5, and

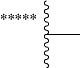

represents a covalent bond to NH; and
L³ⁱ is a bond or —C(O)—;
wherein

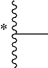

represents a covalent bond to the L′ group of Formula (I), or when L′ is a bond, a covalent bond to the Y group of Formula (I), or when both L′ and Y are a bond, a covalent bond to the T group of Formula (I), and

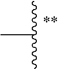

represents a covalent bond to the methylene group of Formula (I).

In another embodiment, L is a divalent linker of Formula (L-i) selected from the group consisting of:

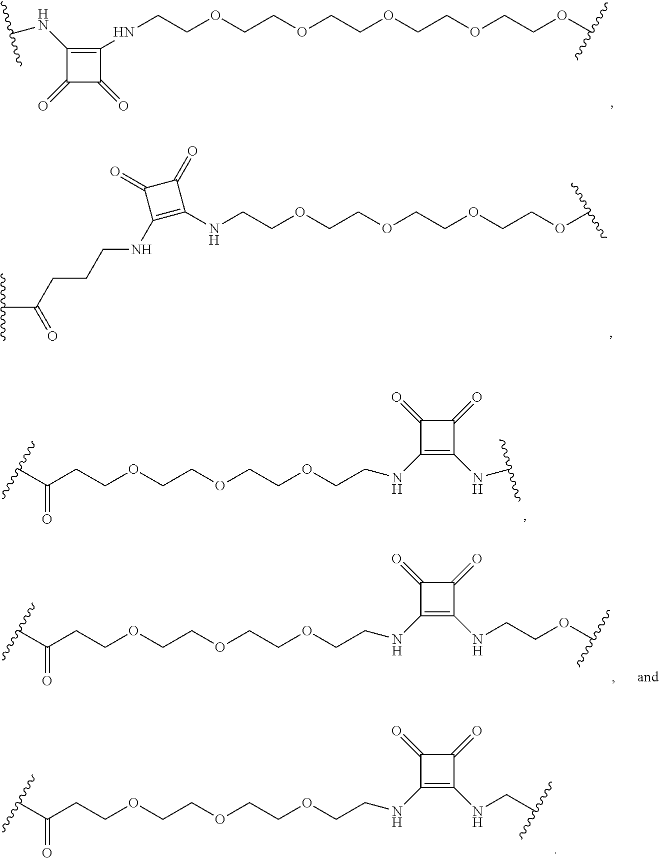 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

In another embodiment, L is a divalent linker of Formula (L-j):

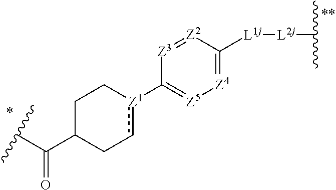

or a stereoisomer thereof,
wherein:
Z¹ is C, CH, or N;
each of Z², Z³, Z⁴ and Z⁵ is independently CH or N, provided that no more than two of Z², Z³, Z⁴ and Z⁵ are N;
L^{1j} is —NH—, —C(O)NH—, —NHC(O)—, or —O—;
L^{2j} is C₁₋₆ linear alkylene or

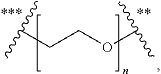

wherein n is 1 or 2, and

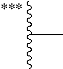

represents a covalent bond to L^{1j}; and
[symbol] represents a single bond or a double bond;
[symbol] wherein

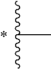

represents a covalent bond to the L′ group of Formula (I), or when L′ is a bond, a covalent bond to the Y group of Formula (I), or when both L′ and Y are a bond, a covalent bond to the T group of Formula (I), and

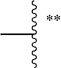

represents a covalent bond to the methylene group of Formula (I).

In another embodiment, L is a divalent linker of Formula (L-j) selected from the group consisting of:

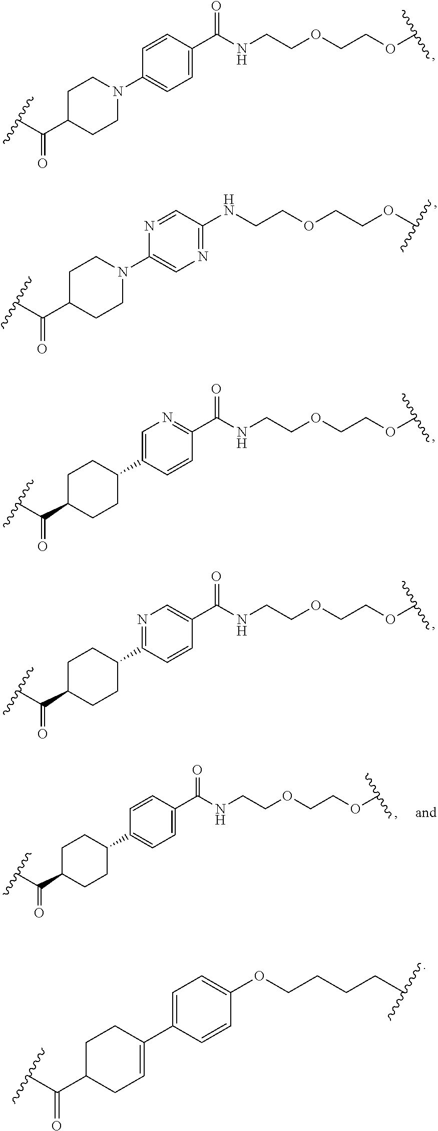 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

In another embodiment, L is a divalent linker of Formula (L-k):

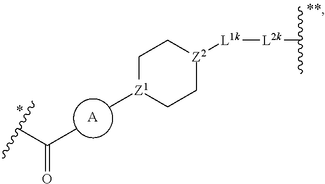

or a stereoisomer thereof,
wherein:
Ring A is phenyl or a 5 or 6 membered heteroarylene having 1 or 2 nitrogen ring atoms;
each of Z¹ and Z² is independently CH or N;
L^{1k} is a bond, —C(O)—, —C(O)NH— or —NHC(O)—; and
L^{2k} is a C₃₋₈ straight chain alkylene or

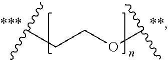

wherein n is 1, 2, or 3, and

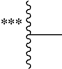

represents a covalent bond to L^{1k};
wherein

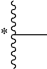

represents a covalent bond to the L′ group of Formula (I), or when L′ is a bond, a covalent bond to the Y group of Formula (I), or when both L′ and Y are a bond, a covalent bond to the T group of Formula (I), and

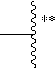

represents a covalent bond to the methylene group of Formula (I).

In another embodiment, L is a divalent linker of Formula (L-k) selected from the group consisting of:

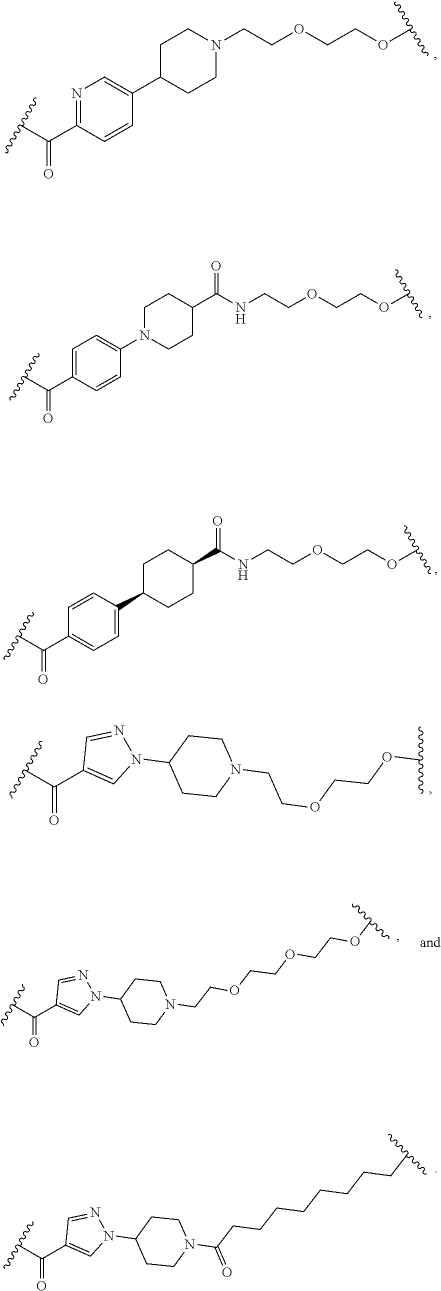 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

In another embodiment, L is a divalent linker of Formula (L-m):

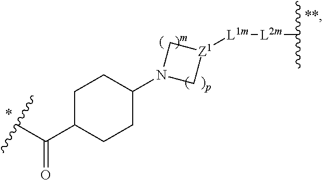

or a stereoisomer thereof,
wherein:
Z¹ is CH or N;
m is 1 or 2;
p is 1 or 2; 0, 1, or 2 hydrogen atoms of

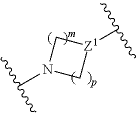

are replaced with F;
L^{1m} is a bond, —C(O)—, —C(O)NH—, —NHC(O)—, —S(O)₂NH— or —NHS(O)₂—; and
L^{2m} is C₃₋₆ linear alkylene, C₃₋₆ cycloalkylene, or

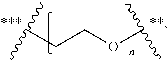

wherein n is 1 or 2, and

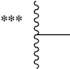

represents a covalent bond to L^{1m};
wherein

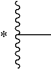

represents a covalent bond to the L′ group of Formula (I), or when L′ is a bond, a covalent bond to the Y group of Formula (I), or when both L′ and Y are a bond, a covalent bond to the T group of Formula (I), and

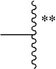

represents a covalent bond to the methylene group of Formula (I).

In another embodiment, L is a divalent linker of Formula (L-m) selected from the group consisting of:

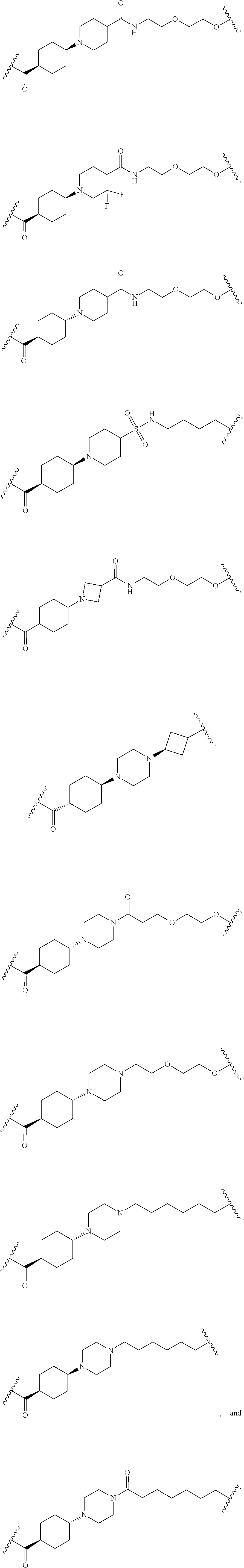

In another embodiment, L is a divalent linker of Formula (L-n-i):

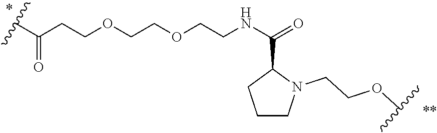 [SMILES: *OCCN1CCC[C@H]1C(=O)NCCOCCOCCC(*)=O]

wherein

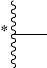

represents a covalent bond to the L′ group of Formula (I), or when L′ is a bond, a covalent bond to the Y group of Formula (I), or when both L′ and Y are a bond, a covalent bond to the T group of Formula (I), and

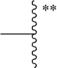

represents a covalent bond to the methylene group of Formula (I).

In another embodiment, L is a divalent linker of Formula (L-n-ii):

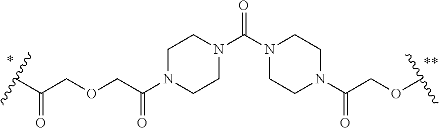 [SMILES: *OCC(=O)N1CCN(C(=O)N2CCN(C(=O)COCC(*)=O)CC2)CC1]

wherein

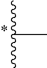

represents a covalent bond to the L′ group of Formula (I), or when L′ is a bond, a covalent bond to the Y group of Formula (I), or when both L′ and Y are a bond, a covalent bond to the T group of Formula (I), and

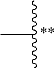

represents a covalent bond to the methylene group of Formula (I).

In another embodiment, L is a divalent linker of Formula (L-n-iii):

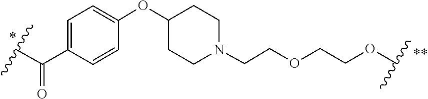 [SMILES: *OCCOCCN1CCC(Oc2ccc(C(*)=O)cc2)CC1]

wherein

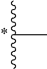

represents a covalent bond to the L′ group of Formula (I), or when L′ is a bond, a covalent bond to the Y group of Formula (I), or when both L′ and Y are a bond, a covalent bond to the T group of Formula (I), and

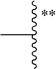

represents a covalent bond to the methylene group of Formula (I).

In another embodiment, L is a divalent linker of Formula (L-n-iv):

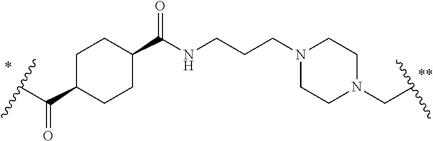 [SMILES: *CN1CCN(CCCNC(=O)[C@H]2CC[C@@H](C(*)=O)CC2)CC1]

wherein

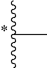

represents a covalent bond to the L′ group of Formula (I), or when L′ is a bond, a covalent bond to the Y group of Formula (I), or when both L′ and Y are a bond, a covalent bond to the T group of Formula (I), and

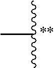

represents a covalent bond to the methylene group of Formula (I).

In another embodiment, L is a divalent linker of Formula (L-p):

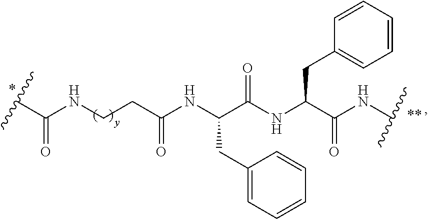

or a stereoisomer thereof, wherein y is an integer of 1 to 9;
wherein

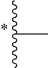

represents a covalent bond to the L′ group of Formula (I), or when L′ is a bond, a covalent bond to the Y group of Formula (I), or when both L′ and Y are a bond, a covalent bond to the T group of Formula (I), and

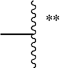

represents a covalent bond to the methylene group of Formula (I).

In another embodiment, L is a divalent linker of Formula (L-q):

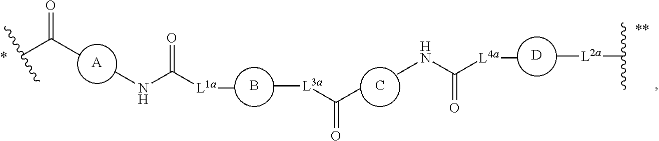

or a stereoisomer thereof,
wherein:
Ring A, Ring B, Ring C, and Ring D are each independently C₄₋₆ cycloalkylene;
L^{1a}, L^{3a}, and L^{4a} are each independently C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—;
wherein

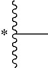

represents a covalent bond to the L′ group of Formula (I), or when L′ is a bond, a covalent bond to the Y group of Formula (I), or when both L′ and Y are a bond, a covalent bond to the T group of Formula (I), and

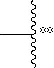

represents a covalent bond to the methylene group of Formula (I).

In another embodiment, L is a divalent linker of Formula (L-q-i):

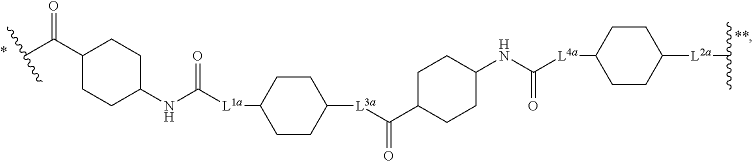

or a stereoisomer thereof,
wherein:
L^{1a}, L^{3a}, and L^{4a} are each independently C₃₋₅ linear alkylene, wherein 1 or 2 methylene units are replaced with —O— or —NR^{a}—
each R^{a} is independently hydrogen or C₁₋₃ alkyl; and
L^{2a} is —O—, —NHC(O)—, or —CH₂—O—;
wherein

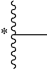

represents a covalent bond to the L′ group of Formula (I), or when L′ is a bond, a covalent bond to the Y group of Formula (I), or when both L′ and Y are a bond, a covalent bond to the T group of Formula (I), and

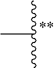

represents a covalent bond to the methylene group of Formula (I).

In another embodiment, L is a divalent linker of Formula (L-q-ii):

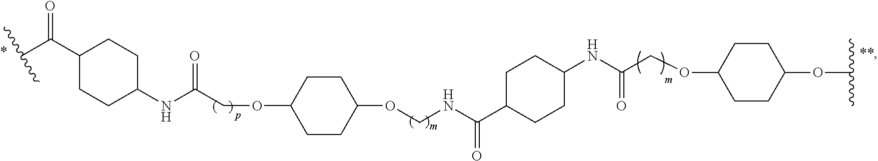

or a stereoisomer thereof,
wherein:
p is 1, 2, or 3;
m is 1, 2, or 3; and
n is 1, 2, or 3;
wherein

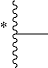

represents a covalent bond to the L′ group of Formula (I), or when L′ is a bond, a covalent bond to the Y group of Formula (I), or when both L′ and Y are a bond, a covalent bond to the T group of Formula (I), and

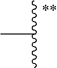

represents a covalent bond to the methylene group of Formula (I).

In another embodiment, L is a divalent linker of Formula (L-q) having the following structure:

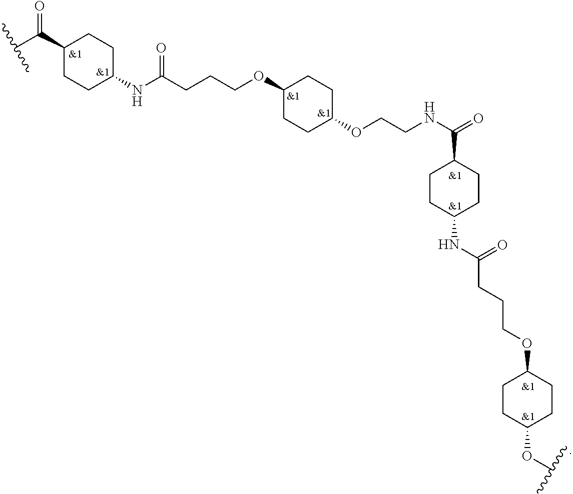 [SMILES: *O[C@H]1CC[C@H](OCCCC(=O)N[C@H]2CC[C@H](C(=O)NCCO[C@H]3CC[C@H](OCCCC(=O)N[C@H]4CC[C@H](C(*)=O)CC4)CC3)CC2)CC1]

In another embodiment, L is a divalent linker of Formula (L-r):

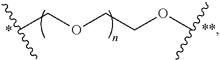

wherein
n is an integer of 10 to 30;
wherein

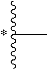

represents a covalent bond to the L′ group of Formula (I), or when L′ is a bond, a covalent bond to the Y group of Formula (I), or when both L′ and Y are a bond, a covalent bond to the T group of Formula (I), and

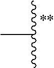

represents a covalent bond to the methylene group of Formula (I).

In another embodiment, n of Formula (L-r) is 10 to 20, 10 to 18, 12 to 16, or 13 to 15. In another embodiment, n of Formula (L-r) is 10, 11, 12, 13, 14, 15, 16, 17, 18, 19, 20, 21, 22, 23, 24, 25, 26, 27, 28, 29, or 30. In another embodiment, n of Formula (L-r) is 14.

In another embodiment, L is a divalent linker of Formula (L-s):

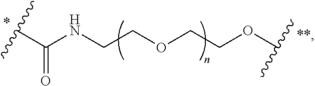

wherein
n is an integer of 10 to 30;
wherein

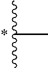

represents a covalent bond to the L′ group of Formula (I), or when L′ is a bond, a covalent bond to the Y group of Formula (I), or when both L′ and Y are a bond, a covalent bond to the T group of Formula (I), and

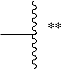

represents a covalent bond to the methylene group of Formula (I).

In another embodiment, n of Formula (L-s) is 10 to 20, 10 to 18, 12 to 16, or 13 to 15. In another embodiment, n of Formula (L-s) is 15 to 30, 17 to 28, 18 to 26, 19 to 25, 20 to 24, or 21 to 23. In another embodiment, n of Formula (L-s) is 10, 11, 12, 13, 14, 15, 16, 17, 18, 19, 20, 21, 22, 23, 24, 25, 26, 27, 28, 29, or 30. In another embodiment, n of Formula (L-s) is 14 or 22.

In one embodiment of the disclosure, Y is selected from a bond; —NH—; —(C₁₋₁₂ alkylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 10-membered heterocycloalkylene, arylene, or heteroarylene; or -(−₂₋₁₂ alkenylene)-, wherein 1, 2, or 3 methylene units are replaced with —O—, —NH—, —N(CH₃)—, —C(O)—, —NHC(O)—, —C(O)NH—, —(C₃₋₆ cycloalkylene)-, —(C₃₋₆ cycloalkenylene)-, 3- to 10-membered heterocycloalkylene, arylene, or heteroarylene.

In another embodiment, Y is selected from a bond; —NH—; —(C₁₋₆ alkylene)-O—; —O—(C₁₋₆ alkylene)-; —(C₂₋₆ alkenylene)-O—; —(C₁₋₆ alkylene)-C(O)—; —(C₂₋₆ alkenylene)-C(O)—; phenylene; piperidinylene; hydroxypiperidinylene; fluoropiperidinylene; azetidinylene; —C(O)— piperazinylene-; —(C₁₋₆ alkylene)-oxopiperazinylene-; pyrrolidinylene; 7- to 9-membered bridged bicyclic heterocycloalkylene; —(C₁₋₆ alkylene)-O-phenylene-; —(C₂₋₆ alkenylene)-O-piperidinylene; —(C₁₋₅ alkylene)-NH—, wherein 0, 1, or 2 methylene units are replaced with —O—; —NH—(C₁₋₅ alkylene)-NH—; —N(CH₃)—(C₁₋₅ alkylene)-NH—; —NH—(C₁₋₅ alkylene)-N(CH₃)—; —N(CH₃)—(C₁₋₅ alkylene)-N(CH₃)—; —(C₃₋₆ cycloalkylene)-NH—; —C(O)NH—(C₁₋₅ alkylene)-NH—; —C(O)NH—(C₃₋₆ cycloalkylene)-NH—; —(C₁₋₅ alkylene)-O—(C₃₋₆ cycloalkylene)-NH—; —(C₃₋₆ cycloalkenylene)-NH—; or

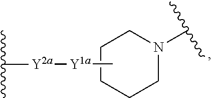

wherein Y^{1a} is a bond, —O—, —NH—, —NHC(O)—, —C(O)NH—, or C₁₋₃ alkylene; and Y^{2a} is a bond, —O—, —NH—, —NHC(O)—, —C(O)NH—, or C₁₋₃ alkylene. In another embodiment, Y is —NH—.

In another embodiment, Y is selected from the group consisting of:

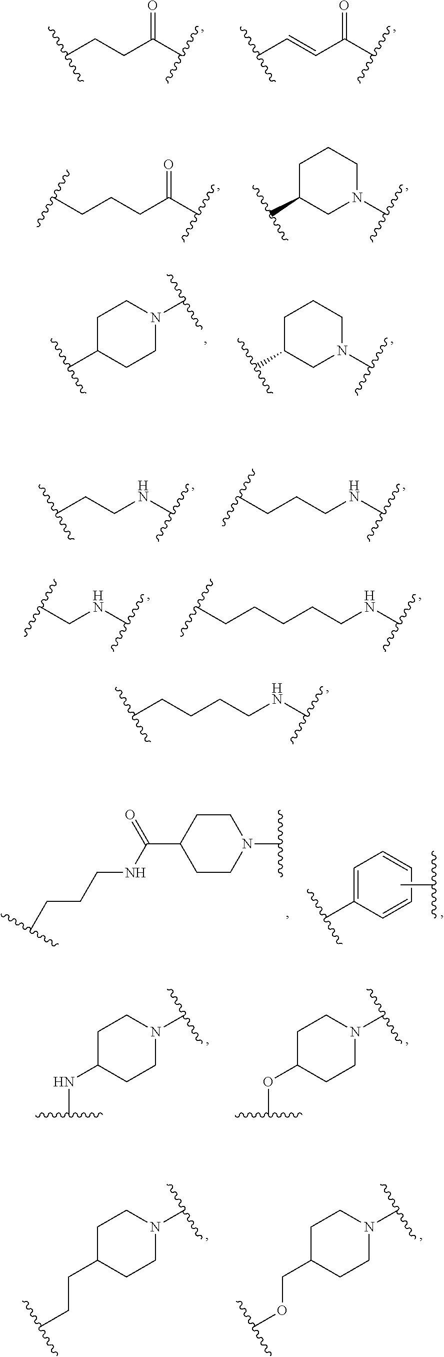

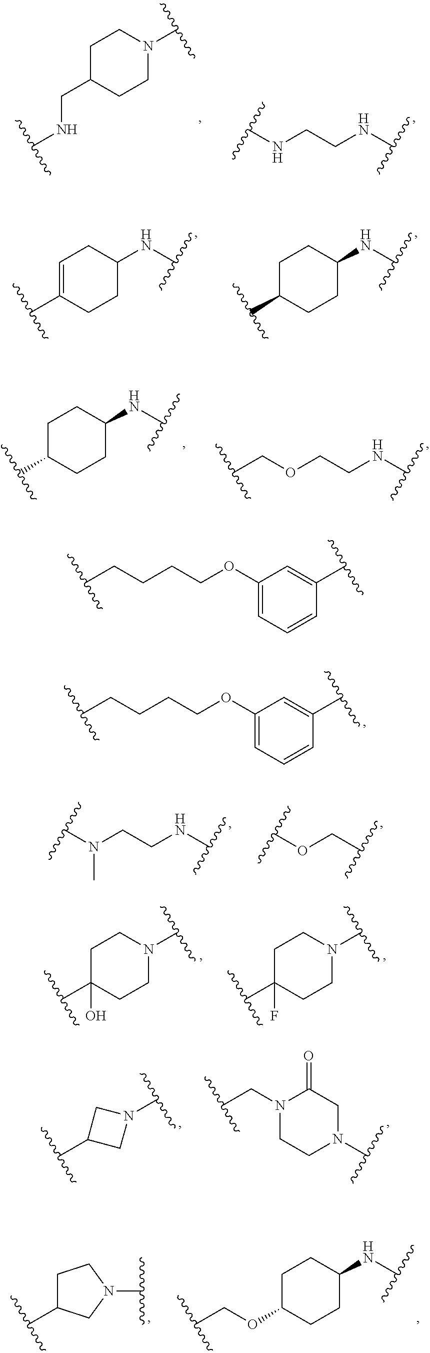

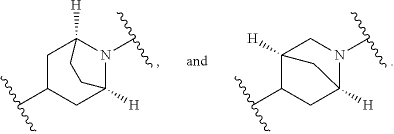 [SMILES: *C1C[C@H]2CC[C@@H](C1)N2* ; *C1C[C@H]2C[C@@H]1CN2*]

In another embodiment, Y is a bond. In another embodiment, Y is

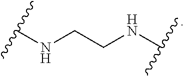 [SMILES: *NCCN*]

In another embodiment, Y is:

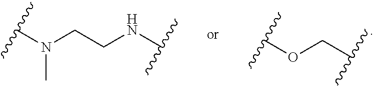

In another embodiment, R¹ is methyl, ethyl, 1-propyl, 2-propyl, 1-butyl, 2-butyl, or t-butyl. In another embodiment, R¹ is methyl. In another embodiment, R¹ is ethyl. In another embodiment, R¹ is cyclopropyl, cyclobutyl, cyclopentyl, or cyclohexyl.

In another embodiment, y of L′ is 2 to 8, 3 to 7, 4 to 7, or 5 to 7. In another embodiment, y of L′ is 1, 2, 3, 4, 5, 6, 7, 8, or 9.

In another embodiment, w of L′ is 0 to 4, 0 to 3, 0 to 2, or 1 to 2. In another embodiment, w of L′ is 0, 1, 2, 3, 4, or 5.

In another embodiment, L′ is

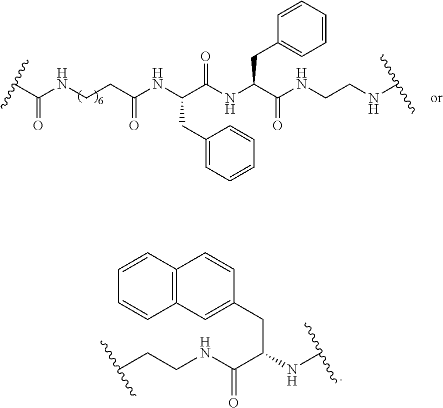

In another embodiment, L′ is bond.

In another embodiment, T is

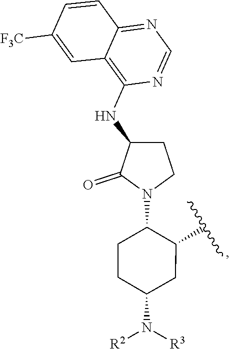

wherein R² of Formula A is hydrogen or C₁₋₄ alkyl; and R³ of Formula A is hydrogen or C₁₋₄ alkyl. In another embodiment, R² and R³ of Formula A are each independently hydrogen, methyl, ethyl, 1-propyl, 2-propyl, 1-butyl, 2-butyl, or t-butyl. In another embodiment, R² of Formula A is isopropyl and R³ of Formula A is methyl. In another embodiment, R² of Formula A is t-butyl and R³ of Formula A is hydrogen. In another embodiment, T is

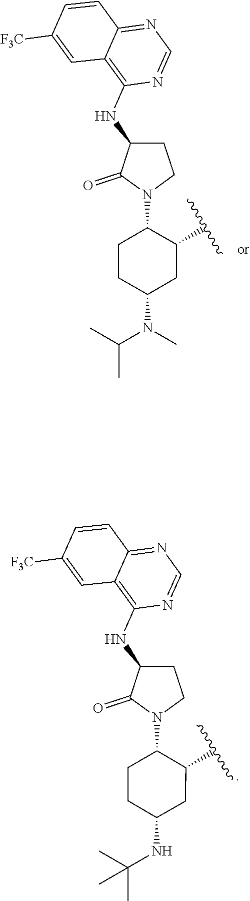 [SMILES: *[C@@H]1C[C@H](N(C)C(C)C)CC[C@@H]1N1CC[C@H](Nc2ncnc3ccc(C(F)(F)F)cc23)C1=O ; *[C@@H]1C[C@H](NC(C)(C)C)CC[C@@H]1N1CC[C@H](Nc2ncnc3ccc(C(F)(F)F)cc23)C1=O]

In another embodiment, T is:

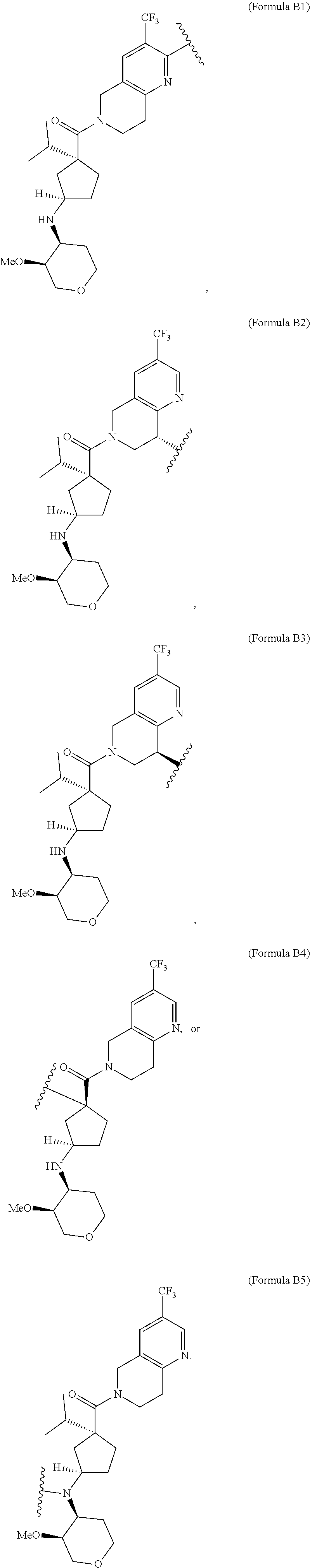 [SMILES: *c1nc2c(cc1C(F)(F)F)CN(C(=O)[C@@]1(C(C)C)CC[C@@H](N[C@H]3CCOC[C@H]3OC)C1)CC2 ; *[C@@H]1CN(C(=O)[C@@]2(C(C)C)CC[C@@H](N[C@H]3CCOC[C@H]3OC)C2)Cc2cc(C(F)(F)F)cnc21 ; *[C@H]1CN(C(=O)[C@@]2(C(C)C)CC[C@@H](N[C@H]3CCOC[C@H]3OC)C2)Cc2cc(C(F)(F)F)cnc21 ; *[C@]1(C(=O)N2CCc3ncc(C(F)(F)F)cc3C2)CC[C@@H](N[C@H]2CCOC[C@H]2OC)C1 ; *N([C@@H]1CC[C@@](C(=O)N2CCc3ncc(C(F)(F)F)cc3C2)(C(C)C)C1)[C@H]1CCOC[C@H]1OC]

In another embodiment, T is

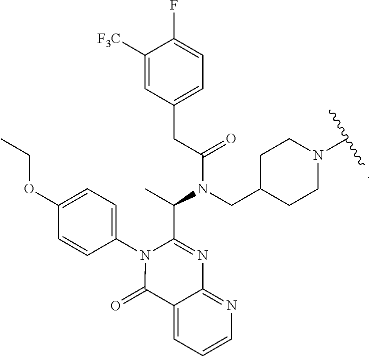 [SMILES: *N1CCC(CN(C(=O)Cc2ccc(F)c(C(F)(F)F)c2)[C@H](C)c2nc3ncccc3c(=O)n2-c2ccc(OCC)cc2)CC1]

In another embodiment, T is

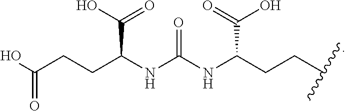 [SMILES: *CC[C@H](NC(=O)N[C@@H](CCC(=O)O)C(=O)O)C(=O)O]

In another embodiment, T is

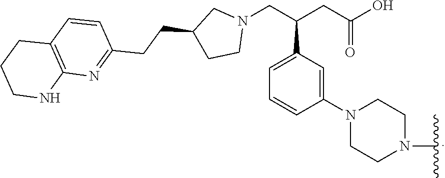 [SMILES: *N1CCN(c2cccc([C@H](CC(=O)O)CN3CC[C@@H](CCc4ccc5c(n4)NCCC5)C3)c2)CC1]

In another embodiment, T is

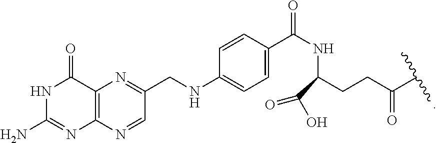 [SMILES: *C(=O)CC[C@H](NC(=O)c1ccc(NCc2cnc3nc(N)[nH]c(=O)c3n2)cc1)C(=O)O]

In another embodiment, T is:

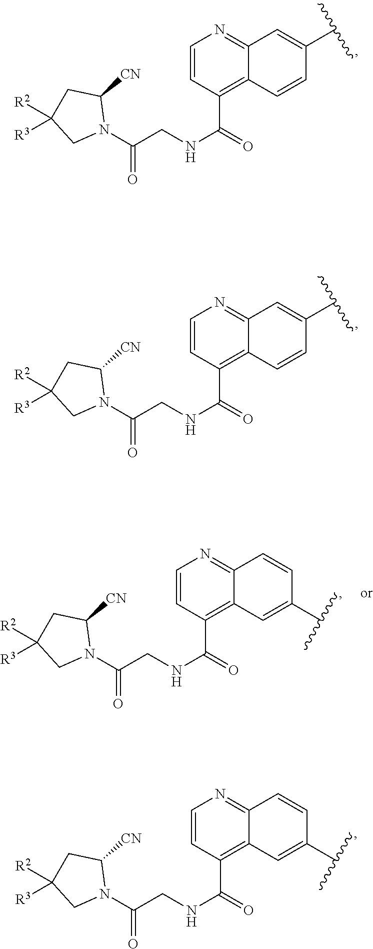

wherein R² and R³ of Formula G1, Formula G2, Formula G3, and Formula G4 are each independently F or H.

In another embodiment, T is

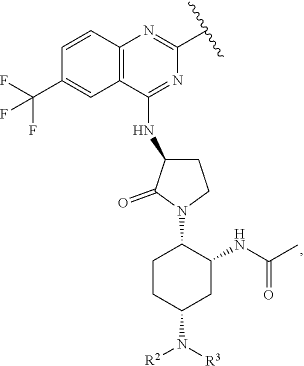

wherein R² of Formula H is hydrogen or C₁₋₄ alkyl; and R³ of Formula H is hydrogen or C₁₋₄ alkyl. In another embodiment, R² and R³ of Formula H are each independently hydrogen, methyl, ethyl, 1-propyl, 2-propyl, 1-butyl, 2-butyl, or t-butyl. In another embodiment, R² of Formula H is isopropyl and R³ of Formula H is methyl. In another embodiment, R² of Formula H is t-butyl and R³ of Formula H is hydrogen.

In another embodiment, T is

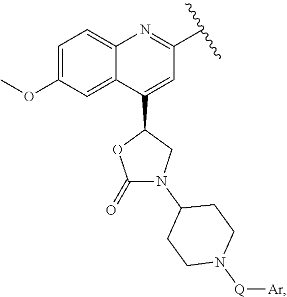

wherein Q is C₁₋₅ alkylene, wherein 0, 1, or 2 methylene units are replaced with —O—; and Ar is an optionally substituted 5- to 10-membered aromatic ring or 9- or 10-membered unsaturated fused bicyclic ring.

In another embodiment, Q is —CH₂—, —CH(CH₃)—, —CH₂CH₂CH₂—, —CH(CH₂CH₃)—, or —CH₂CH₂O—. In another embodiment, Q is —CH₂— or —CH(CH₃)—. In another embodiment, Q is —CH(CH₃)—.

In another embodiment, Ar is an optionally substituted 5-, 6-, 7-, 8-, 9-, or 10-membered aromatic ring. In another embodiment, Ar is an optionally substituted 6-membered aromatic ring. In another embodiment, Ar is an optionally substituted 9-membered aromatic ring. In another embodiment, Ar is an optionally substituted 9- or 10-membered unsaturated fused bicyclic ring. In another embodiment, Ar is an optionally substituted 9-membered unsaturated fused bicyclic ring.

In another embodiment, Ar is phenyl, pyridinyl, indolyl, indolinyl, dihydrobenzofuranyl, or benzofuranyl, and each Ar is substituted with 0, 1, or 2 substituent groups. In another embodiment, Ar is phenyl, 2-pyridinyl, 3-pyridinyl, 4-pyridinyl, 2-indolyl, 3-indolyl, 4-indolyl, 5-indolyl, 6-indolyl, 7-indolyl, 2-indolinyl, 3-indolinyl, 4-indolinyl, 5-indolinyl, 6-indolinyl, 7-indolinyl, 2-dihydrobenzofuranyl, 3-dihydrobenzofuranyl, 4-dihydrobenzofuranyl, 5-dihydrobenzofuranyl, 6-dihydrobenzofuranyl, 7-dihydrobenzofuranyl, 2-benzofuranyl, 3-benzofuranyl, 4-benzofuranyl, 5-benzofuranyl, 6-benzofuranyl, or 7-benzofuranyl, and each Ar is substituted with 0, 1, or 2 substituent groups.

In another embodiment, the Ar substituent groups are independently selected from C₁-3 alkyl, C₁₋₃ alkoxy, C₁₋₃ haloalkyl, C₁₋₃ haloalkoxy, or halo. In another embodiment, the Ar substituent groups are independently selected from methyl, ethyl, methoxy, ethoxy, bromo, chloro, or trifluoromethyl.

In another embodiment, Ar is

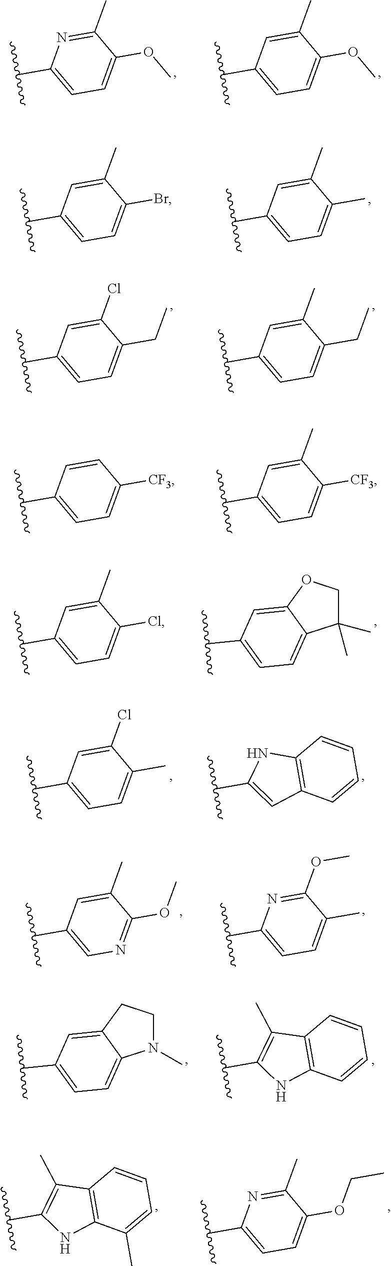

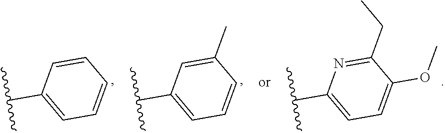

In another embodiment, the compound of Formula (I) is selected from a compound as listed in Table 2:

TABLE 2

| Cpd. | |
|---|---|
| No. | Structure |
| 1 | 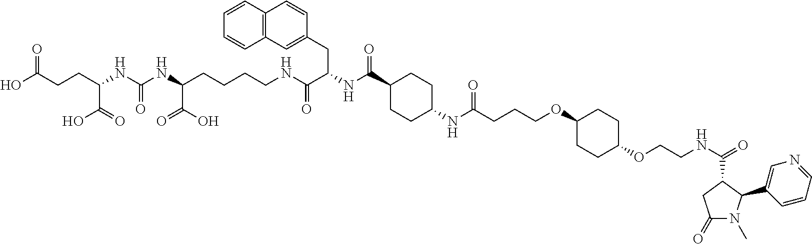 [SMILES: CN1C(=O)C[C@H](C(=O)NCCO[C@H]2CC[C@H](OCCCC(=O)N[C@H]3CC[C@H](C(=O)N[C@@H](Cc4ccc5ccccc5c4)C(=O)NCCCC[C@H](NC(=O)N[C@@H](CCC(=O)O)C(=O)O)C(=O)O)CC3)CC2)[C@H]1c1cccnc1] |
| 2 | 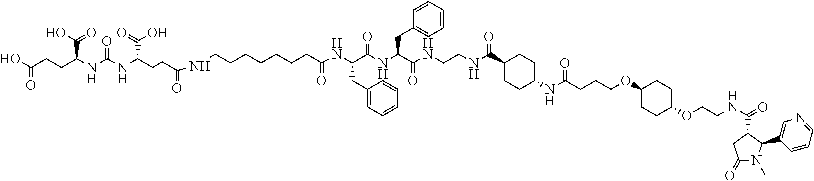 [SMILES: CN1C(=O)C[C@H](C(=O)NCCO[C@H]2CC[C@H](OCCCC(=O)N[C@H]3CC[C@H](C(=O)NCCNC(=O)[C@H](Cc4ccccc4)NC(=O)[C@H](Cc4ccccc4)NC(=O)CCCCCCCNC(=O)CC[C@H](NC(=O)N[C@@H](CCC(=O)O)C(=O)O)C(=O)O)CC3)CC2)[C@H]1c1cccnc1] |
| 3 | 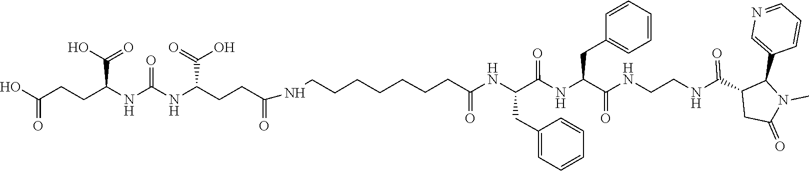 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| 4 | 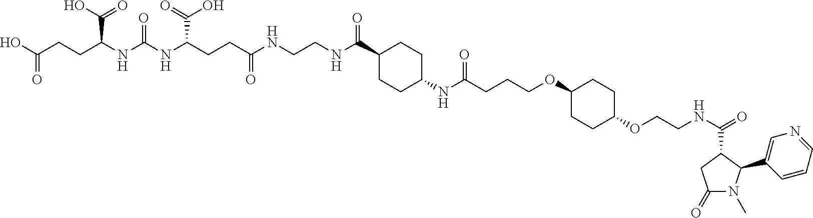 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)N[C@@H](CCC(=O)O)C(=O)O)C(=O)O)CC3)CC2)[C@H]1c1cccnc1] |
| 5 | 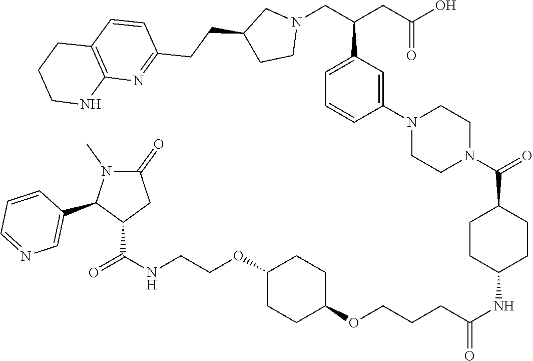 [SMILES: CN1C(=O)C[C@H](C(=O)NCCO[C@H]2CC[C@H](OCCCC(=O)N[C@H]3CC[C@H](C(=O)N4CCN(c5cccc([C@H](CC(=O)O)CN6CC[C@@H](CCc7ccc8c(n7)NCCC8)C6)c5)CC4)CC3)CC2)[C@H]1c1cccnc1] |
| 6 | 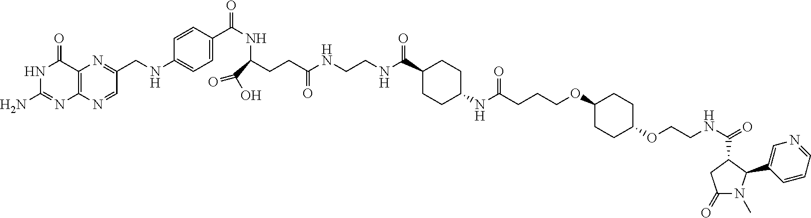 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)c4ccc(NCc5cnc6nc(N)[nH]c(=O)c6n5)cc4)C(=O)O)CC3)CC2)[C@H]1c1cccnc1] |
| 7 | 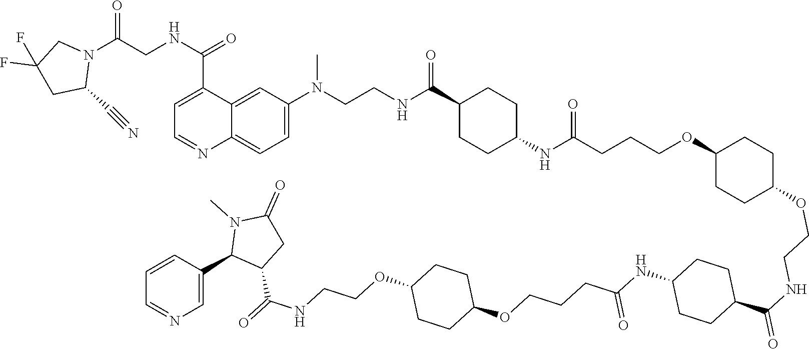 [SMILES: CN(CCNC(=O)[C@H]1CC[C@H](NC(=O)CCCO[C@H]2CC[C@H](OCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)CC1)c1ccc2nccc(C(=O)NCC(=O)N3CC(F)(F)C[C@H]3C#N)c2c1] |
| 8 | 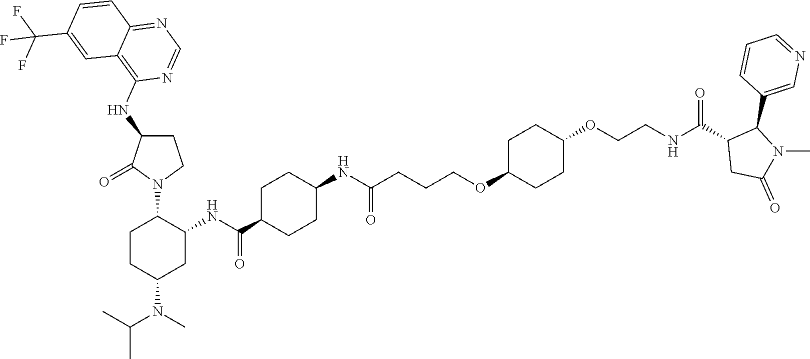 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)C1] |

In another embodiment, the compound of Formula (I) is selected from a compound as listed in Table 3:

TABLE 3

| Com- | |
|---|---|
| pound | Structure |
| A1 | 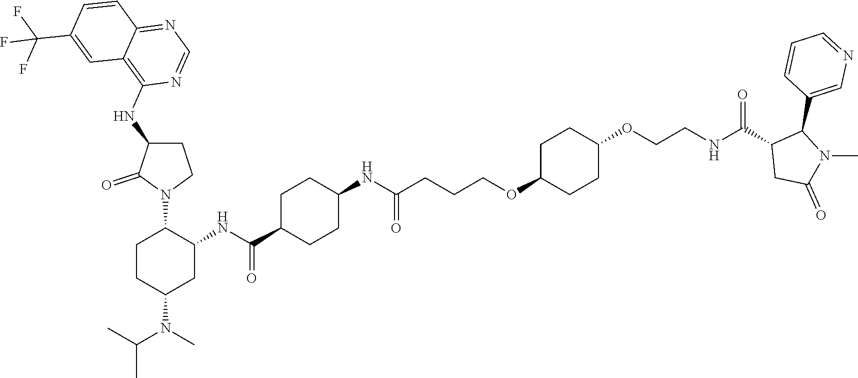 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)C1] |
| A2 | 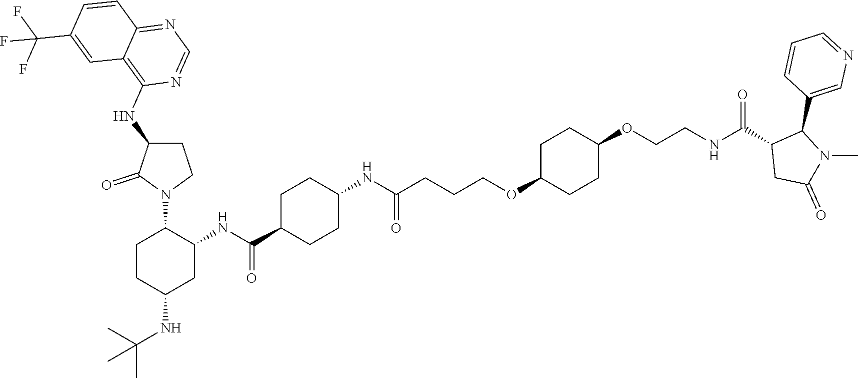 [SMILES: CN1C(=O)C[C@H](C(=O)NCCO[C@H]2CC[C@@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A3 | 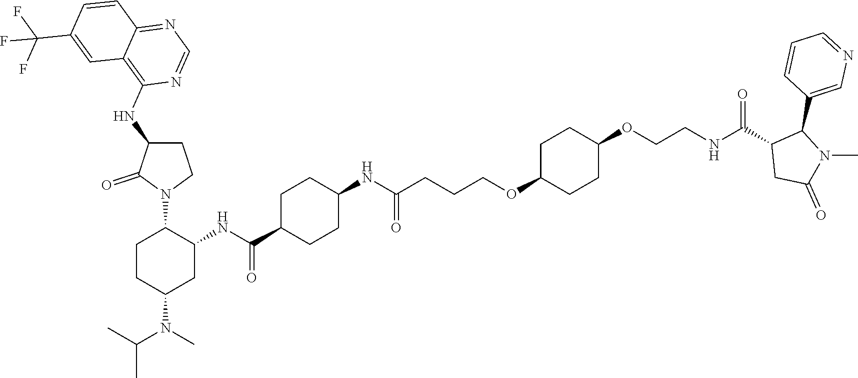 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)C1] |
| A4 | 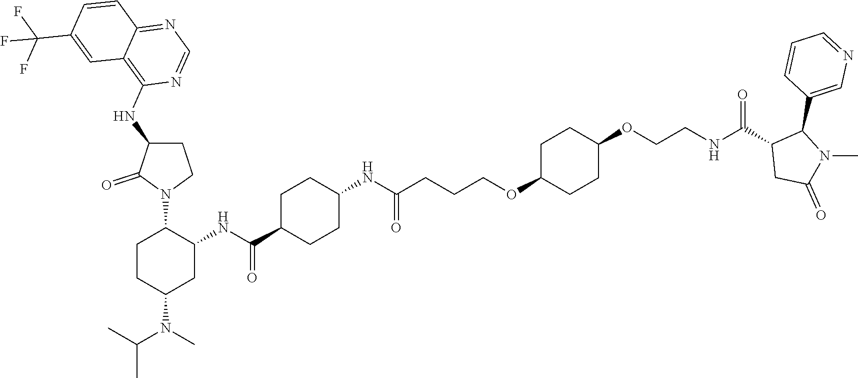 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)C1] |
| A5 | 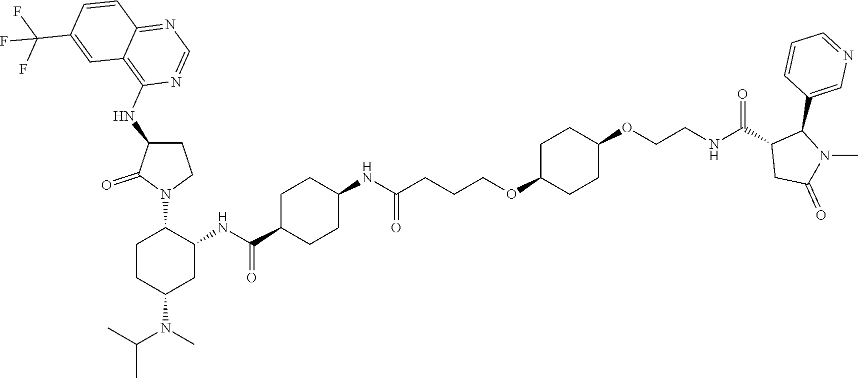 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)C1] |
| A6 | 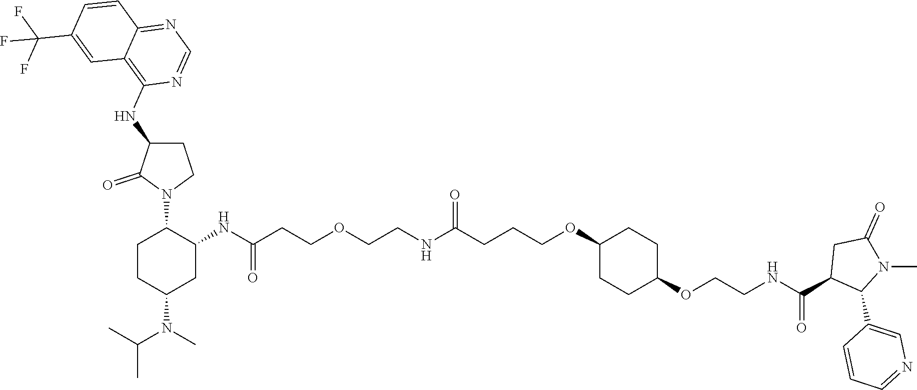 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCCO[C@H]2CC[C@@H](OCCNC(=O)[C@H]3CC(=O)N(C)[C@@H]3c3cccnc3)CC2)C1] |
| A7 | 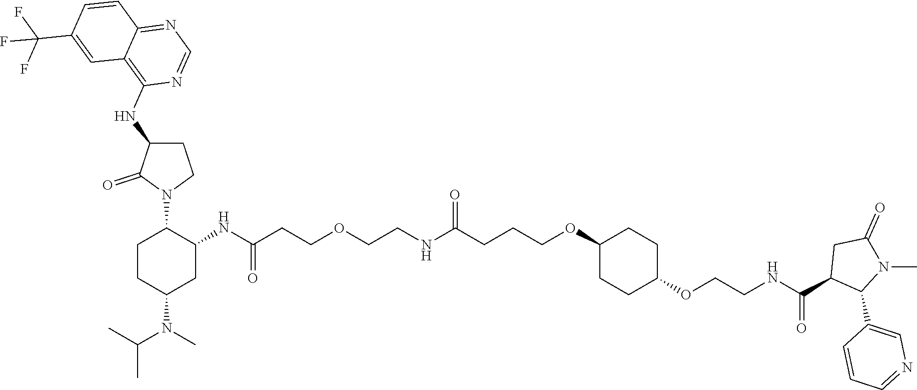 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCCO[C@H]2CC[C@H](OCCNC(=O)[C@H]3CC(=O)N(C)[C@@H]3c3cccnc3)CC2)C1] |
| A8 | 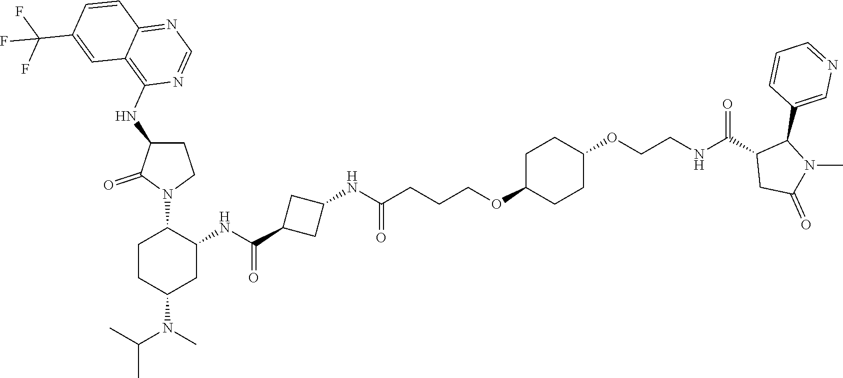 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCCO[C@H]3CC[C@H](OCCNC(=O)[C@H]4CC(=O)N(C)[C@@H]4c4cccnc4)CC3)C2)C1] |
| A9 | 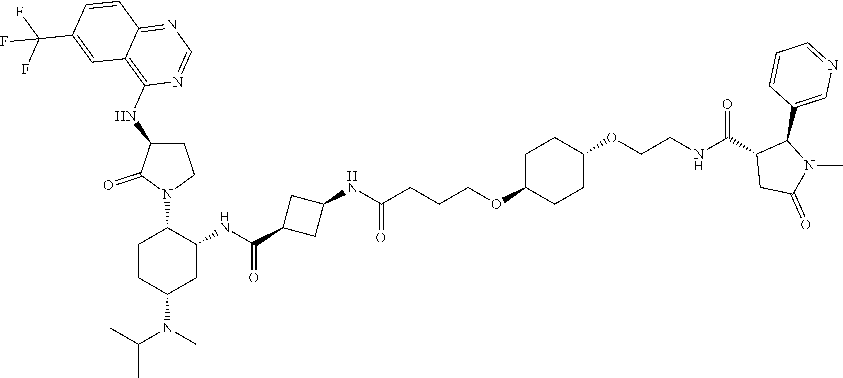 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCCO[C@H]3CC[C@H](OCCNC(=O)[C@H]4CC(=O)N(C)[C@@H]4c4cccnc4)CC3)C2)C1] |
| A10 | 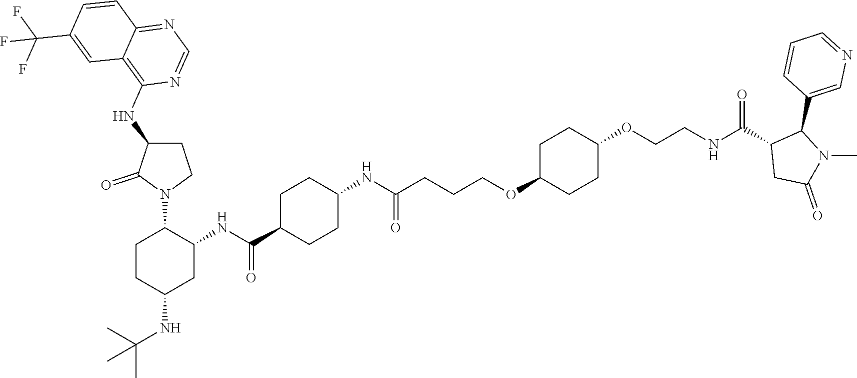 [SMILES: CN1C(=O)C[C@H](C(=O)NCCO[C@H]2CC[C@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A11 | 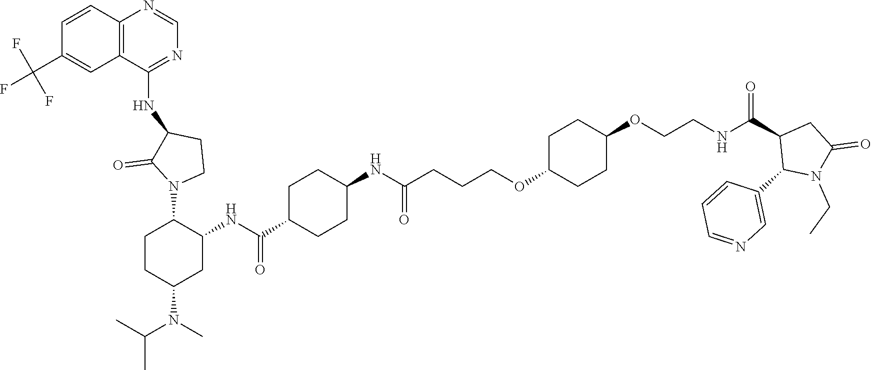 [SMILES: CCN1C(=O)C[C@H](C(=O)NCCO[C@H]2CC[C@H](OCCCC(=O)N[C@H]3CC[C@H](C(=O)N[C@@H]4C[C@H](N(C)C(C)C)CC[C@@H]4N4CC[C@H](Nc5ncnc6ccc(C(F)(F)F)cc56)C4=O)CC3)CC2)[C@H]1c1cccnc1] |
| A12 | 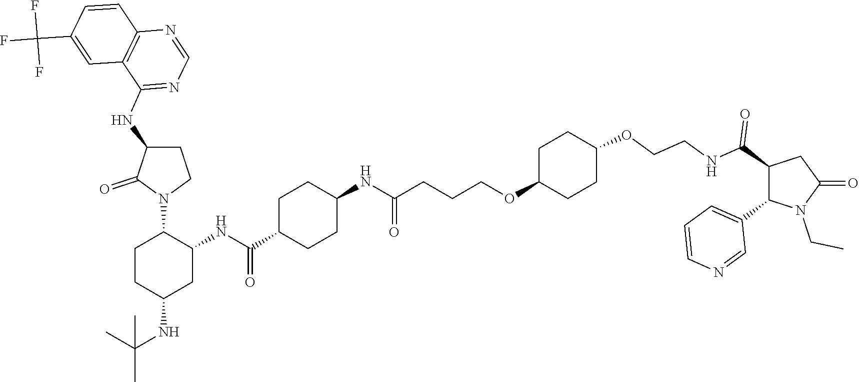 [SMILES: CCN1C(=O)C[C@H](C(=O)NCCO[C@H]2CC[C@H](OCCCC(=O)N[C@H]3CC[C@H](C(=O)N[C@@H]4C[C@H](NC(C)(C)C)CC[C@@H]4N4CC[C@H](Nc5ncnc6ccc(C(F)(F)F)cc56)C4=O)CC3)CC2)[C@H]1c1cccnc1] |
| A13 | 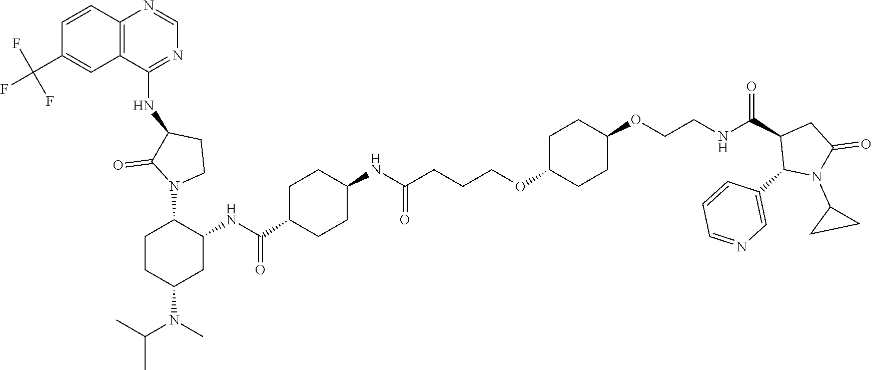 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CC5)[C@@H]4c4cccnc4)CC3)CC2)C1] |
| A14 | 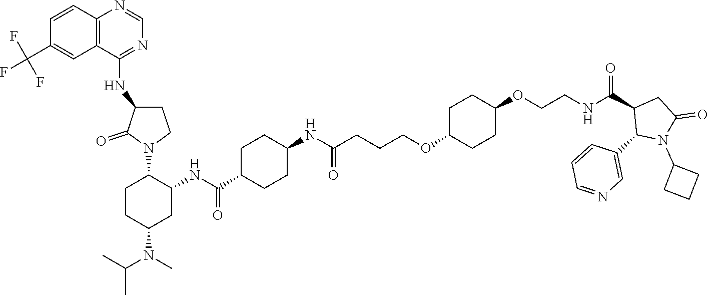 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CCC5)[C@@H]4c4cccnc4)CC3)CC2)C1] |
| A15 | 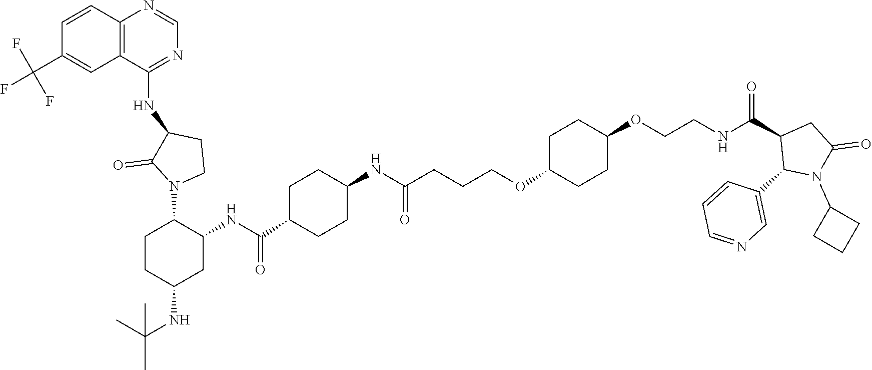 [SMILES: CC(C)(C)N[C@@H]1CC[C@H](N2CC[C@H](Nc3ncnc4ccc(C(F)(F)F)cc34)C2=O)[C@H](NC(=O)[C@H]2CC[C@H](NC(=O)CCCO[C@H]3CC[C@H](OCCNC(=O)[C@H]4CC(=O)N(C5CCC5)[C@@H]4c4cccnc4)CC3)CC2)C1] |
| A15a | 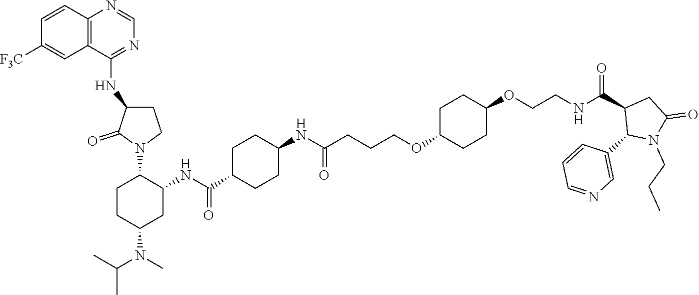 [SMILES: CCCN1C(=O)C[C@H](C(=O)NCCO[C@H]2CC[C@H](OCCCC(=O)N[C@H]3CC[C@H](C(=O)N[C@@H]4C[C@H](N(C)C(C)C)CC[C@@H]4N4CC[C@H](Nc5ncnc6ccc(C(F)(F)F)cc56)C4=O)CC3)CC2)[C@H]1c1cccnc1] |
| A16 | 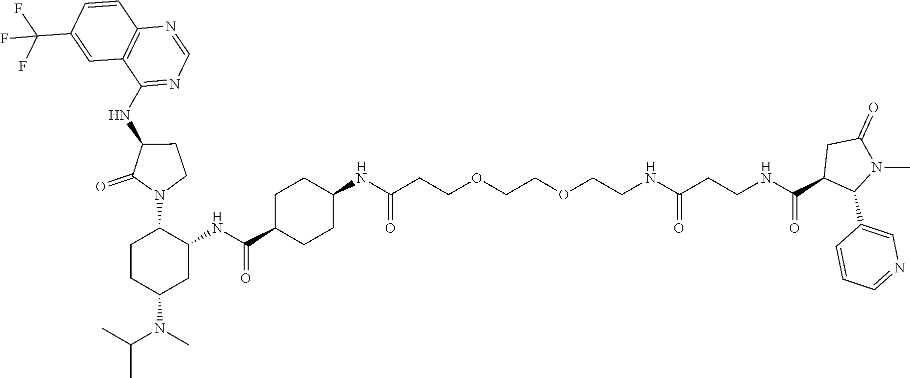 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCNC(=O)CCNC(=O)[C@H]3CC(=O)N(C)[C@@H]3c3cccnc3)CC2)C1] |
| A17 | 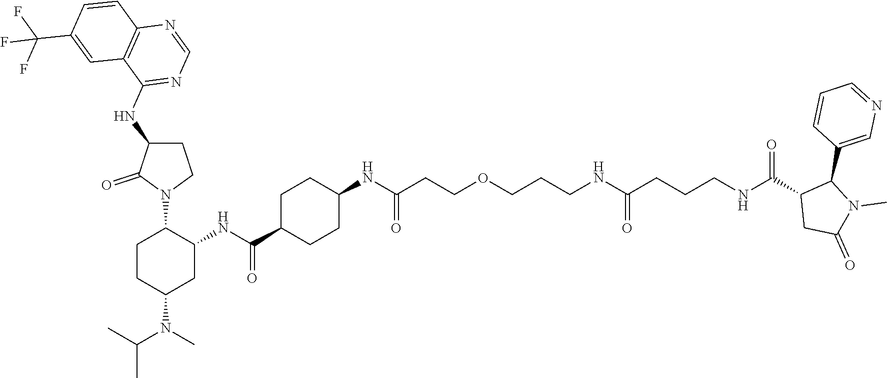 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCCNC(=O)CCCNC(=O)[C@H]3CC(=O)N(C)[C@@H]3c3cccnc3)CC2)C1] |
| A18 | 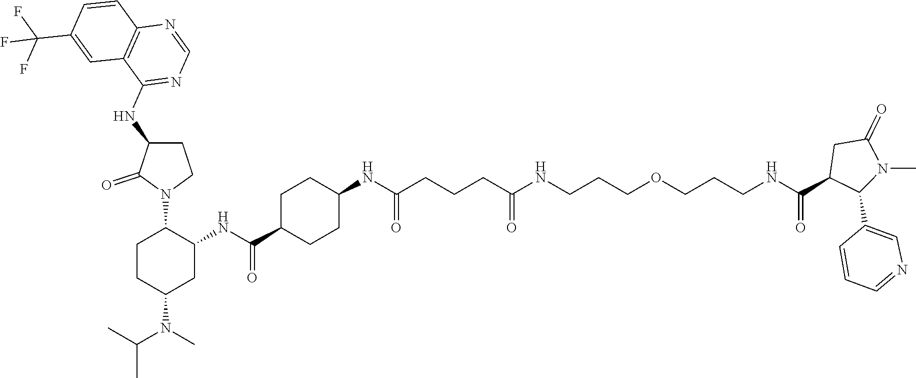 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCC(=O)NCCCOCCCNC(=O)[C@H]3CC(=O)N(C)[C@@H]3c3cccnc3)CC2)C1] |
| A19 | 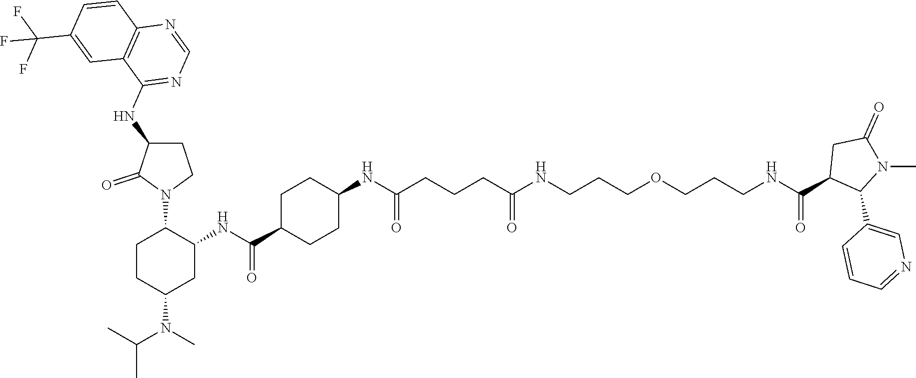 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCC(=O)NCCCOCCCNC(=O)[C@H]3CC(=O)N(C)[C@@H]3c3cccnc3)CC2)C1] |
| A20 | 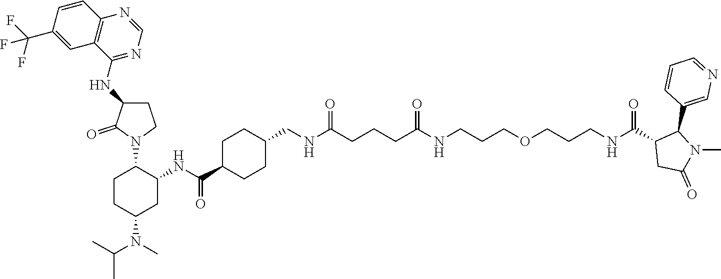 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](CNC(=O)CCCC(=O)NCCCOCCCNC(=O)[C@H]3CC(=O)N(C)[C@@H]3c3cccnc3)CC2)C1] |
| A21 | 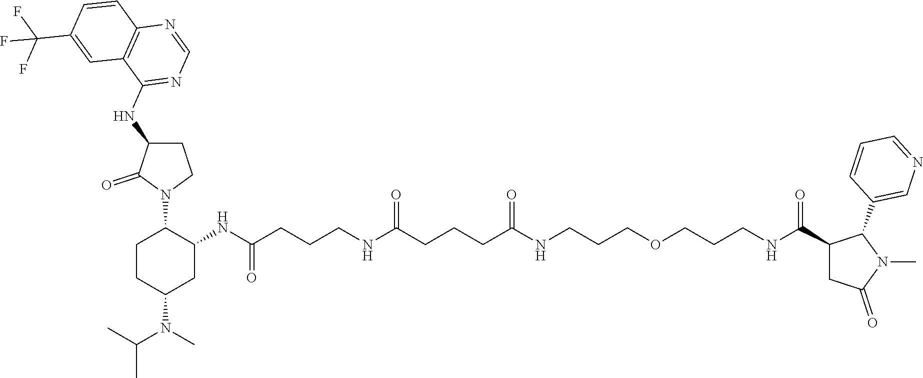 [SMILES: CC(C)N(C)[C@@H]1CC[C@H](N2CC[C@H](Nc3ncnc4ccc(C(F)(F)F)cc34)C2=O)[C@H](NC(=O)CCCNC(=O)CCCC(=O)NCCCOCCCNC(=O)[C@@H]2CC(=O)N(C)[C@H]2c2cccnc2)C1] |
| A22 | 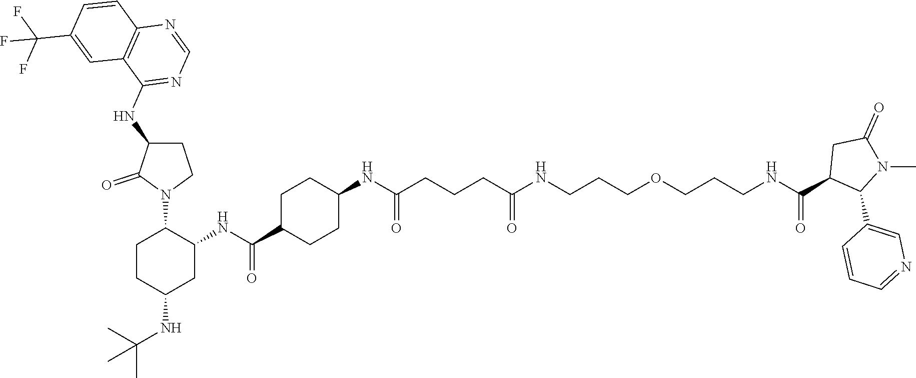 |
| A23 | 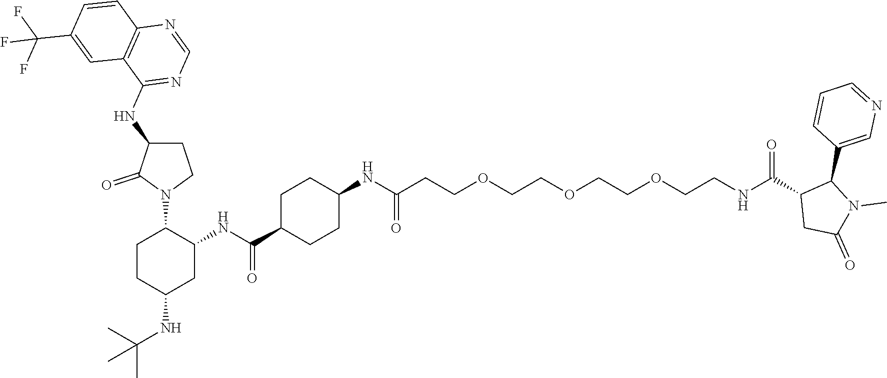 [SMILES: CN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A24 | 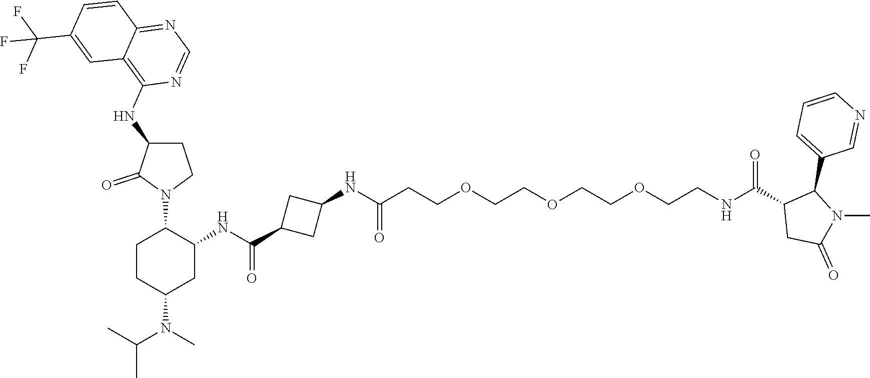 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCOCCNC(=O)[C@H]3CC(=O)N(C)[C@@H]3c3cccnc3)C2)C1] |
| A25 | 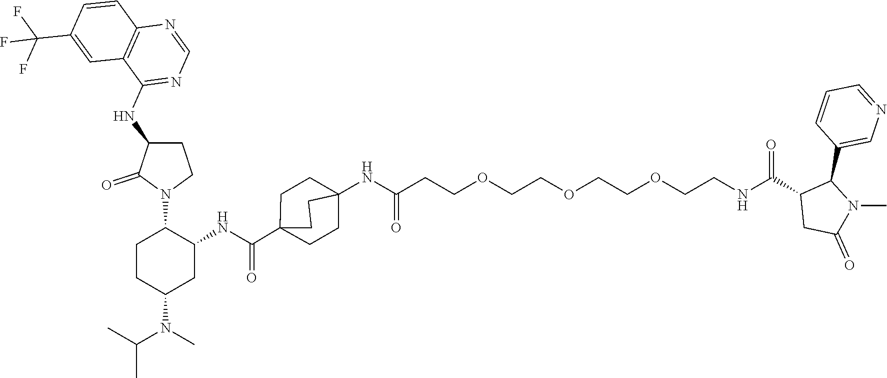 [SMILES: CC(C)N(C)[C@@H]1CC[C@H](N2CC[C@H](Nc3ncnc4ccc(C(F)(F)F)cc34)C2=O)[C@H](NC(=O)C23CCC(NC(=O)CCOCCOCCOCCNC(=O)[C@H]4CC(=O)N(C)[C@@H]4c4cccnc4)(CC2)CC3)C1] |
| A26 | 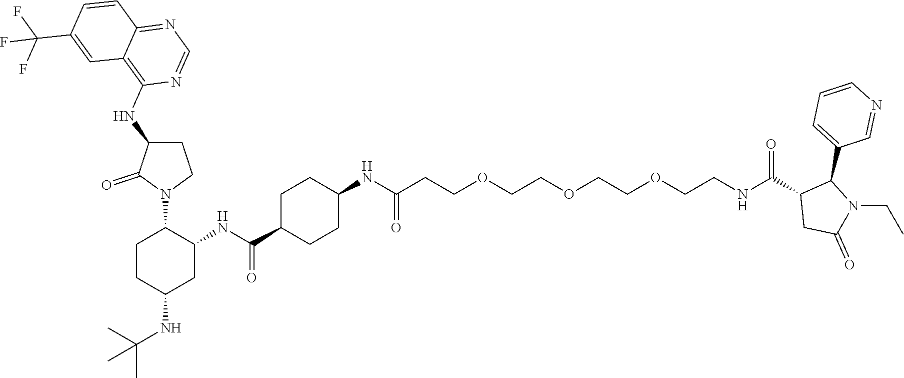 [SMILES: CCN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A27 | 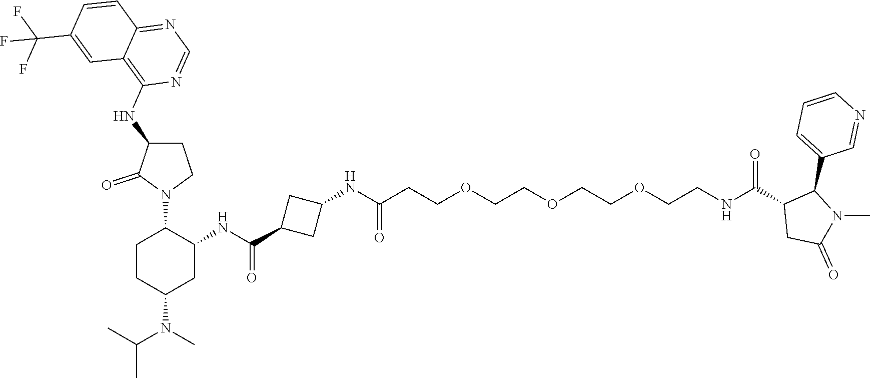 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOCCOCCOCCNC(=O)[C@H]3CC(=O)N(C)[C@@H]3c3cccnc3)C2)C1] |
| A28 | 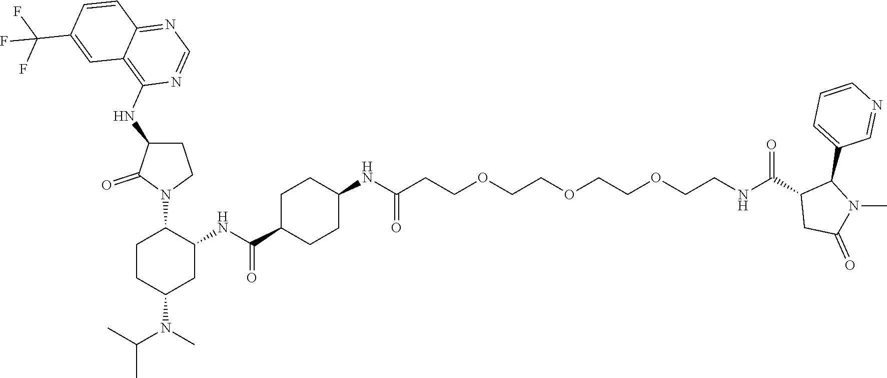 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCOCCNC(=O)[C@H]3CC(=O)N(C)[C@@H]3c3cccnc3)CC2)C1] |
| A29 | 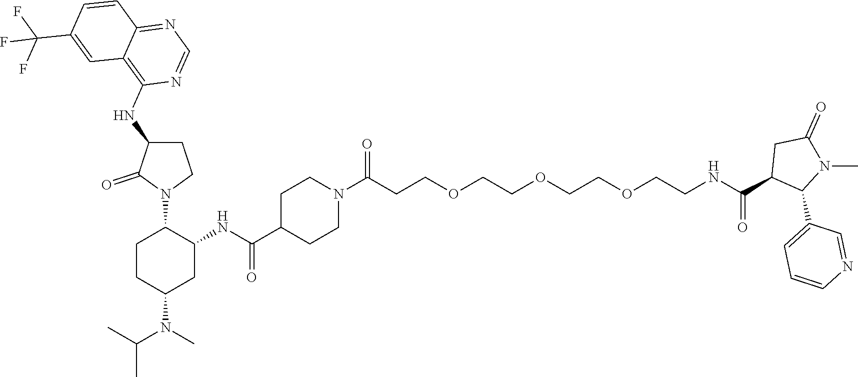 [SMILES: CC(C)N(C)[C@@H]1CC[C@H](N2CC[C@H](Nc3ncnc4ccc(C(F)(F)F)cc34)C2=O)[C@H](NC(=O)C2CCN(C(=O)CCOCCOCCOCCNC(=O)[C@H]3CC(=O)N(C)[C@@H]3c3cccnc3)CC2)C1] |
| A30 | 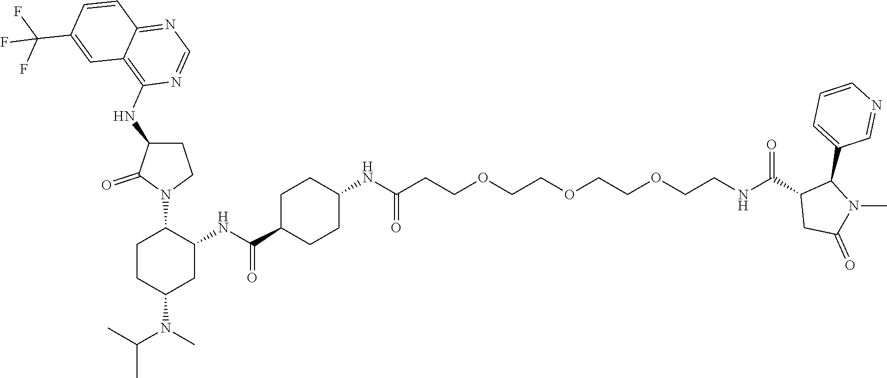 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCOCCOCCOCCNC(=O)[C@H]3CC(=O)N(C)[C@@H]3c3cccnc3)CC2)C1] |
| A31 | 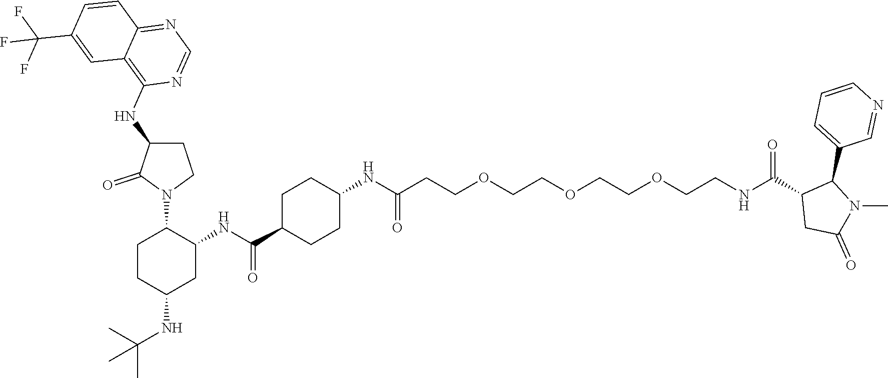 [SMILES: CN1C(=O)C[C@H](C(=O)NCCOCCOCCOCCC(=O)N[C@H]2CC[C@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A32 | 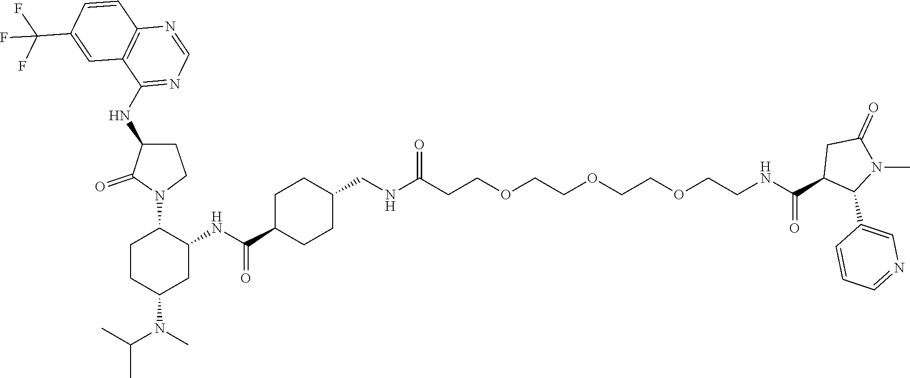 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](CNC(=O)CCOCCOCCOCCNC(=O)[C@H]3CC(=O)N(C)[C@@H]3c3cccnc3)CC2)C1] |
| A33 | 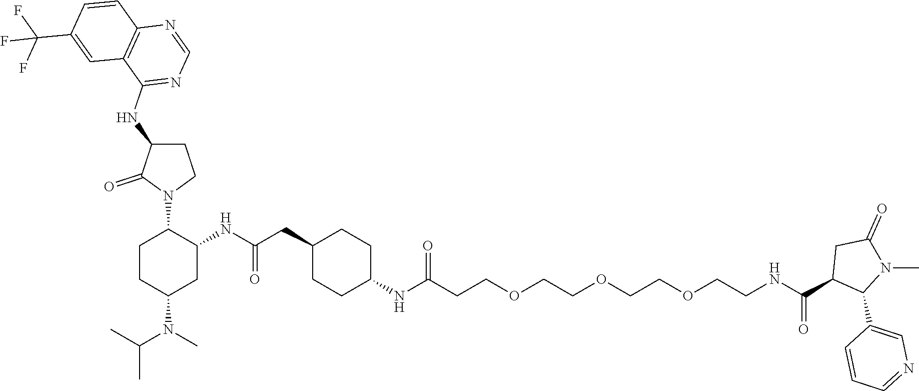 [SMILES: CC(C)N(C)[C@@H]1CC[C@H](N2CC[C@H](Nc3ncnc4ccc(C(F)(F)F)cc34)C2=O)[C@H](NC(=O)C[C@H]2CC[C@H](NC(=O)CCOCCOCCOCCNC(=O)[C@H]3CC(=O)N(C)[C@@H]3c3cccnc3)CC2)C1] |
| A34 | 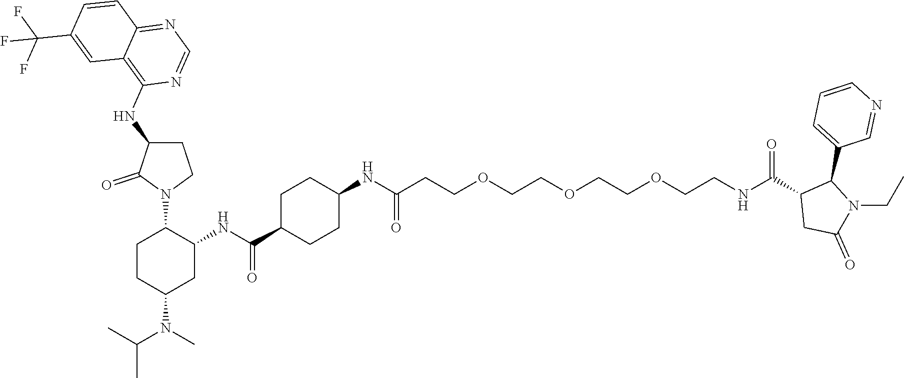 [SMILES: CCN1C(=O)C[C@H](C(=O)NCCOCCOCCOCCC(=O)N[C@H]2CC[C@@H](C(=O)N[C@@H]3C[C@H](N(C)C(C)C)CC[C@@H]3N3CC[C@H](Nc4ncnc5ccc(C(F)(F)F)cc45)C3=O)CC2)[C@H]1c1cccnc1] |
| A35 | 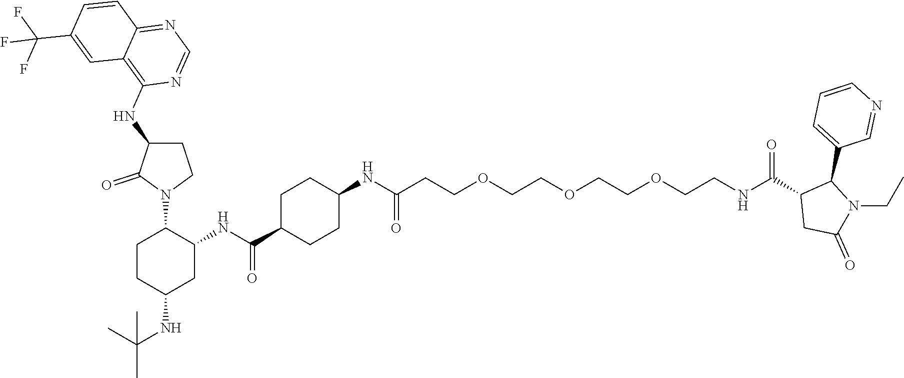 [SMILES: CCN1C(=O)C[C@H](C(=O)NCCOCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A36 | 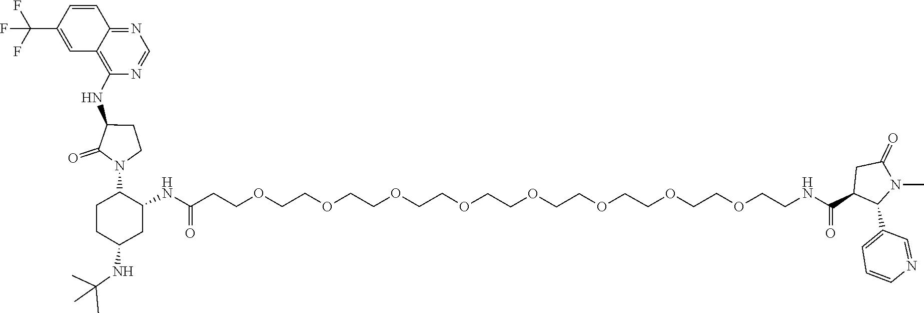 |
| A37 | 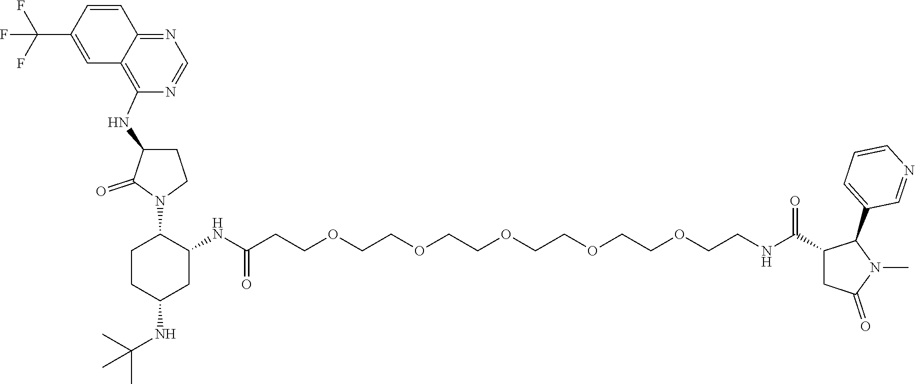 [SMILES: CN1C(=O)C[C@H](C(=O)NCCOCCOCCOCCOCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A38 | 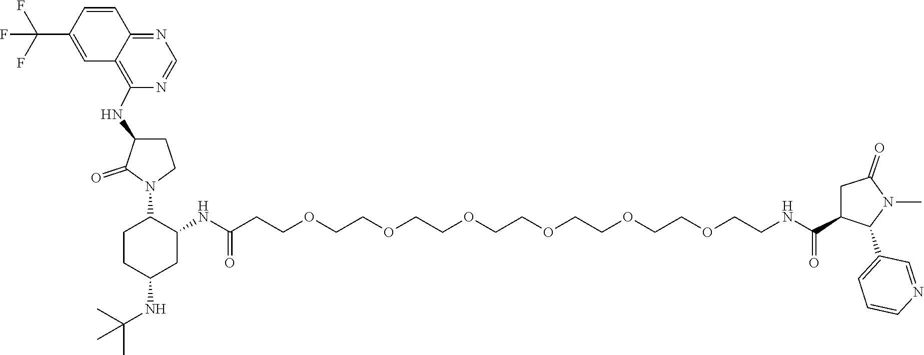 |
| A39 | 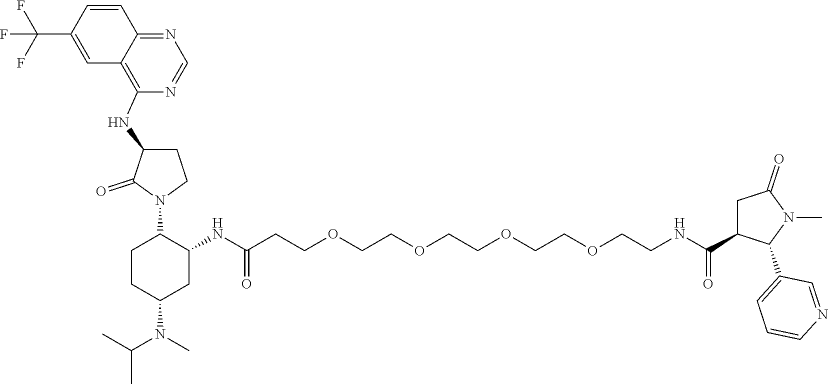 |
| A40 | 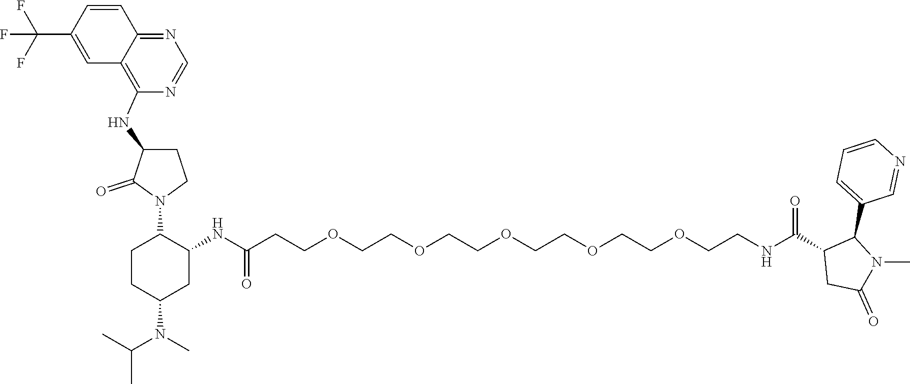 |
| A41 | 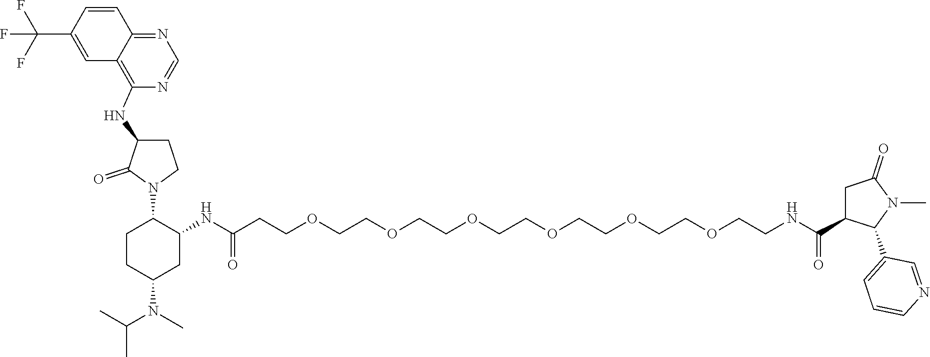 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCOCCOCCOCCNC(=O)[C@H]2CC(=O)N(C)[C@@H]2c2cccnc2)C1] |
| A42 | 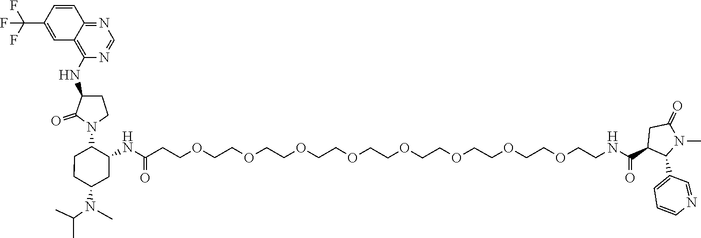 |
| A43 | 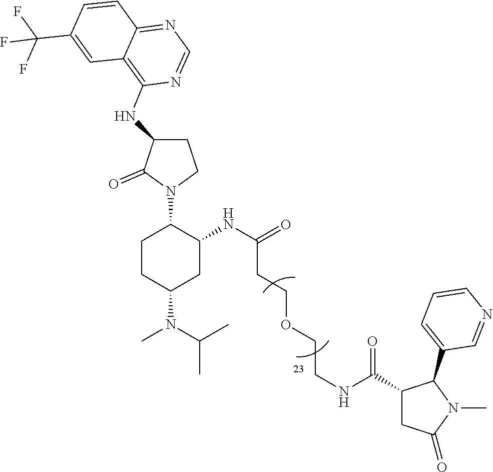 |
| A44 | 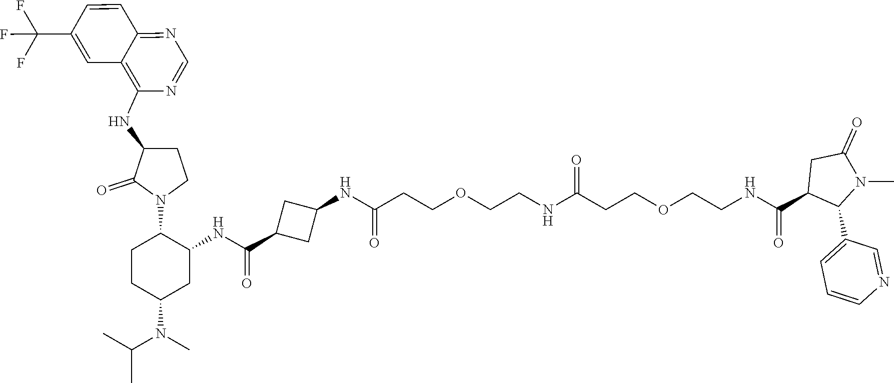 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCNC(=O)CCOCCNC(=O)[C@H]3CC(=O)N(C)[C@@H]3c3cccnc3)C2)C1] |
| A45 | 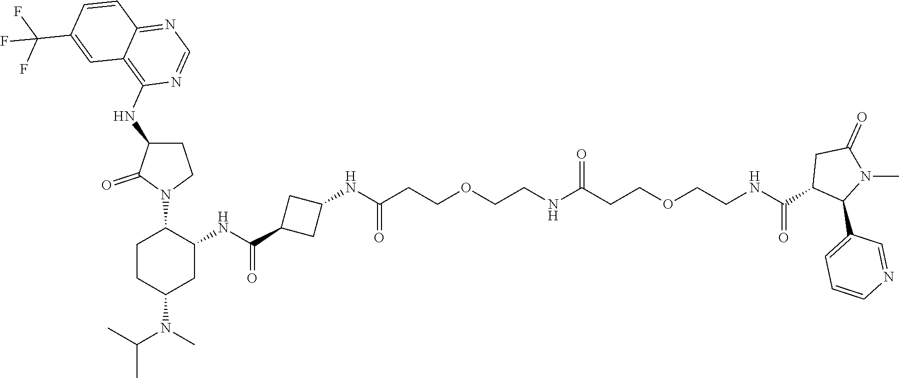 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOCCNC(=O)CCOCCNC(=O)[C@@H]3CC(=O)N(C)[C@H]3c3cccnc3)C2)C1] |
| A46 | 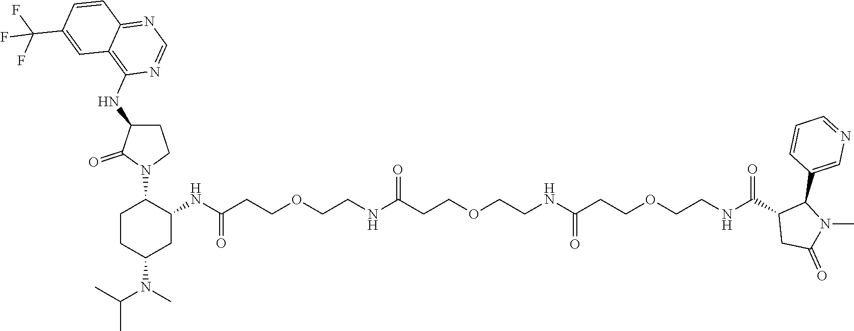 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCOCCNC(=O)CCOCCNC(=O)[C@H]2CC(=O)N(C)[C@@H]2c2cccnc2)C1] |
| A47 | 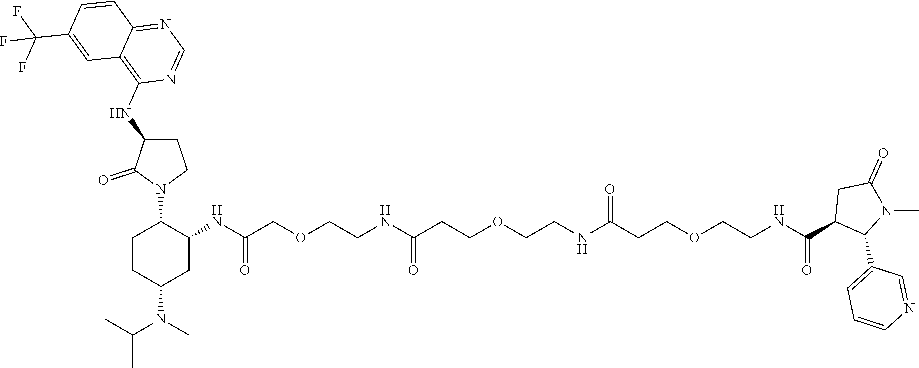 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCNC(=O)CCOCCNC(=O)[C@H]2CC(=O)N(C)[C@@H]2c2cccnc2)C1] |
| A48 | 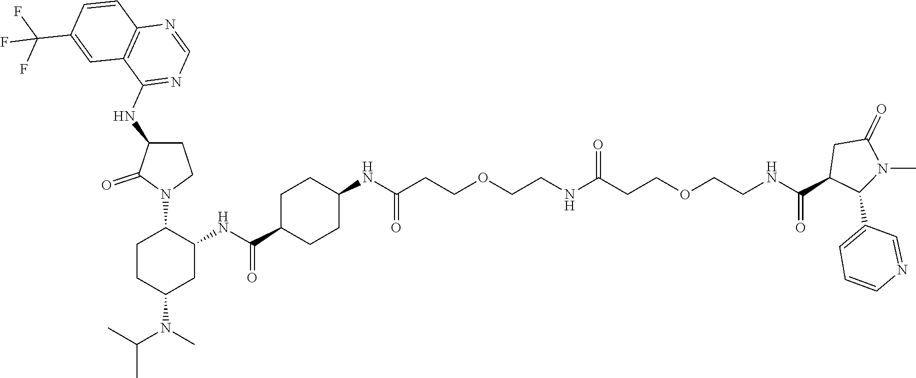 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCOCCNC(=O)[C@H]3CC(=O)N(C)[C@@H]3c3cccnc3)CC2)C1] |
| A49 | 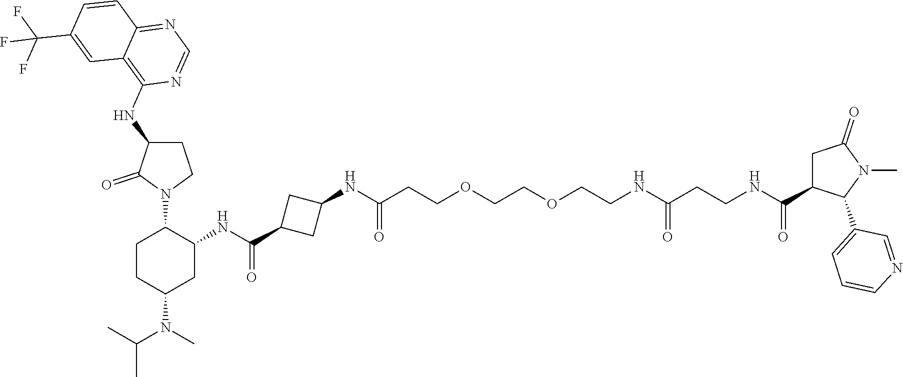 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCNC(=O)CCNC(=O)[C@H]3CC(=O)N(C)[C@@H]3c3cccnc3)C2)C1] |
| A50 | 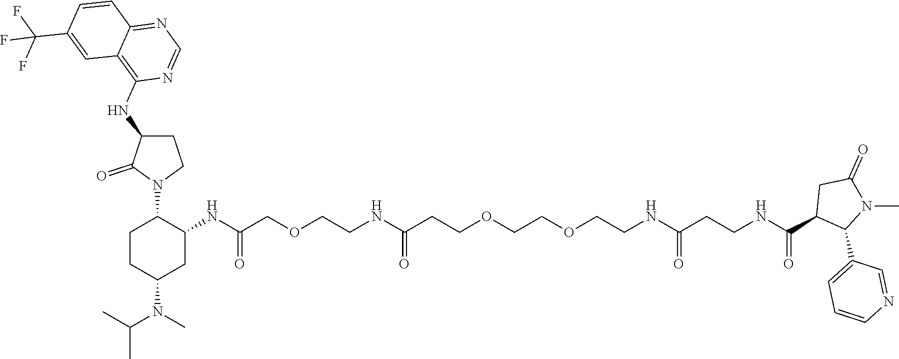 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCOCCNC(=O)CCNC(=O)[C@H]2CC(=O)N(C)[C@@H]2c2cccnc2)C1] |
| A51 | 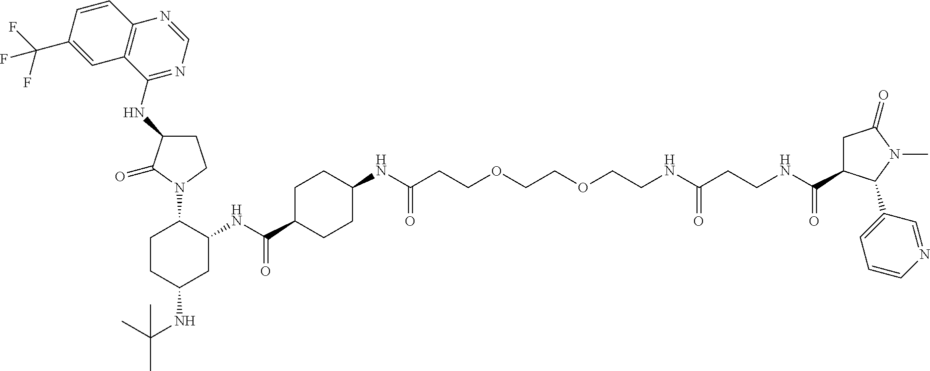 [SMILES: CN1C(=O)C[C@H](C(=O)NCCC(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A52 | 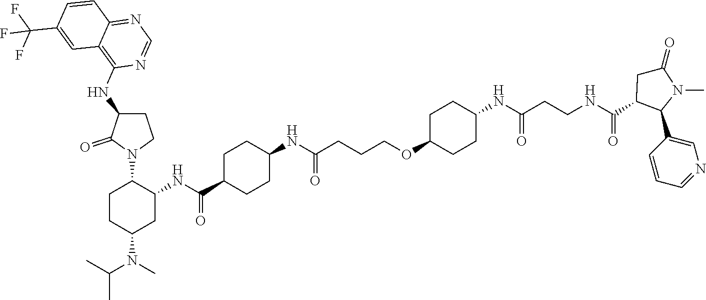 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCO[C@H]3CC[C@H](NC(=O)CCNC(=O)[C@@H]4CC(=O)N(C)[C@H]4c4cccnc4)CC3)CC2)C1] |
| A53 | 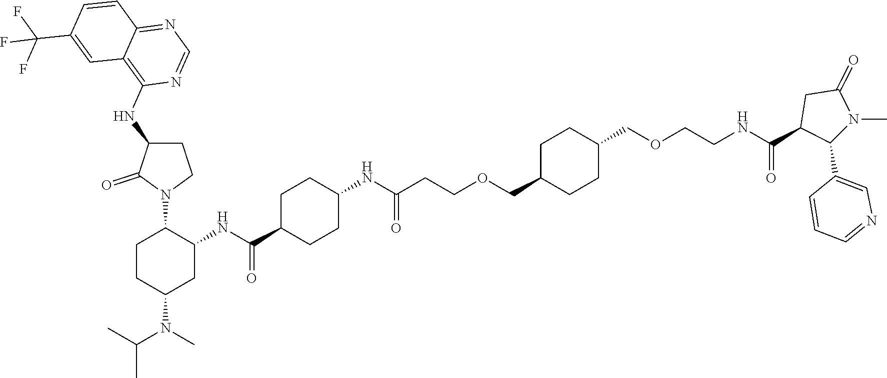 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCOC[C@H]3CC[C@H](COCCNC(=O)[C@H]4CC(=O)N(C)[C@@H]4c4cccnc4)CC3)CC2)C1] |
| A54 | 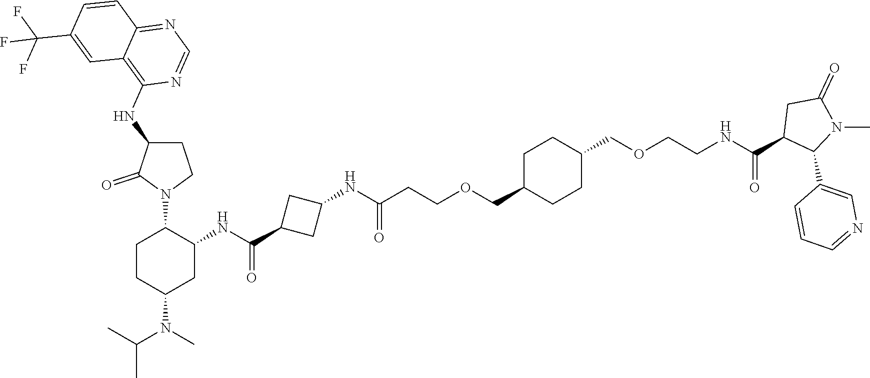 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCOC[C@H]3CC[C@H](COCCNC(=O)[C@H]4CC(=O)N(C)[C@@H]4c4cccnc4)CC3)C2)C1] |
| A55 | 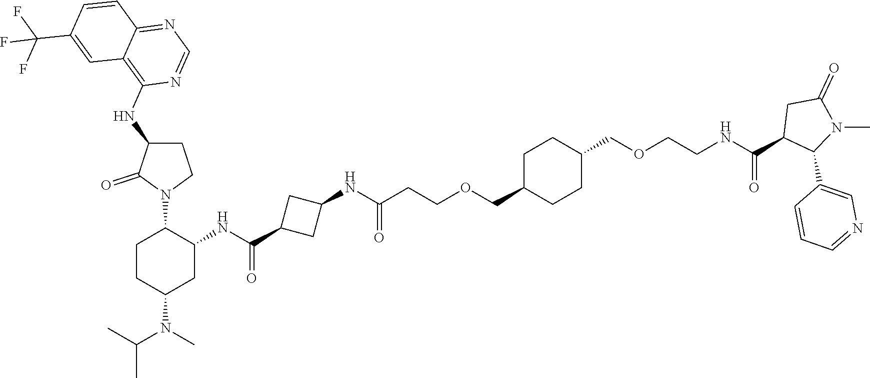 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOC[C@H]3CC[C@H](COCCNC(=O)[C@H]4CC(=O)N(C)[C@@H]4c4cccnc4)CC3)C2)C1] |
| A56 | 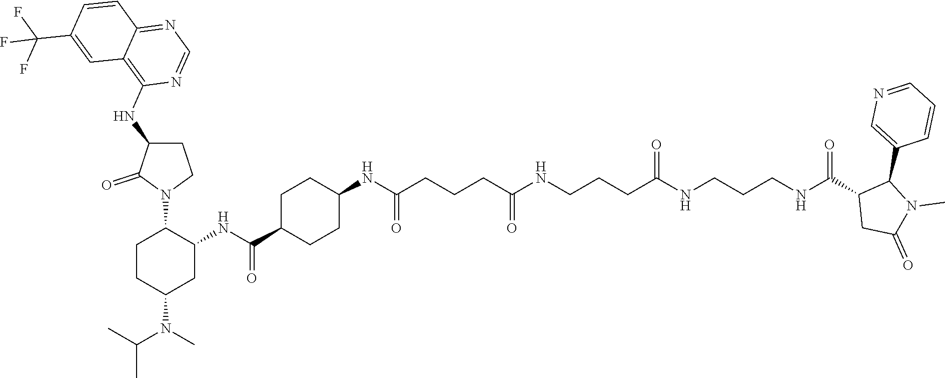 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCC(=O)NCCCC(=O)NCCCNC(=O)[C@H]3CC(=O)N(C)[C@@H]3c3cccnc3)CC2)C1] |
| A57 | 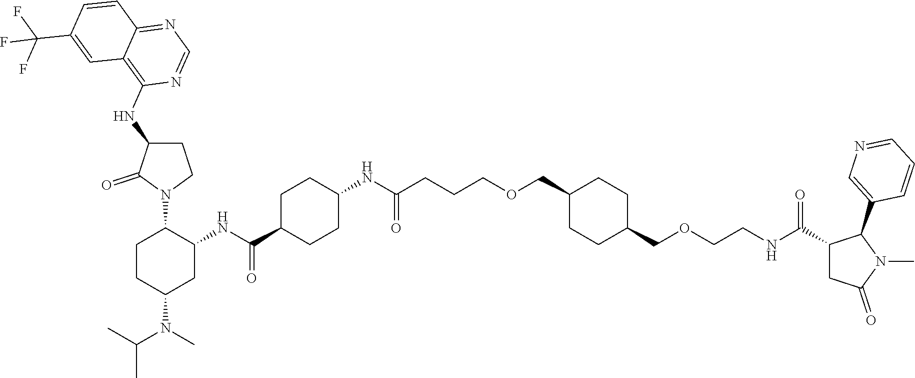 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCOC[C@H]3CC[C@@H](COCCNC(=O)[C@H]4CC(=O)N(C)[C@@H]4c4cccnc4)CC3)CC2)C1] |
| A58 | 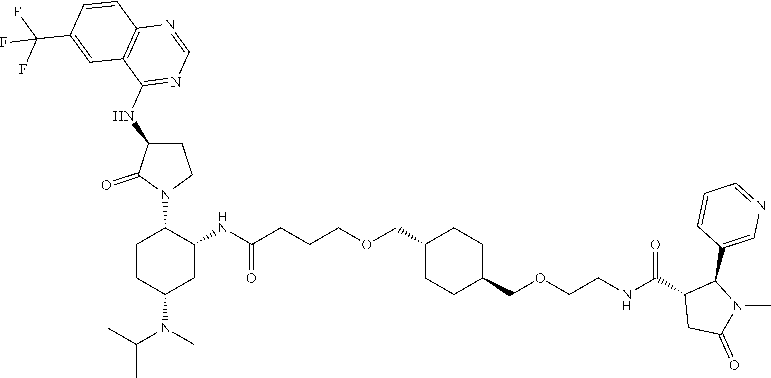 [SMILES: CC(C)N(C)[C@@H]1CC[C@H](N2CC[C@H](Nc3ncnc4ccc(C(F)(F)F)cc34)C2=O)[C@H](NC(=O)CCCOC[C@H]2CC[C@H](COCCNC(=O)[C@H]3CC(=O)N(C)[C@@H]3c3cccnc3)CC2)C1] |
| A59 | 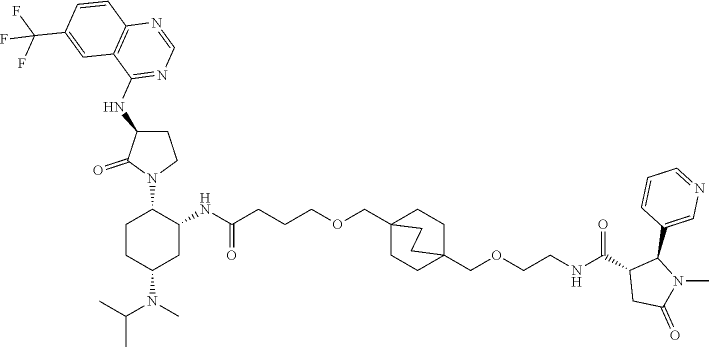 [SMILES: CC(C)N(C)[C@@H]1CC[C@H](N2CC[C@H](Nc3ncnc4ccc(C(F)(F)F)cc34)C2=O)[C@H](NC(=O)CCCOCC23CCC(COCCNC(=O)[C@H]4CC(=O)N(C)[C@@H]4c4cccnc4)(CC2)CC3)C1] |
| A60 | 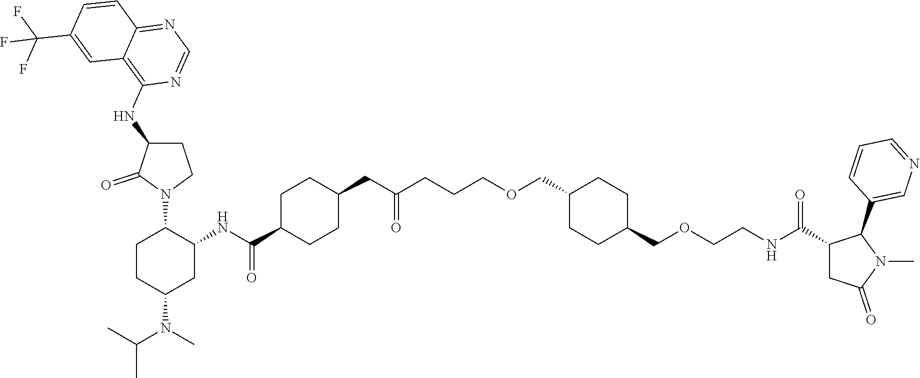 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](CC(=O)CCCOC[C@H]3CC[C@H](COCCNC(=O)[C@H]4CC(=O)N(C)[C@@H]4c4cccnc4)CC3)CC2)C1] |
| A61 | 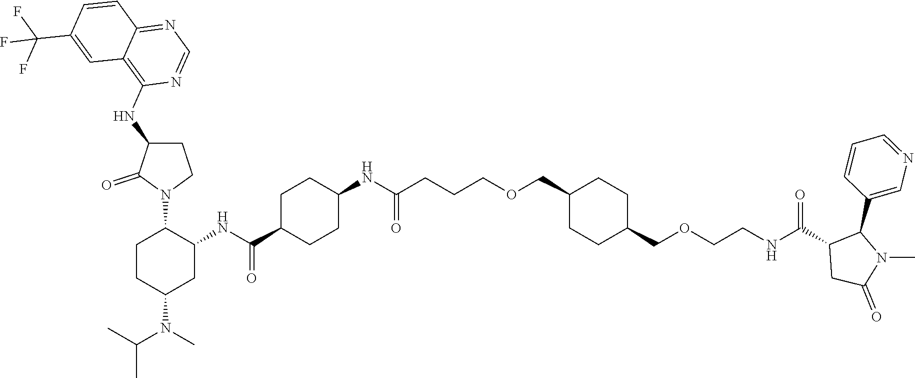 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOC[C@H]3CC[C@@H](COCCNC(=O)[C@H]4CC(=O)N(C)[C@@H]4c4cccnc4)CC3)CC2)C1] |
| A62 | 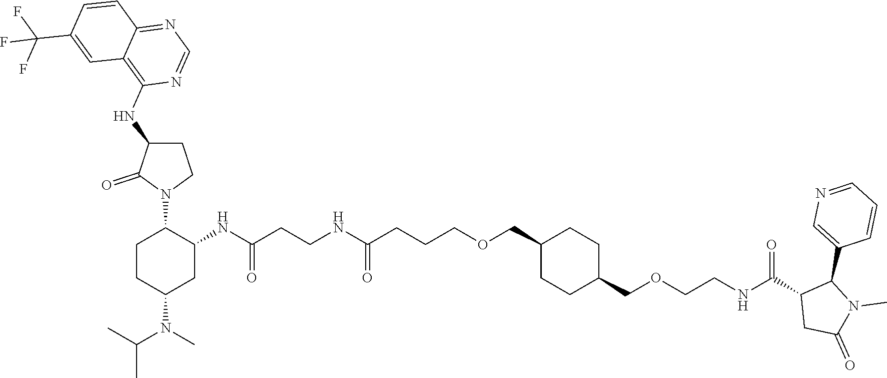 [SMILES: CC(C)N(C)[C@@H]1CC[C@H](N2CC[C@H](Nc3ncnc4ccc(C(F)(F)F)cc34)C2=O)[C@H](NC(=O)CCNC(=O)CCCOC[C@H]2CC[C@@H](COCCNC(=O)[C@H]3CC(=O)N(C)[C@@H]3c3cccnc3)CC2)C1] |
| A63 | 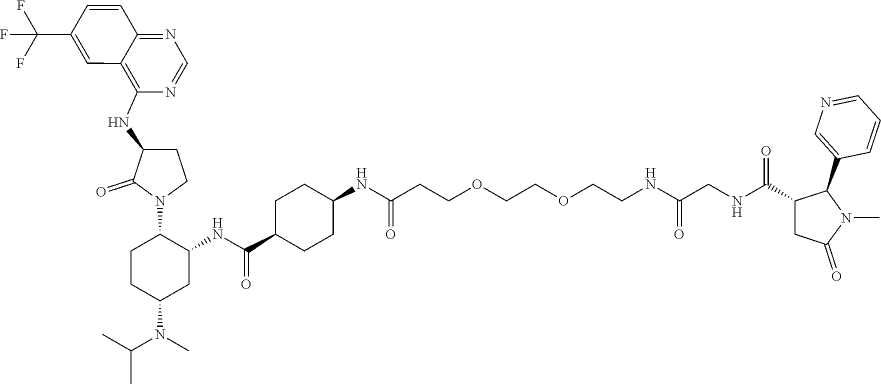 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCNC(=O)CNC(=O)[C@H]3CC(=O)N(C)[C@@H]3c3cccnc3)CC2)C1] |
| A64 | 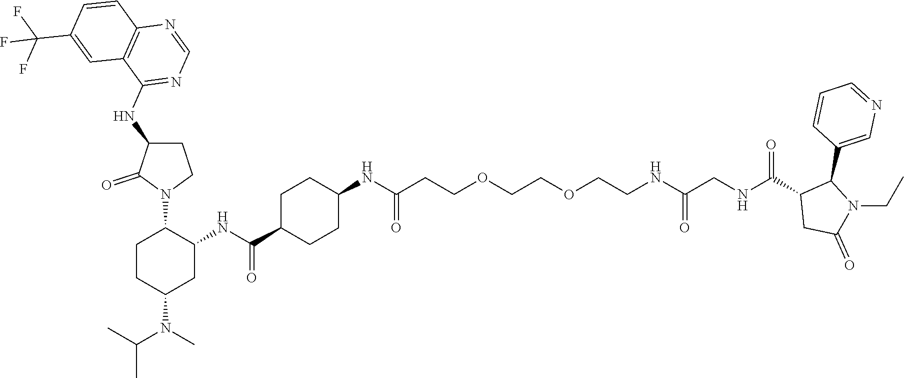 [SMILES: CCN1C(=O)C[C@H](C(=O)NCC(=O)NCCOCCOCCC(=O)N[C@H]2CC[C@@H](C(=O)N[C@@H]3C[C@H](N(C)C(C)C)CC[C@@H]3N3CC[C@H](Nc4ncnc5ccc(C(F)(F)F)cc45)C3=O)CC2)[C@H]1c1cccnc1] |
| A65 | 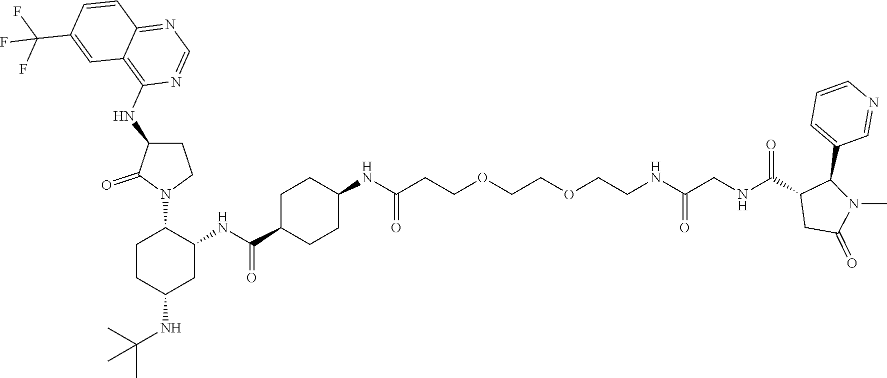 [SMILES: CN1C(=O)C[C@H](C(=O)NCC(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A66 | 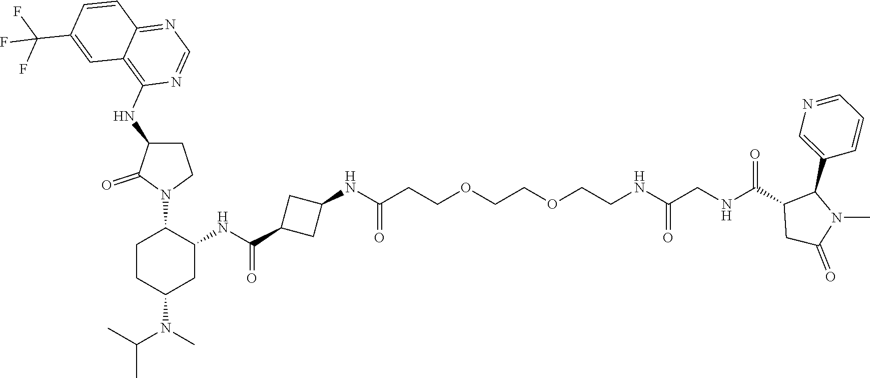 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCOCCOCCNC(=O)CNC(=O)[C@H]3CC(=O)N(C)[C@@H]3c3cccnc3)C2)C1] |
| A67 | 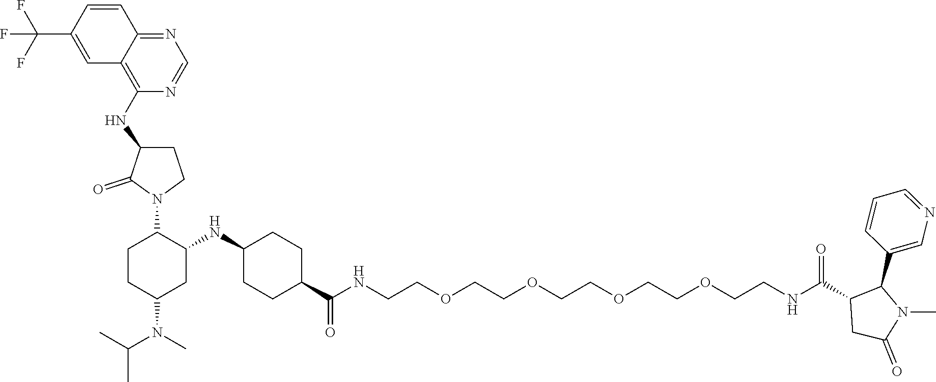 |
| A68 | 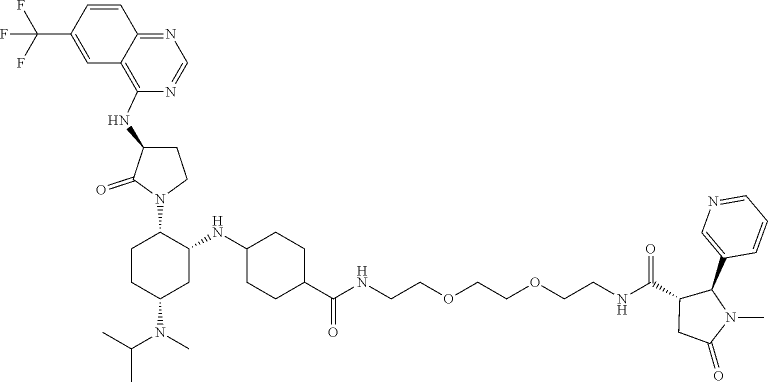 [SMILES: CC(C)N(C)[C@@H]1CC[C@H](N2CC[C@H](Nc3ncnc4ccc(C(F)(F)F)cc34)C2=O)[C@H](NC2CCC(C(=O)NCCOCCOCCNC(=O)[C@H]3CC(=O)N(C)[C@@H]3c3cccnc3)CC2)C1] |
| A69 | 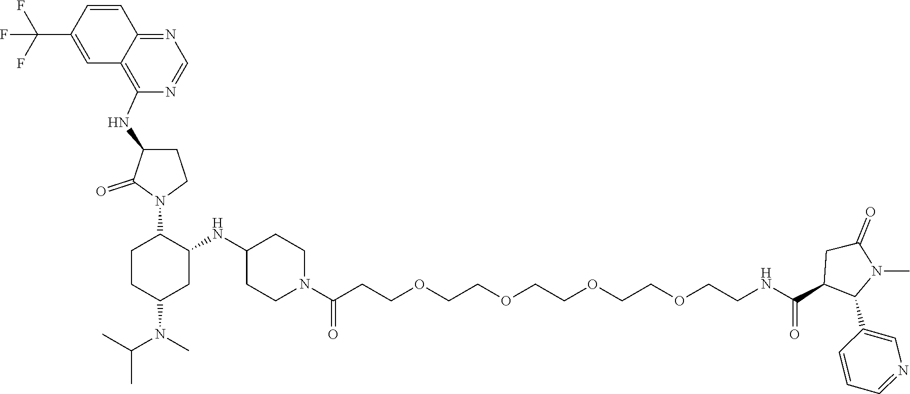 |
| A70 | 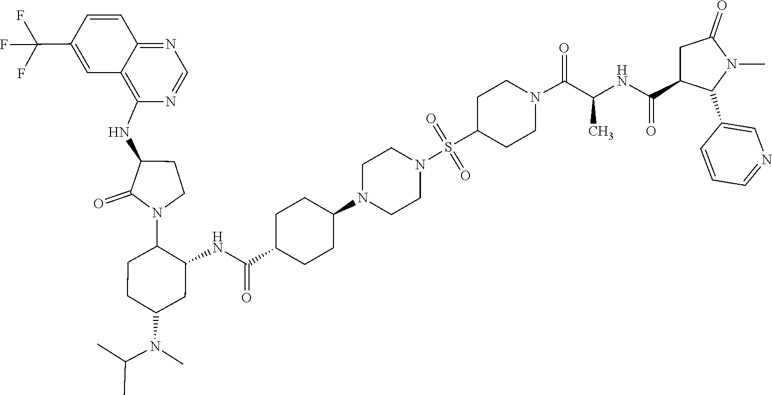 |
| A71 | 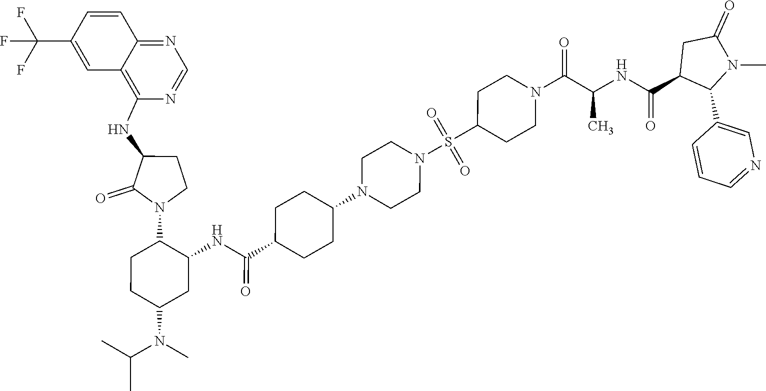 |
| A72 | 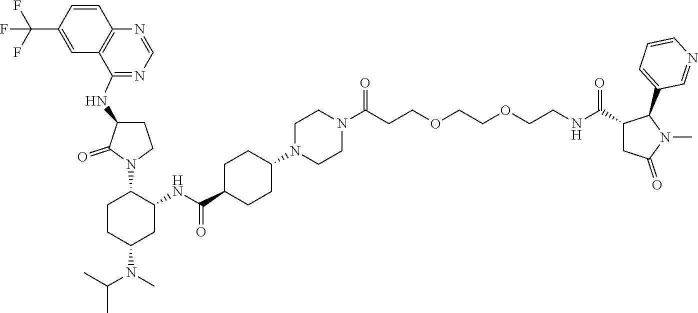 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C(=O)CCOCCOCCNC(=O)[C@H]4CC(=O)N(C)[C@@H]4c4cccnc4)CC3)CC2)C1] |
| A73 | 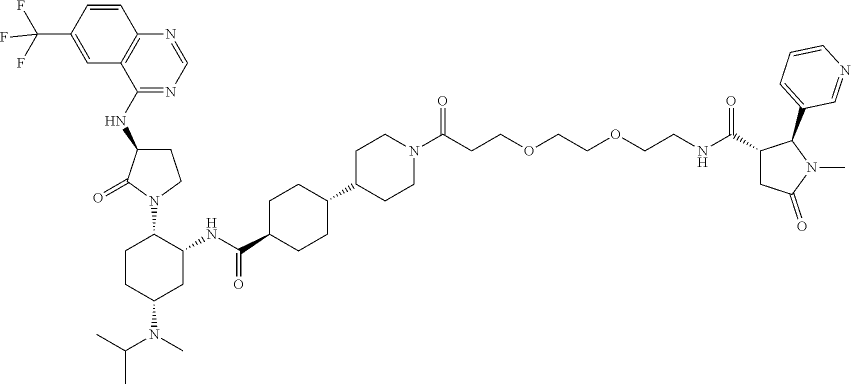 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C3CCN(C(=O)CCOCCOCCNC(=O)[C@H]4CC(=O)N(C)[C@@H]4c4cccnc4)CC3)CC2)C1] |
| A74 | 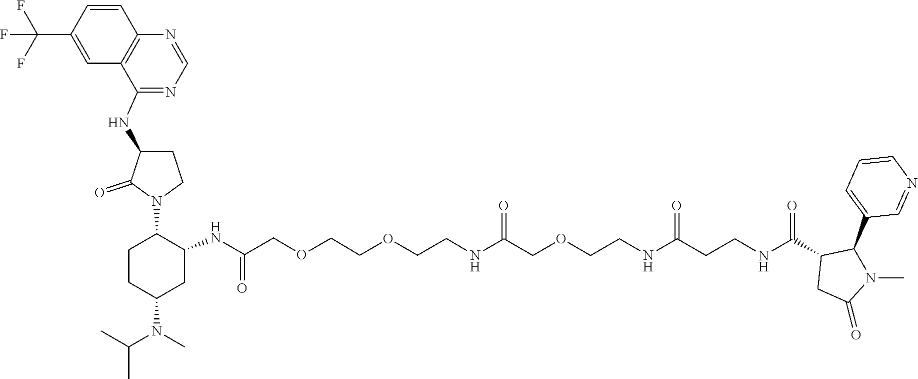 [SMILES: CC(C)N(C)[C@@H]1CC[C@H](N2CC[C@H](Nc3ncnc4ccc(C(F)(F)F)cc34)C2=O)[C@H](NC(=O)COCCOCCNC(=O)COCCNC(=O)CCNC(=O)[C@H]2CC(=O)N(C)[C@@H]2c2cccnc2)C1] |
| A75 | 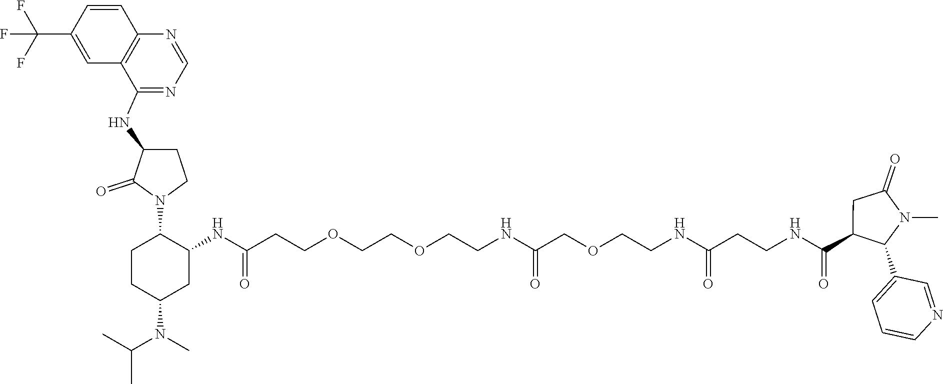 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)COCCNC(=O)CCNC(=O)[C@H]2CC(=O)N(C)[C@@H]2c2cccnc2)C1] |
| A76 | + 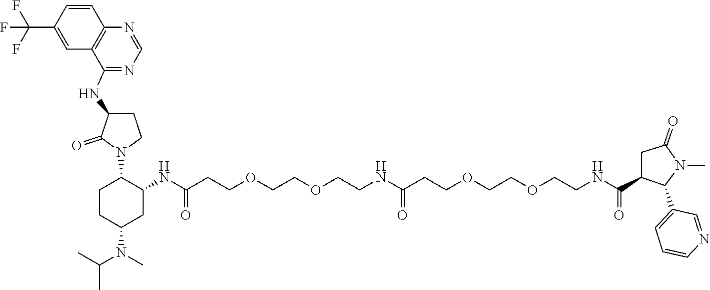 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)CCOCCOCCNC(=O)[C@H]2CC(=O)N(C)[C@@H]2c2cccnc2)C1] |
| A77 | 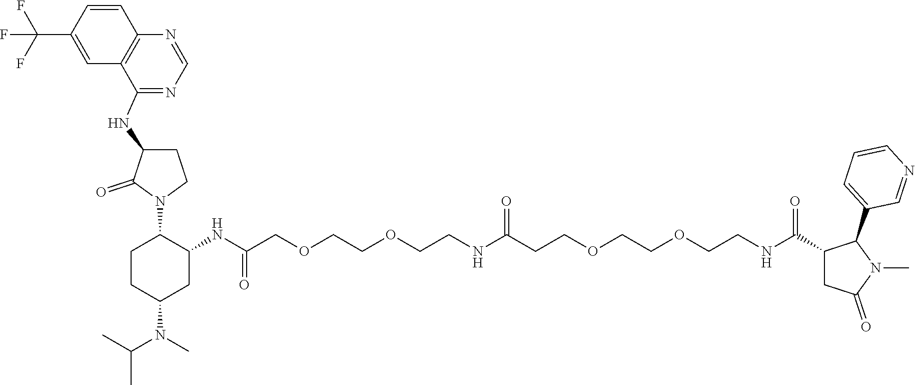 [SMILES: CC(C)N(C)[C@@H]1CC[C@H](N2CC[C@H](Nc3ncnc4ccc(C(F)(F)F)cc34)C2=O)[C@H](NC(=O)COCCOCCNC(=O)CCOCCOCCNC(=O)[C@H]2CC(=O)N(C)[C@@H]2c2cccnc2)C1] |
| A78 | 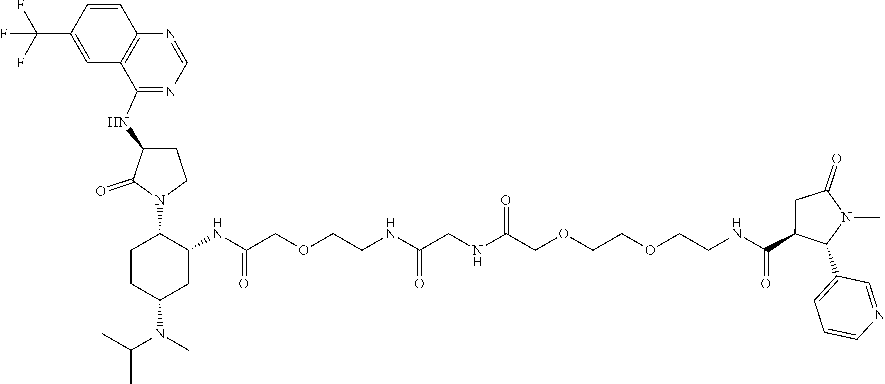 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CNC(=O)COCCOCCNC(=O)[C@H]2CC(=O)N(C)[C@@H]2c2cccnc2)C1] |
| A79 | 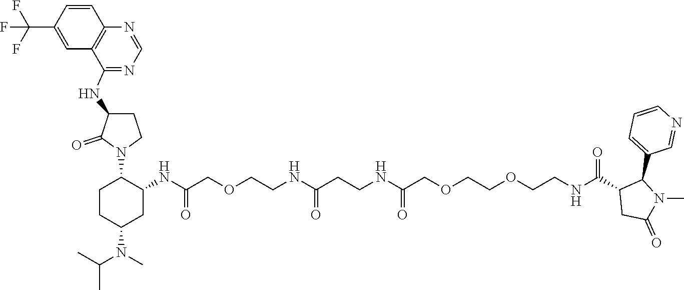 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCNC(=O)COCCOCCNC(=O)[C@H]2CC(=O)N(C)[C@@H]2c2cccnc2)C1] |
| A80 | 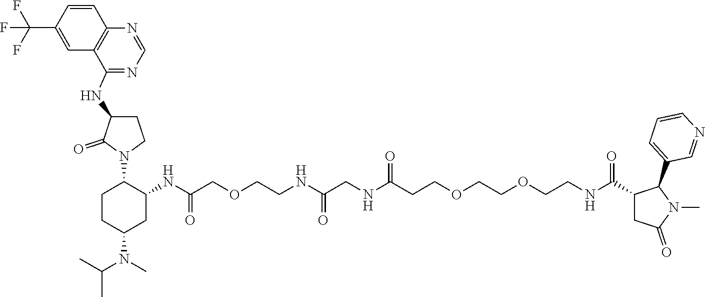 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CNC(=O)CCOCCOCCNC(=O)[C@H]2CC(=O)N(C)[C@@H]2c2cccnc2)C1] |
| A81 | 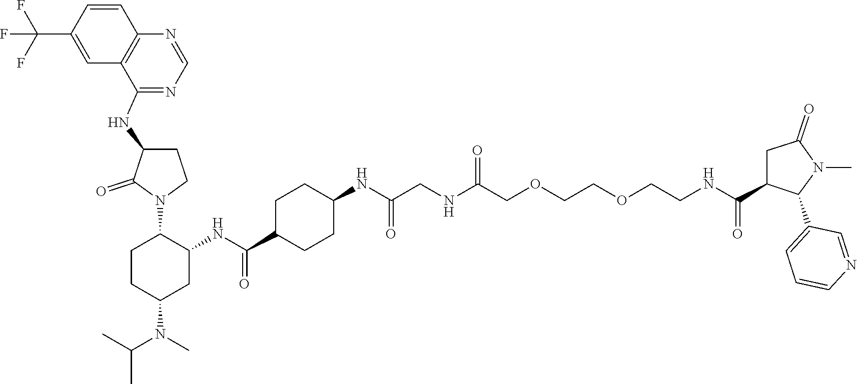 |
| A82 | 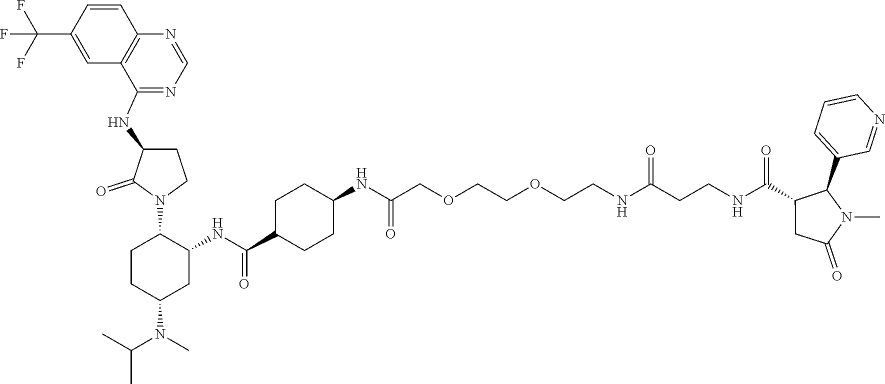 |
| A83 | 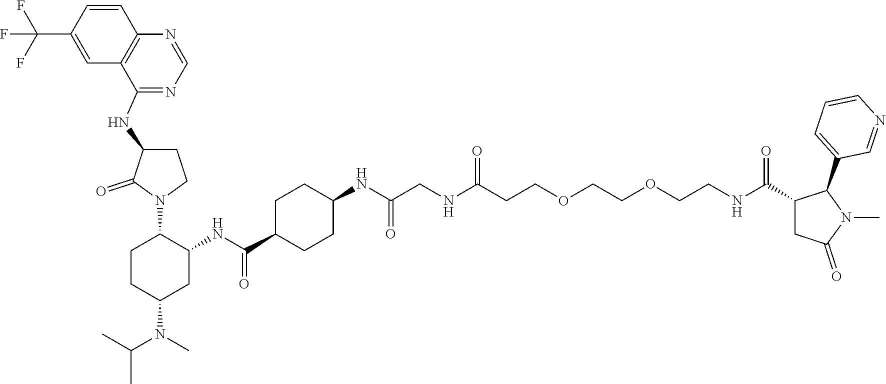 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CNC(=O)CCOCCOCCNC(=O)[C@H]3CC(=O)N(C)[C@@H]3c3cccnc3)CC2)C1] |
| A84 | 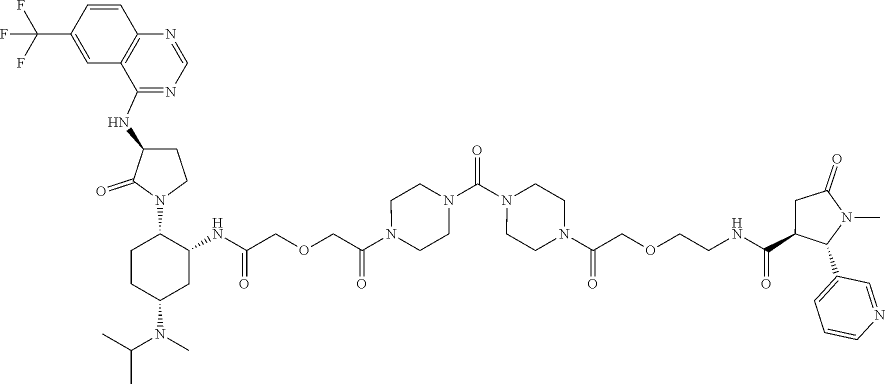 [SMILES: CC(C)N(C)[C@@H]1CC[C@H](N2CC[C@H](Nc3ncnc4ccc(C(F)(F)F)cc34)C2=O)[C@H](NC(=O)COCC(=O)N2CCN(C(=O)N3CCN(C(=O)COCCNC(=O)[C@H]4CC(=O)N(C)[C@@H]4c4cccnc4)CC3)CC2)C1] |
| A85 | 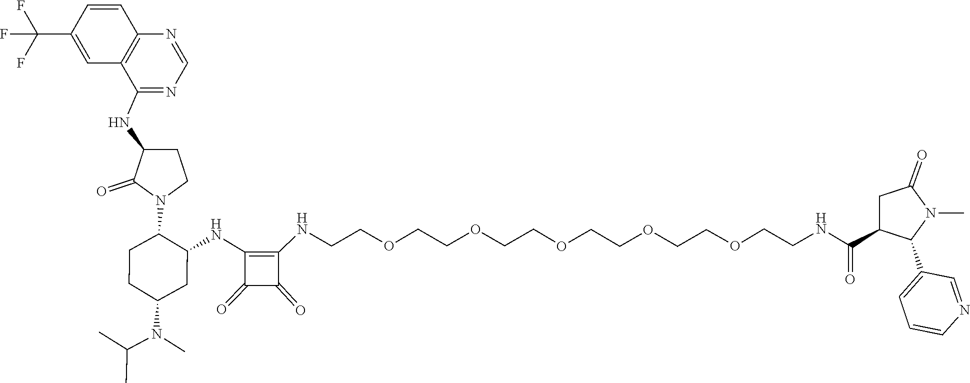 [SMILES: CC(C)N(C)[C@@H]1CC[C@H](N2CC[C@H](Nc3ncnc4ccc(C(F)(F)F)cc34)C2=O)[C@H](Nc2c(NCCOCCOCCOCCOCCOCCNC(=O)[C@H]3CC(=O)N(C)[C@@H]3c3cccnc3)c(=O)c2=O)C1] |
| A86 | 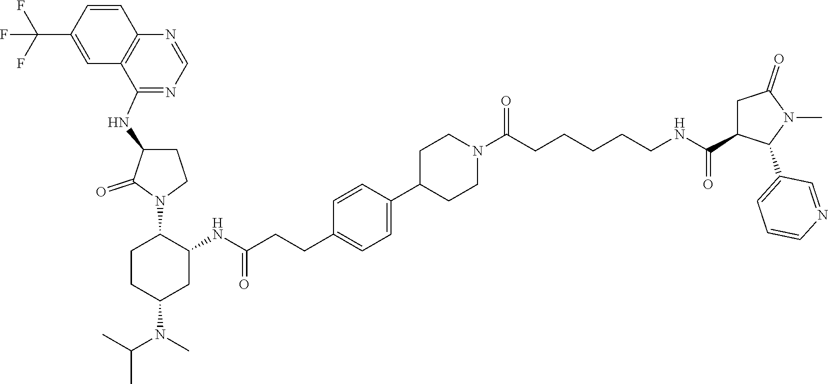 [SMILES: CC(C)N(C)[C@@H]1CC[C@H](N2CC[C@H](Nc3ncnc4ccc(C(F)(F)F)cc34)C2=O)[C@H](NC(=O)CCc2ccc(C3CCN(C(=O)CCCCCNC(=O)[C@H]4CC(=O)N(C)[C@@H]4c4cccnc4)CC3)cc2)C1] |
| A87 | 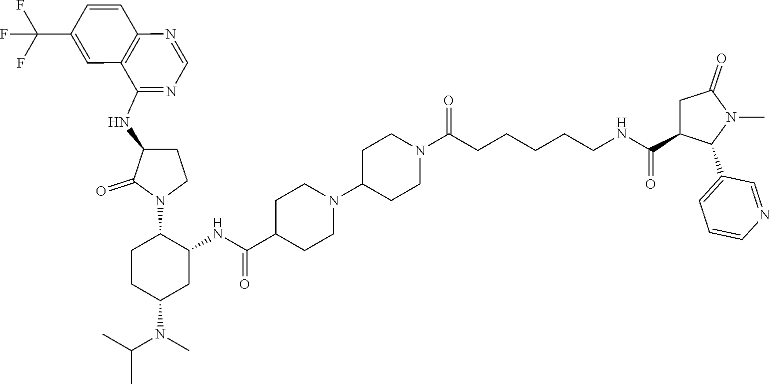 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCNC(=O)[C@H]4CC(=O)N(C)[C@@H]4c4cccnc4)CC3)CC2)C1] |
| A88 | 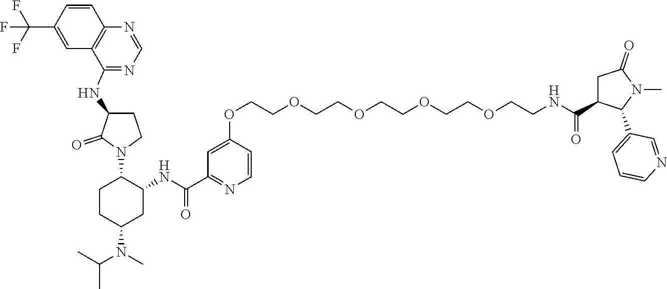 [SMILES: CC(C)N(C)[C@@H]1CC[C@H](N2CC[C@H](Nc3ncnc4ccc(C(F)(F)F)cc34)C2=O)[C@H](NC(=O)c2cc(OCCOCCOCCOCCOCCNC(=O)[C@H]3CC(=O)N(C)[C@@H]3c3cccnc3)ccn2)C1] |
| A89 | 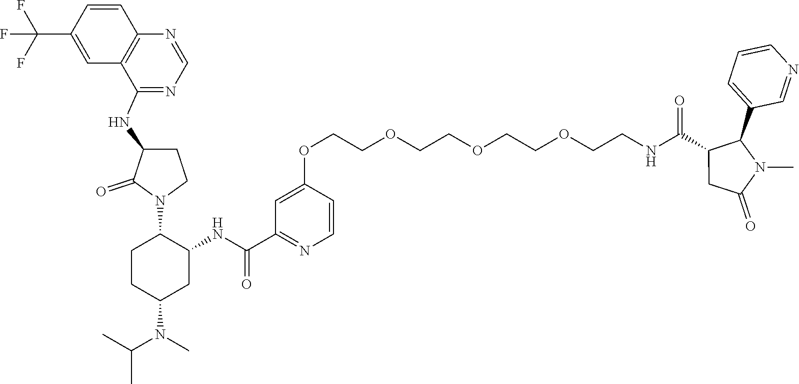 [SMILES: CC(C)N(C)[C@@H]1CC[C@H](N2CC[C@H](Nc3ncnc4ccc(C(F)(F)F)cc34)C2=O)[C@H](NC(=O)c2cc(OCCOCCOCCOCCNC(=O)[C@H]3CC(=O)N(C)[C@@H]3c3cccnc3)ccn2)C1] |
| A90 | 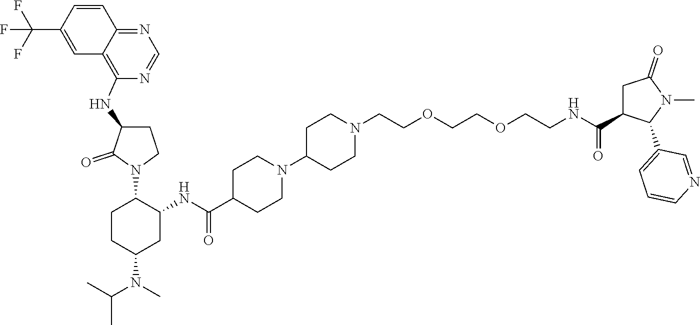 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(CCOCCOCCNC(=O)[C@H]4CC(=O)N(C)[C@@H]4c4cccnc4)CC3)CC2)C1] |
| A91 | 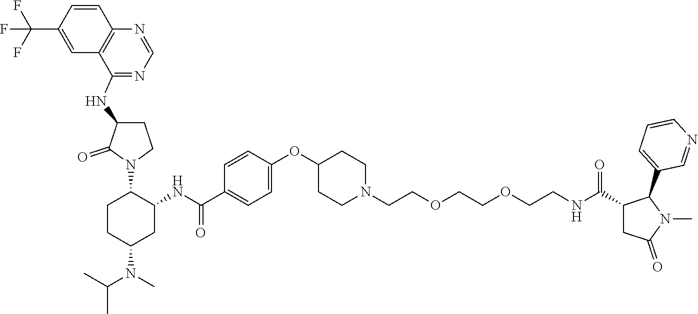 [SMILES: CC(C)N(C)[C@@H]1CC[C@H](N2CC[C@H](Nc3ncnc4ccc(C(F)(F)F)cc34)C2=O)[C@H](NC(=O)c2ccc(OC3CCN(CCOCCOCCNC(=O)[C@H]4CC(=O)N(C)[C@@H]4c4cccnc4)CC3)cc2)C1] |
| A92 | 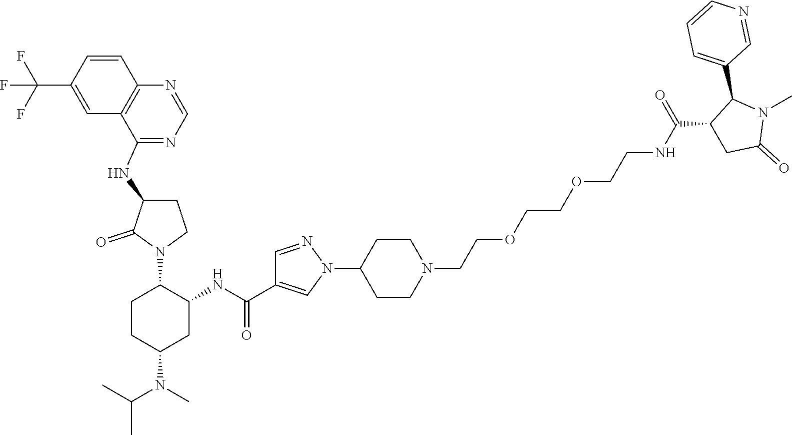 [SMILES: CC(C)N(C)[C@@H]1CC[C@H](N2CC[C@H](Nc3ncnc4ccc(C(F)(F)F)cc34)C2=O)[C@H](NC(=O)c2cnn(C3CCN(CCOCCOCCNC(=O)[C@H]4CC(=O)N(C)[C@@H]4c4cccnc4)CC3)c2)C1] |
| A93 | 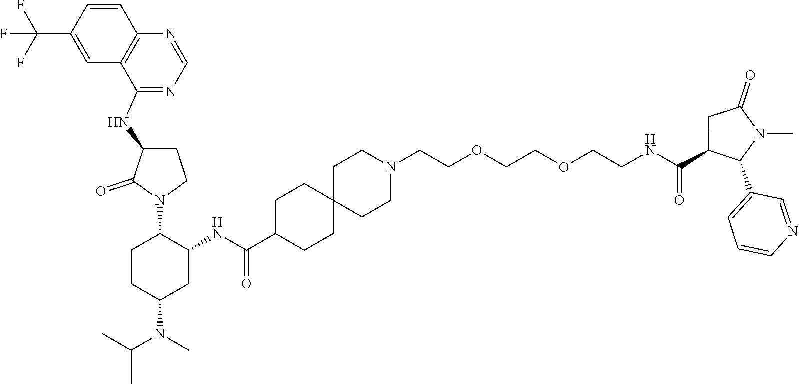 [SMILES: CC(C)N(C)[C@@H]1CC[C@H](N2CC[C@H](Nc3ncnc4ccc(C(F)(F)F)cc34)C2=O)[C@H](NC(=O)C2CCC3(CC2)CCN(CCOCCOCCNC(=O)[C@H]2CC(=O)N(C)[C@@H]2c2cccnc2)CC3)C1] |
| A94 | 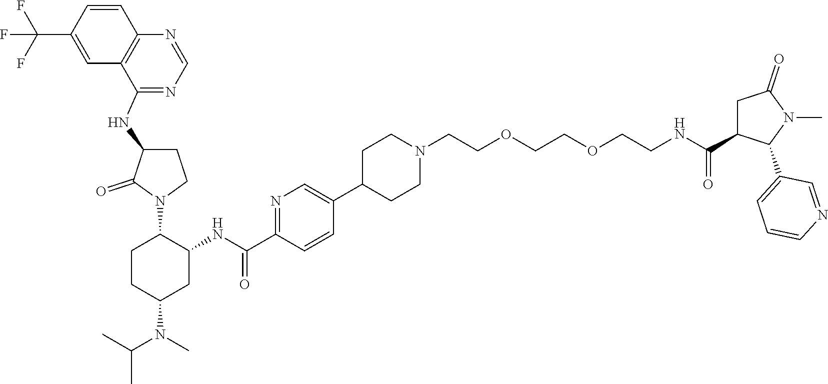 [SMILES: CC(C)N(C)[C@@H]1CC[C@H](N2CC[C@H](Nc3ncnc4ccc(C(F)(F)F)cc34)C2=O)[C@H](NC(=O)c2ccc(C3CCN(CCOCCOCCNC(=O)[C@H]4CC(=O)N(C)[C@@H]4c4cccnc4)CC3)cn2)C1] |
| A95 | 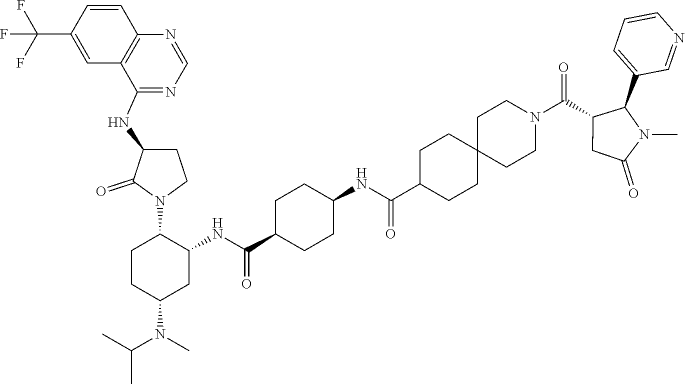 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)C3CCC4(CC3)CCN(C(=O)[C@H]3CC(=O)N(C)[C@@H]3c3cccnc3)CC4)CC2)C1] |
| A96 | 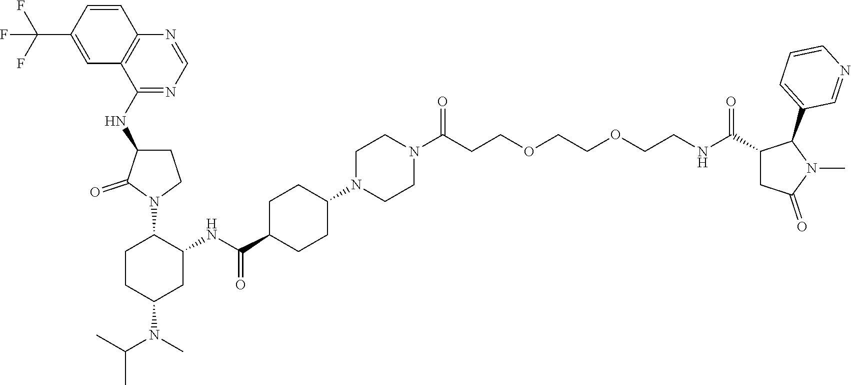 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C(=O)CCOCCOCCNC(=O)[C@H]4CC(=O)N(C)[C@@H]4c4cccnc4)CC3)CC2)C1] |
| A97 | 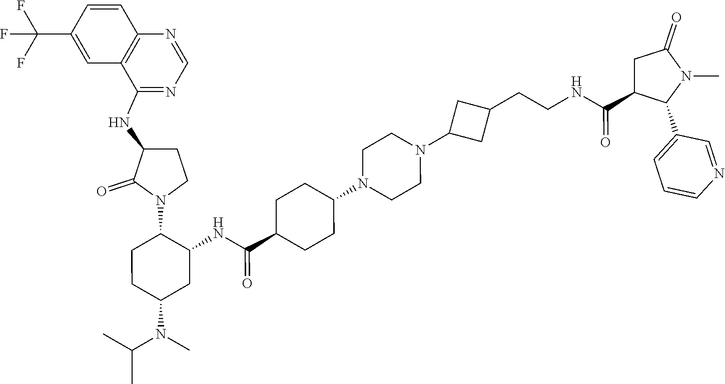 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C4CC(CCNC(=O)[C@H]5CC(=O)N(C)[C@@H]5c5cccnc5)C4)CC3)CC2)C1] |
| A98 | 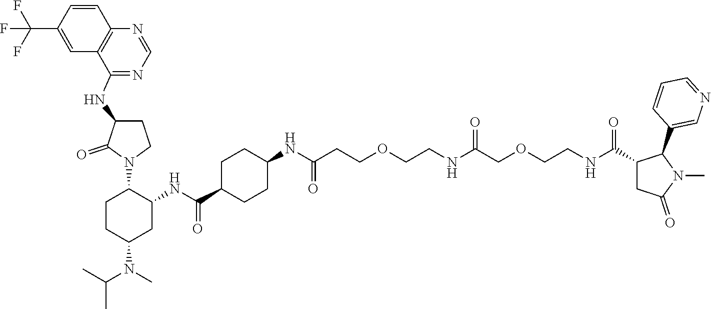 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)COCCNC(=O)[C@H]3CC(=O)N(C)[C@@H]3c3cccnc3)CC2)C1] |
| A99 | 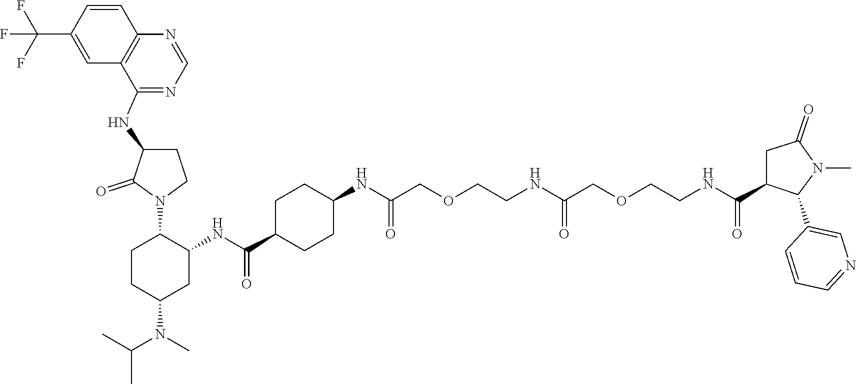 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCNC(=O)COCCNC(=O)[C@H]3CC(=O)N(C)[C@@H]3c3cccnc3)CC2)C1] |
| A100 | 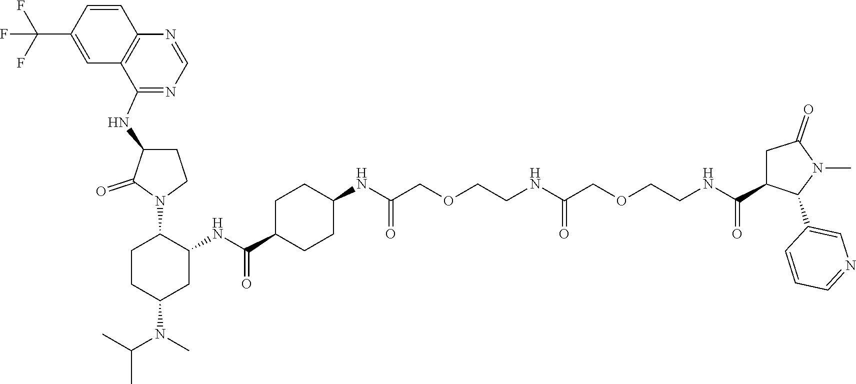 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCNC(=O)COCCNC(=O)[C@H]3CC(=O)N(C)[C@@H]3c3cccnc3)CC2)C1] |
| A101 | 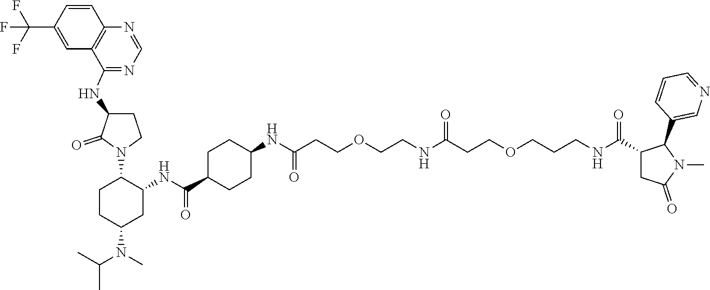 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCOCCCNC(=O)[C@H]3CC(=O)N(C)[C@@H]3c3cccnc3)CC2)C1] |
| A102 | 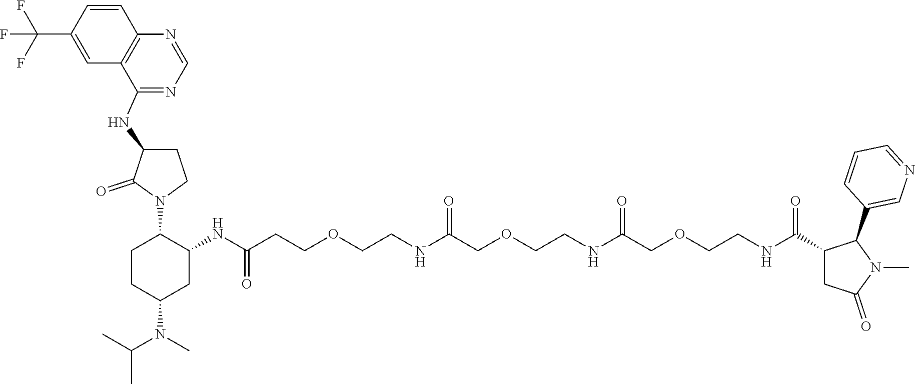 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)COCCNC(=O)COCCNC(=O)[C@H]2CC(=O)N(C)[C@@H]2c2cccnc2)C1] |
| A103 | 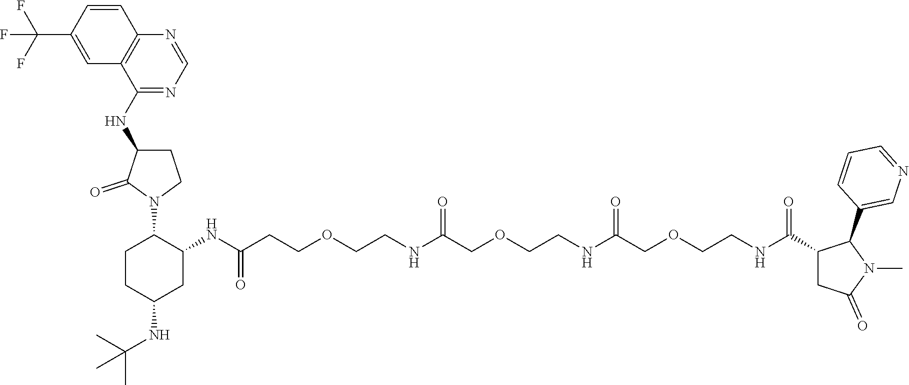 [SMILES: CN1C(=O)C[C@H](C(=O)NCCOCC(=O)NCCOCC(=O)NCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A104 | 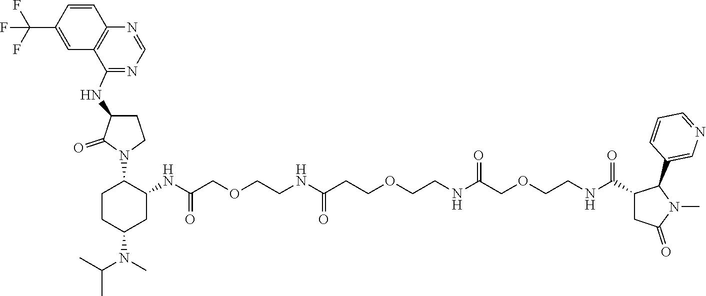 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCOCCNC(=O)COCCNC(=O)[C@H]2CC(=O)N(C)[C@@H]2c2cccnc2)C1] |
| A105 | 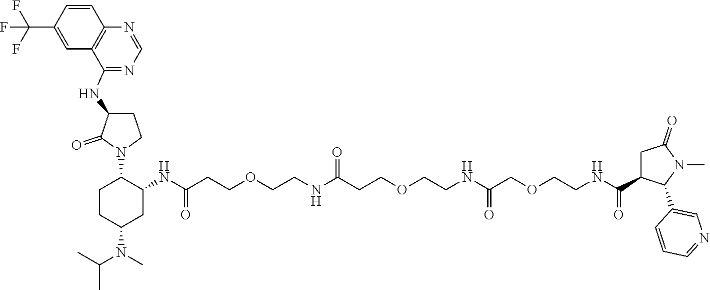 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)CCOCCNC(=O)COCCNC(=O)[C@H]2CC(=O)N(C)[C@@H]2c2cccnc2)C1] |
| A106 | 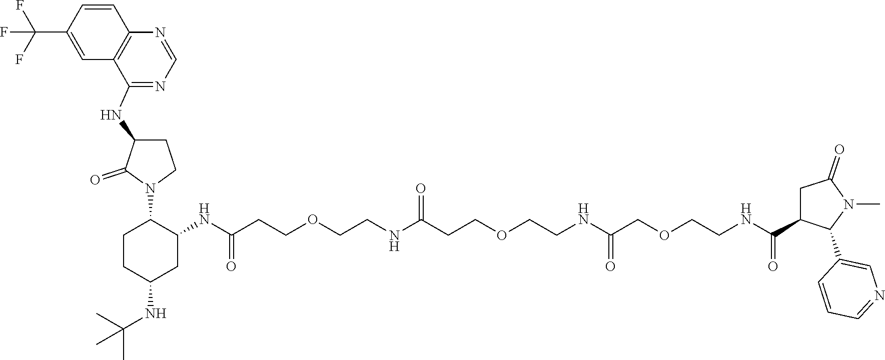 [SMILES: CN1C(=O)C[C@H](C(=O)NCCOCC(=O)NCCOCCC(=O)NCCOCCC(=O)N[C@@H]2C[C@H](NC(C)(C)C)CC[C@@H]2N2CC[C@H](Nc3ncnc4ccc(C(F)(F)F)cc34)C2=O)[C@H]1c1cccnc1] |
| A107 | 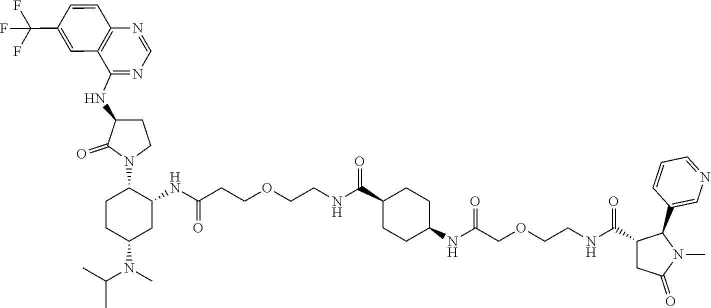 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@@H](NC(=O)COCCNC(=O)[C@H]3CC(=O)N(C)[C@@H]3c3cccnc3)CC2)C1] |
| A108 | 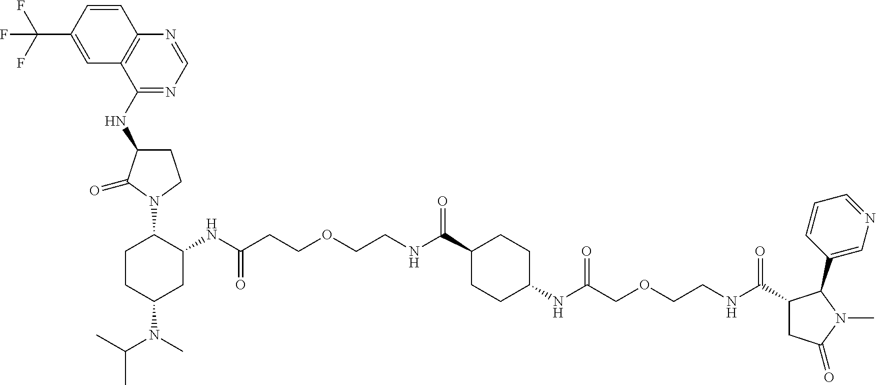 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@H](NC(=O)COCCNC(=O)[C@H]3CC(=O)N(C)[C@@H]3c3cccnc3)CC2)C1] |
| A109 | 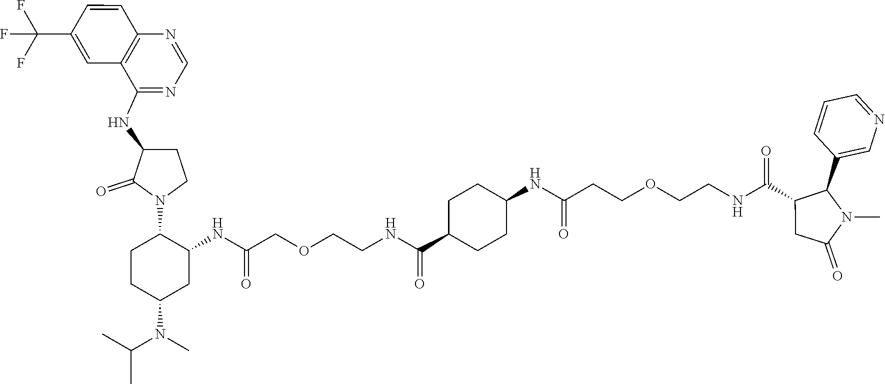 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)[C@H]2CC[C@@H](NC(=O)CCOCCNC(=O)[C@H]3CC(=O)N(C)[C@@H]3c3cccnc3)CC2)C1] |
| A110 | 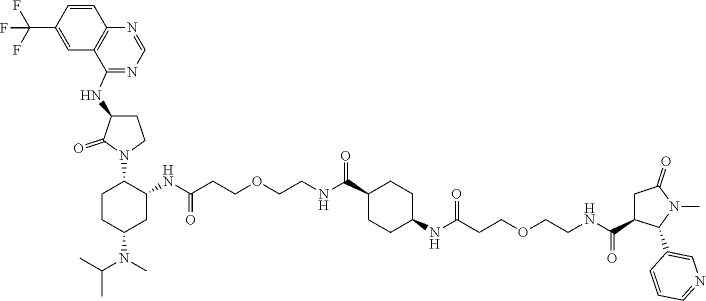 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)[C@H]2CC[C@@H](NC(=O)CCOCCNC(=O)[C@H]3CC(=O)N(C)[C@@H]3c3cccnc3)CC2)C1] |
| A111 | 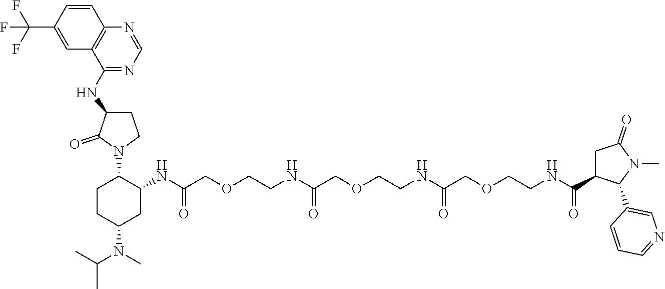 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCNC(=O)COCCNC(=O)[C@H]2CC(=O)N(C)[C@@H]2c2cccnc2)C1] |
| A112 | 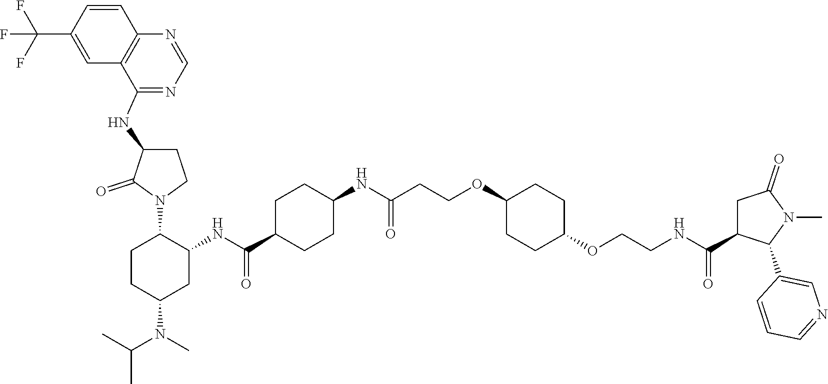 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCO[C@H]3CC[C@H](OCCNC(=O)[C@H]4CC(=O)N(C)[C@@H]4c4cccnc4)CC3)CC2)C1] |
| A113 | 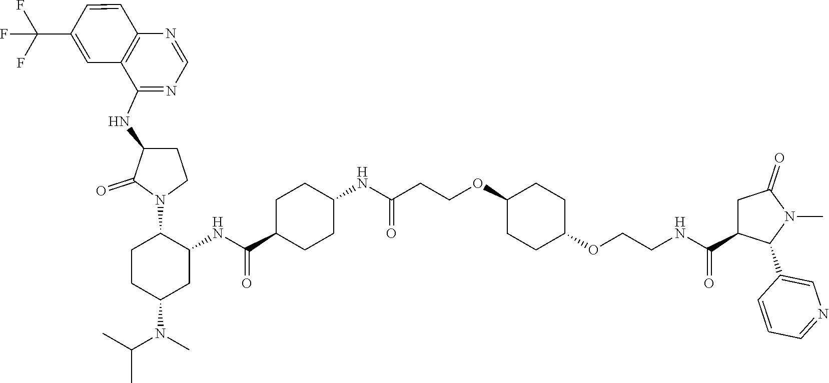 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCO[C@H]3CC[C@H](OCCNC(=O)[C@H]4CC(=O)N(C)[C@@H]4c4cccnc4)CC3)CC2)C1] |
| A114 | 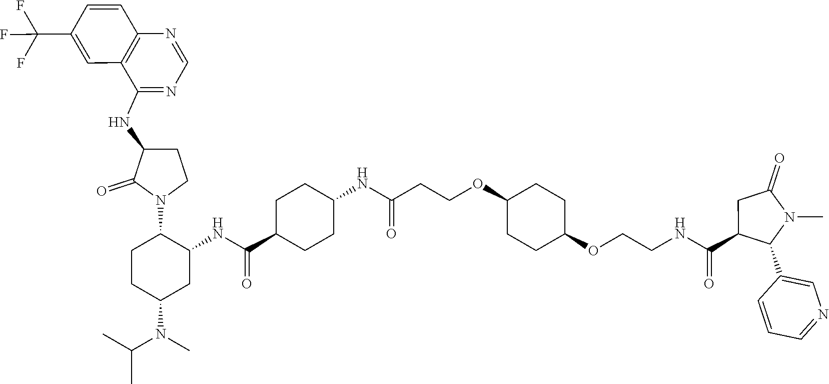 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCO[C@H]3CC[C@@H](OCCNC(=O)[C@H]4CC(=O)N(C)[C@@H]4c4cccnc4)CC3)CC2)C1] |
| A115 | 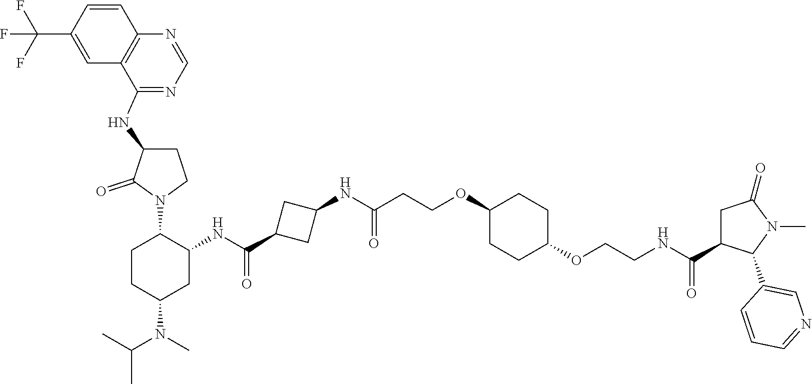 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@@H](NC(=O)CCO[C@H]3CC[C@H](OCCNC(=O)[C@H]4CC(=O)N(C)[C@@H]4c4cccnc4)CC3)C2)C1] |
| A116 | 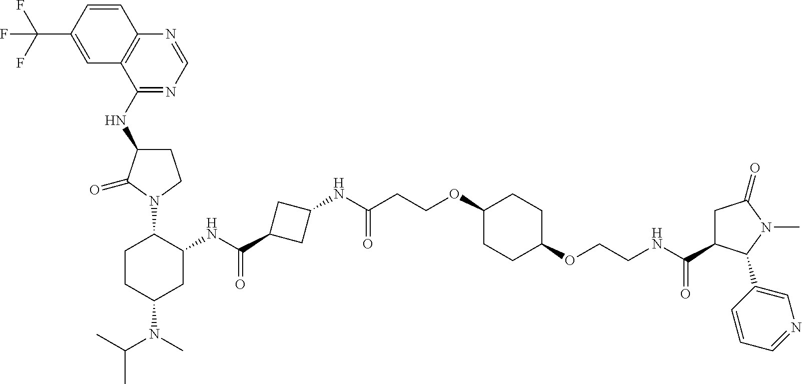 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCO[C@H]3CC[C@@H](OCCNC(=O)[C@H]4CC(=O)N(C)[C@@H]4c4cccnc4)CC3)C2)C1] |
| A117 | 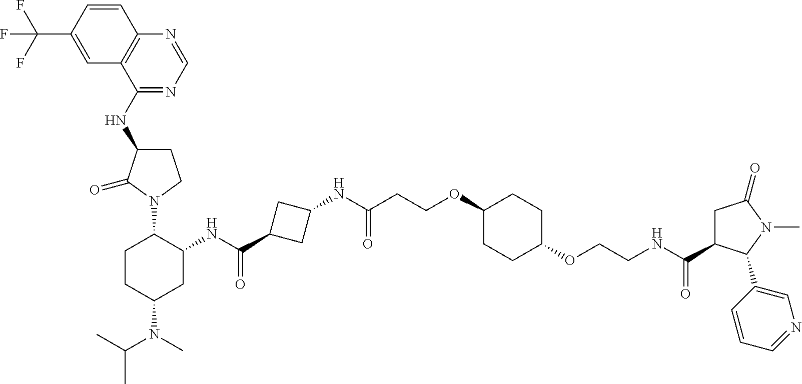 [SMILES: CC(C)N(C)[C@@H]1CC[C@H](N2CC[C@H](Nc3ncnc4ccc(C(F)(F)F)cc34)C2=O)[C@H](NC(=O)[C@H]2C[C@H](NC(=O)CCO[C@H]3CC[C@H](OCCNC(=O)[C@H]4CC(=O)N(C)[C@@H]4c4cccnc4)CC3)C2)C1] |
| A118 | 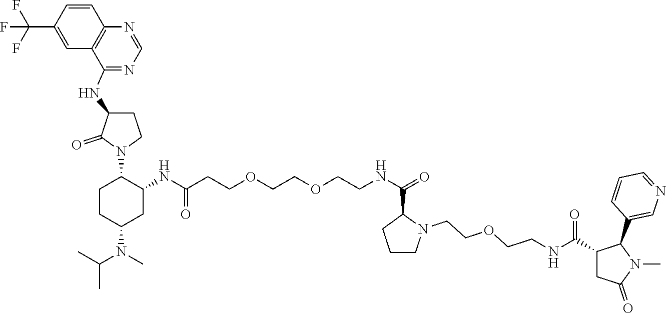 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)[C@@H]2CCCN2CCOCCNC(=O)[C@H]2CC(=O)N(C)[C@@H]2c2cccnc2)C1] |
| A119 | 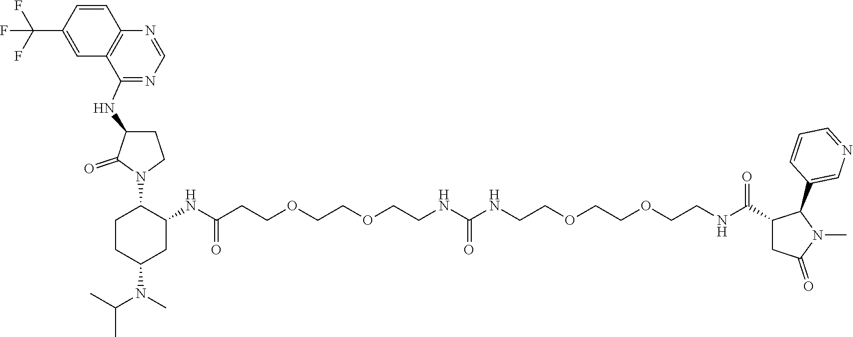 [SMILES: CC(C)N(C)[C@@H]1CC[C@H](N2CC[C@H](Nc3ncnc4ccc(C(F)(F)F)cc34)C2=O)[C@H](NC(=O)CCOCCOCCNC(=O)NCCOCCOCCNC(=O)[C@H]2CC(=O)N(C)[C@@H]2c2cccnc2)C1] |
| A120 | 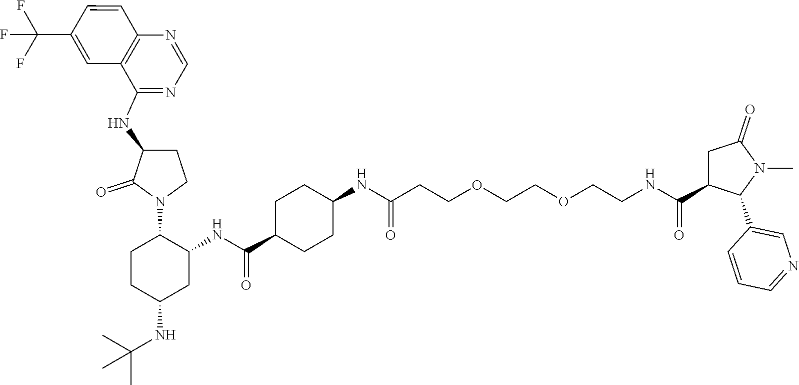 [SMILES: CN1C(=O)C[C@H](C(=O)NCCOCCOCCC(=O)N[C@H]2CC[C@@H](C(=O)N[C@@H]3C[C@H](NC(C)(C)C)CC[C@@H]3N3CC[C@H](Nc4ncnc5ccc(C(F)(F)F)cc45)C3=O)CC2)[C@H]1c1cccnc1] |
| A121 | 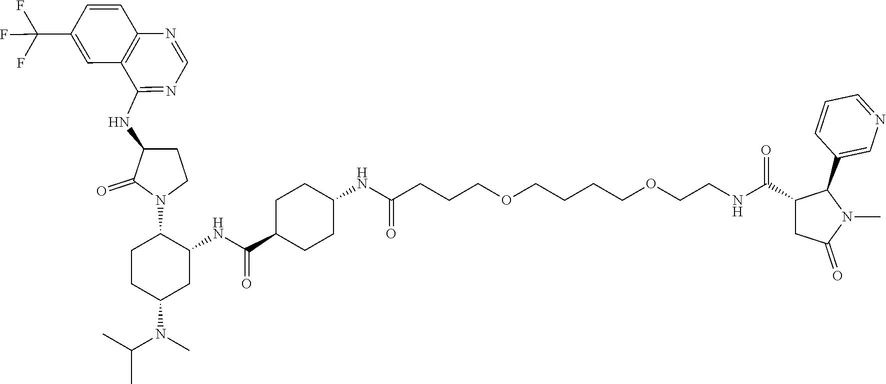 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](NC(=O)CCCOCCCCOCCNC(=O)[C@H]3CC(=O)N(C)[C@@H]3c3cccnc3)CC2)C1] |
| A122 | 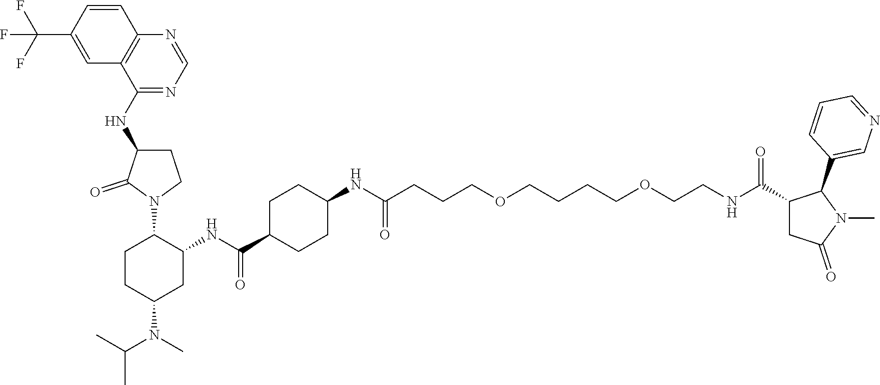 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOCCCCOCCNC(=O)[C@H]3CC(=O)N(C)[C@@H]3c3cccnc3)CC2)C1] |
| A123 | 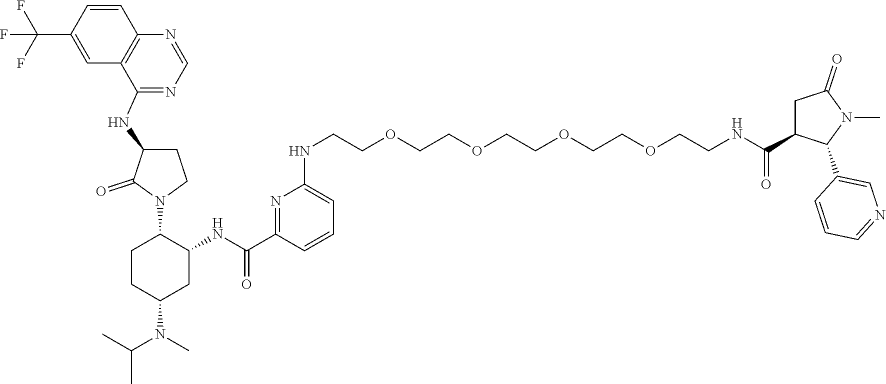 [SMILES: CC(C)N(C)[C@@H]1CC[C@H](N2CC[C@H](Nc3ncnc4ccc(C(F)(F)F)cc34)C2=O)[C@H](NC(=O)c2cccc(NCCOCCOCCOCCOCCNC(=O)[C@H]3CC(=O)N(C)[C@@H]3c3cccnc3)n2)C1] |
| A124 | 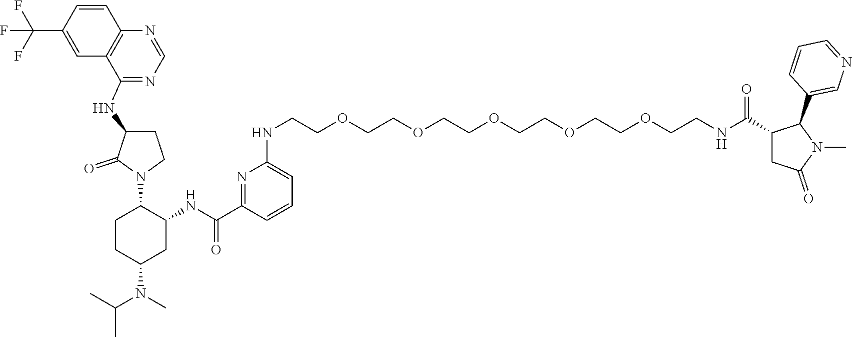 [SMILES: CC(C)N(C)[C@@H]1CC[C@H](N2CC[C@H](Nc3ncnc4ccc(C(F)(F)F)cc34)C2=O)[C@H](NC(=O)c2cccc(NCCOCCOCCOCCOCCOCCNC(=O)[C@H]3CC(=O)N(C)[C@@H]3c3cccnc3)n2)C1] |
| A125 | 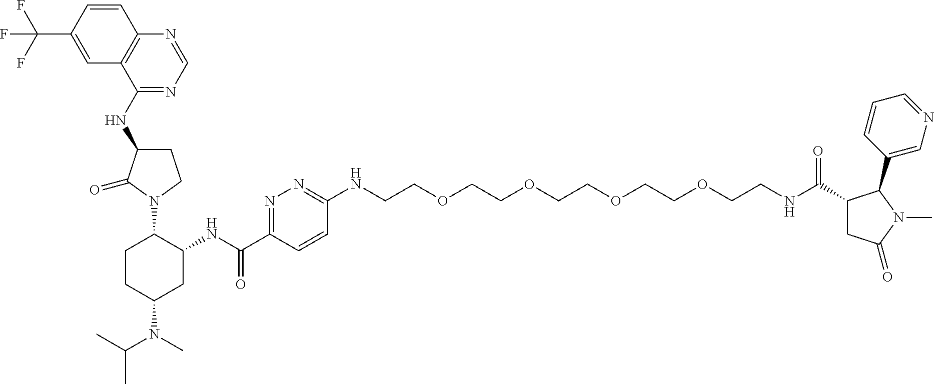 [SMILES: CC(C)N(C)[C@@H]1CC[C@H](N2CC[C@H](Nc3ncnc4ccc(C(F)(F)F)cc34)C2=O)[C@H](NC(=O)c2ccc(NCCOCCOCCOCCOCCNC(=O)[C@H]3CC(=O)N(C)[C@@H]3c3cccnc3)nn2)C1] |
| A126 | 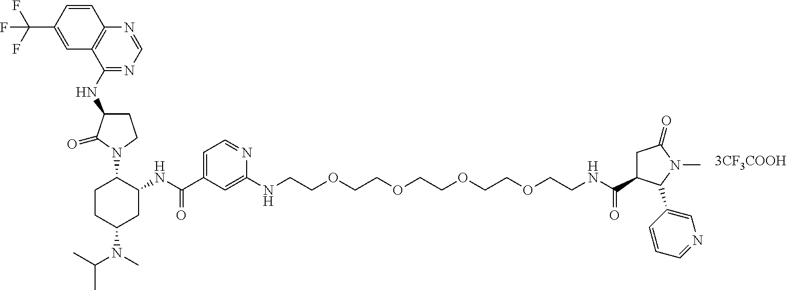 |
| A127 | 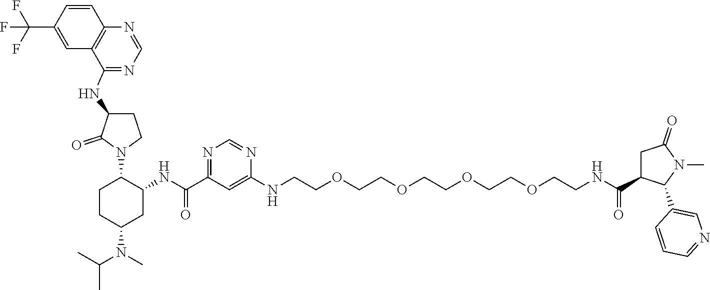 [SMILES: CC(C)N(C)[C@@H]1CC[C@H](N2CC[C@H](Nc3ncnc4ccc(C(F)(F)F)cc34)C2=O)[C@H](NC(=O)c2cc(NCCOCCOCCOCCOCCNC(=O)[C@H]3CC(=O)N(C)[C@@H]3c3cccnc3)ncn2)C1] |
| A128 | 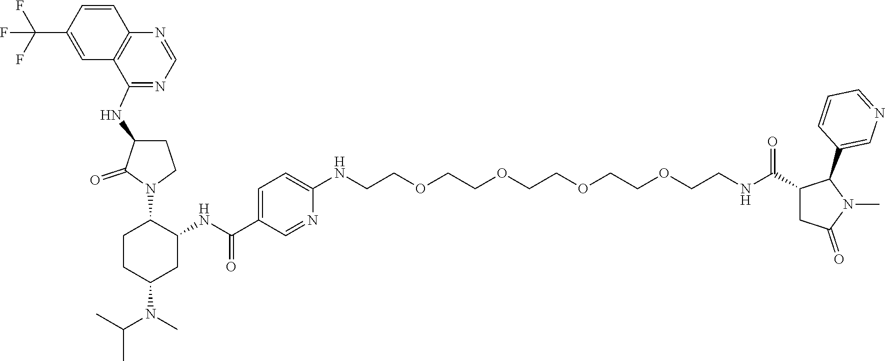 [SMILES: CC(C)N(C)[C@@H]1CC[C@H](N2CC[C@H](Nc3ncnc4ccc(C(F)(F)F)cc34)C2=O)[C@H](NC(=O)c2ccc(NCCOCCOCCOCCOCCNC(=O)[C@H]3CC(=O)N(C)[C@@H]3c3cccnc3)nc2)C1] |
| A129 | 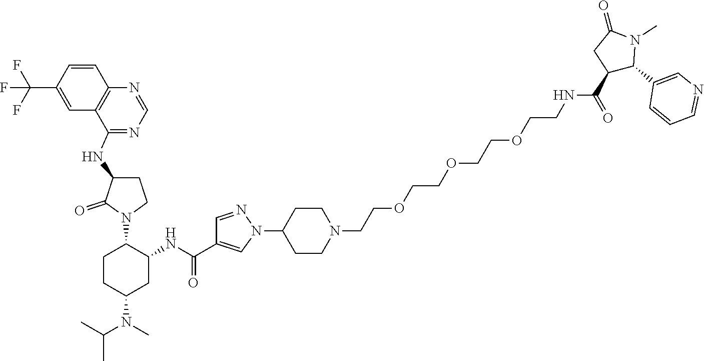 [SMILES: CC(C)N(C)[C@@H]1CC[C@H](N2CC[C@H](Nc3ncnc4ccc(C(F)(F)F)cc34)C2=O)[C@H](NC(=O)c2cnn(C3CCN(CCOCCOCCOCCNC(=O)[C@H]4CC(=O)N(C)[C@@H]4c4cccnc4)CC3)c2)C1] |
| A130 | 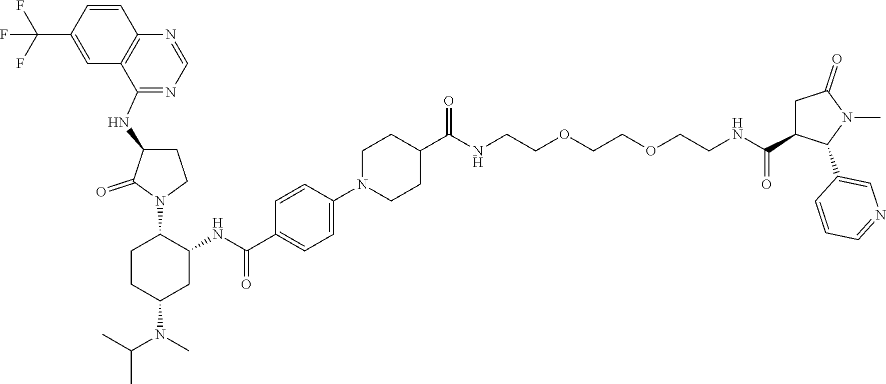 [SMILES: CC(C)N(C)[C@@H]1CC[C@H](N2CC[C@H](Nc3ncnc4ccc(C(F)(F)F)cc34)C2=O)[C@H](NC(=O)c2ccc(N3CCC(C(=O)NCCOCCOCCNC(=O)[C@H]4CC(=O)N(C)[C@@H]4c4cccnc4)CC3)cc2)C1] |
| A131 | 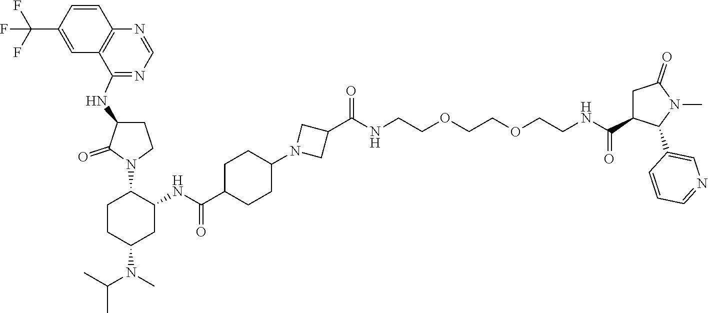 [SMILES: CC(C)N(C)[C@@H]1CC[C@H](N2CC[C@H](Nc3ncnc4ccc(C(F)(F)F)cc34)C2=O)[C@H](NC(=O)C2CCC(N3CC(C(=O)NCCOCCOCCNC(=O)[C@H]4CC(=O)N(C)[C@@H]4c4cccnc4)C3)CC2)C1] |
| A132 | 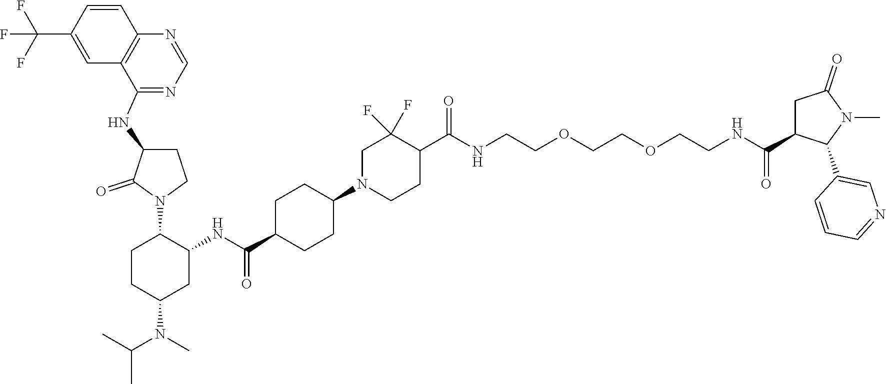 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCC(C(=O)NCCOCCOCCNC(=O)[C@H]4CC(=O)N(C)[C@@H]4c4cccnc4)C(F)(F)C3)CC2)C1] |
| A133 | 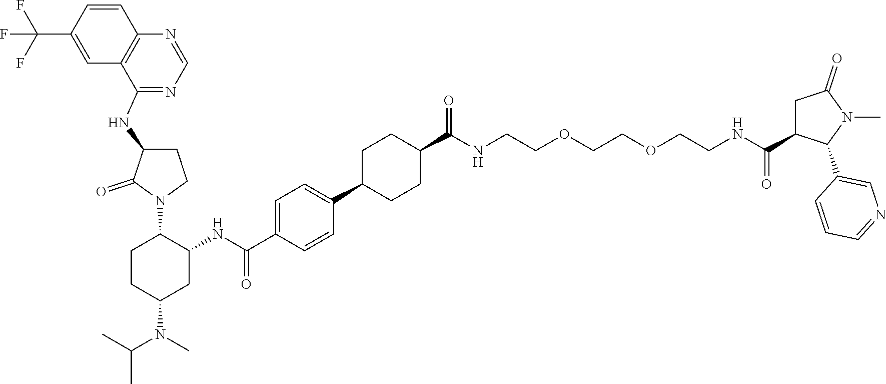 [SMILES: CC(C)N(C)[C@@H]1CC[C@H](N2CC[C@H](Nc3ncnc4ccc(C(F)(F)F)cc34)C2=O)[C@H](NC(=O)c2ccc([C@H]3CC[C@@H](C(=O)NCCOCCOCCNC(=O)[C@H]4CC(=O)N(C)[C@@H]4c4cccnc4)CC3)cc2)C1] |
| A134 | 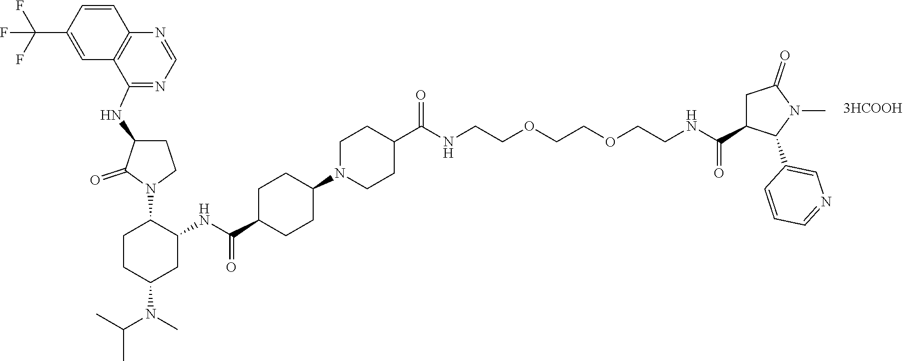 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCC(C(=O)NCCOCCOCCNC(=O)[C@H]4CC(=O)N(C)[C@@H]4c4cccnc4)CC3)CC2)C1] |
| A135 | 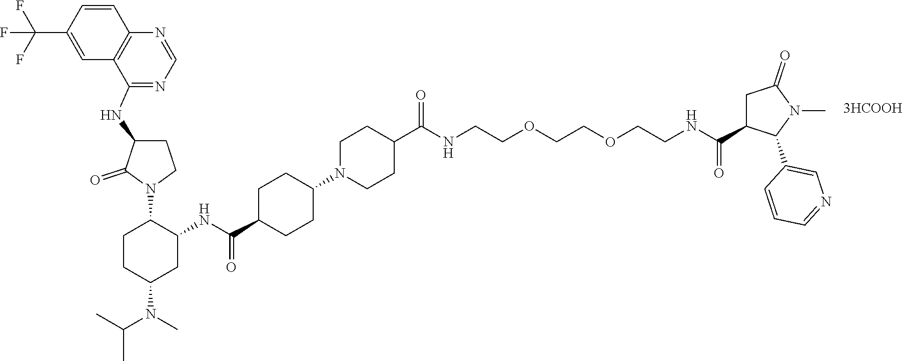 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCC(C(=O)NCCOCCOCCNC(=O)[C@H]4CC(=O)N(C)[C@@H]4c4cccnc4)CC3)CC2)C1] |
| A136 | 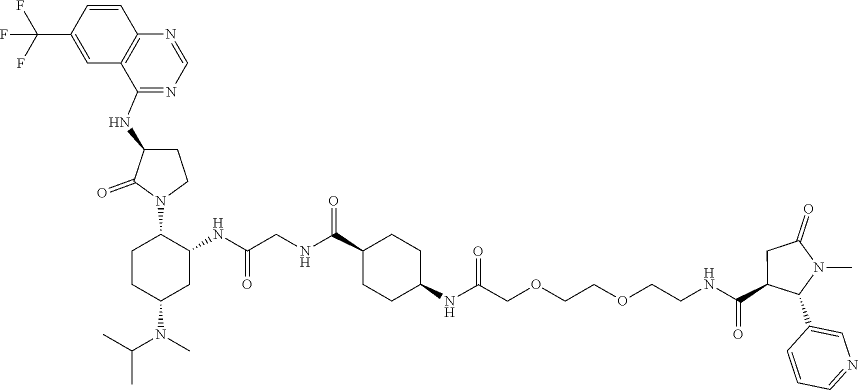 [SMILES: CC(C)N(C)[C@@H]1CC[C@H](N2CC[C@H](Nc3ncnc4ccc(C(F)(F)F)cc34)C2=O)[C@H](NC(=O)CNC(=O)[C@H]2CC[C@@H](NC(=O)COCCOCCNC(=O)[C@H]3CC(=O)N(C)[C@@H]3c3cccnc3)CC2)C1] |
| A137 | 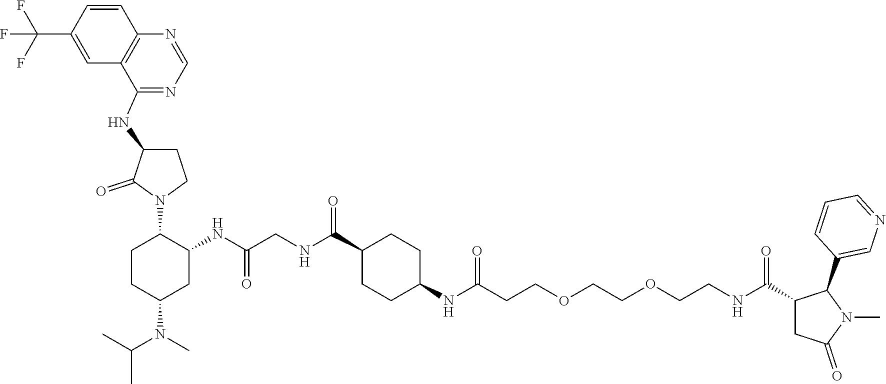 [SMILES: CC(C)N(C)[C@@H]1CC[C@H](N2CC[C@H](Nc3ncnc4ccc(C(F)(F)F)cc34)C2=O)[C@H](NC(=O)CNC(=O)[C@H]2CC[C@@H](NC(=O)CCOCCOCCNC(=O)[C@H]3CC(=O)N(C)[C@@H]3c3cccnc3)CC2)C1] |
| A138 | 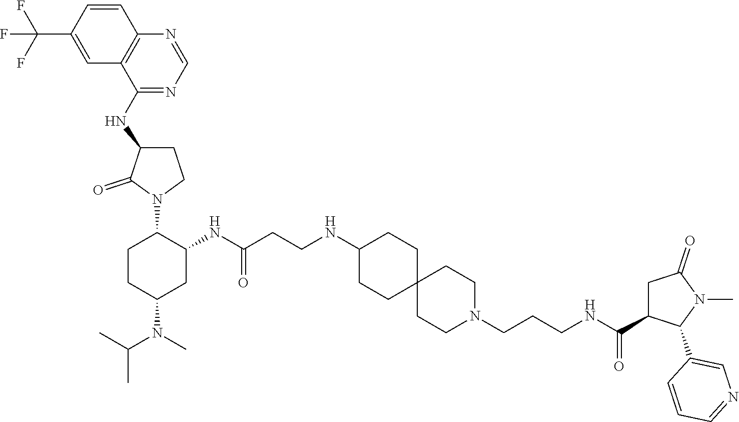 [SMILES: CC(C)N(C)[C@@H]1CC[C@H](N2CC[C@H](Nc3ncnc4ccc(C(F)(F)F)cc34)C2=O)[C@H](NC(=O)CCNC2CCC3(CC2)CCN(CCCNC(=O)[C@H]2CC(=O)N(C)[C@@H]2c2cccnc2)CC3)C1] |
| A139 | 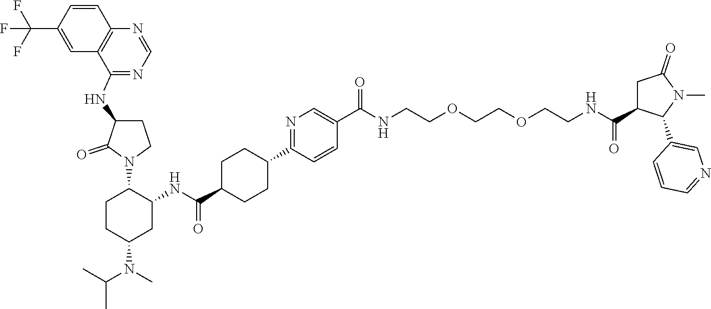 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)cn3)CC2)C1] |
| A140 | 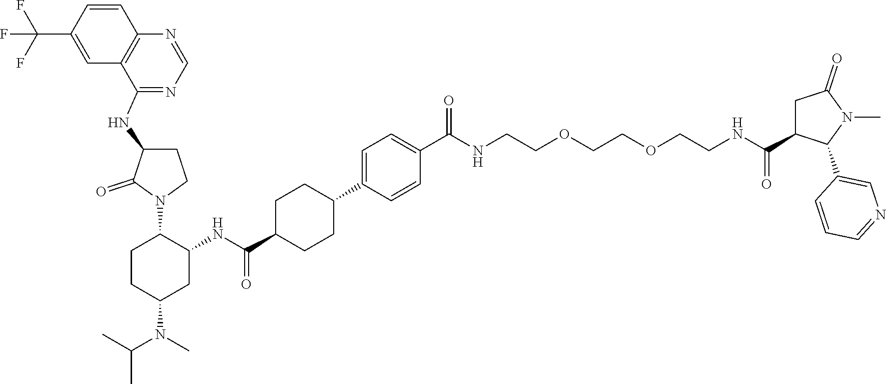 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)cc3)CC2)C1] |
| A141 | 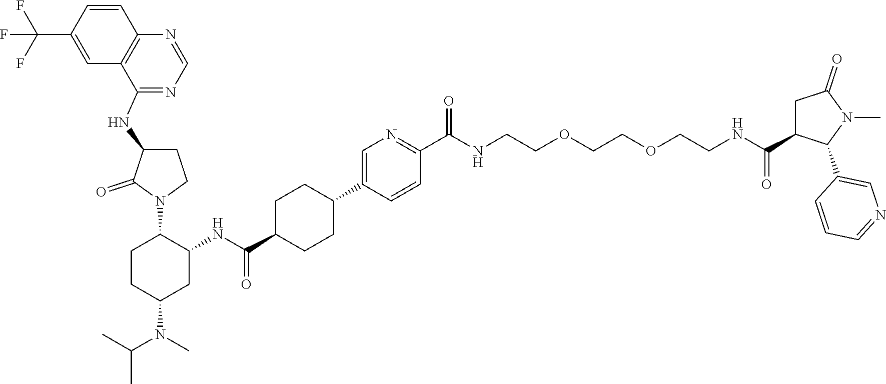 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](c3ccc(C(=O)NCCOCCOCCNC(=O)[C@H]4CC(=O)N(C)[C@@H]4c4cccnc4)nc3)CC2)C1] |
| A142 | 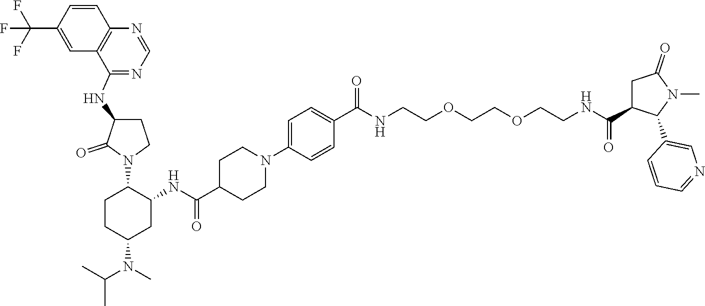 [SMILES: CC(C)N(C)[C@@H]1CC[C@H](N2CC[C@H](Nc3ncnc4ccc(C(F)(F)F)cc34)C2=O)[C@H](NC(=O)C2CCN(c3ccc(C(=O)NCCOCCOCCNC(=O)[C@H]4CC(=O)N(C)[C@@H]4c4cccnc4)cc3)CC2)C1] |
| A143 | 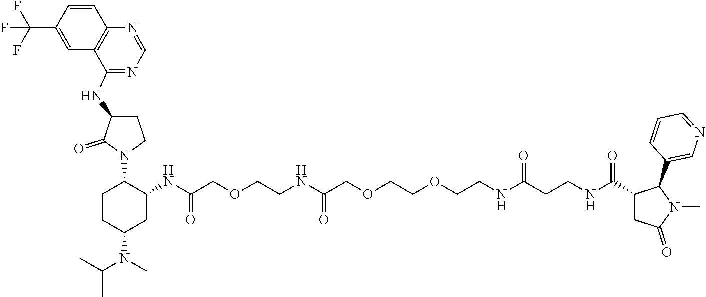 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCOCCNC(=O)CCNC(=O)[C@H]2CC(=O)N(C)[C@@H]2c2cccnc2)C1] |
| A144 | 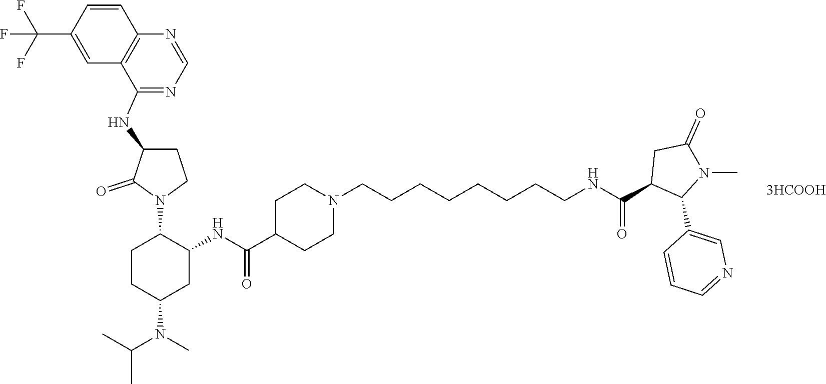 [SMILES: CC(C)N(C)[C@@H]1CC[C@H](N2CC[C@H](Nc3ncnc4ccc(C(F)(F)F)cc34)C2=O)[C@H](NC(=O)C2CCN(CCCCCCCCNC(=O)[C@H]3CC(=O)N(C)[C@@H]3c3cccnc3)CC2)C1] |
| A145 | 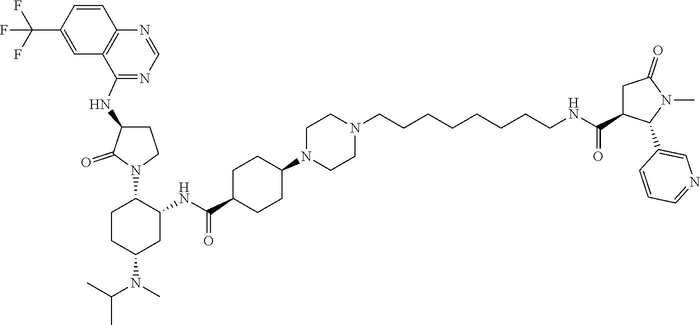 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](N3CCN(CCCCCCCCNC(=O)[C@H]4CC(=O)N(C)[C@@H]4c4cccnc4)CC3)CC2)C1] |
| A146 | 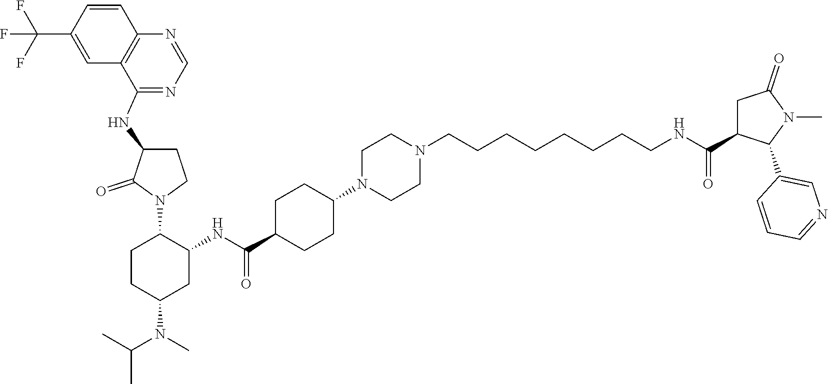 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(CCCCCCCCNC(=O)[C@H]4CC(=O)N(C)[C@@H]4c4cccnc4)CC3)CC2)C1] |
| A147 | 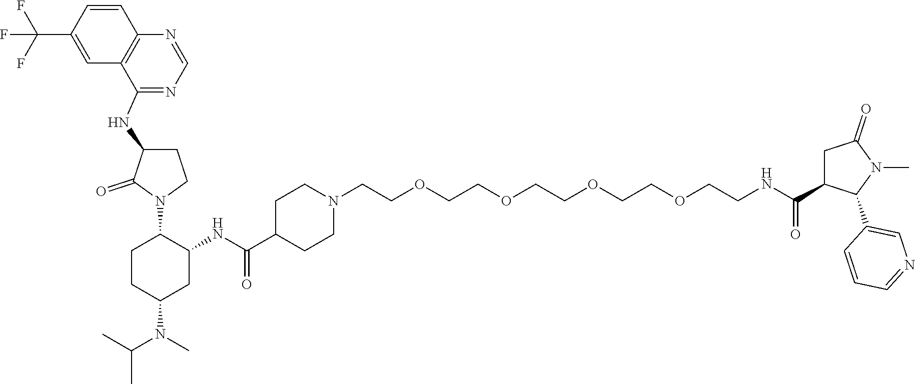 [SMILES: CC(C)N(C)[C@@H]1CC[C@H](N2CC[C@H](Nc3ncnc4ccc(C(F)(F)F)cc34)C2=O)[C@H](NC(=O)C2CCN(CCOCCOCCOCCOCCNC(=O)[C@H]3CC(=O)N(C)[C@@H]3c3cccnc3)CC2)C1] |
| A148 | 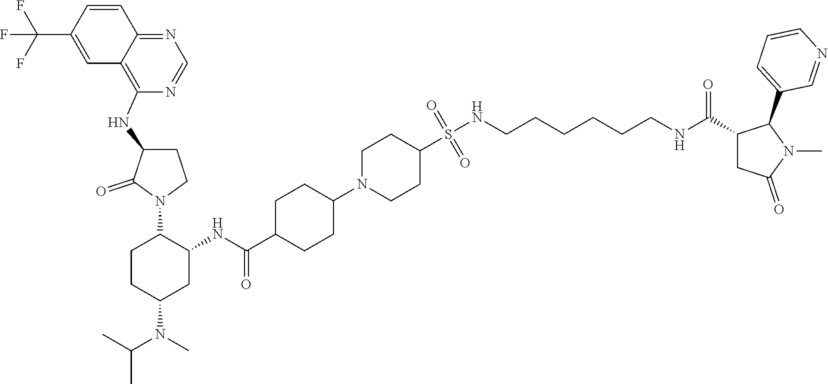 |
| A149 | 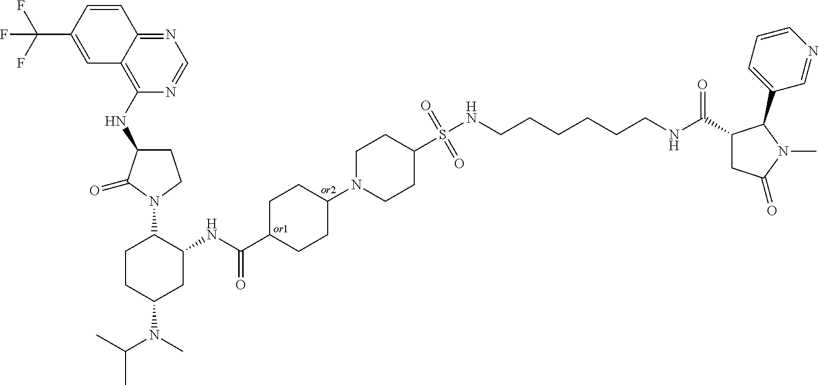 |
| A150 | 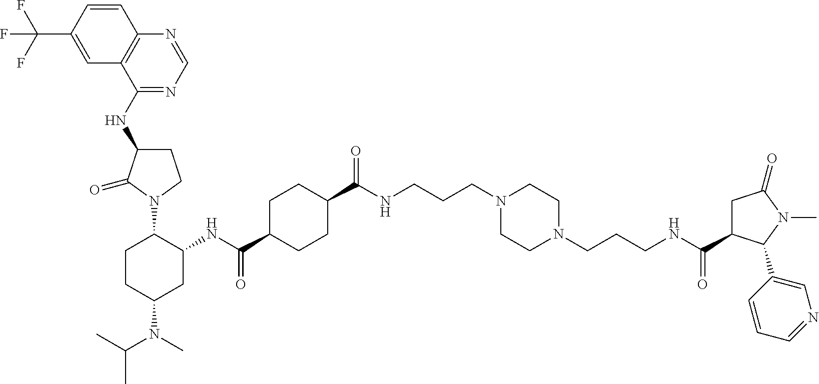 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](C(=O)NCCCN3CCN(CCCNC(=O)[C@H]4CC(=O)N(C)[C@@H]4c4cccnc4)CC3)CC2)C1] |
| A151 | 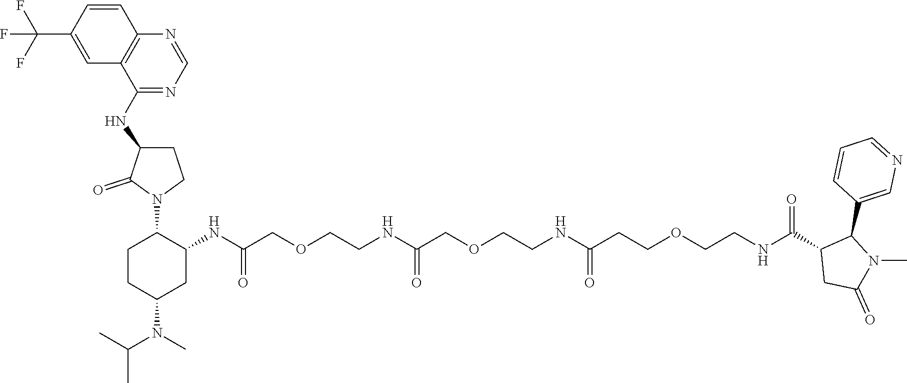 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)COCCNC(=O)CCOCCNC(=O)[C@H]2CC(=O)N(C)[C@@H]2c2cccnc2)C1] |
| A152 | 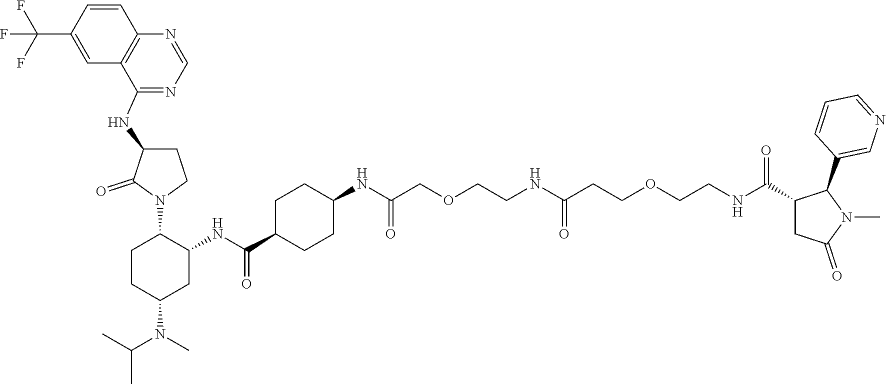 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCNC(=O)CCOCCNC(=O)[C@H]3CC(=O)N(C)[C@@H]3c3cccnc3)CC2)C1] |
| A153 | 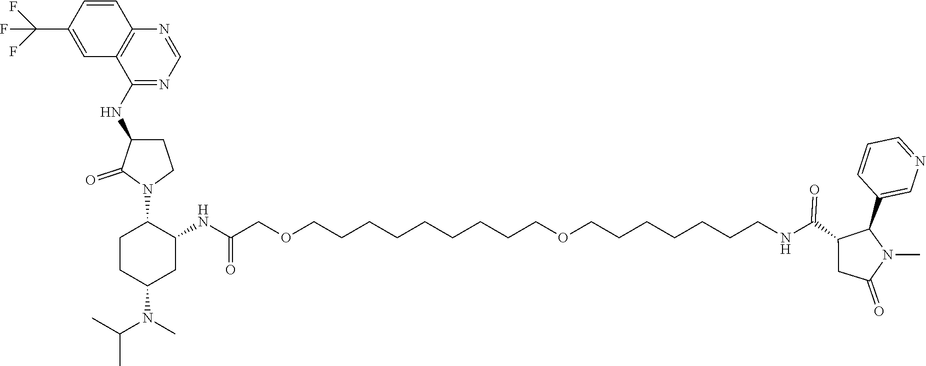 |
| A154 | 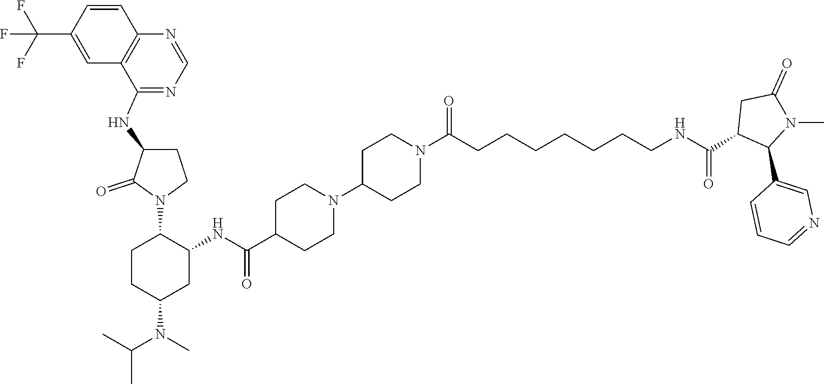 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCCCNC(=O)[C@@H]4CC(=O)N(C)[C@H]4c4cccnc4)CC3)CC2)C1] |
| A155 | 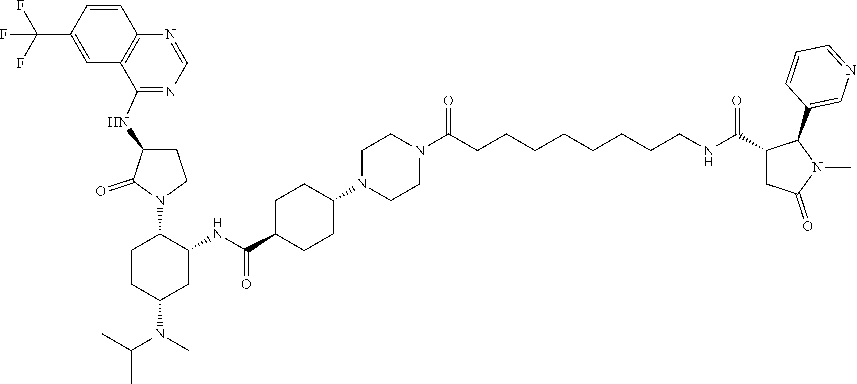 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(C(=O)CCCCCCCCNC(=O)[C@H]4CC(=O)N(C)[C@@H]4c4cccnc4)CC3)CC2)C1] |
| A156 | 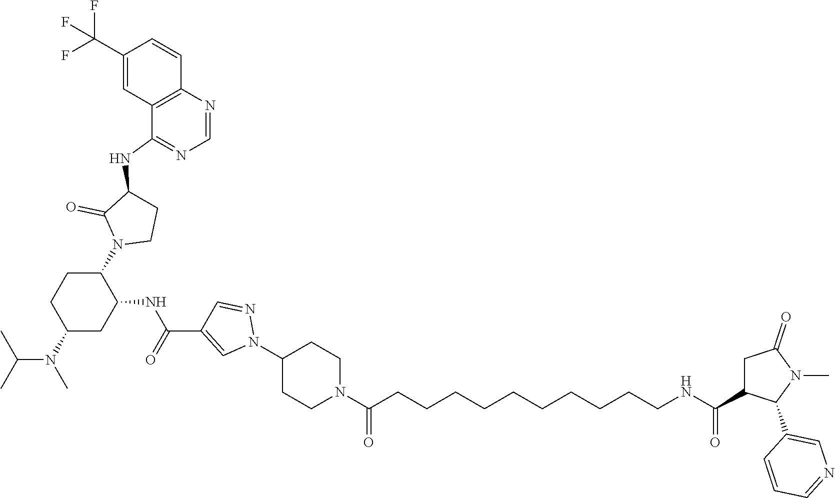 |
| A157 | 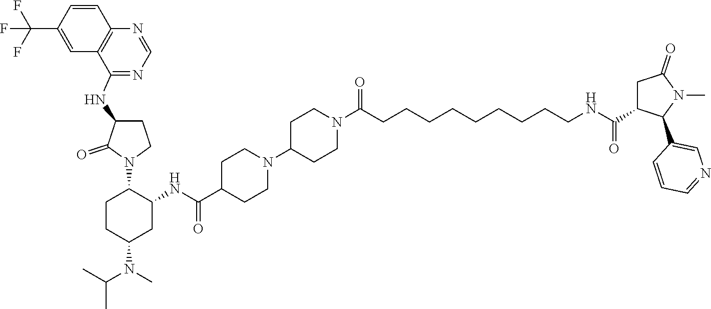 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCCCCCCCCNC(=O)[C@@H]4CC(=O)N(C)[C@H]4c4cccnc4)CC3)CC2)C1] |
| A158 | 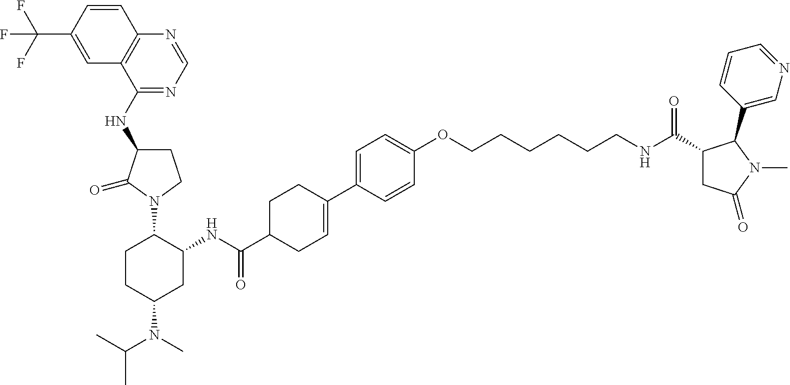 [SMILES: CC(C)N(C)[C@@H]1CC[C@H](N2CC[C@H](Nc3ncnc4ccc(C(F)(F)F)cc34)C2=O)[C@H](NC(=O)C2CC=C(c3ccc(OCCCCCCNC(=O)[C@H]4CC(=O)N(C)[C@@H]4c4cccnc4)cc3)CC2)C1] |
| A159 | 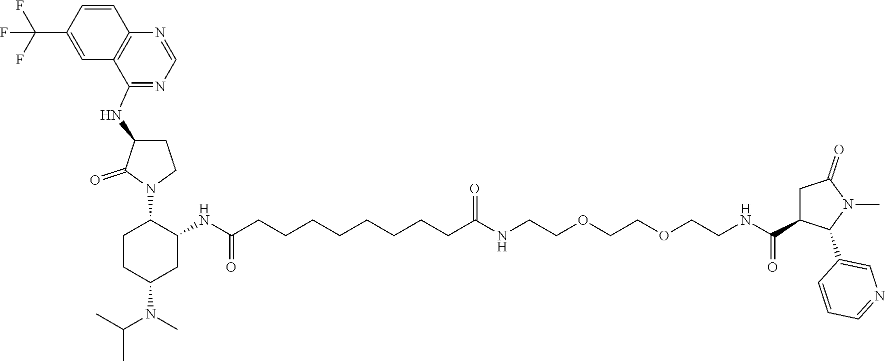 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCOCCOCCNC(=O)[C@H]2CC(=O)N(C)[C@@H]2c2cccnc2)C1] |
| A160 | 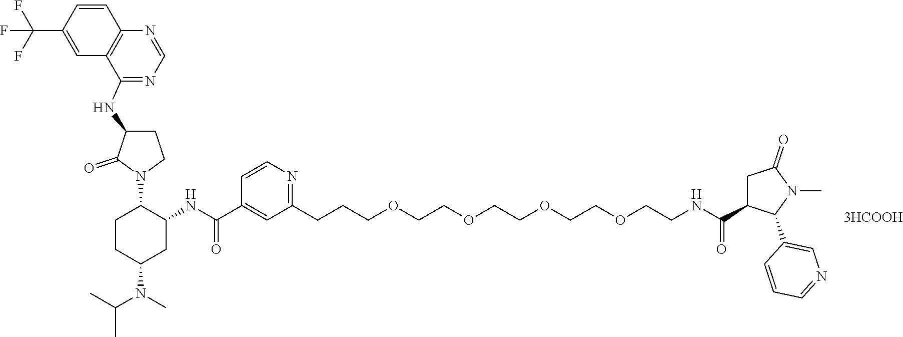 [SMILES: CC(C)N(C)[C@@H]1CC[C@H](N2CC[C@H](Nc3ncnc4ccc(C(F)(F)F)cc34)C2=O)[C@H](NC(=O)c2ccnc(CCCOCCOCCOCCOCCNC(=O)[C@H]3CC(=O)N(C)[C@@H]3c3cccnc3)c2)C1] |
| A161 | 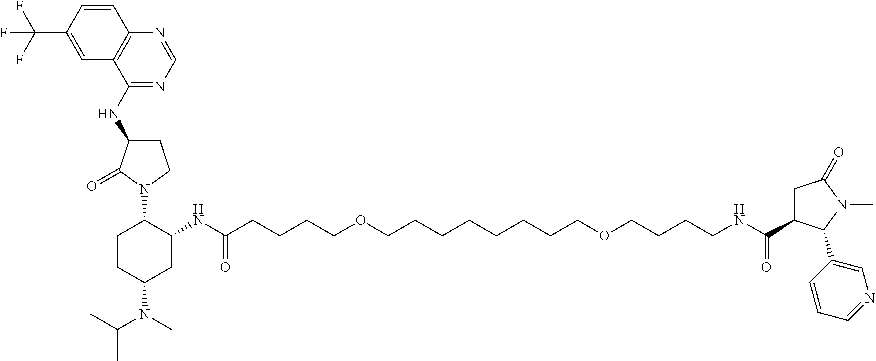 [SMILES: CC(C)N(C)[C@@H]1CC[C@H](N2CC[C@H](Nc3ncnc4ccc(C(F)(F)F)cc34)C2=O)[C@H](NC(=O)CCCCOCCCCCCCCOCCCCNC(=O)[C@H]2CC(=O)N(C)[C@@H]2c2cccnc2)C1] |
| A162 | 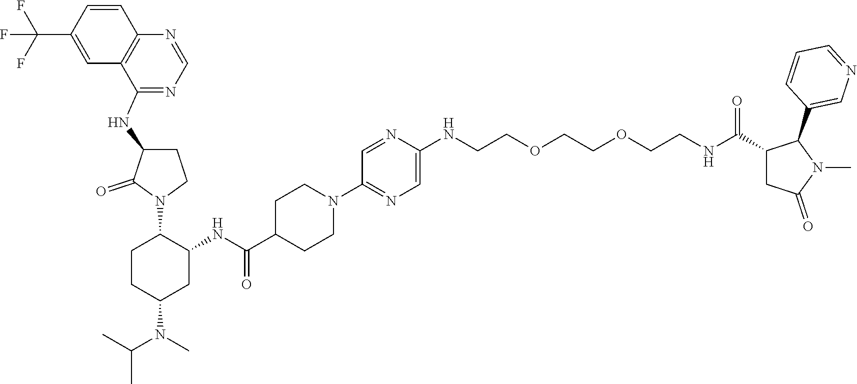 [SMILES: CC(C)N(C)[C@@H]1CC[C@H](N2CC[C@H](Nc3ncnc4ccc(C(F)(F)F)cc34)C2=O)[C@H](NC(=O)C2CCN(c3cnc(NCCOCCOCCNC(=O)[C@H]4CC(=O)N(C)[C@@H]4c4cccnc4)cn3)CC2)C1] |
| A163 | 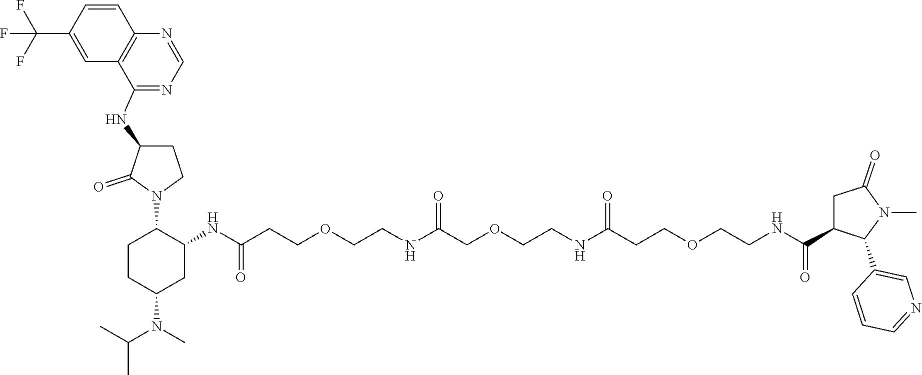 [SMILES: CC(C)N(C)[C@@H]1CC[C@H](N2CC[C@H](Nc3ncnc4ccc(C(F)(F)F)cc34)C2=O)[C@H](NC(=O)CCOCCNC(=O)COCCNC(=O)CCOCCNC(=O)[C@H]2CC(=O)N(C)[C@@H]2c2cccnc2)C1] |
| A164 | 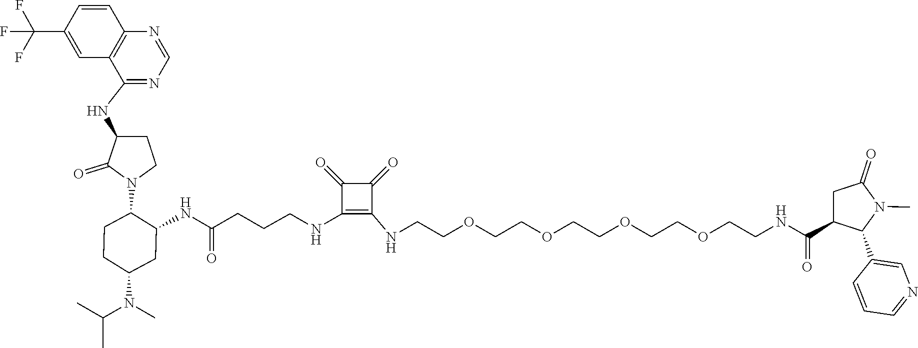 [SMILES: CC(C)N(C)[C@@H]1CC[C@H](N2CC[C@H](Nc3ncnc4ccc(C(F)(F)F)cc34)C2=O)[C@H](NC(=O)CCCNc2c(NCCOCCOCCOCCOCCNC(=O)[C@H]3CC(=O)N(C)[C@@H]3c3cccnc3)c(=O)c2=O)C1] |
| A165 | 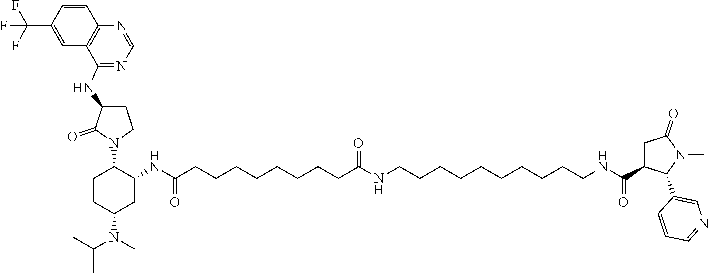 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCCCCCCCCCNC(=O)[C@H]2CC(=O)N(C)[C@@H]2c2cccnc2)C1] |
| A166 | 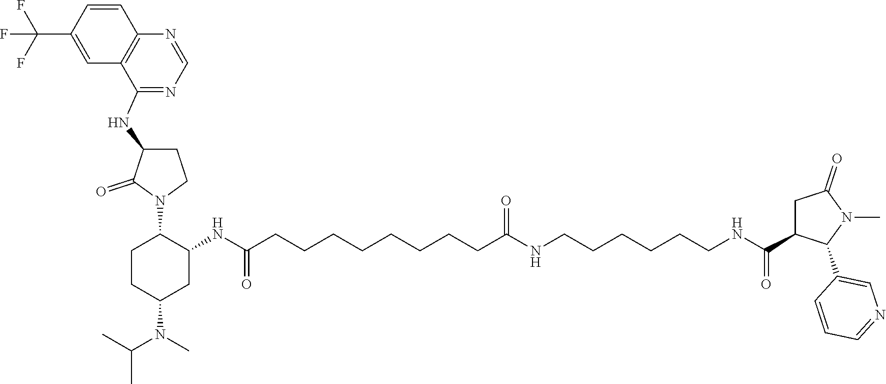 [SMILES: CC(C)N(C)[C@@H]1CC[C@H](N2CC[C@H](Nc3ncnc4ccc(C(F)(F)F)cc34)C2=O)[C@H](NC(=O)CCCCCCCCC(=O)NCCCCCCNC(=O)[C@H]2CC(=O)N(C)[C@@H]2c2cccnc2)C1] |
| A167 | 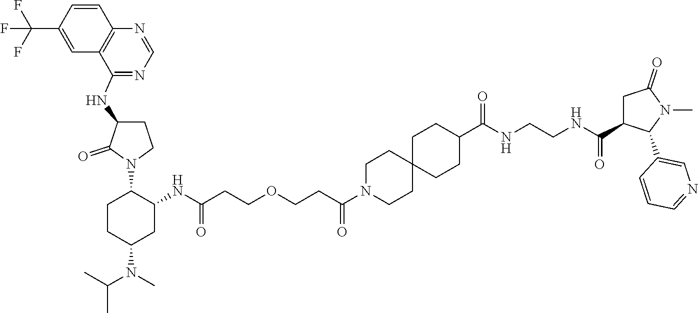 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N2CCC3(CCC(C(=O)NCCNC(=O)[C@H]4CC(=O)N(C)[C@@H]4c4cccnc4)CC3)CC2)C1] |
| A168 | 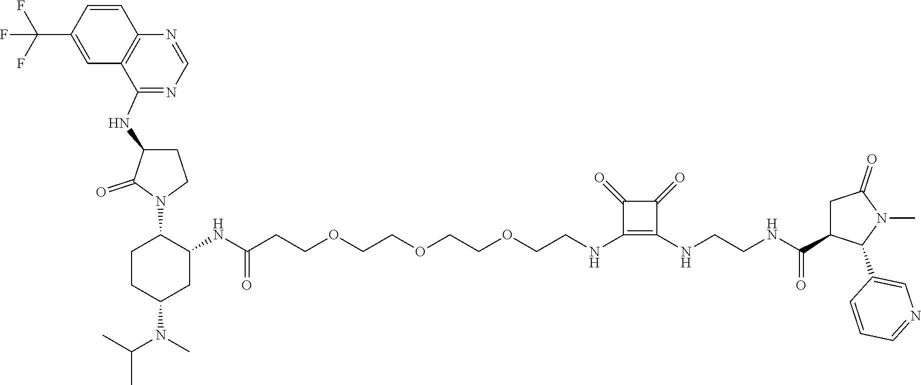 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCNc2c(NCCNC(=O)[C@H]3CC(=O)N(C)[C@@H]3c3cccnc3)c(=O)c2=O)C1] |
| A169 | 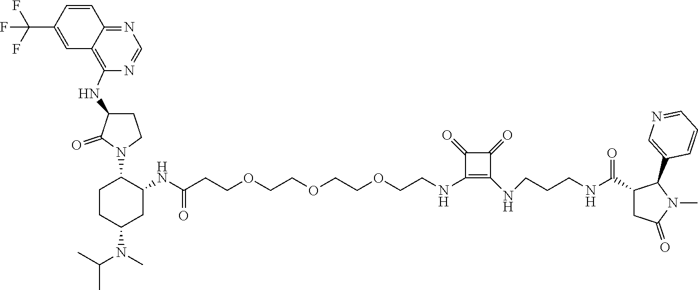 [SMILES: CC(C)N(C)[C@@H]1CC[C@H](N2CC[C@H](Nc3ncnc4ccc(C(F)(F)F)cc34)C2=O)[C@H](NC(=O)CCOCCOCCOCCNc2c(NCCCNC(=O)[C@H]3CC(=O)N(C)[C@@H]3c3cccnc3)c(=O)c2=O)C1] |
| A170 | 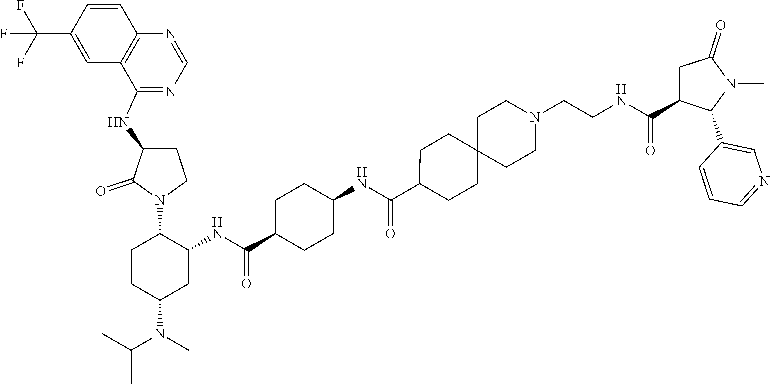 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)C3CCC4(CC3)CCN(CCNC(=O)[C@H]3CC(=O)N(C)[C@@H]3c3cccnc3)CC4)CC2)C1] |
| A171 | 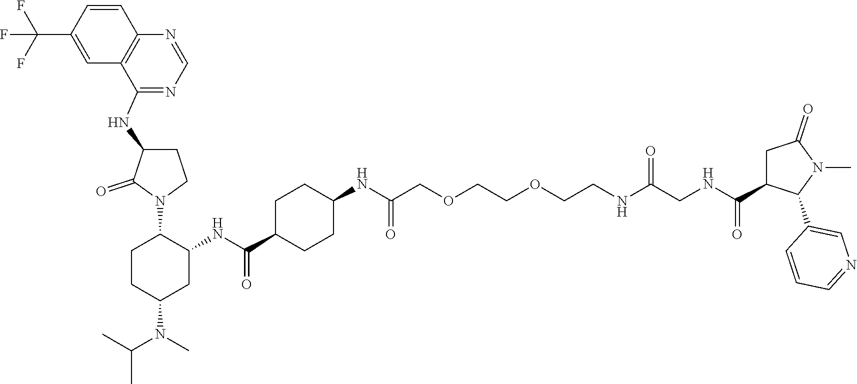 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)COCCOCCNC(=O)CNC(=O)[C@H]3CC(=O)N(C)[C@@H]3c3cccnc3)CC2)C1] |
| A172 | 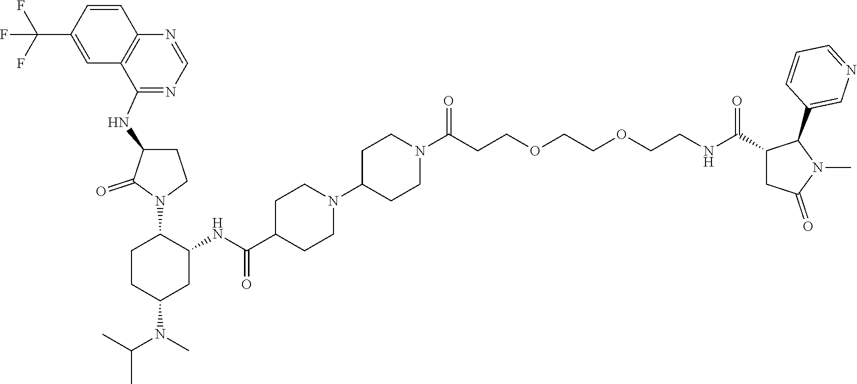 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C(=O)CCOCCOCCNC(=O)[C@H]4CC(=O)N(C)[C@@H]4c4cccnc4)CC3)CC2)C1] |
| A173 | 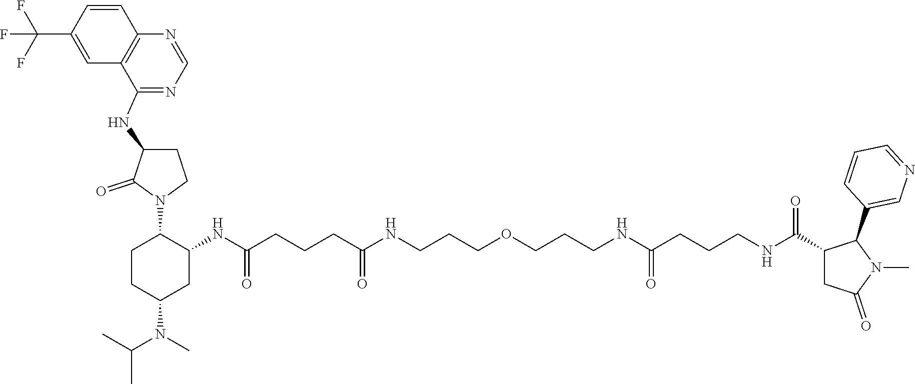 [SMILES: CC(C)N(C)[C@@H]1CC[C@H](N2CC[C@H](Nc3ncnc4ccc(C(F)(F)F)cc34)C2=O)[C@H](NC(=O)CCCC(=O)NCCCOCCCNC(=O)CCCNC(=O)[C@H]2CC(=O)N(C)[C@@H]2c2cccnc2)C1] |
| A175 | 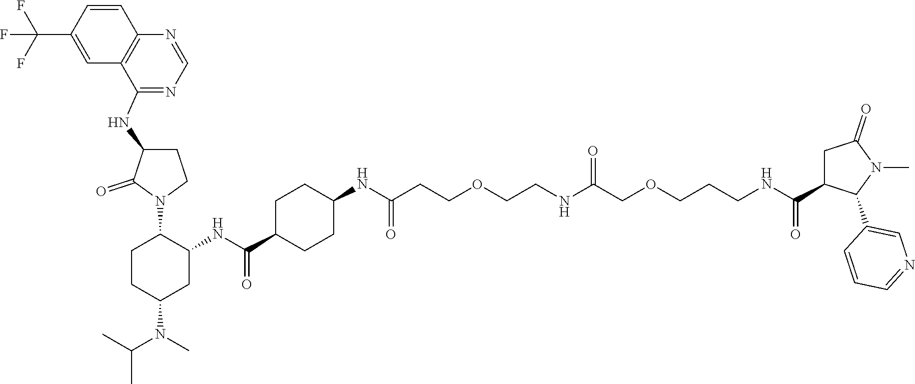 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)COCCCNC(=O)[C@H]3CC(=O)N(C)[C@@H]3c3cccnc3)CC2)C1] |
| A176 | 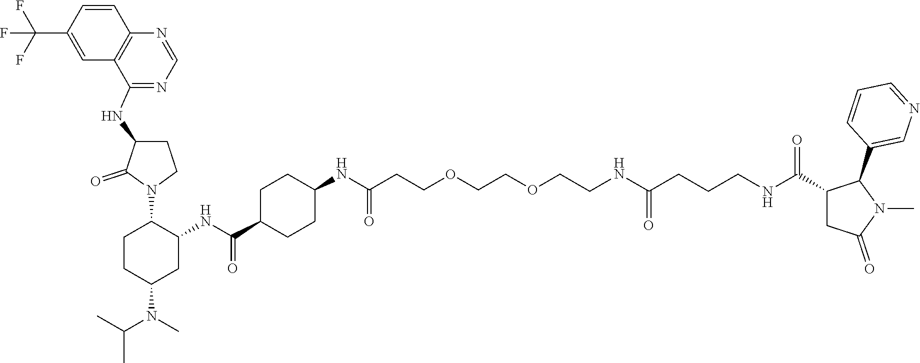 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCOCCNC(=O)CCCNC(=O)[C@H]3CC(=O)N(C)[C@@H]3c3cccnc3)CC2)C1] |
| A177 | 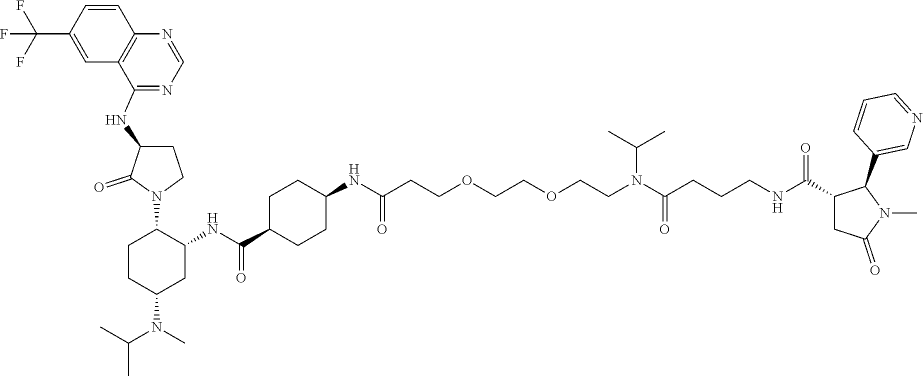 [SMILES: CC(C)N(CCOCCOCCC(=O)N[C@H]1CC[C@@H](C(=O)N[C@@H]2C[C@H](N(C)C(C)C)CC[C@@H]2N2CC[C@H](Nc3ncnc4ccc(C(F)(F)F)cc34)C2=O)CC1)C(=O)CCCNC(=O)[C@H]1CC(=O)N(C)[C@@H]1c1cccnc1] |
| A178 | 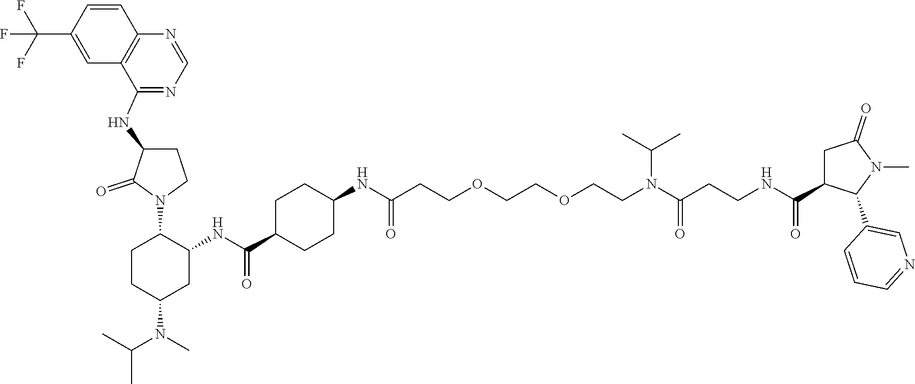 [SMILES: CC(C)N(CCOCCOCCC(=O)N[C@H]1CC[C@@H](C(=O)N[C@@H]2C[C@H](N(C)C(C)C)CC[C@@H]2N2CC[C@H](Nc3ncnc4ccc(C(F)(F)F)cc34)C2=O)CC1)C(=O)CCNC(=O)[C@H]1CC(=O)N(C)[C@@H]1c1cccnc1] |
| A179 | 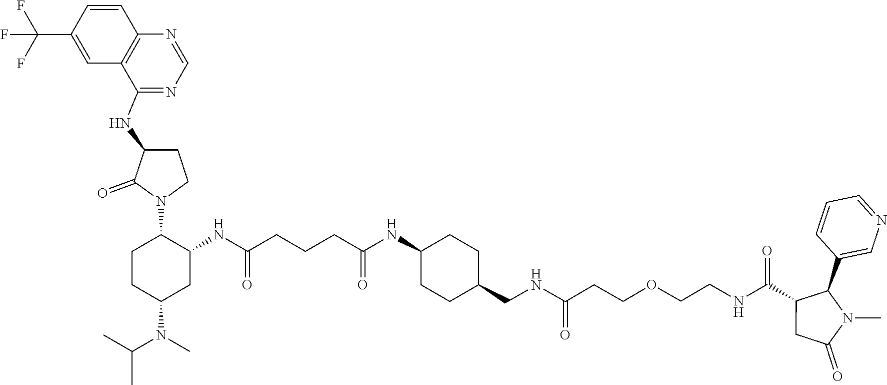 [SMILES: CC(C)N(C)[C@@H]1CC[C@H](N2CC[C@H](Nc3ncnc4ccc(C(F)(F)F)cc34)C2=O)[C@H](NC(=O)CCCC(=O)N[C@H]2CC[C@@H](CNC(=O)CCOCCNC(=O)[C@H]3CC(=O)N(C)[C@@H]3c3cccnc3)CC2)C1] |
| A180 | 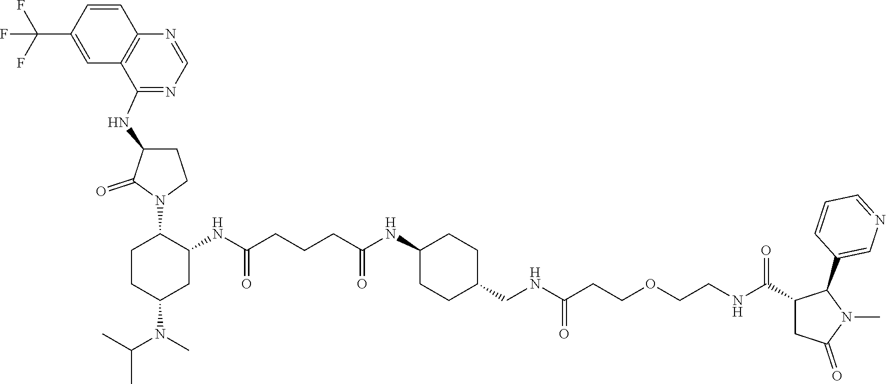 [SMILES: CC(C)N(C)[C@@H]1CC[C@H](N2CC[C@H](Nc3ncnc4ccc(C(F)(F)F)cc34)C2=O)[C@H](NC(=O)CCCC(=O)N[C@H]2CC[C@H](CNC(=O)CCOCCNC(=O)[C@H]3CC(=O)N(C)[C@@H]3c3cccnc3)CC2)C1] |
| A181 | 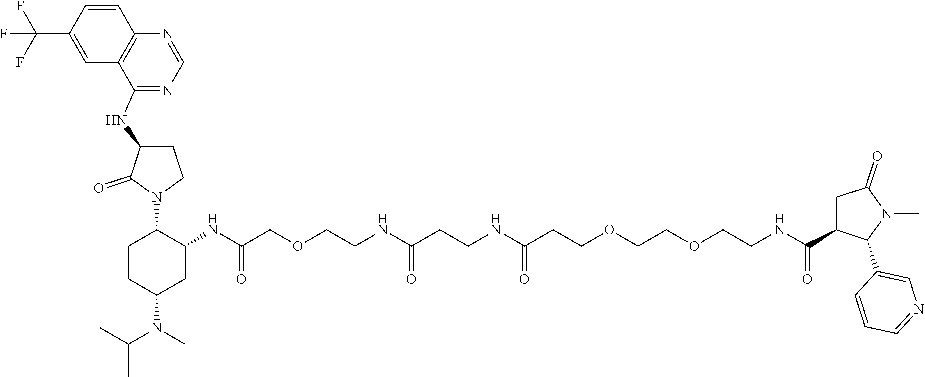 [SMILES: CC(C)N(C)[C@@H]1CC[C@H](N2CC[C@H](Nc3ncnc4ccc(C(F)(F)F)cc34)C2=O)[C@H](NC(=O)COCCNC(=O)CCNC(=O)CCOCCOCCNC(=O)[C@H]2CC(=O)N(C)[C@@H]2c2cccnc2)C1] |
| A182 | 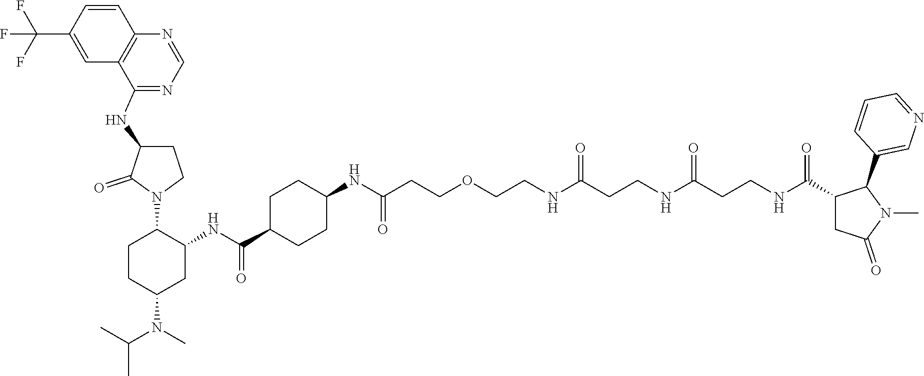 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCNC(=O)CCNC(=O)[C@H]3CC(=O)N(C)[C@@H]3c3cccnc3)CC2)C1] |
| A183 | 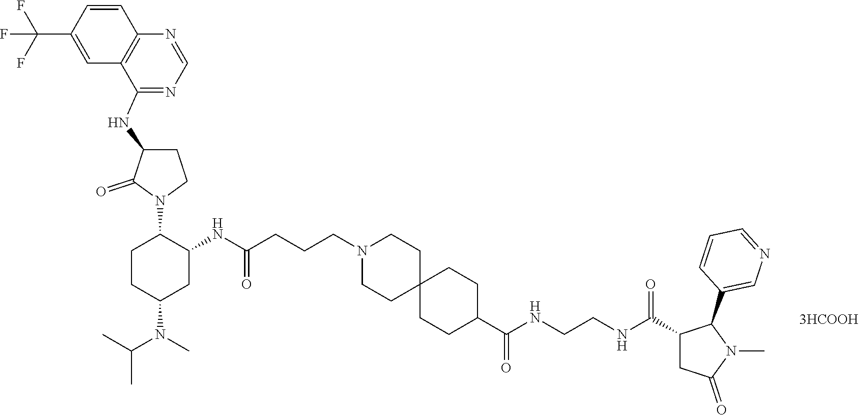 [SMILES: CC(C)N(C)[C@@H]1CC[C@H](N2CC[C@H](Nc3ncnc4ccc(C(F)(F)F)cc34)C2=O)[C@H](NC(=O)CCCN2CCC3(CCC(C(=O)NCCNC(=O)[C@H]4CC(=O)N(C)[C@@H]4c4cccnc4)CC3)CC2)C1] |
| A184 | 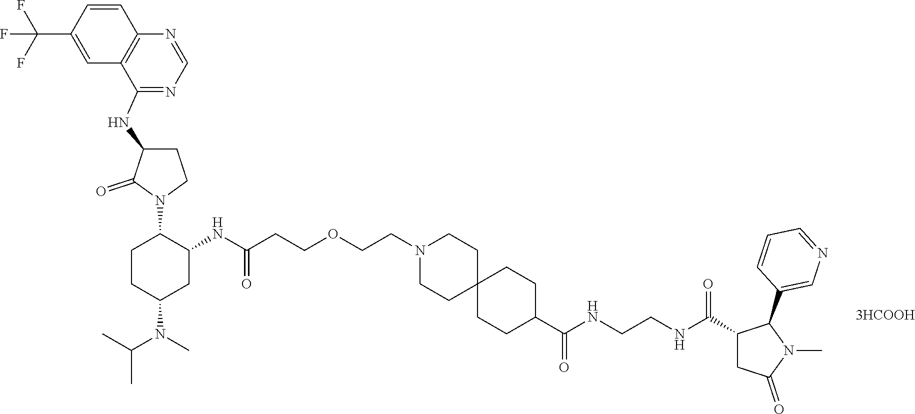 [SMILES: CC(C)N(C)[C@@H]1CC[C@H](N2CC[C@H](Nc3ncnc4ccc(C(F)(F)F)cc34)C2=O)[C@H](NC(=O)CCOCCN2CCC3(CCC(C(=O)NCCNC(=O)[C@H]4CC(=O)N(C)[C@@H]4c4cccnc4)CC3)CC2)C1] |
| A185 | 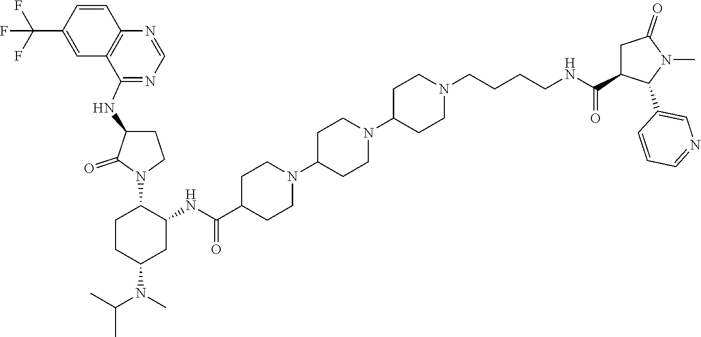 [SMILES: CC(C)N(C)[C@@H]1CC[C@H](N2CC[C@H](Nc3ncnc4ccc(C(F)(F)F)cc34)C2=O)[C@H](NC(=O)C2CCN(C3CCN(C4CCN(CCCCNC(=O)[C@H]5CC(=O)N(C)[C@@H]5c5cccnc5)CC4)CC3)CC2)C1] |
| A186 | 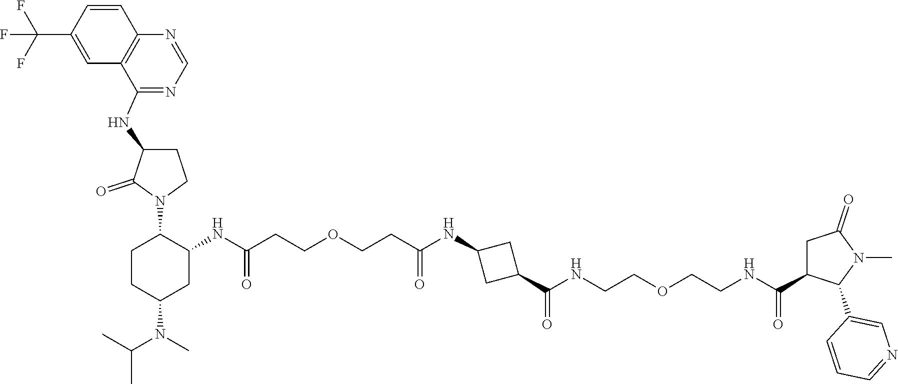 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N[C@H]2C[C@@H](C(=O)NCCOCCNC(=O)[C@H]3CC(=O)N(C)[C@@H]3c3cccnc3)C2)C1] |
| A187 | 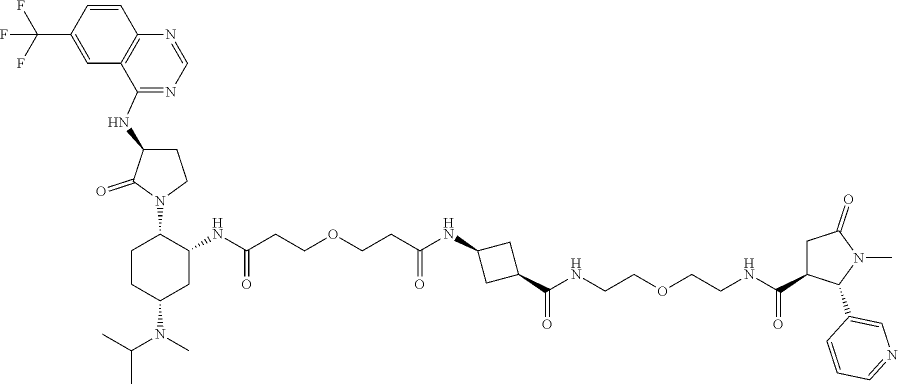 [SMILES: CC(C)N(C)[C@@H]1CC[C@H](N2CC[C@H](Nc3ncnc4ccc(C(F)(F)F)cc34)C2=O)[C@H](NC(=O)CCOCCC(=O)N[C@H]2C[C@@H](C(=O)NCCOCCNC(=O)[C@H]3CC(=O)N(C)[C@@H]3c3cccnc3)C2)C1] |
| A188 | 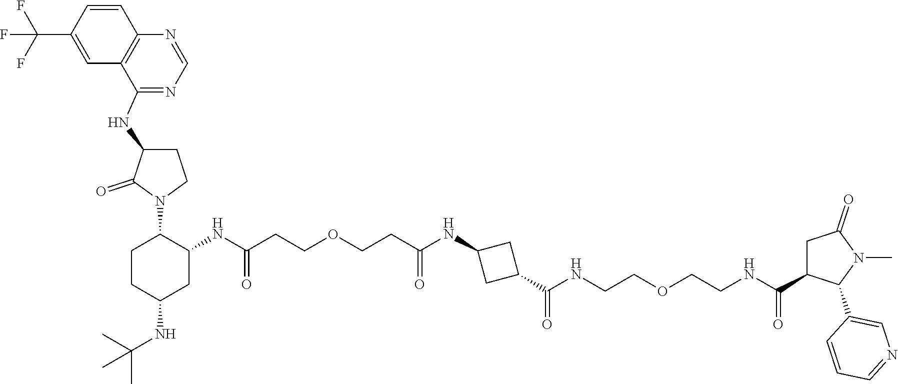 [SMILES: CN1C(=O)C[C@H](C(=O)NCCOCCNC(=O)[C@H]2C[C@H](NC(=O)CCOCCC(=O)N[C@@H]3C[C@H](NC(C)(C)C)CC[C@@H]3N3CC[C@H](Nc4ncnc5ccc(C(F)(F)F)cc45)C3=O)C2)[C@H]1c1cccnc1] |
| A189 | 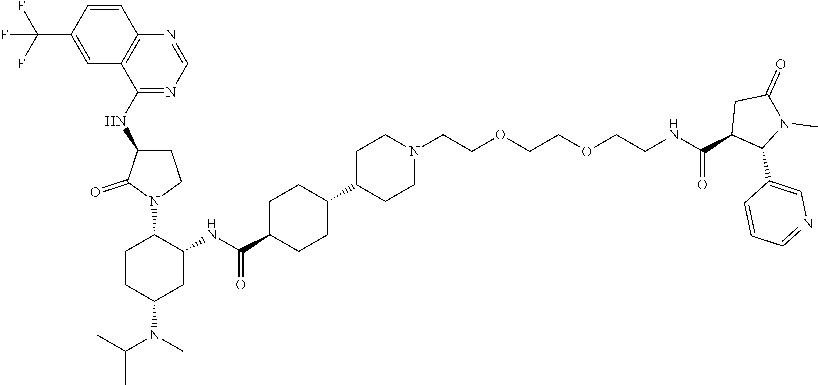 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C3CCN(CCOCCOCCNC(=O)[C@H]4CC(=O)N(C)[C@@H]4c4cccnc4)CC3)CC2)C1] |
| A190 | 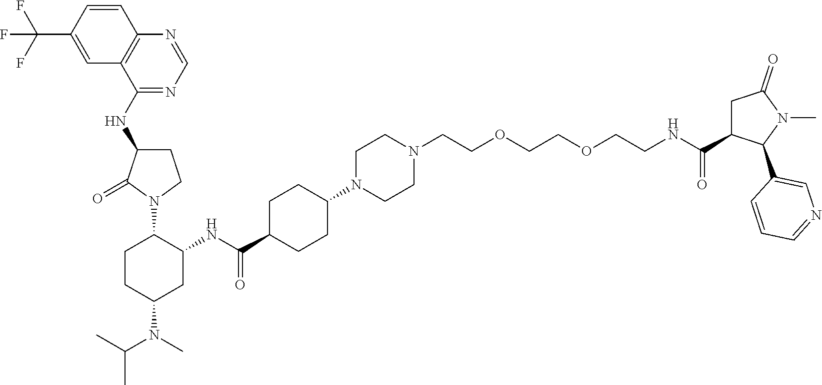 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](N3CCN(CCOCCOCCNC(=O)[C@H]4CC(=O)N(C)[C@H]4c4cccnc4)CC3)CC2)C1] |
| A191 | 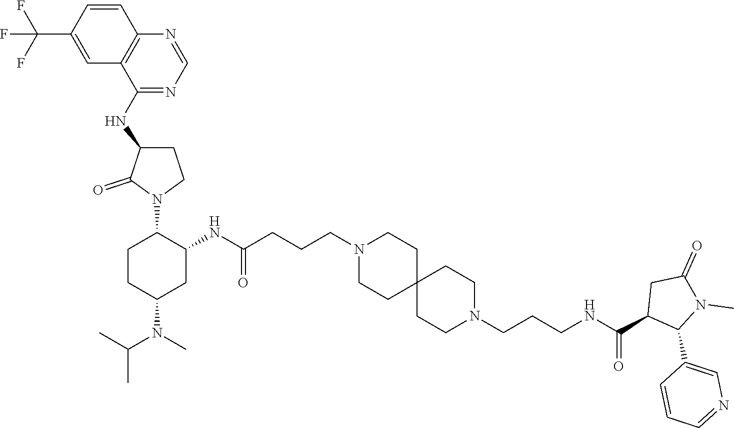 [SMILES: CC(C)N(C)[C@@H]1CC[C@H](N2CC[C@H](Nc3ncnc4ccc(C(F)(F)F)cc34)C2=O)[C@H](NC(=O)CCCN2CCC3(CCN(CCCNC(=O)[C@H]4CC(=O)N(C)[C@@H]4c4cccnc4)CC3)CC2)C1] |
| A192 | 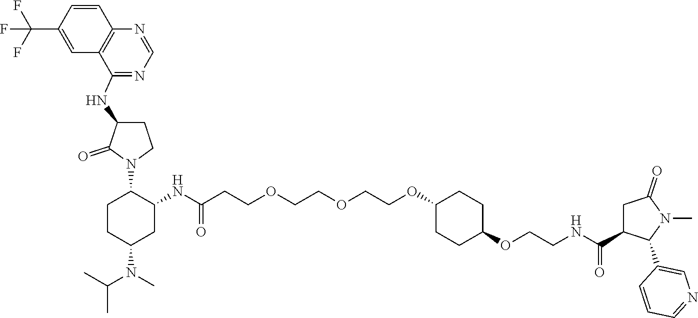 [SMILES: CC(C)N(C)[C@@H]1CC[C@H](N2CC[C@H](Nc3ncnc4ccc(C(F)(F)F)cc34)C2=O)[C@H](NC(=O)CCOCCOCCO[C@H]2CC[C@H](OCCNC(=O)[C@H]3CC(=O)N(C)[C@@H]3c3cccnc3)CC2)C1] |
| A193 | 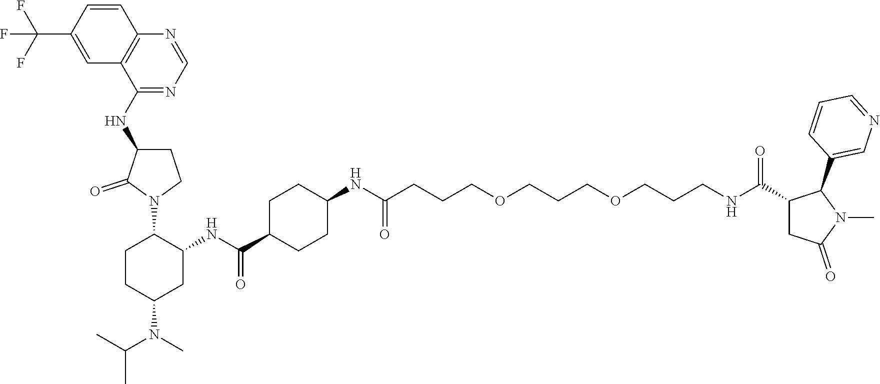 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCCOCCCOCCCNC(=O)[C@H]3CC(=O)N(C)[C@@H]3c3cccnc3)CC2)C1] |
| A194 | 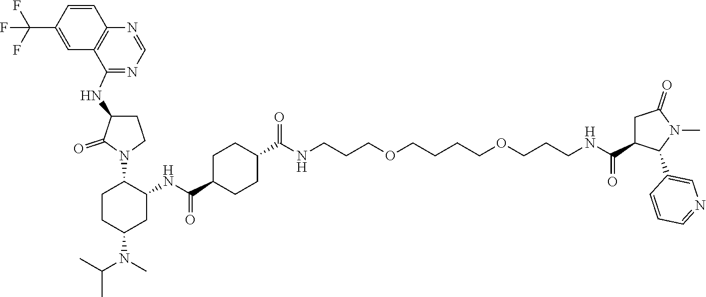 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@H](C(=O)NCCCOCCCCOCCCNC(=O)[C@H]3CC(=O)N(C)[C@@H]3c3cccnc3)CC2)C1] |
| A195 | 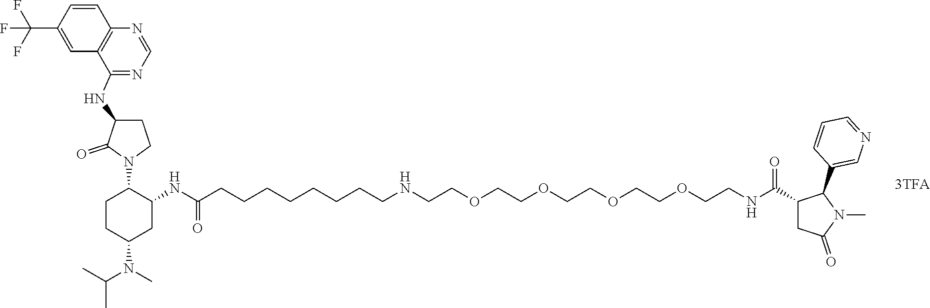 [SMILES: CC(C)N(C)[C@@H]1CC[C@H](N2CC[C@H](Nc3ncnc4ccc(C(F)(F)F)cc34)C2=O)[C@H](NC(=O)CCCCCCCCNCCOCCOCCOCCOCCNC(=O)[C@H]2CC(=O)N(C)[C@@H]2c2cccnc2)C1] |
| A196 | 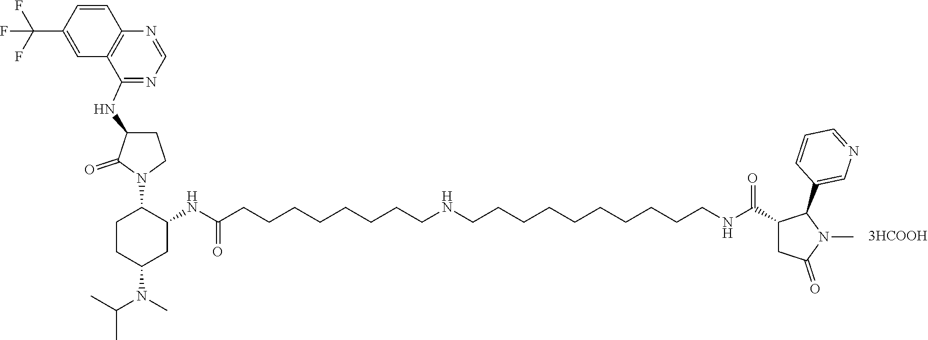 [SMILES: CC(C)N(C)[C@@H]1CC[C@H](N2CC[C@H](Nc3ncnc4ccc(C(F)(F)F)cc34)C2=O)[C@H](NC(=O)CCCCCCCCNCCCCCCCCCCNC(=O)[C@H]2CC(=O)N(C)[C@@H]2c2cccnc2)C1] |
| A197 | 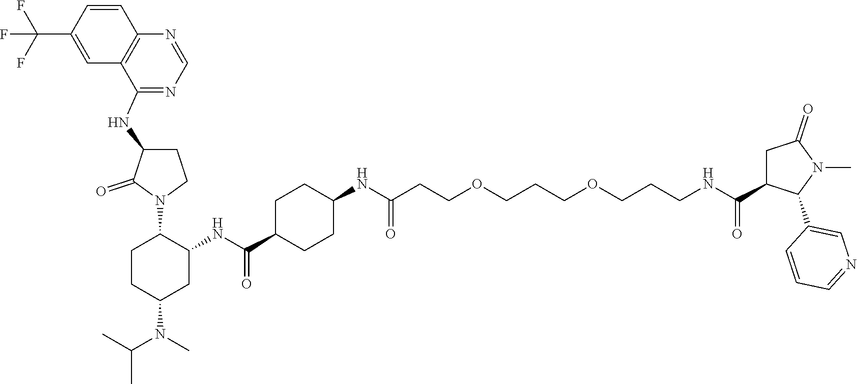 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCCOCCCNC(=O)[C@H]3CC(=O)N(C)[C@@H]3c3cccnc3)CC2)C1] |
| A198 | 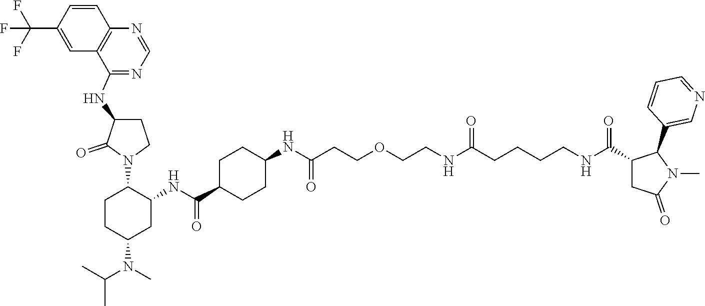 [SMILES: CC(C)N(C)[C@@H]1CC[C@H](N2CC[C@H](Nc3ncnc4ccc(C(F)(F)F)cc34)C2=O)[C@H](NC(=O)[C@H]2CC[C@@H](NC(=O)CCOCCNC(=O)CCCCNC(=O)[C@H]3CC(=O)N(C)[C@@H]3c3cccnc3)CC2)C1] |
| B1 | 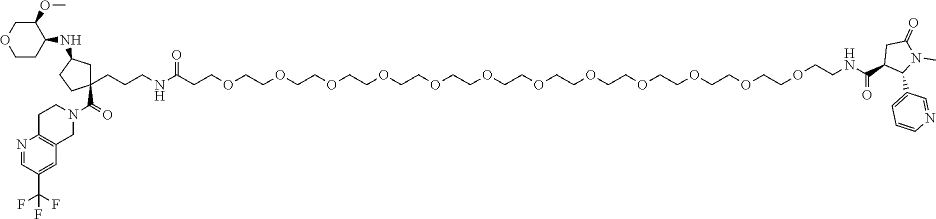 [SMILES: CO[C@@H]1COCC[C@@H]1N[C@@H]1CC[C@](CCCNC(=O)CCOCCOCCOCCOCCOCCOCCOCCOCCOCCOCCOCCOCCNC(=O)[C@H]2CC(=O)N(C)[C@@H]2c2cccnc2)(C(=O)N2CCc3ncc(C(F)(F)F)cc3C2)C1] |
| B2 | 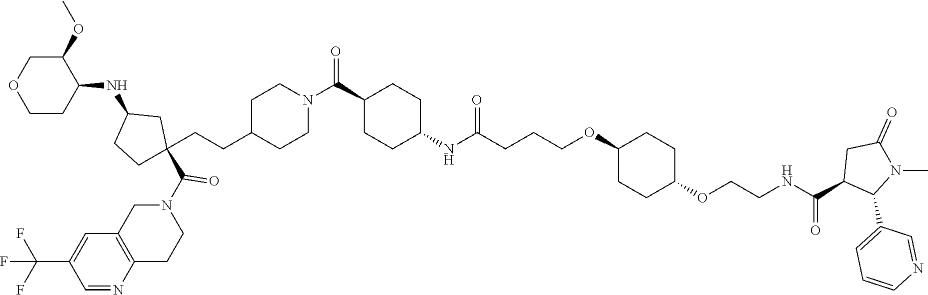 [SMILES: CO[C@@H]1COCC[C@@H]1N[C@@H]1CC[C@](CCC2CCN(C(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)(C(=O)N2CCc3ncc(C(F)(F)F)cc3C2)C1] |
| B3 | 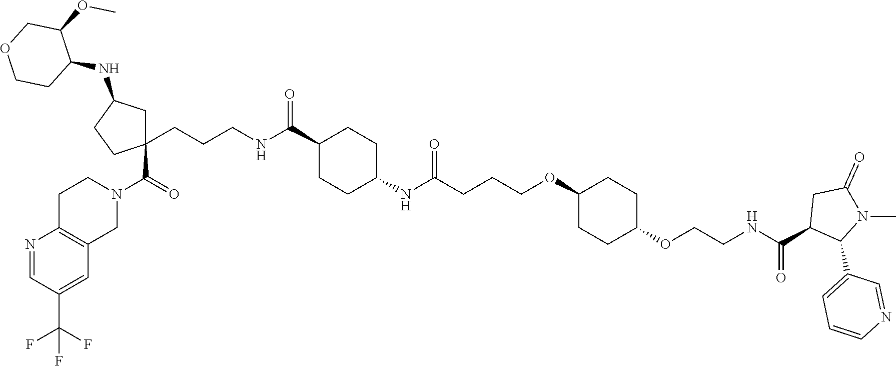 [SMILES: CO[C@@H]1COCC[C@@H]1N[C@@H]1CC[C@](CCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B4 | 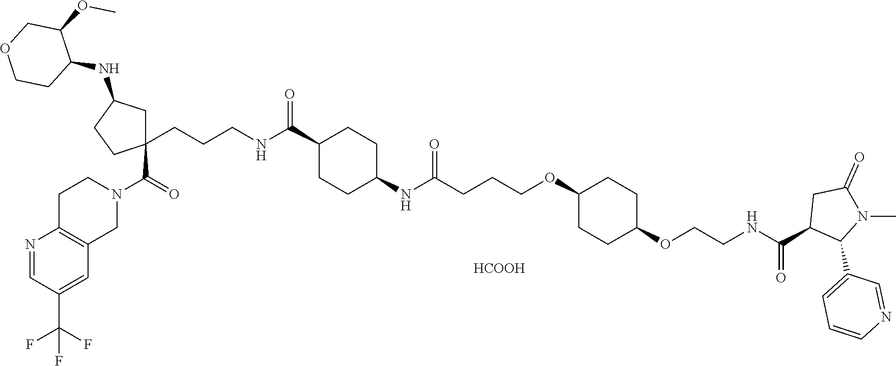 [SMILES: CO[C@@H]1COCC[C@@H]1N[C@@H]1CC[C@](CCCNC(=O)[C@H]2CC[C@@H](NC(=O)CCCO[C@H]3CC[C@@H](OCCNC(=O)[C@H]4CC(=O)N(C)[C@@H]4c4cccnc4)CC3)CC2)(C(=O)N2CCc3ncc(C(F)(F)F)cc3C2)C1 ; "HCOOH"] |
| B5 | 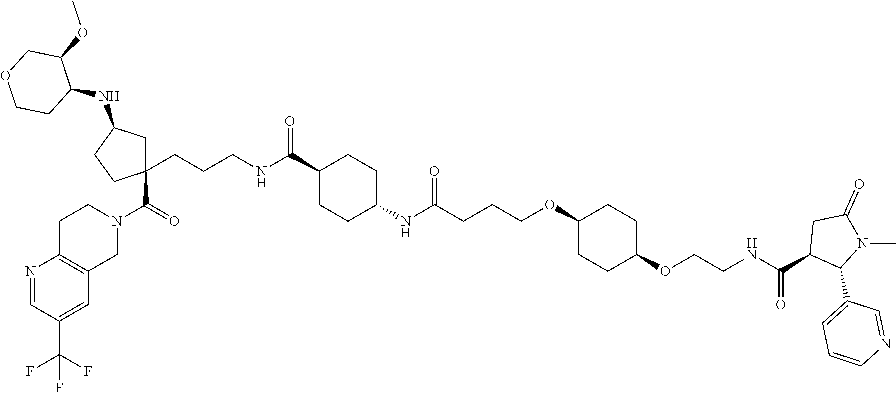 [SMILES: CO[C@@H]1COCC[C@@H]1N[C@@H]1CC[C@](CCCNC(=O)[C@H]2CC[C@H](NC(=O)CCCO[C@H]3CC[C@@H](OCCNC(=O)[C@H]4CC(=O)N(C)[C@@H]4c4cccnc4)CC3)CC2)(C(=O)N2CCc3ncc(C(F)(F)F)cc3C2)C1] |
| B6 | 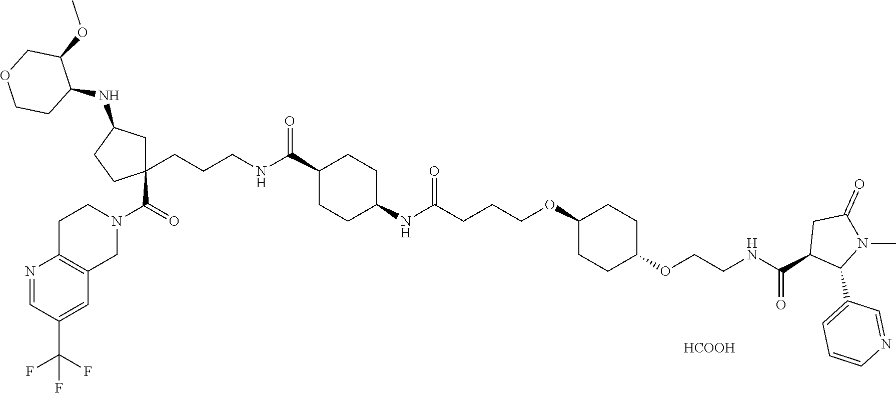 [SMILES: CO[C@@H]1COCC[C@@H]1N[C@@H]1CC[C@](CCCNC(=O)[C@H]2CC[C@@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1 ; "HCOOH"] |
| B7 | 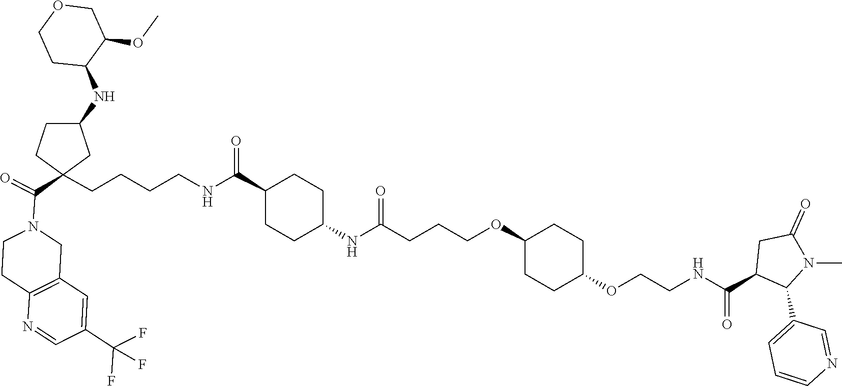 [SMILES: CO[C@@H]1COCC[C@@H]1N[C@@H]1CC[C@](CCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B8 | 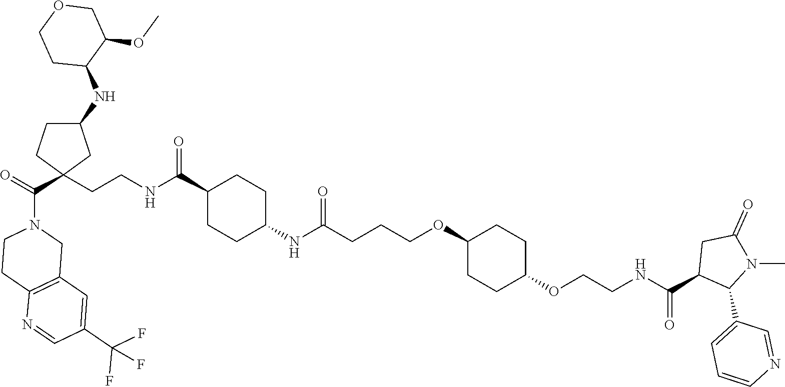 [SMILES: CO[C@@H]1COCC[C@@H]1N[C@@H]1CC[C@](CCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B9 | 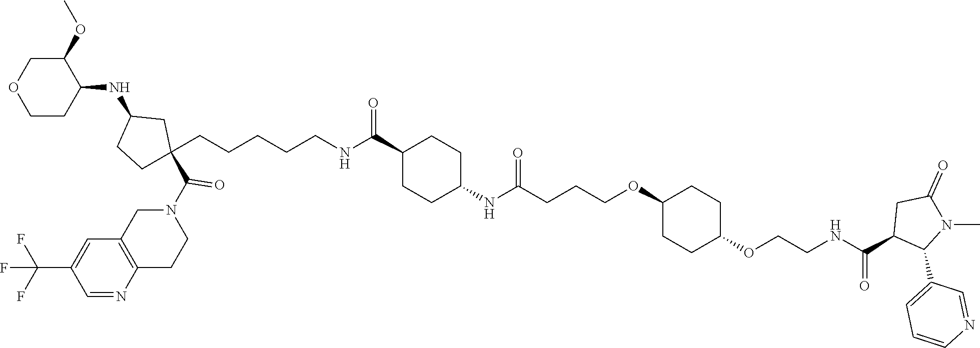 [SMILES: CO[C@@H]1COCC[C@@H]1N[C@@H]1CC[C@](CCCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B10 | 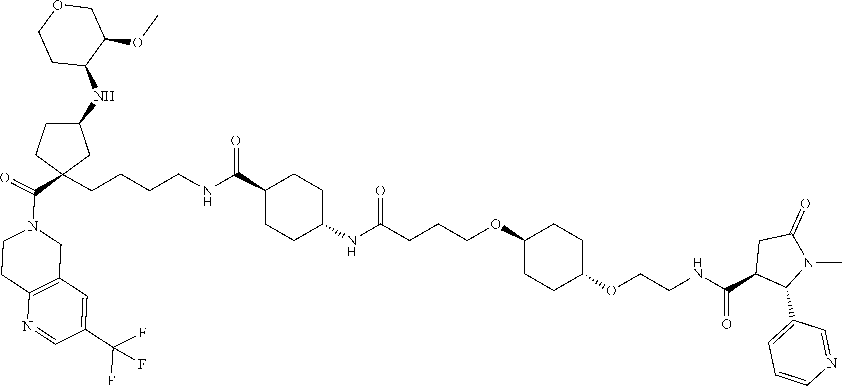 [SMILES: CO[C@@H]1COCC[C@@H]1N[C@@H]1CC[C@](CCCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B11 | 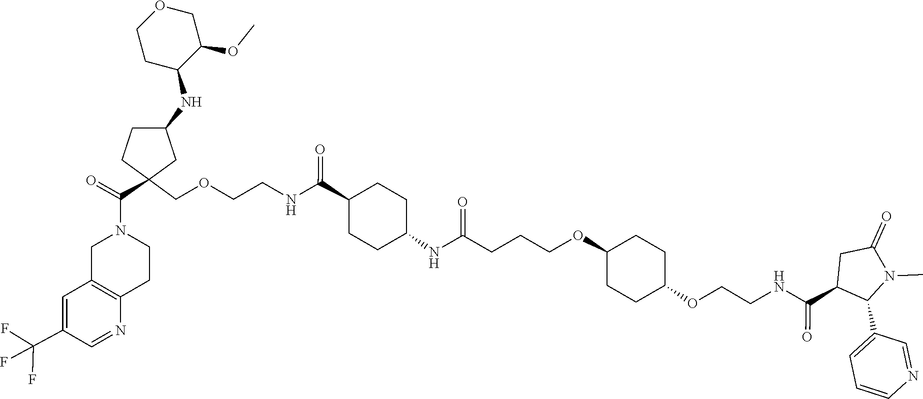 [SMILES: CO[C@@H]1COCC[C@@H]1N[C@@H]1CC[C@](COCCNC(=O)[C@H]2CC[C@H](NC(=O)CCCO[C@H]3CC[C@H](OCCNC(=O)[C@H]4CC(=O)N(C)[C@@H]4c4cccnc4)CC3)CC2)(C(=O)N2CCc3ncc(C(F)(F)F)cc3C2)C1] |
| B12 | 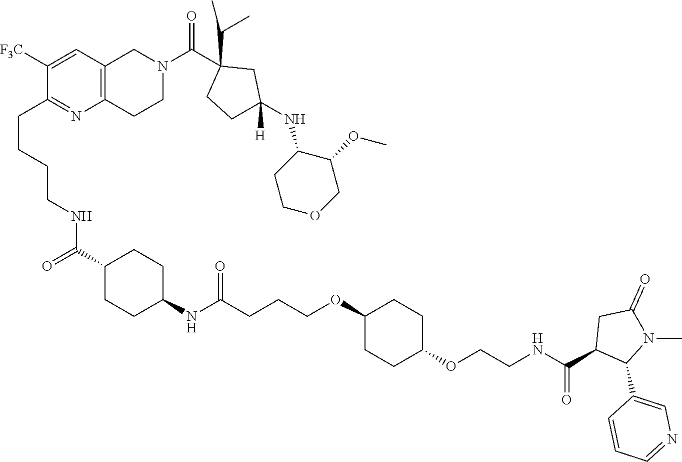 [SMILES: CO[C@@H]1COCC[C@@H]1N[C@@H]1CC[C@@](C(=O)N2CCc3nc(CCCCNC(=O)[C@H]4CC[C@H](NC(=O)CCCO[C@H]5CC[C@H](OCCNC(=O)[C@H]6CC(=O)N(C)[C@@H]6c6cccnc6)CC5)CC4)c(C(F)(F)F)cc3C2)(C(C)C)C1] |
| B13 | 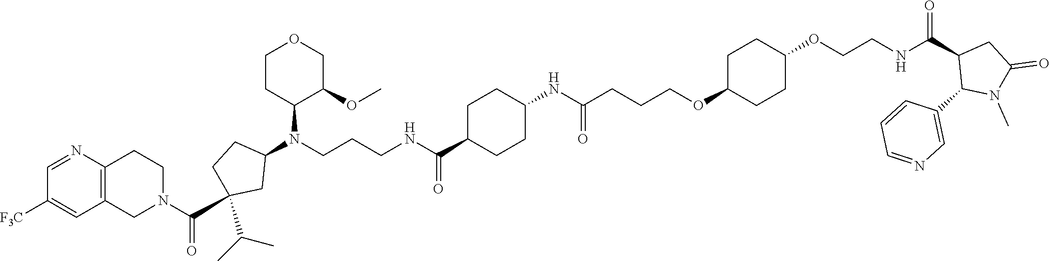 [SMILES: CO[C@@H]1COCC[C@@H]1N(CCCNC(=O)[C@H]1CC[C@H](NC(=O)CCCO[C@H]2CC[C@H](OCCNC(=O)[C@H]3CC(=O)N(C)[C@@H]3c3cccnc3)CC2)CC1)[C@@H]1CC[C@@](C(=O)N2CCc3ncc(C(F)(F)F)cc3C2)(C(C)C)C1] |
| B14 | 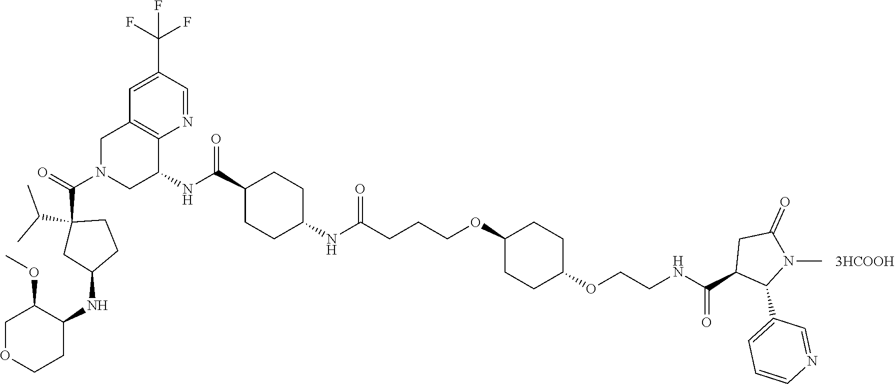 |
| B15 | 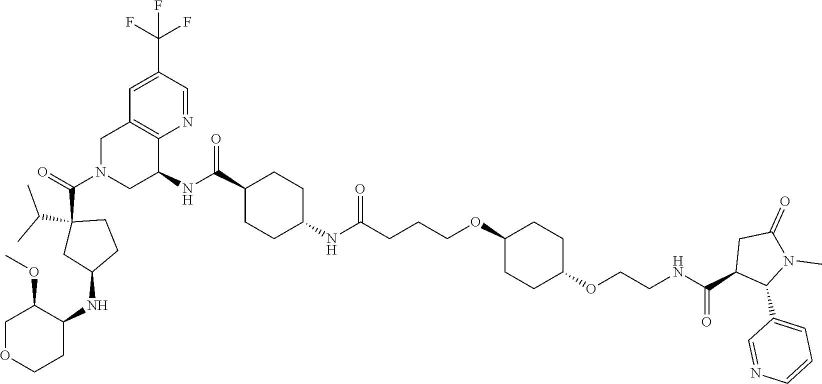 |
| B16 | 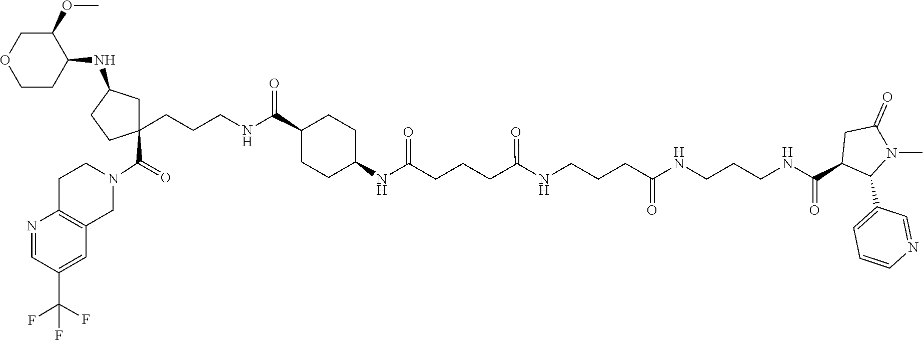 [SMILES: CO[C@@H]1COCC[C@@H]1N[C@@H]1CC[C@](CCCNC(=O)[C@H]2CC[C@@H](NC(=O)CCCC(=O)NCCCC(=O)NCCCNC(=O)[C@H]3CC(=O)N(C)[C@@H]3c3cccnc3)CC2)(C(=O)N2CCc3ncc(C(F)(F)F)cc3C2)C1] |
| B17 | 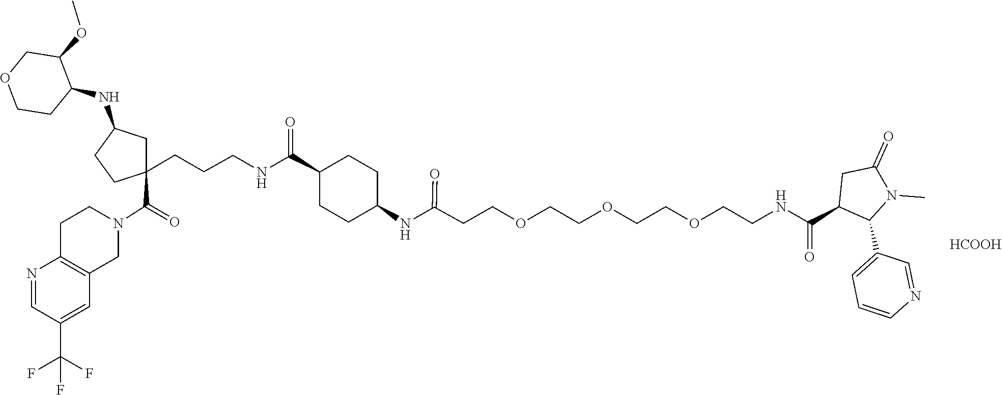 [SMILES: CO[C@@H]1COCC[C@@H]1N[C@@H]1CC[C@](CCCNC(=O)[C@H]2CC[C@@H](NC(=O)CCOCCOCCOCCNC(=O)[C@H]3CC(=O)N(C)[C@@H]3c3cccnc3)CC2)(C(=O)N2CCc3ncc(C(F)(F)F)cc3C2)C1 ; "HCOOH"] |
| B18 | 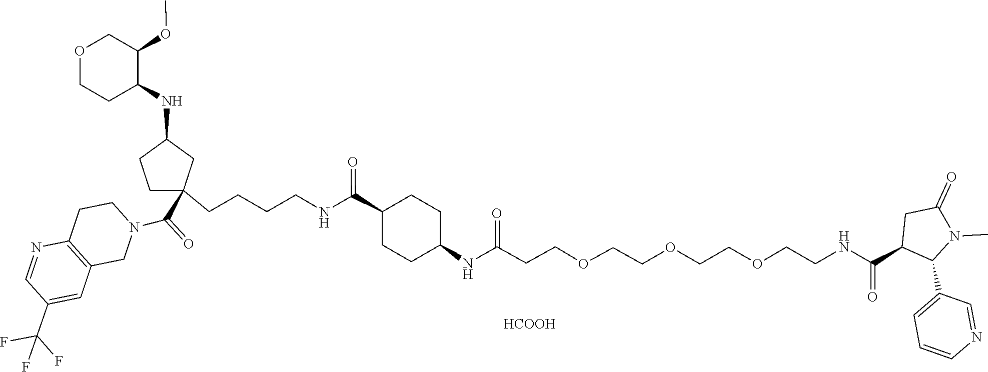 [SMILES: CO[C@@H]1COCC[C@@H]1N[C@@H]1CC[C@](CCCCNC(=O)[C@H]2CC[C@@H](NC(=O)CCOCCOCCOCCNC(=O)[C@H]3CC(=O)N(C)[C@@H]3c3cccnc3)CC2)(C(=O)N2CCc3ncc(C(F)(F)F)cc3C2)C1 ; "HCOOH"] |
| B19 | 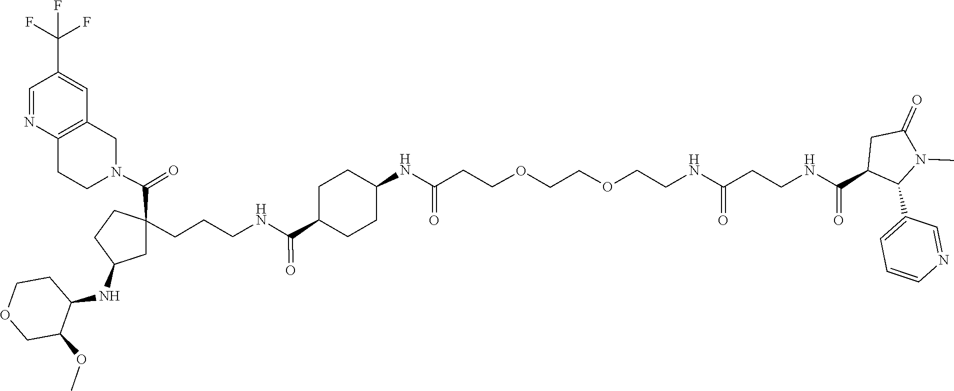 [SMILES: CO[C@H]1COCC[C@H]1N[C@H]1CC[C@@](CCCNC(=O)[C@H]2CC[C@@H](NC(=O)CCOCCOCCNC(=O)CCNC(=O)[C@H]3CC(=O)N(C)[C@@H]3c3cccnc3)CC2)(C(=O)N2CCc3ncc(C(F)(F)F)cc3C2)C1] |
| B20 | 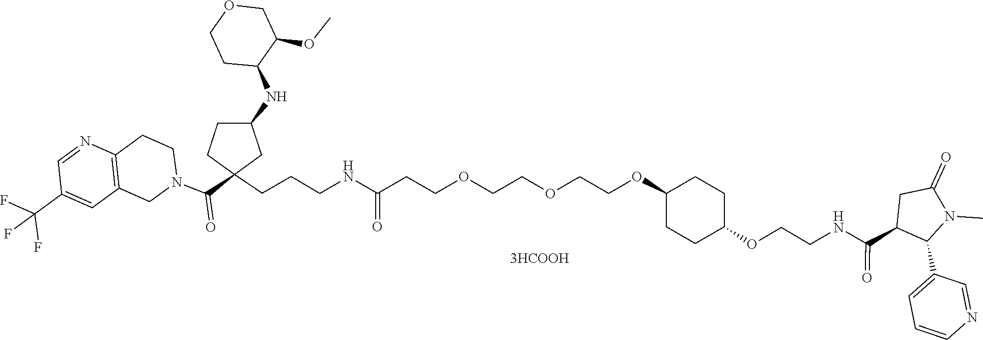 [SMILES: CO[C@@H]1COCC[C@@H]1N[C@@H]1CC[C@](CCCNC(=O)CCOCCOCCO[C@H]2CC[C@H](OCCNC(=O)[C@H]3CC(=O)N(C)[C@@H]3c3cccnc3)CC2)(C(=O)N2CCc3ncc(C(F)(F)F)cc3C2)C1] |
| B21 | 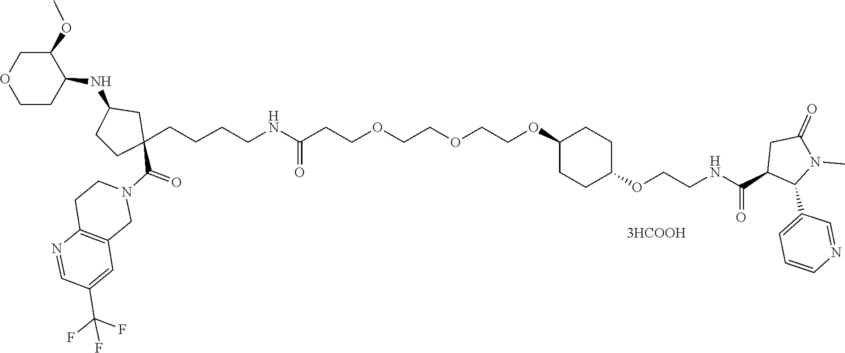 [SMILES: CO[C@@H]1COCC[C@@H]1N[C@@H]1CC[C@](CCCCNC(=O)CCOCCOCCO[C@H]2CC[C@H](OCCNC(=O)[C@H]3CC(=O)N(C)[C@@H]3c3cccnc3)CC2)(C(=O)N2CCc3ncc(C(F)(F)F)cc3C2)C1 ; "3HCOOH"] |
| B22 | 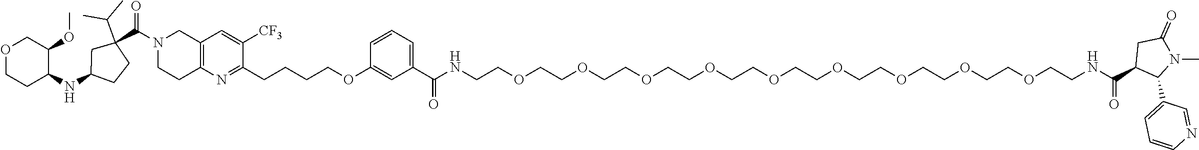 [SMILES: CO[C@@H]1COCC[C@@H]1N[C@@H]1CC[C@@](C(=O)N2CCc3nc(CCCCOc4cccc(C(=O)NCCOCCOCCOCCOCCOCCOCCOCCOCCOCCNC(=O)[C@H]5CC(=O)N(C)[C@@H]5c5cccnc5)c4)c(C(F)(F)F)cc3C2)(C(C)C)C1] |
| B23 | 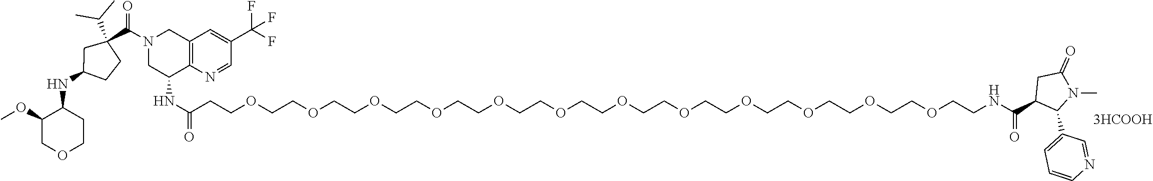 |
| B24 | 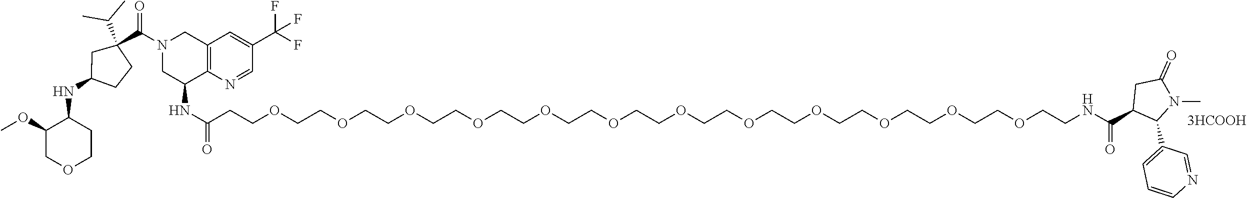 |
| C1 | 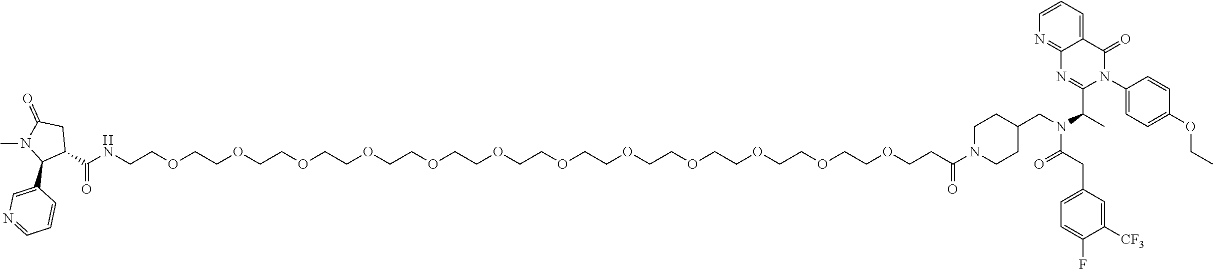 [SMILES: CCOc1ccc(-n2c([C@@H](C)N(CC3CCN(C(=O)CCOCCOCCOCCOCCOCCOCCOCCOCCOCCOCCOCCOCCNC(=O)[C@H]4CC(=O)N(C)[C@@H]4c4cccnc4)CC3)C(=O)Cc3ccc(F)c(C(F)(F)F)c3)nc3ncccc3c2=O)cc1] |
| C2 | 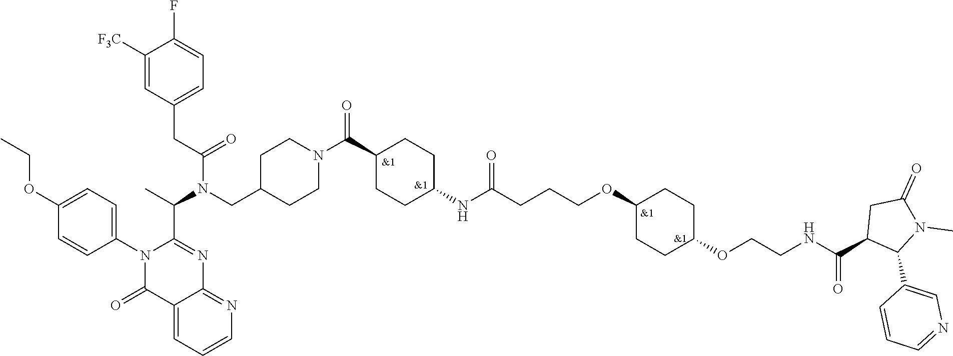 [SMILES: CCOc1ccc(-n2c([C@@H](C)N(CC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)C(=O)Cc3ccc(F)c(C(F)(F)F)c3)nc3ncccc3c2=O)cc1] |
| C3 | 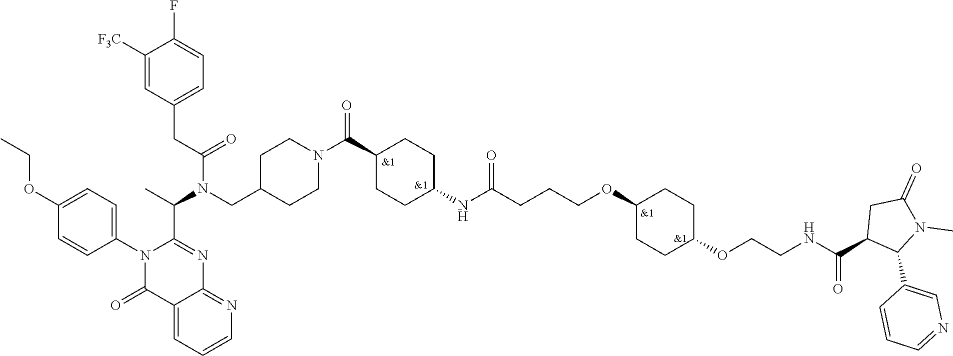 [SMILES: CCOc1ccc(-n2c([C@@H](C)N(CC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)C(=O)Cc3ccc(F)c(C(F)(F)F)c3)nc3ncccc3c2=O)cc1] |
| D1 | 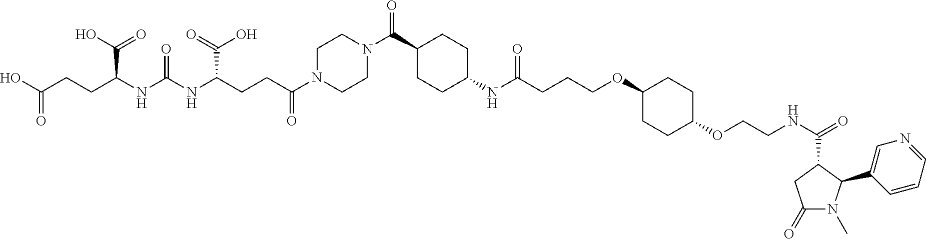 [SMILES: CN1C(=O)C[C@H](C(=O)NCCO[C@H]2CC[C@H](OCCCC(=O)N[C@H]3CC[C@H](C(=O)N4CCN(C(=O)CC[C@H](NC(=O)N[C@@H](CCC(=O)O)C(=O)O)C(=O)O)CC4)CC3)CC2)[C@H]1c1cccnc1] |
| D2 | 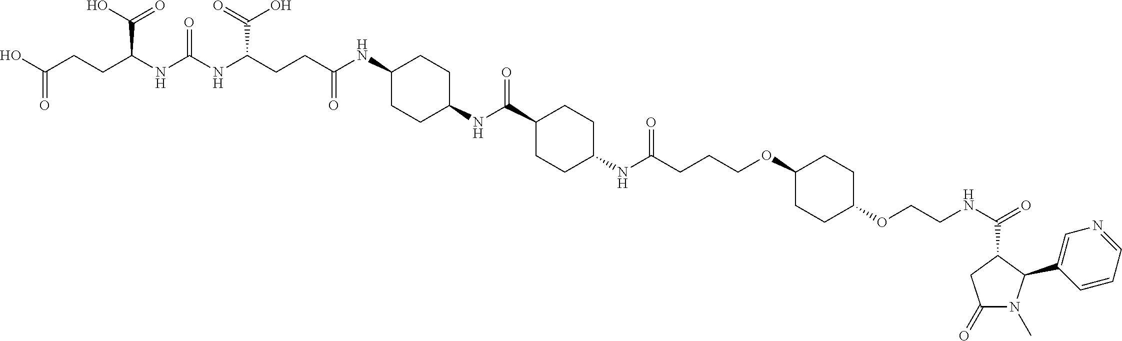 [SMILES: CN1C(=O)C[C@H](C(=O)NCCO[C@H]2CC[C@H](OCCCC(=O)N[C@H]3CC[C@H](C(=O)N[C@H]4CC[C@@H](NC(=O)CC[C@H](NC(=O)N[C@@H](CCC(=O)O)C(=O)O)C(=O)O)CC4)CC3)CC2)[C@H]1c1cccnc1] |
| D3 | 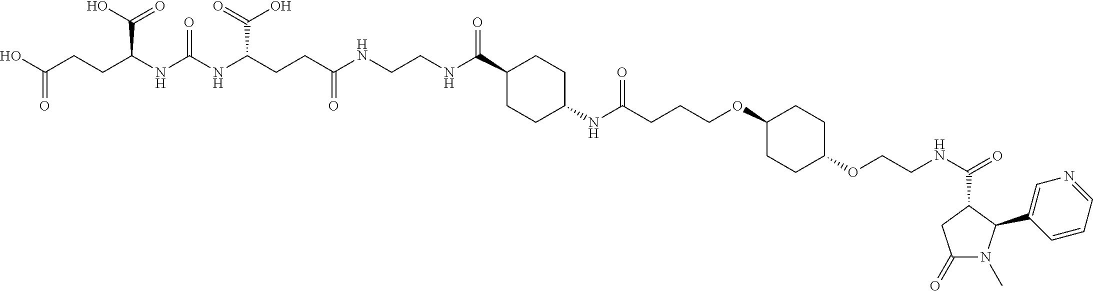 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)N[C@@H](CCC(=O)O)C(=O)O)C(=O)O)CC3)CC2)[C@H]1c1cccnc1] |
| D4 | 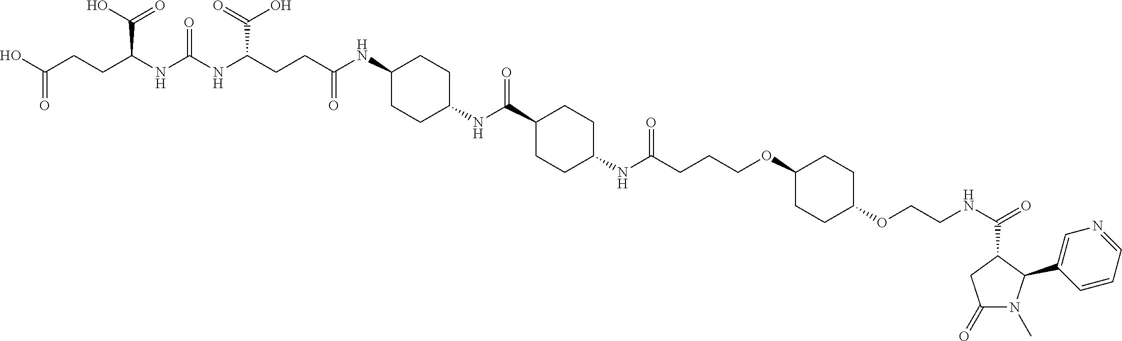 [SMILES: CN1C(=O)C[C@H](C(=O)NCCO[C@H]2CC[C@H](OCCCC(=O)N[C@H]3CC[C@H](C(=O)N[C@H]4CC[C@H](NC(=O)CC[C@H](NC(=O)N[C@@H](CCC(=O)O)C(=O)O)C(=O)O)CC4)CC3)CC2)[C@H]1c1cccnc1] |
| D5 | 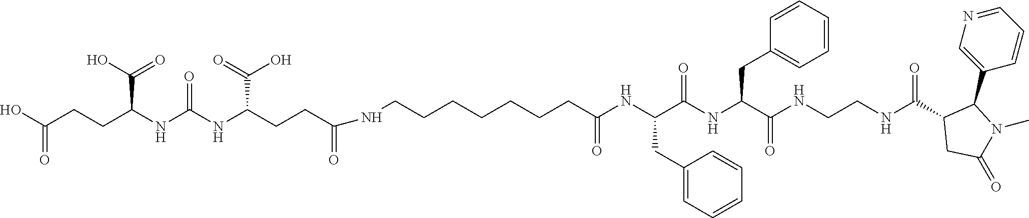 [SMILES: CN1C(=O)C[C@H](C(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| D6 | 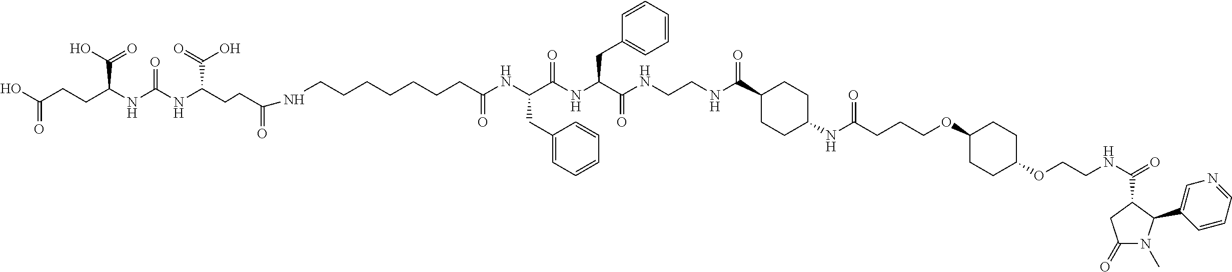 [SMILES: CN1C(=O)C[C@H](C(=O)NCCO[C@H]2CC[C@H](OCCCC(=O)N[C@H]3CC[C@H](C(=O)NCCNC(=O)[C@H](Cc4ccccc4)NC(=O)[C@H](Cc4ccccc4)NC(=O)CCCCCCCNC(=O)CC[C@H](NC(=O)N[C@@H](CCC(=O)O)C(=O)O)C(=O)O)CC3)CC2)[C@H]1c1cccnc1] |
| D7 | 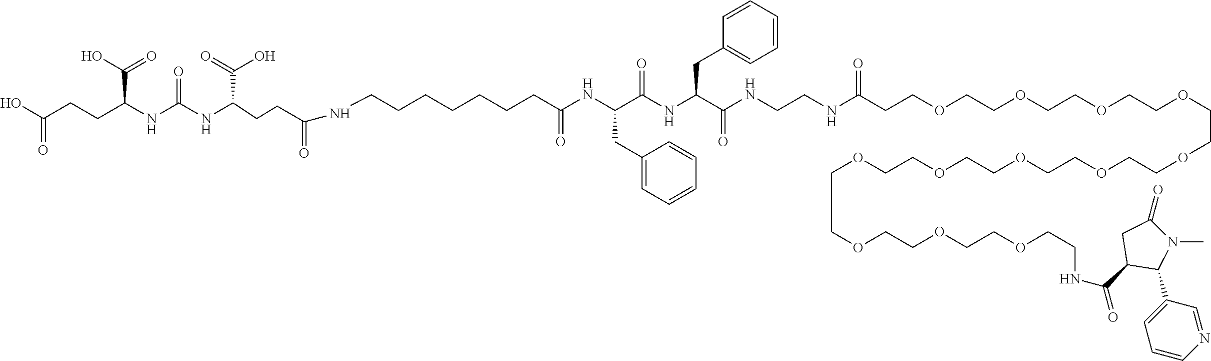 [SMILES: CN1C(=O)C[C@H](C(=O)NCCOCCOCCOCCOCCOCCOCCOCCOCCOCCOCCOCCOCCC(=O)NCCNC(=O)[C@H](Cc2ccccc2)NC(=O)[C@H](Cc2ccccc2)NC(=O)CCCCCCCNC(=O)CC[C@H](NC(=O)N[C@@H](CCC(=O)O)C(=O)O)C(=O)O)[C@H]1c1cccnc1] |
| D8 | 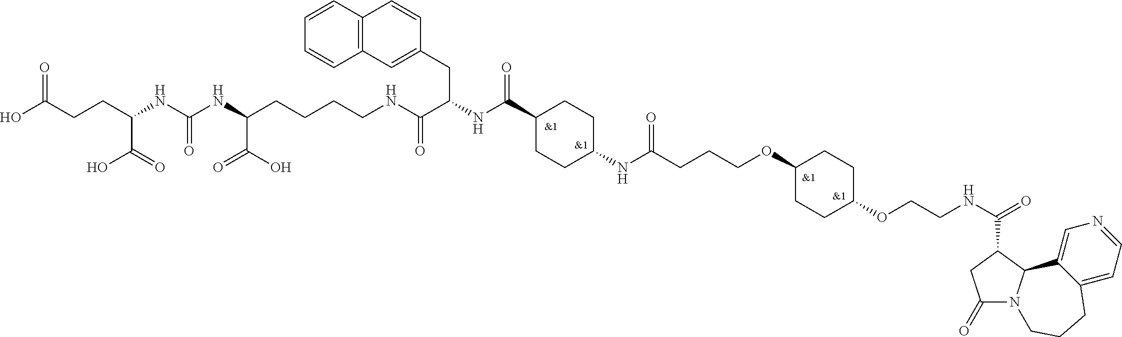 [SMILES: O=C(O)CC[C@H](NC(=O)N[C@@H](CCCCNC(=O)[C@H](Cc1ccc2ccccc2c1)NC(=O)[C@H]1CC[C@H](NC(=O)CCCO[C@H]2CC[C@H](OCCNC(=O)[C@H]3CC(=O)N4CCCc5ccncc5[C@H]34)CC2)CC1)C(=O)O)C(=O)O] |
| D9 | 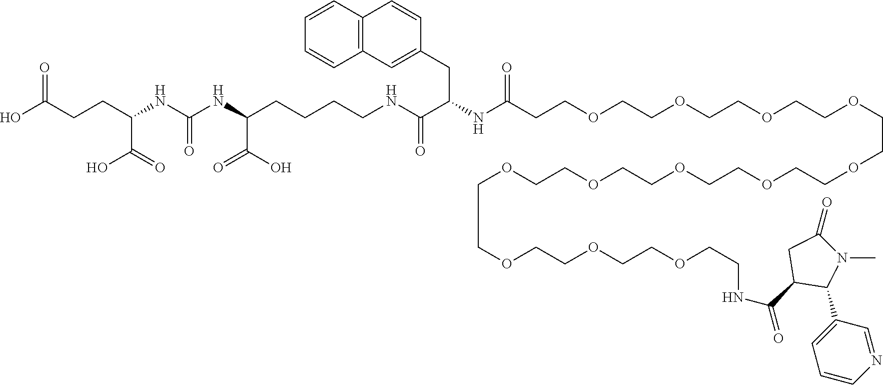 [SMILES: CN1C(=O)C[C@H](C(=O)NCCOCCOCCOCCOCCOCCOCCOCCOCCOCCOCCOCCOCCC(=O)N[C@@H](Cc2ccc3ccccc3c2)C(=O)NCCCC[C@H](NC(=O)N[C@@H](CCC(=O)O)C(=O)O)C(=O)O)[C@H]1c1cccnc1] |
| E1 | 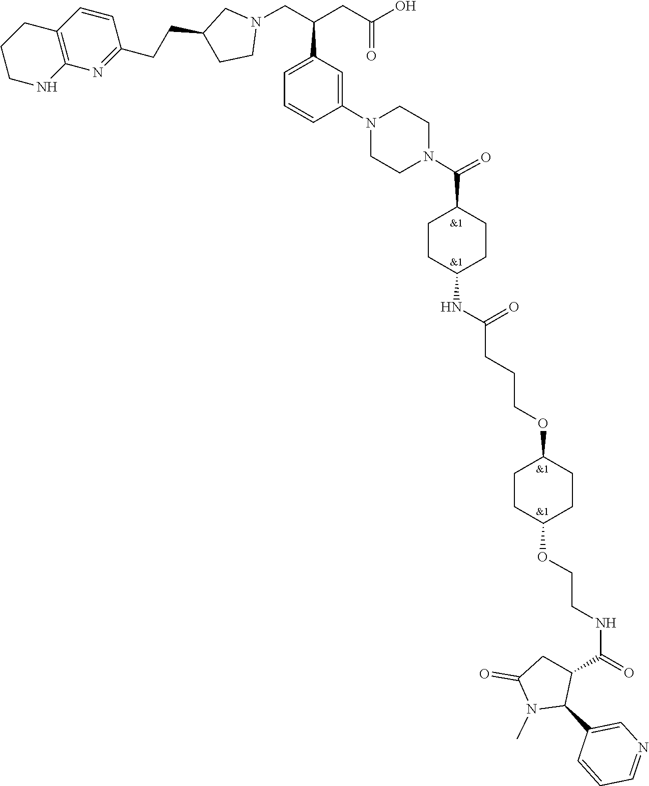 [SMILES: CN1C(=O)C[C@H](C(=O)NCCO[C@H]2CC[C@H](OCCCC(=O)N[C@H]3CC[C@H](C(=O)N4CCN(c5cccc([C@H](CC(=O)O)CN6CC[C@@H](CCc7ccc8c(n7)NCCC8)C6)c5)CC4)CC3)CC2)[C@H]1c1cccnc1] |
| E2 | 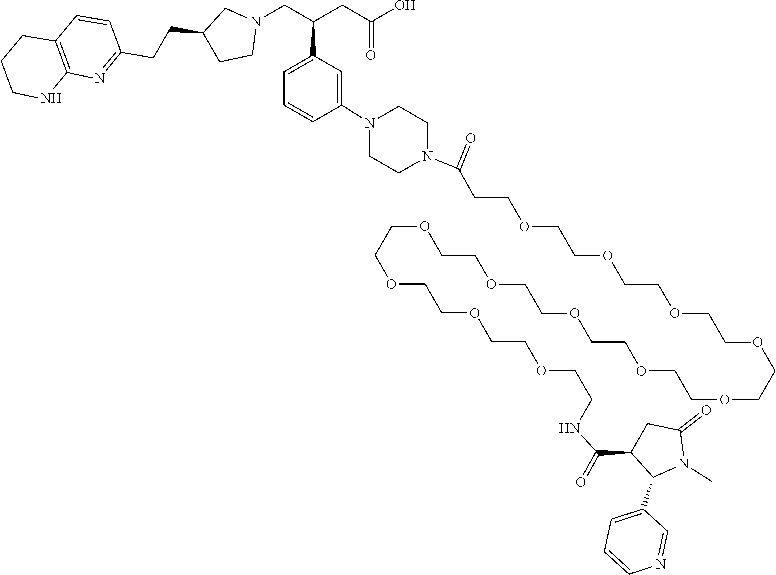 [SMILES: CN1C(=O)C[C@H](C(=O)NCCOCCOCCOCCOCCOCCOCCOCCOCCOCCOCCOCCOCCC(=O)N2CCN(c3cccc([C@H](CC(=O)O)CN4CC[C@@H](CCc5ccc6c(n5)NCCC6)C4)c3)CC2)[C@H]1c1cccnc1] |
| E3 | 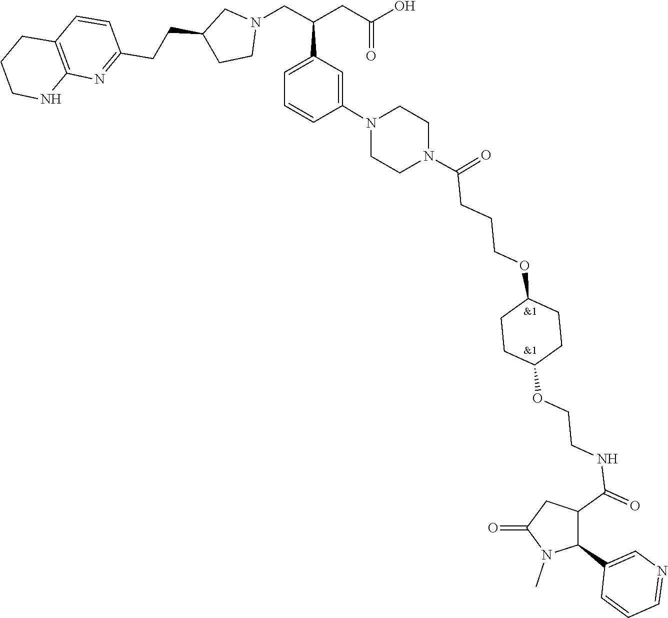 [SMILES: CN1C(=O)CC(C(=O)NCCO[C@H]2CC[C@H](OCCCC(=O)N3CCN(c4cccc([C@H](CC(=O)O)CN5CC[C@@H](CCc6ccc7c(n6)NCCC7)C5)c4)CC3)CC2)[C@H]1c1cccnc1] |
| F2 | 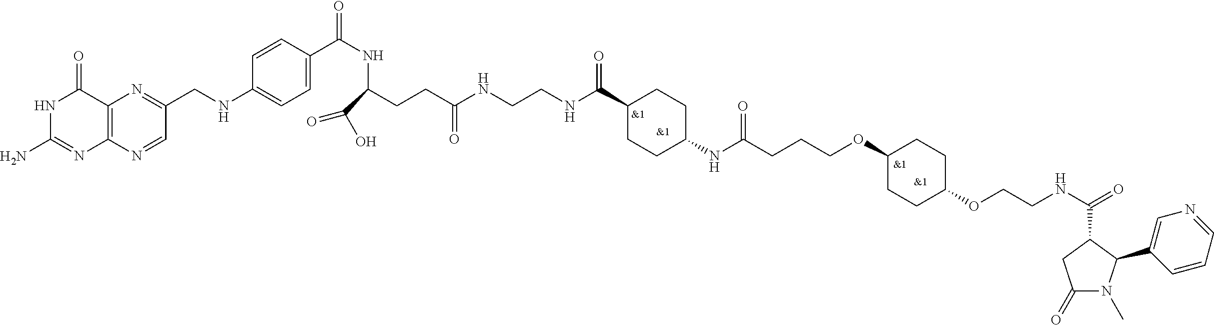 [SMILES: CN1C(=O)C[C@H](C(=O)NCCO[C@H]2CC[C@H](OCCCC(=O)N[C@H]3CC[C@H](C(=O)NCCNC(=O)CC[C@H](NC(=O)c4ccc(NCc5cnc6nc(N)[nH]c(=O)c6n5)cc4)C(=O)O)CC3)CC2)[C@H]1c1cccnc1] |
| F3 | 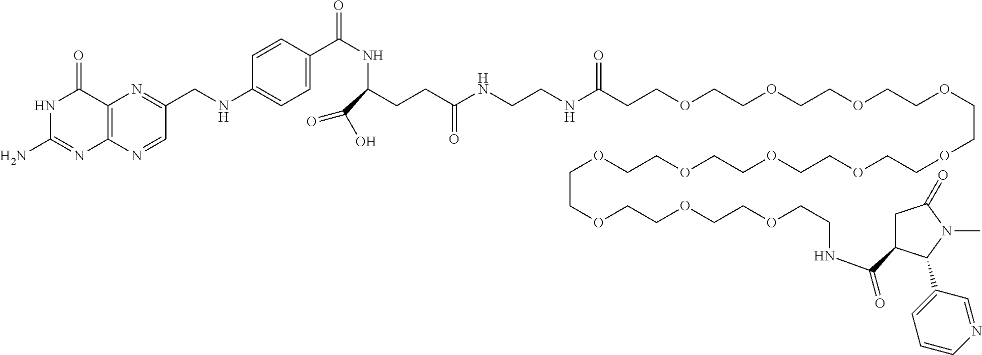 [SMILES: CN1C(=O)C[C@H](C(=O)NCCOCCOCCOCCOCCOCCOCCOCCOCCOCCOCCOCCOCCC(=O)NCCNC(=O)CC[C@H](NC(=O)c2ccc(NCc3cnc4nc(N)[nH]c(=O)c4n3)cc2)C(=O)O)[C@H]1c1cccnc1] |
| G1 | 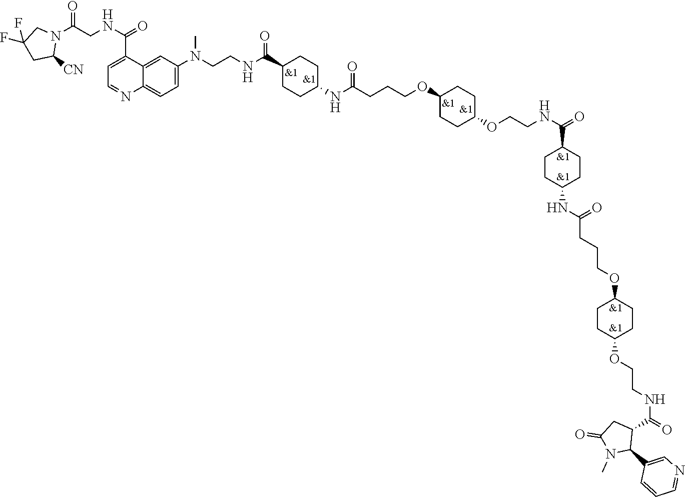 [SMILES: CN(CCNC(=O)[C@H]1CC[C@H](NC(=O)CCCO[C@H]2CC[C@H](OCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)CC2)CC1)c1ccc2nccc(C(=O)NCC(=O)N3CC(F)(F)C[C@@H]3C#N)c2c1] |
| G6 | 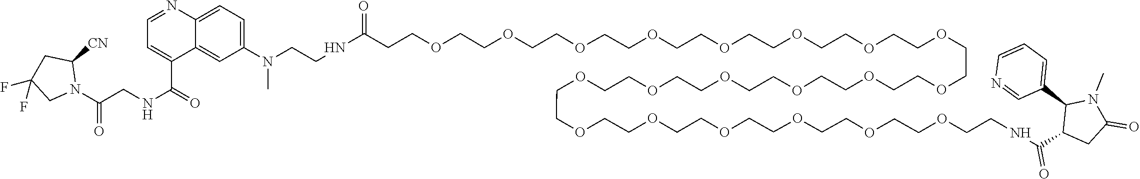 [SMILES: CN(CCNC(=O)CCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| G7 | 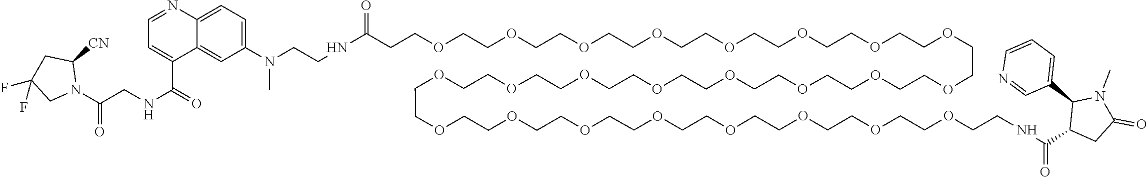 [SMILES: CN(CCNC(=O)CCOCCOCCOCCOCCOCCOCCOCCOCCOCCOCCOCCOCCOCCOCCOCCOCCOCCOCCOCCOCCOCCOCCOCCOCCNC(=O)[C@H]1CC(=O)N(C)[C@@H]1c1cccnc1)c1ccc2nccc(C(=O)NCC(=O)N3CC(F)(F)C[C@H]3C#N)c2c1] |
| G8 | 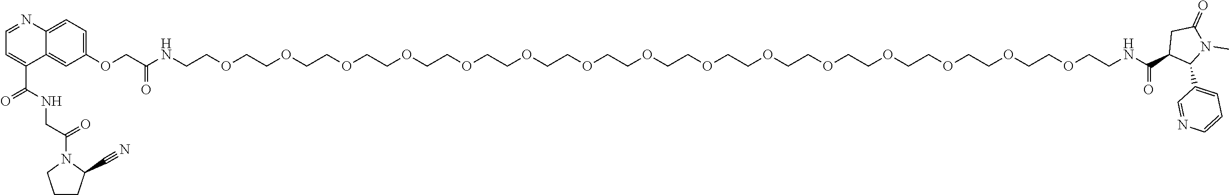 [SMILES: CN1C(=O)C[C@H](C(=O)NCCOCCOCCOCCOCCOCCOCCOCCOCCOCCOCCOCCOCCOCCOCCOCCNC(=O)COc2ccc3nccc(C(=O)NCC(=O)N4CCC[C@@H]4C#N)c3c2)[C@H]1c1cccnc1] |
| G9 | 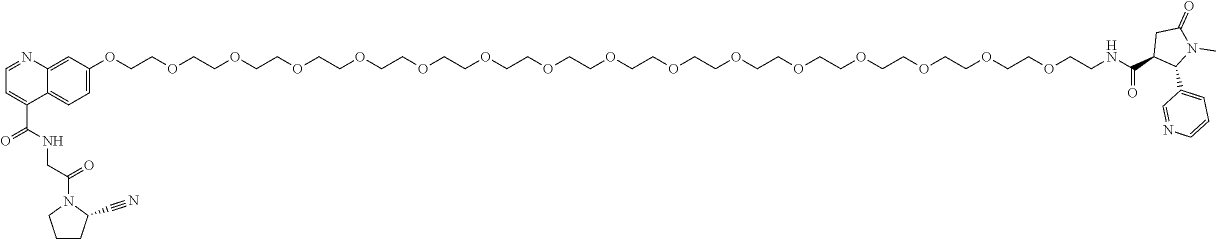 [SMILES: CN1C(=O)C[C@H](C(=O)NCCOCCOCCOCCOCCOCCOCCOCCOCCOCCOCCOCCOCCOCCOCCOCCOc2ccc3c(C(=O)NCC(=O)N4CCC[C@H]4C#N)ccnc3c2)[C@H]1c1cccnc1] |
| G10 | 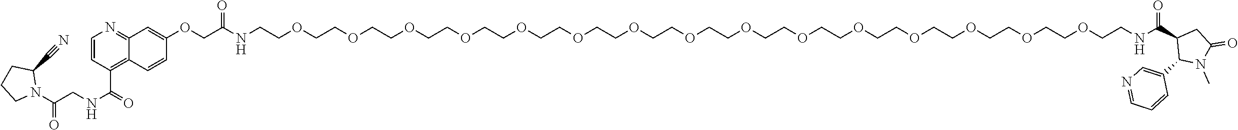 [SMILES: CN1C(=O)C[C@H](C(=O)NCCOCCOCCOCCOCCOCCOCCOCCOCCOCCOCCOCCOCCOCCOCCOCCNC(=O)COc2ccc3c(C(=O)NCC(=O)N4CCC[C@H]4C#N)ccnc3c2)[C@H]1c1cccnc1] |
| G11 | 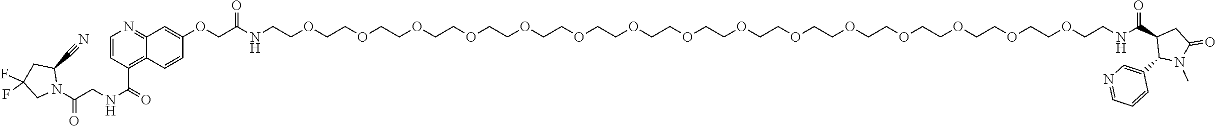 [SMILES: CN1C(=O)C[C@H](C(=O)NCCOCCOCCOCCOCCOCCOCCOCCOCCOCCOCCOCCOCCOCCOCCOCCNC(=O)COc2ccc3c(C(=O)NCC(=O)N4CC(F)(F)C[C@H]4C#N)ccnc3c2)[C@H]1c1cccnc1] |
| G12 | 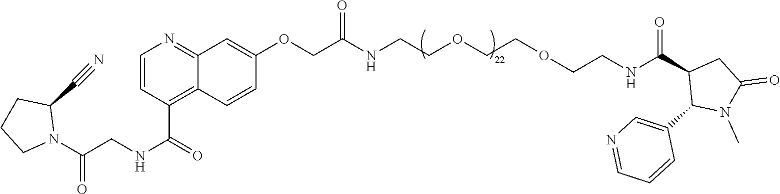 |
| G13 | 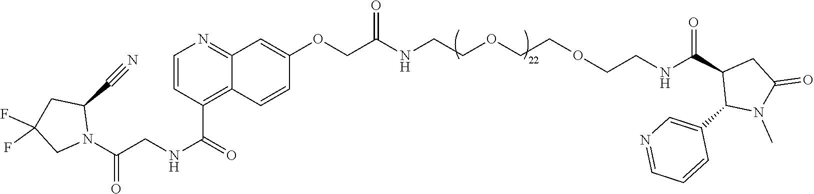 |
| H1 | 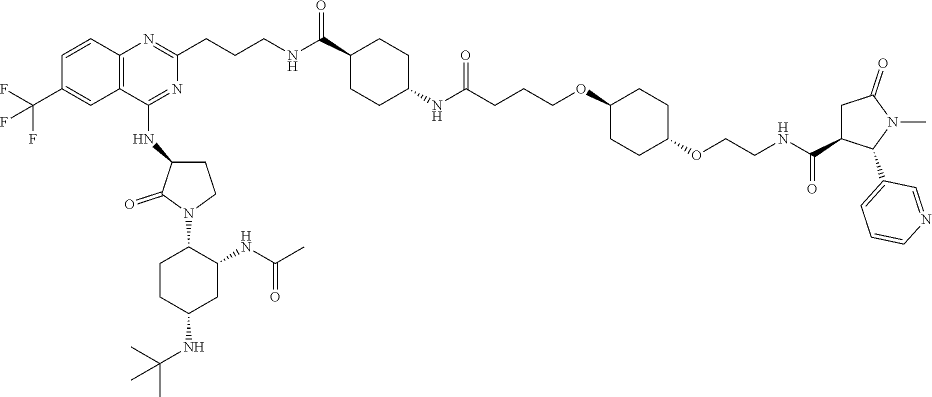 [SMILES: CC(=O)N[C@@H]1C[C@H](NC(C)(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H2 | 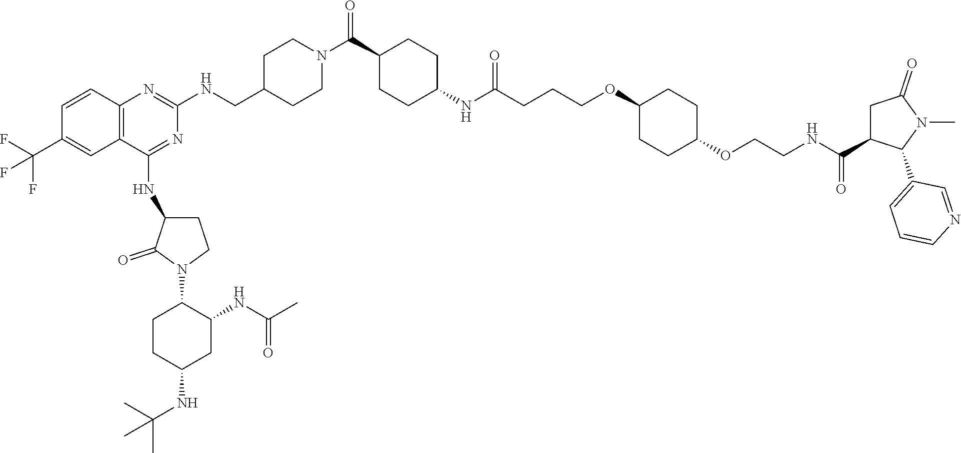 [SMILES: CC(=O)N[C@@H]1C[C@H](NC(C)(C)C)CC[C@@H]1N1CC[C@H](Nc2nc(NCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H3 | 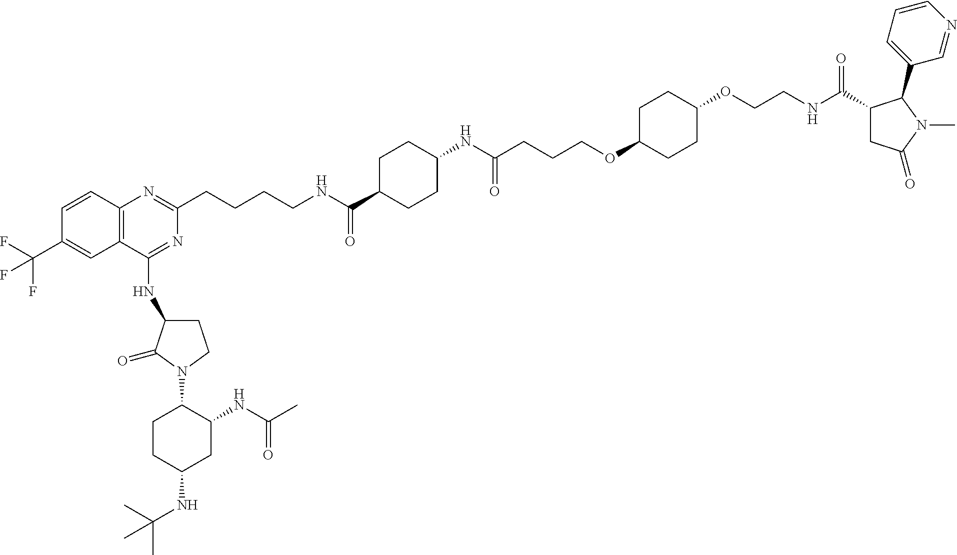 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H4 | 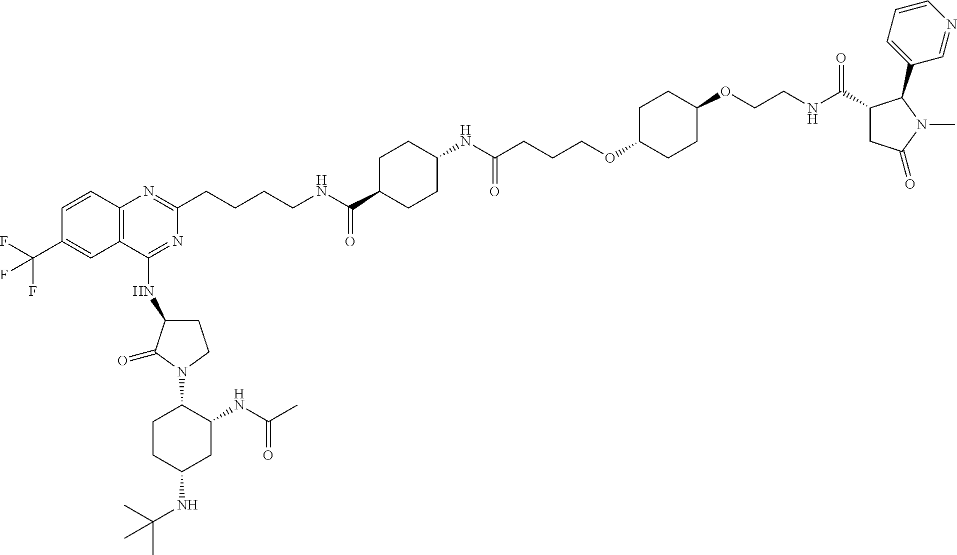 [SMILES: CC(=O)N[C@@H]1C[C@H](NC(C)(C)C)CC[C@@H]1N1CC[C@H](Nc2nc(CCCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H5 | 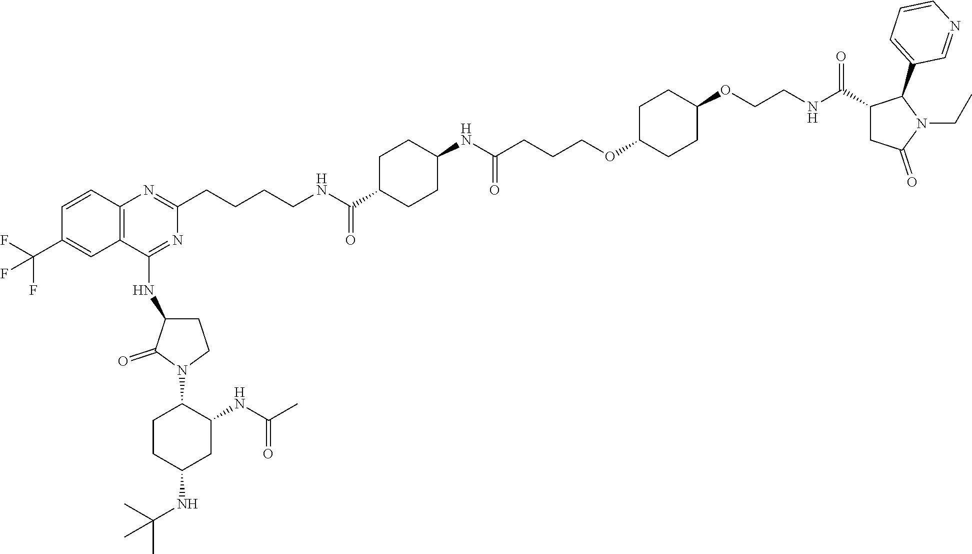 [SMILES: CCN1C(=O)C[C@H](C(=O)NCCO[C@H]2CC[C@H](OCCCC(=O)N[C@H]3CC[C@H](C(=O)NCCCCc4nc(N[C@H]5CCN([C@H]6CC[C@@H](NC(C)(C)C)C[C@H]6NC(C)=O)C5=O)c5cc(C(F)(F)F)ccc5n4)CC3)CC2)[C@H]1c1cccnc1] |
| H6 | 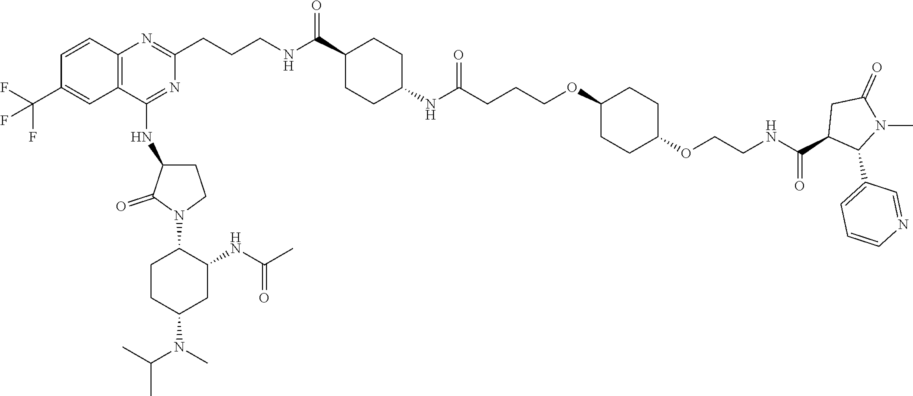 [SMILES: CC(=O)N[C@@H]1C[C@H](N(C)C(C)C)CC[C@@H]1N1CC[C@H](Nc2nc(CCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H7 | 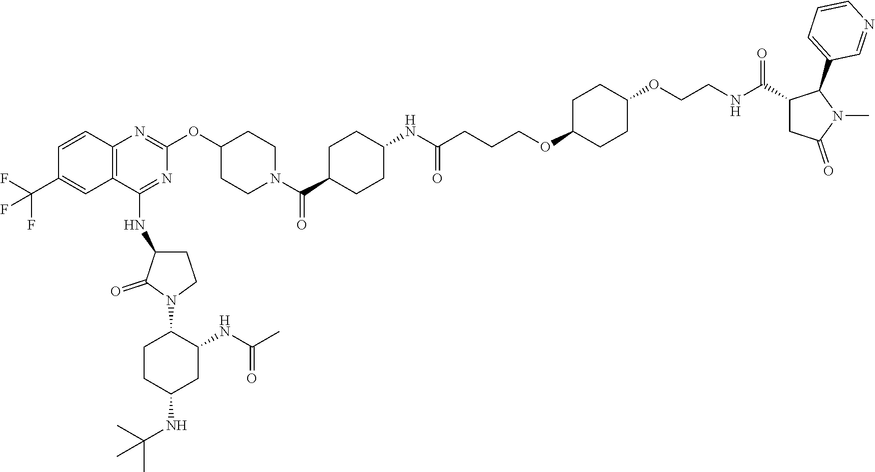 [SMILES: CC(=O)N[C@@H]1C[C@H](NC(C)(C)C)CC[C@@H]1N1CC[C@H](Nc2nc(OC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H8 | 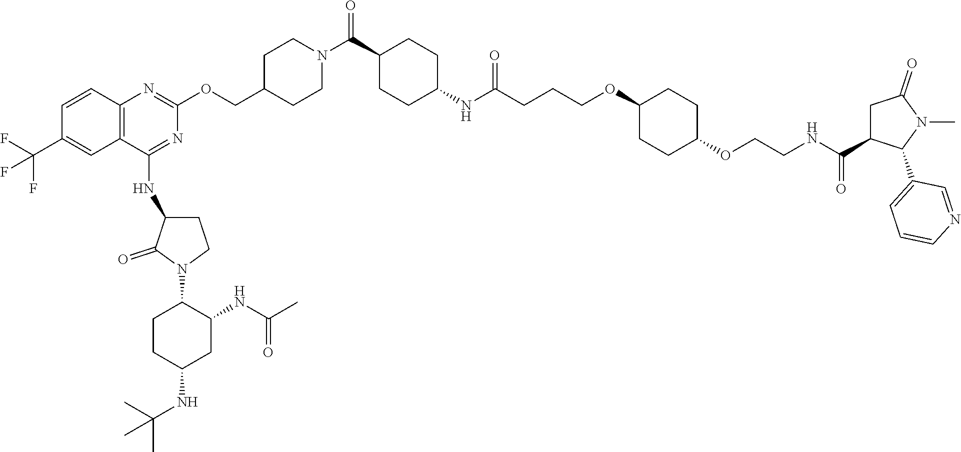 [SMILES: CC(=O)N[C@@H]1C[C@H](NC(C)(C)C)CC[C@@H]1N1CC[C@H](Nc2nc(OCC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H9 | 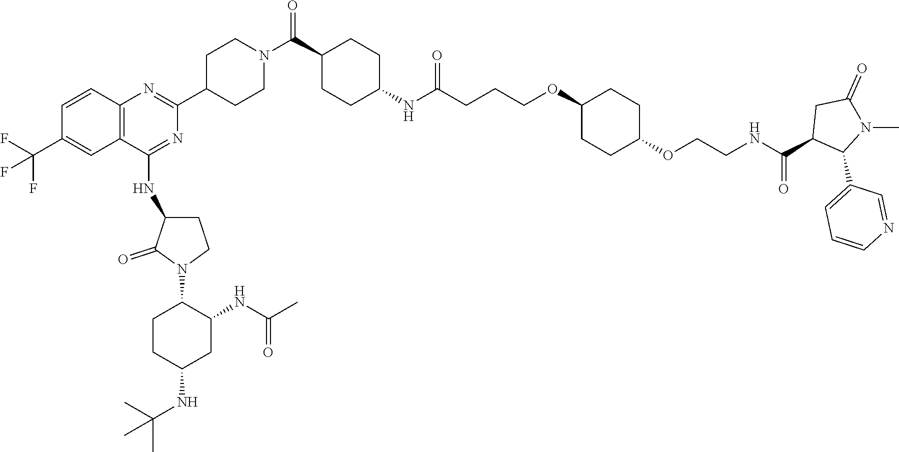 [SMILES: CC(=O)N[C@@H]1C[C@H](NC(C)(C)C)CC[C@@H]1N1CC[C@H](Nc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| h10 | 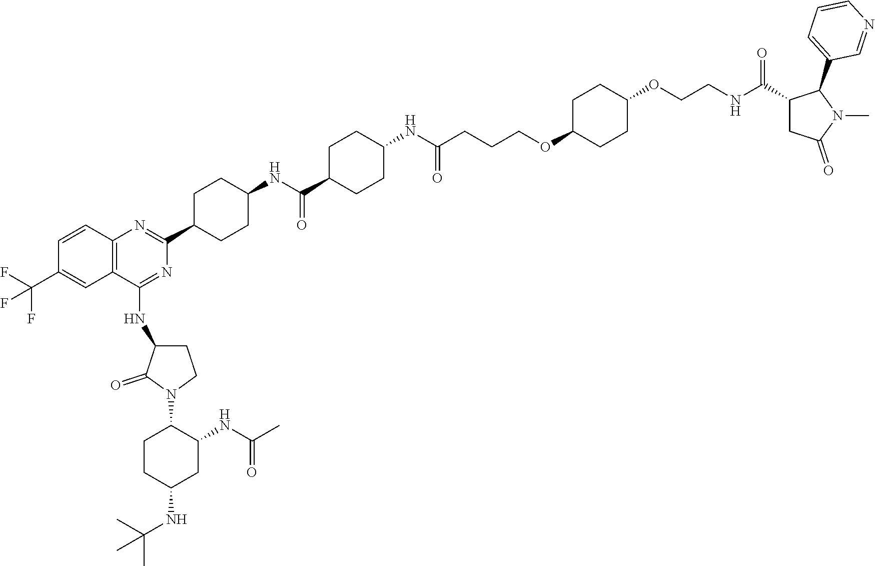 [SMILES: CC(=O)N[C@@H]1C[C@H](NC(C)(C)C)CC[C@@H]1N1CC[C@H](Nc2nc([C@H]3CC[C@@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H11 | 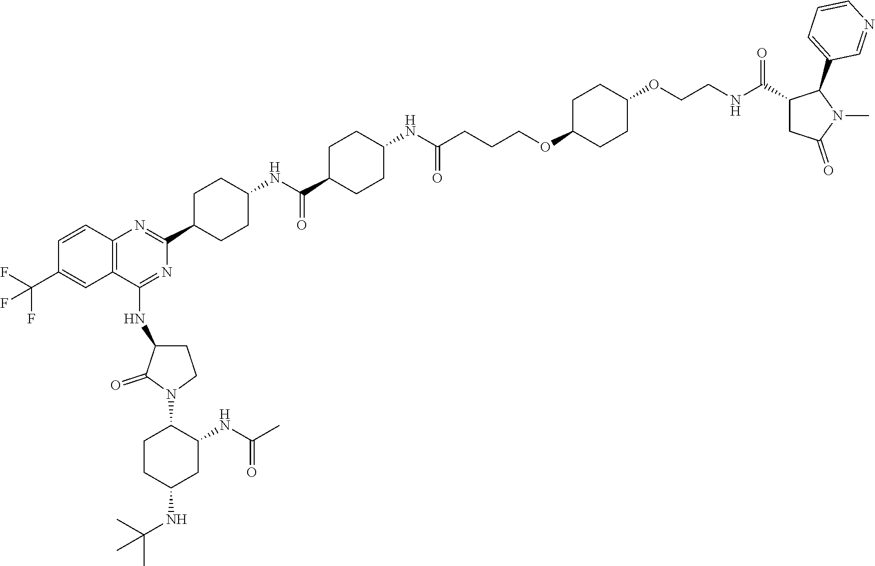 [SMILES: CC(=O)N[C@@H]1C[C@H](NC(C)(C)C)CC[C@@H]1N1CC[C@H](Nc2nc([C@H]3CC[C@H](NC(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H12 | 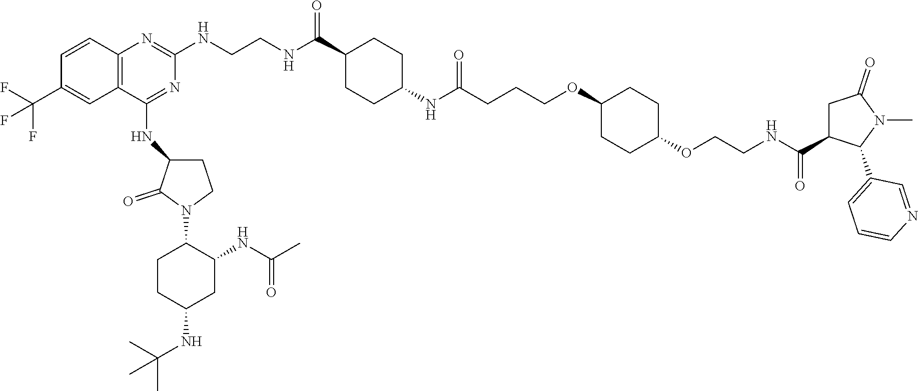 [SMILES: CC(=O)N[C@@H]1C[C@H](NC(C)(C)C)CC[C@@H]1N1CC[C@H](Nc2nc(NCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| H13 | 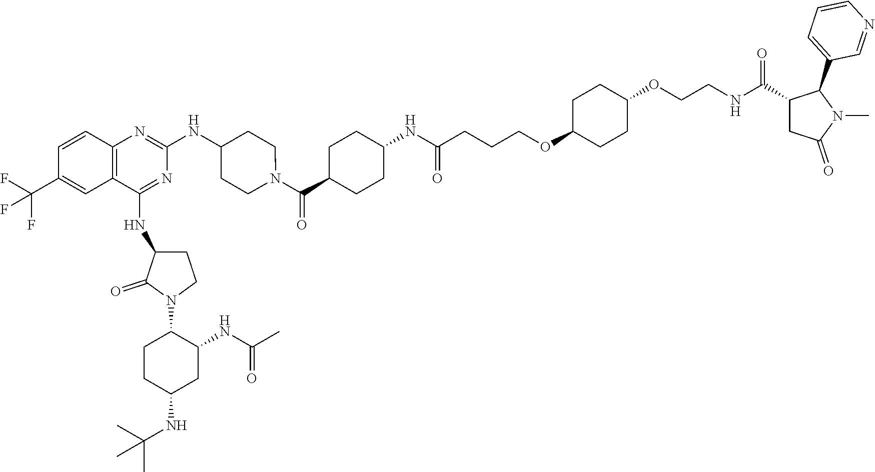 [SMILES: CC(=O)N[C@@H]1C[C@H](NC(C)(C)C)CC[C@@H]1N1CC[C@H](Nc2nc(NC3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)CC3)nc3ccc(C(F)(F)F)cc23)C1=O] |
| H14 | 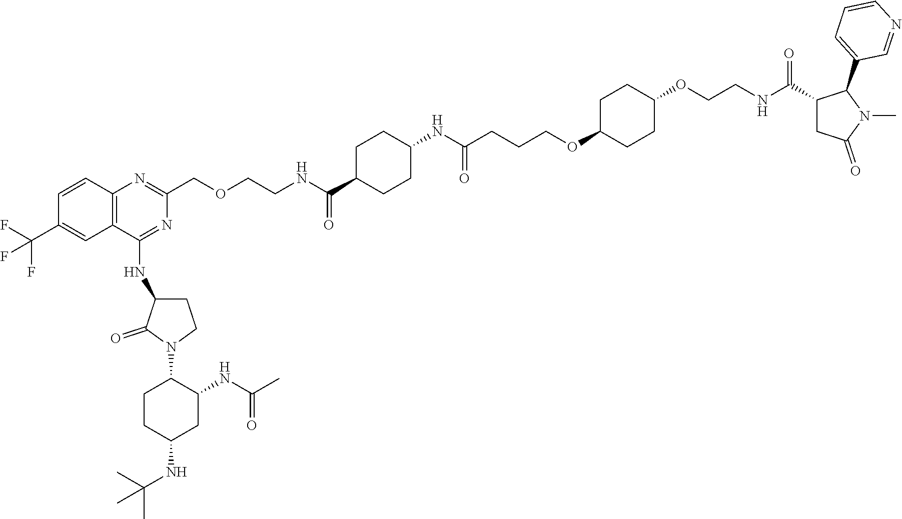 [SMILES: CC(=O)N[C@@H]1C[C@H](NC(C)(C)C)CC[C@@H]1N1CC[C@H](Nc2nc(COCCNC(=O)[C@H]3CC[C@H](NC(=O)CCCO[C@H]4CC[C@H](OCCNC(=O)[C@H]5CC(=O)N(C)[C@@H]5c5cccnc5)CC4)CC3)nc3ccc(C(F)(F)F)cc23)C1=O] |
| J1 | 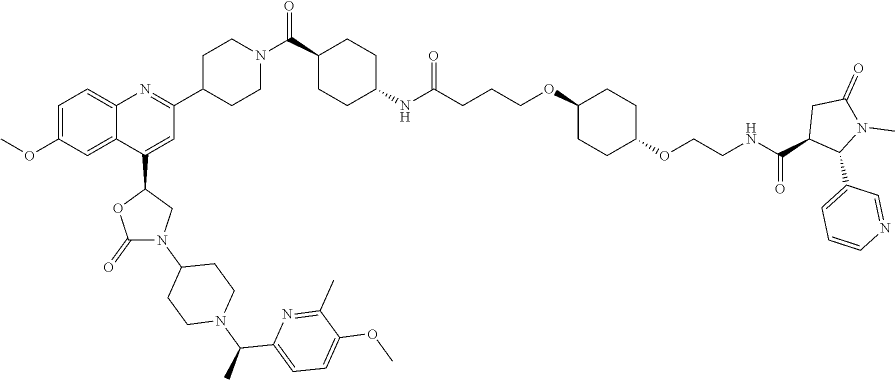 |
| J2 | 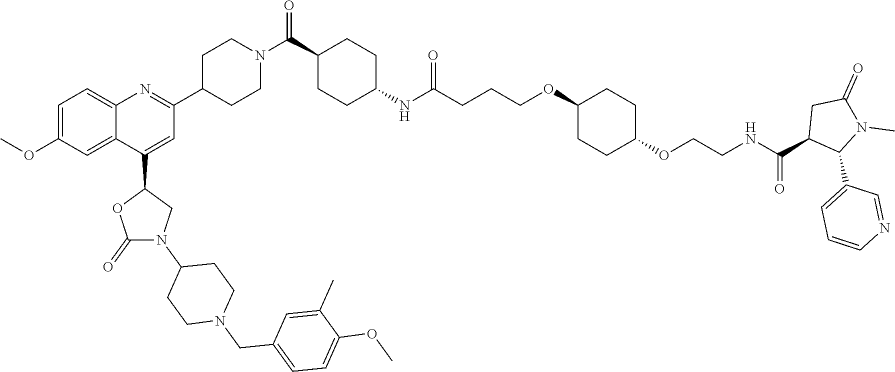 |
| J3 | 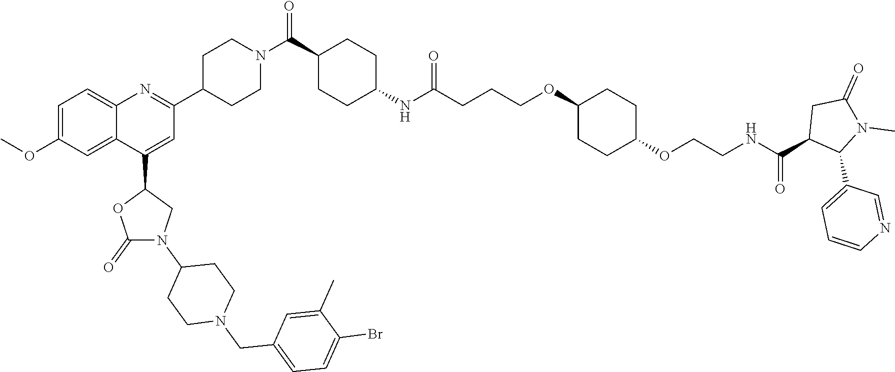 |
| J4 | 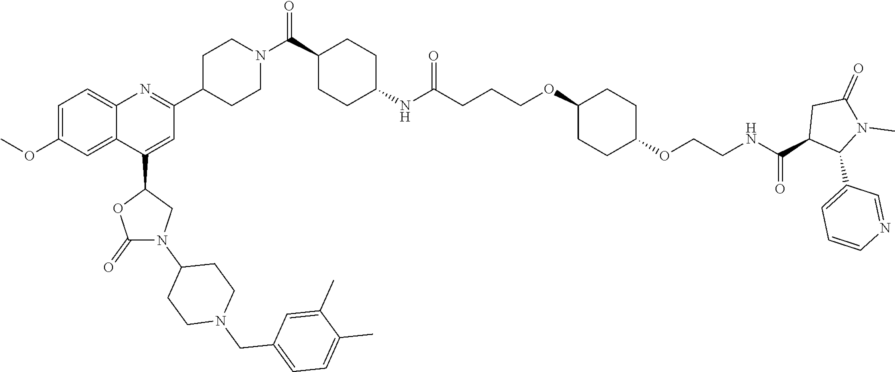 |
| J5 | 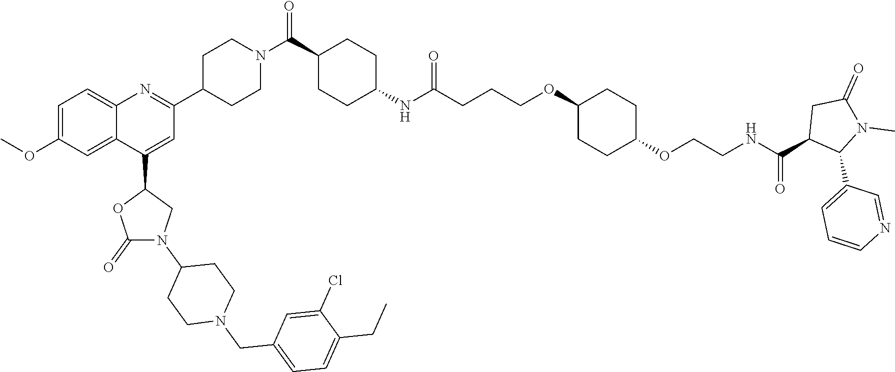 |
| J6 | 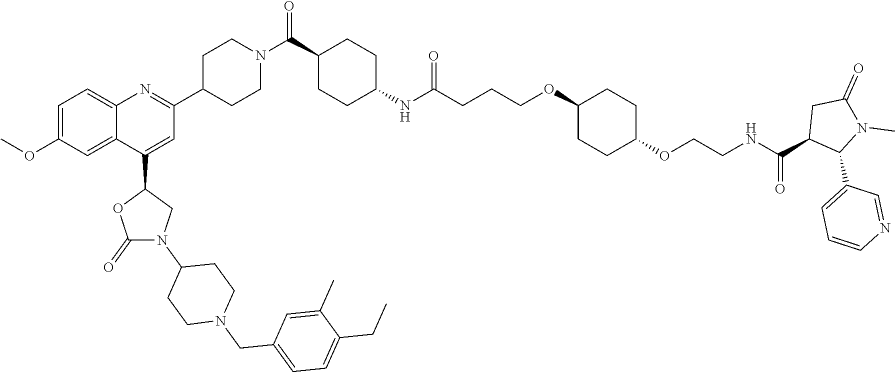 |
| J7 | 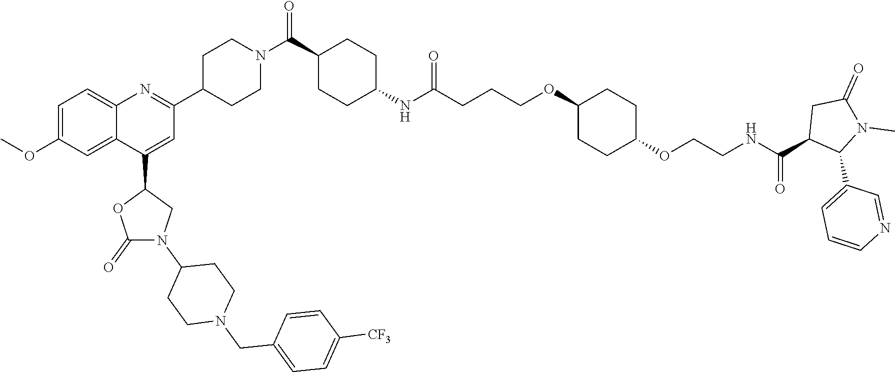 |
| J8 | 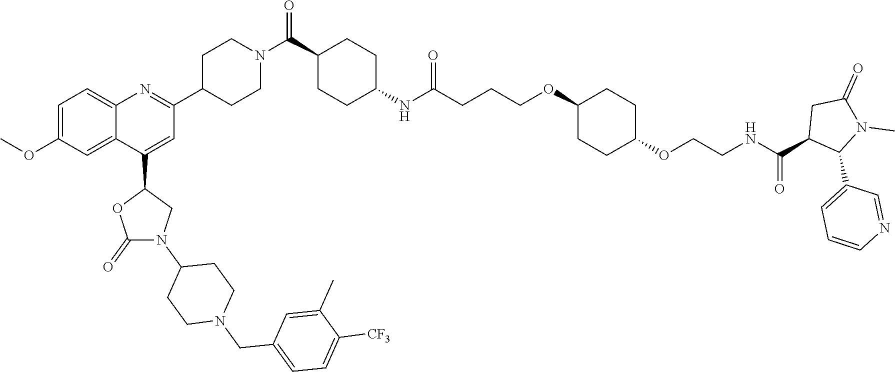 |
| J9 | 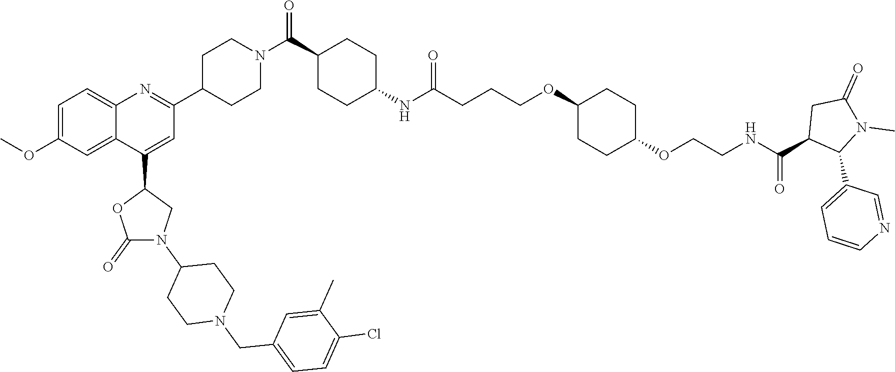 |
| J10 | 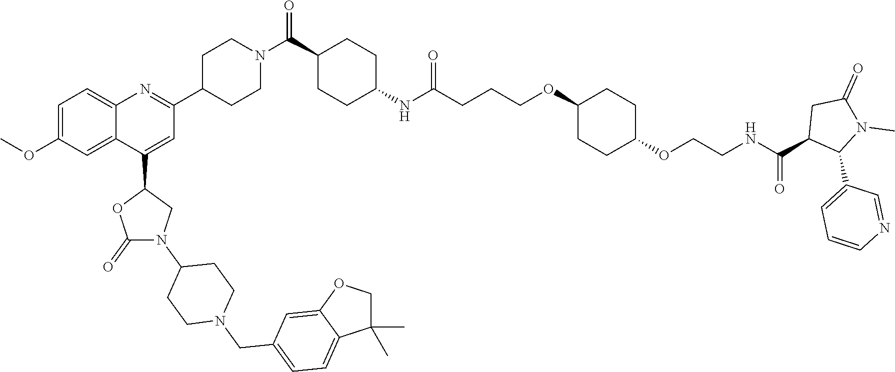 |
| J11 | 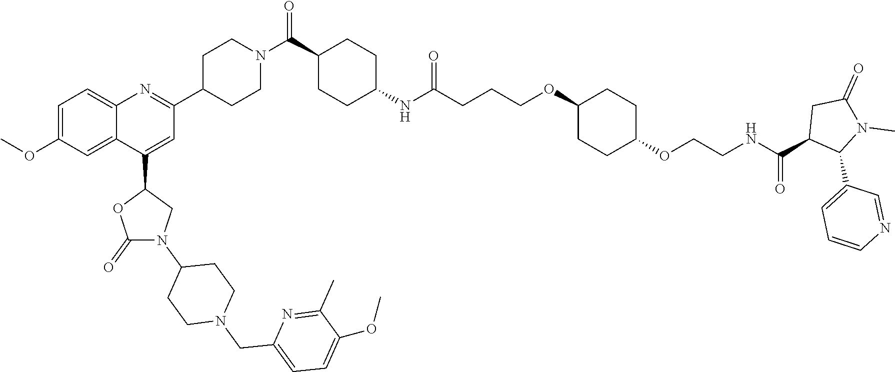 |
| J12 | 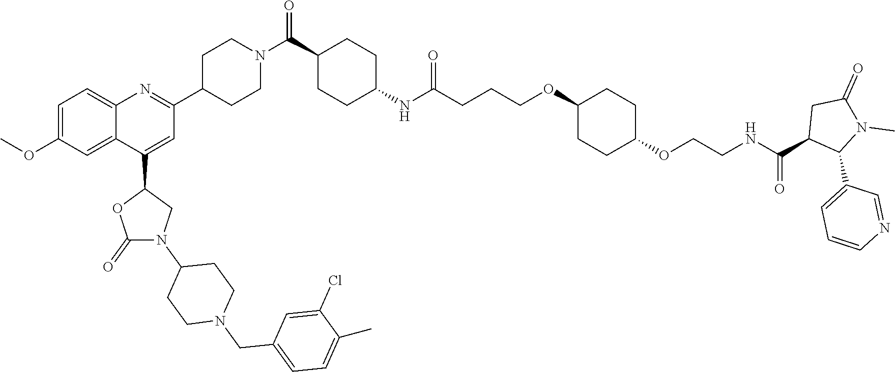 |
| J13 | 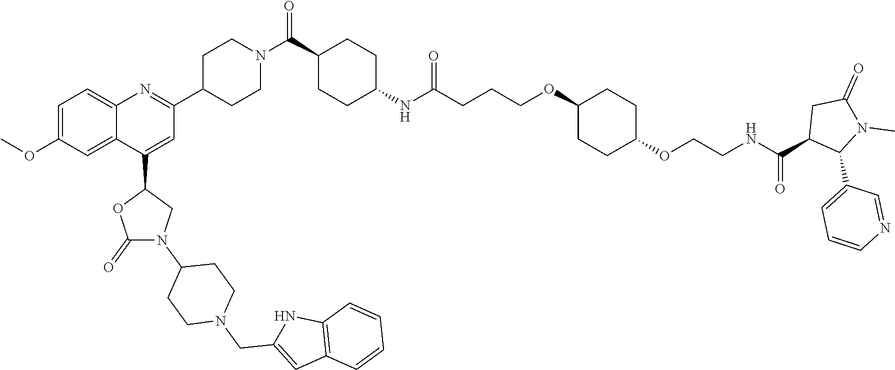 |
| J14 | 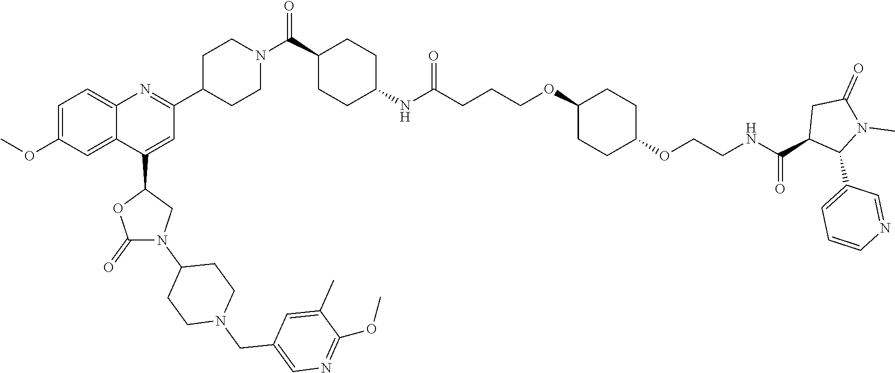 |
| J15 | 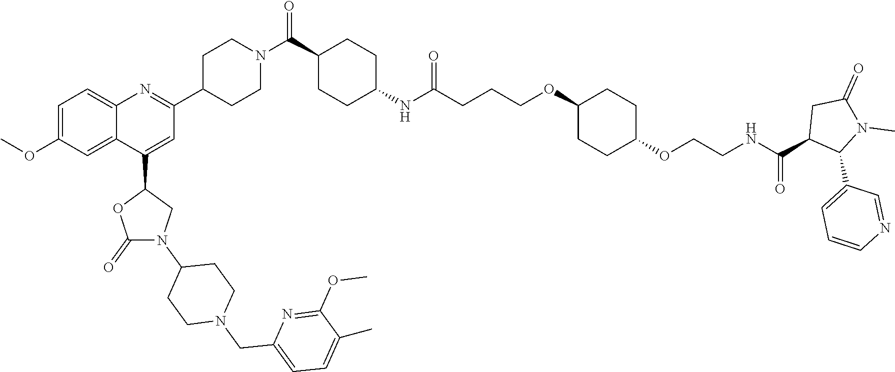 |
| J16 | 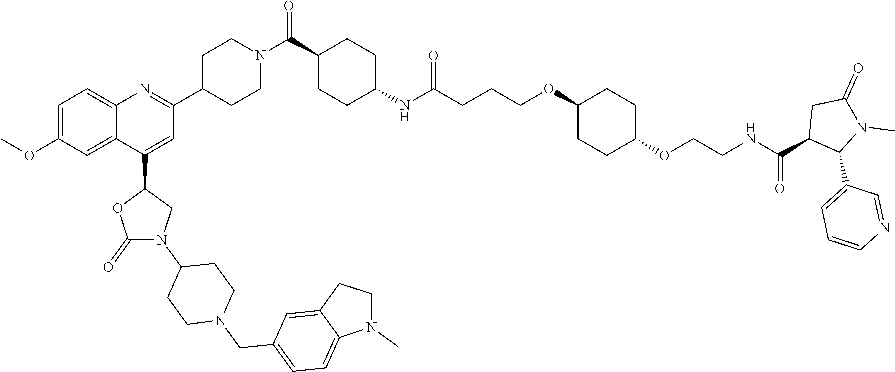 |
| J17 | 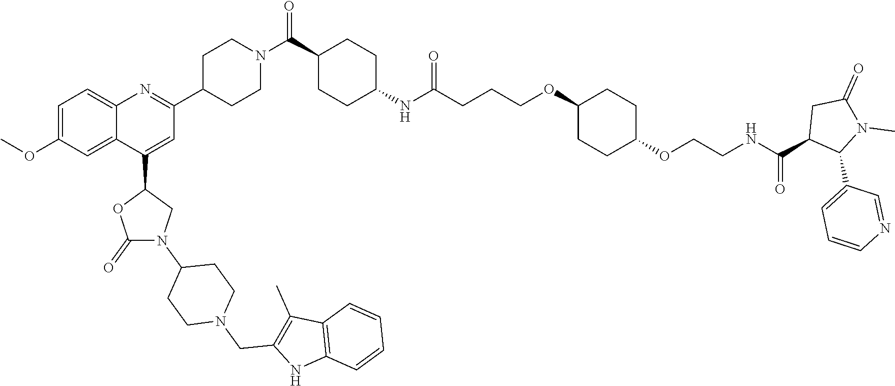 |
| J18 | 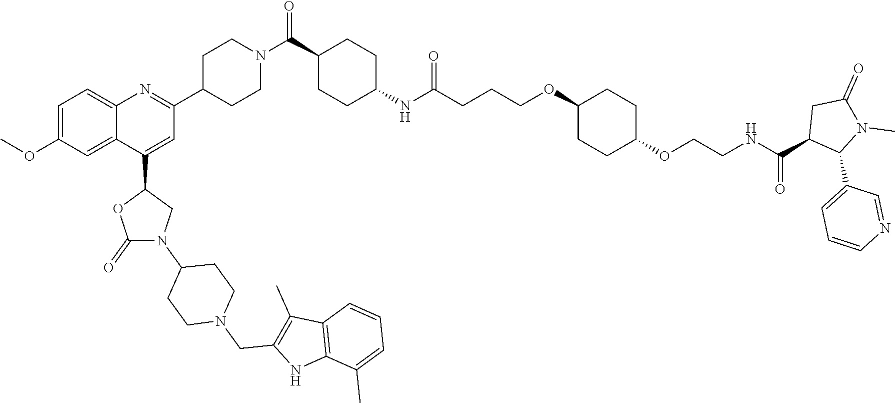 |
| J19 | 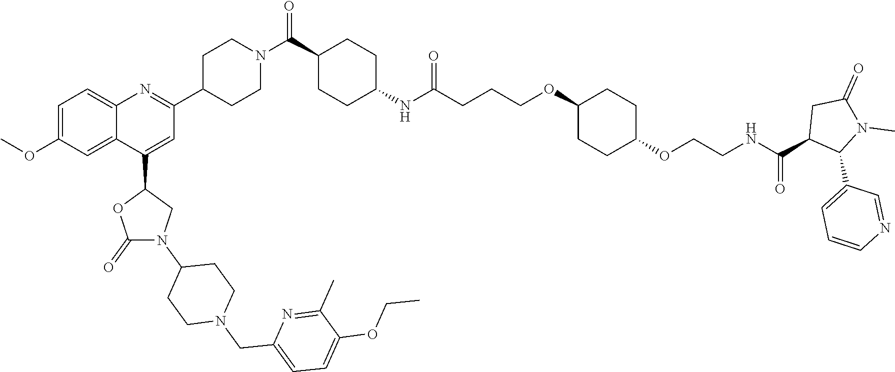 |
| J20 | 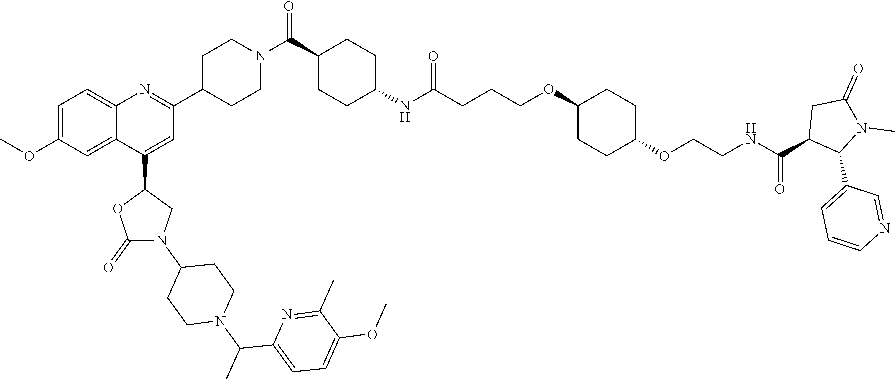 |
| J21 | 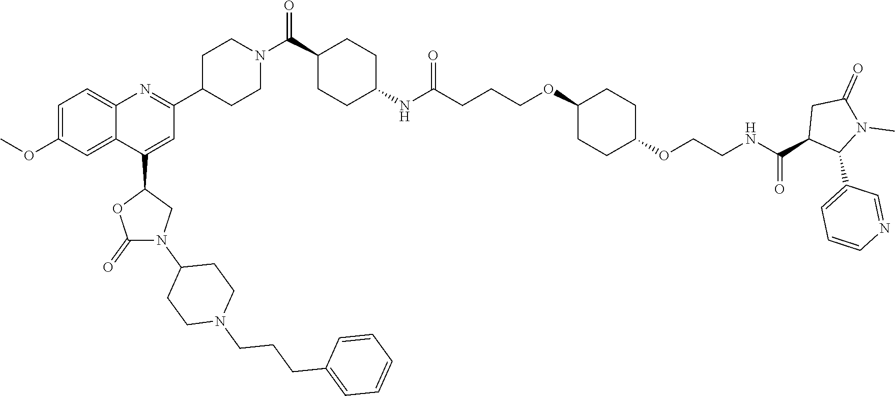 |
| J22 | 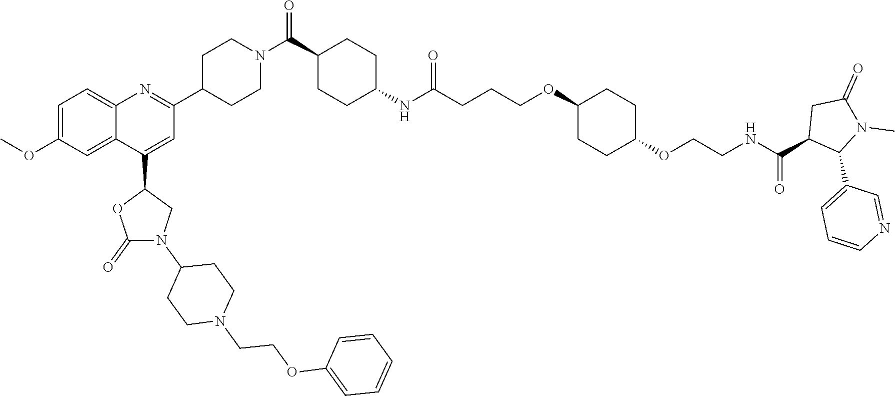 |
| J23 | 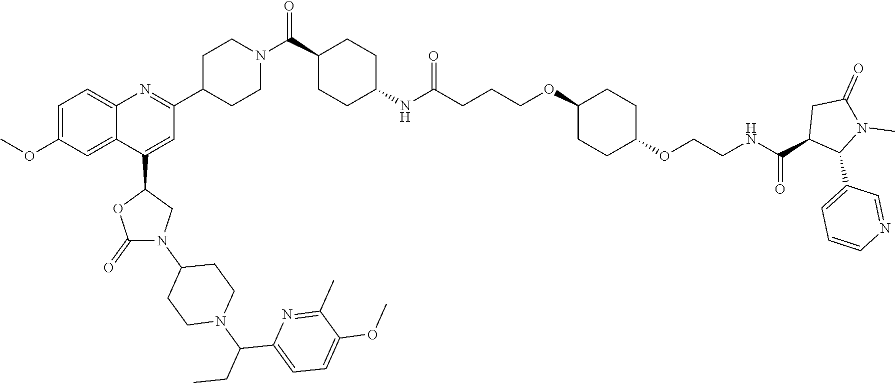 |
| J24 | 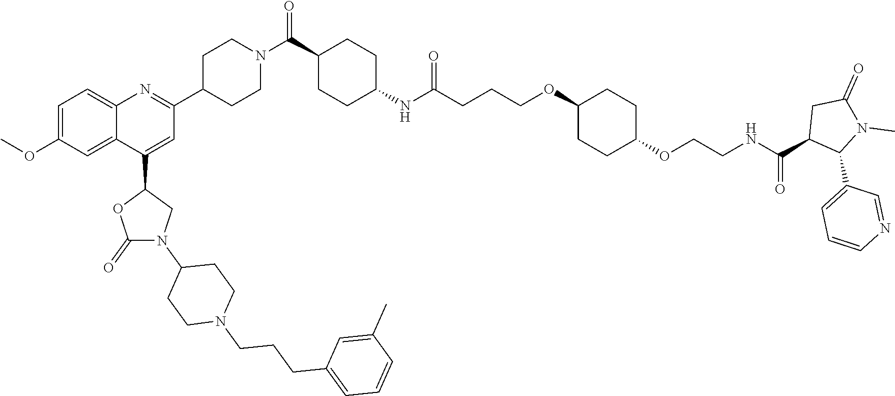 |
| J25 | 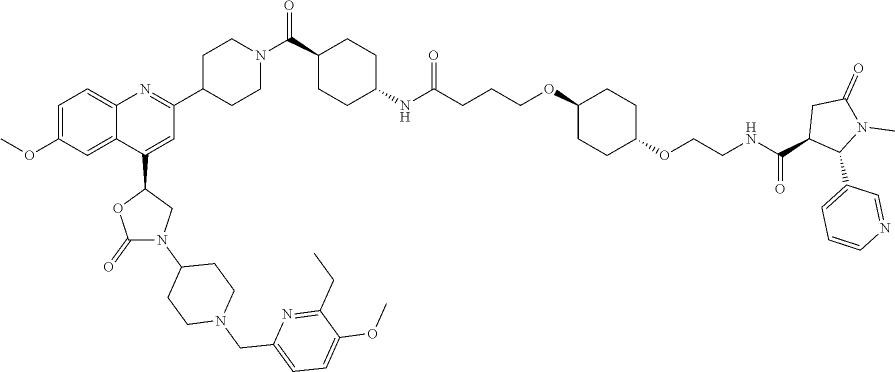 |
| J26 | 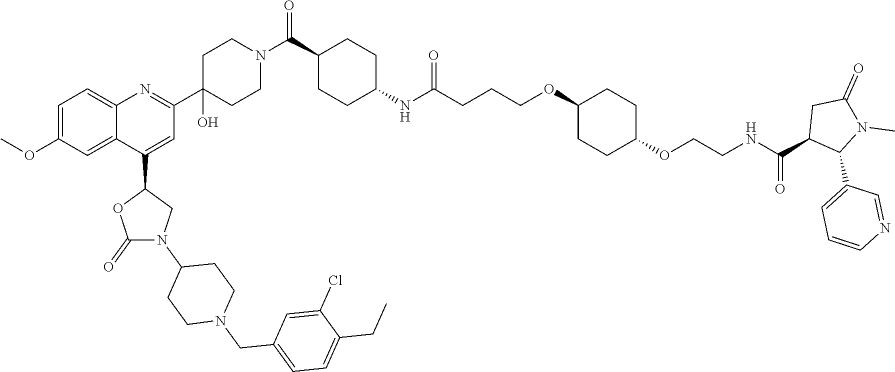 |
| J27 | 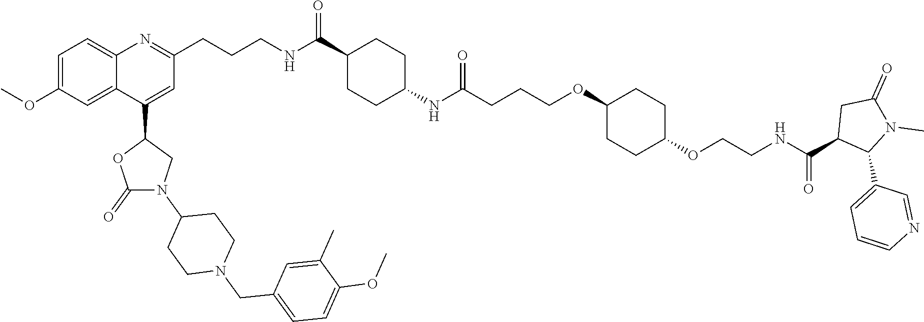 |
| J28 | 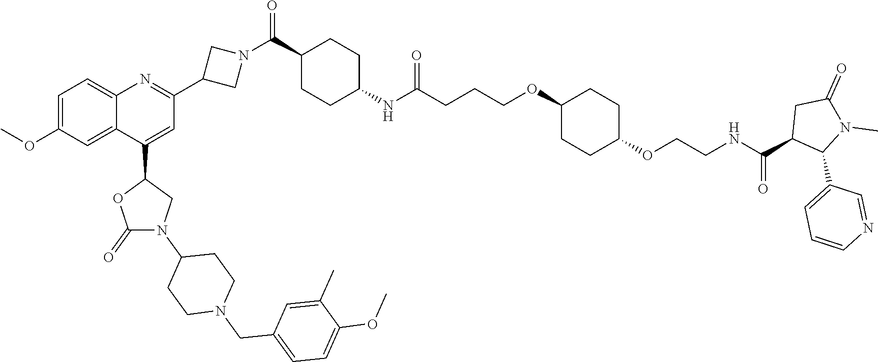 |
| J29 | 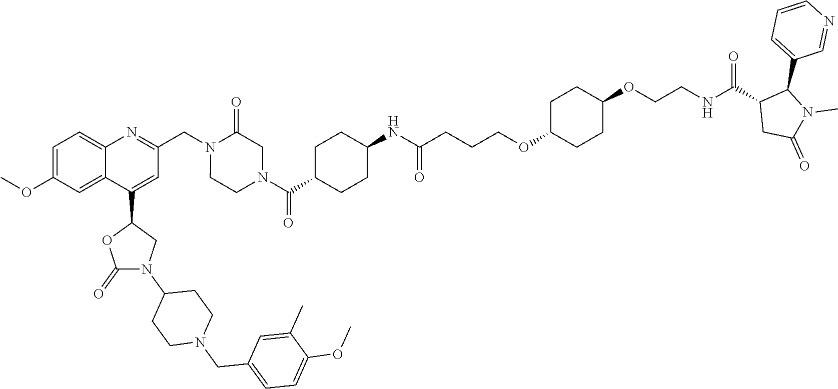 |
| J30 | 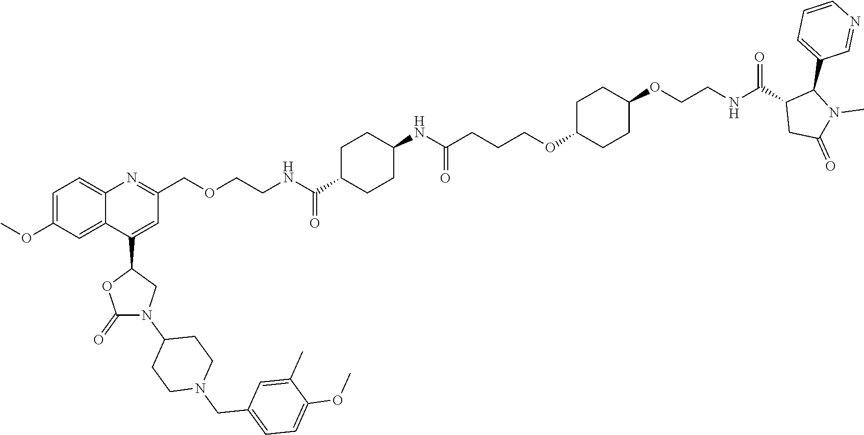 |
| J31 | 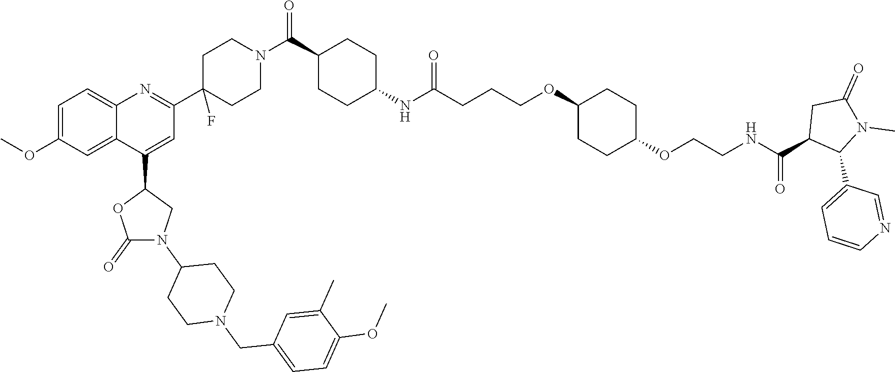 |
| J32 | 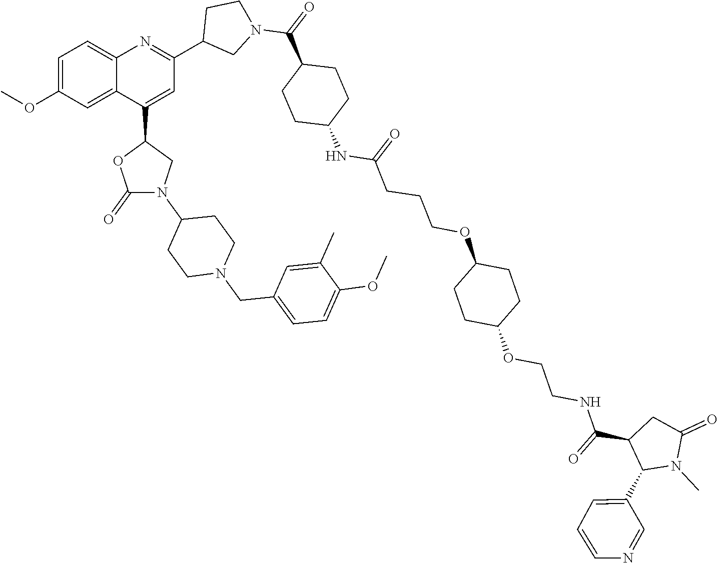 [SMILES: COc1ccc2nc(C3CCN(C(=O)[C@H]4CC[C@H](NC(=O)CCCO[C@H]5CC[C@H](OCCNC(=O)[C@H]6CC(=O)N(C)[C@@H]6c6cccnc6)CC5)CC4)C3)cc([C@H]3CN(C4CCN(Cc5ccc(OC)c(C)c5)CC4)C(=O)O3)c2c1] |
| J33 | 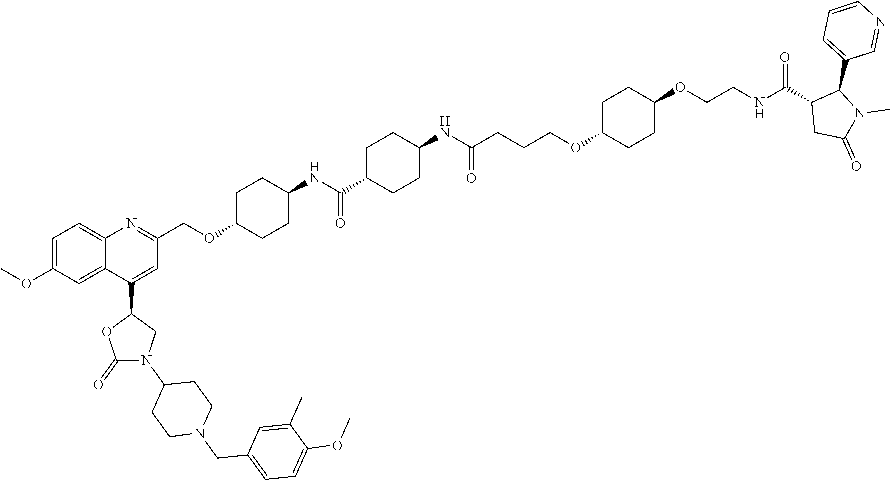 |
| J34 | 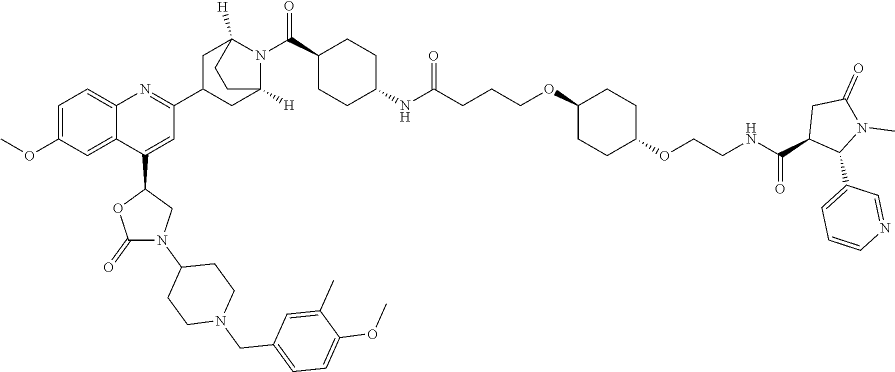 |
| J35 | 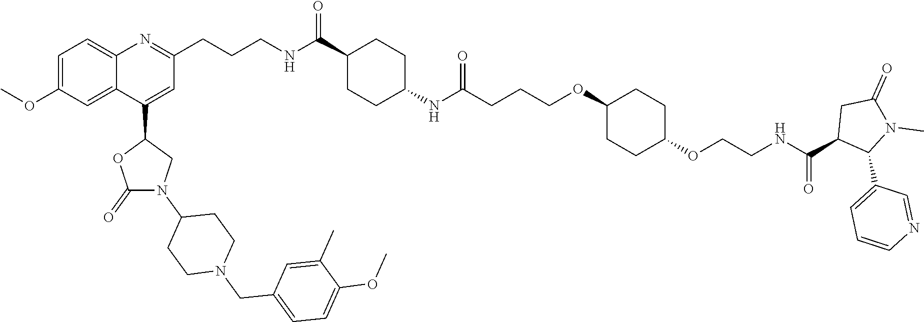 |
| J36 | 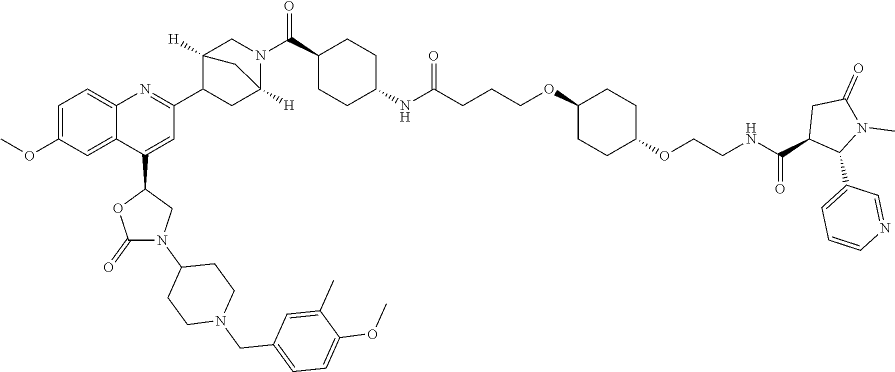 |
| J37 | 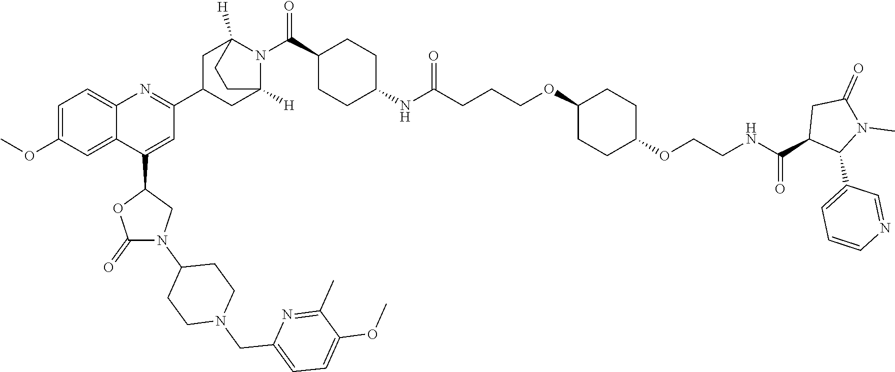 |
| J38 | 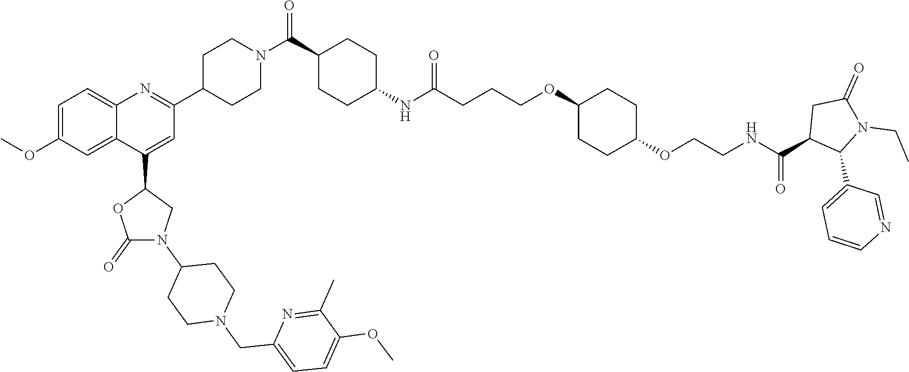 [SMILES: CCN1C(=O)C[C@H](C(=O)NCCO[C@H]2CC[C@H](OCCCC(=O)N[C@H]3CC[C@H](C(=O)N4CCC(c5cc([C@H]6CN(C7CCN(Cc8ccc(OC)c(C)n8)CC7)C(=O)O6)c6cc(OC)ccc6n5)CC4)CC3)CC2)[C@H]1c1cccnc1] |
| J39 | 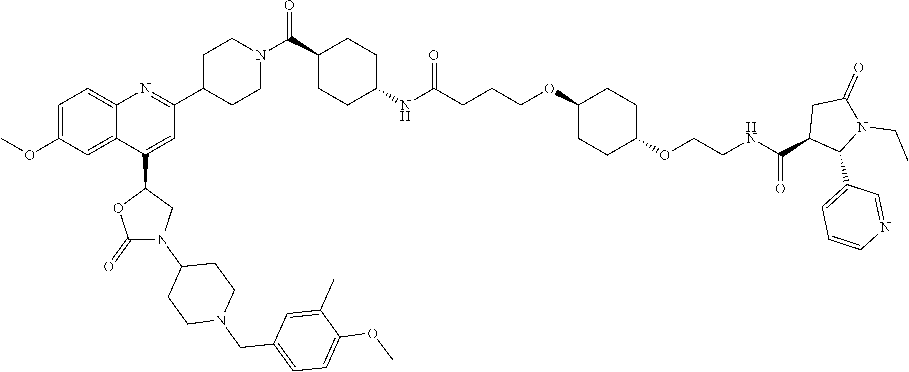 [SMILES: CCN1C(=O)C[C@H](C(=O)NCCO[C@H]2CC[C@H](OCCCC(=O)N[C@H]3CC[C@H](C(=O)N4CCC(c5cc([C@H]6CN(C7CCN(Cc8ccc(OC)c(C)c8)CC7)C(=O)O6)c6cc(OC)ccc6n5)CC4)CC3)CC2)[C@H]1c1cccnc1] |
| J40 | 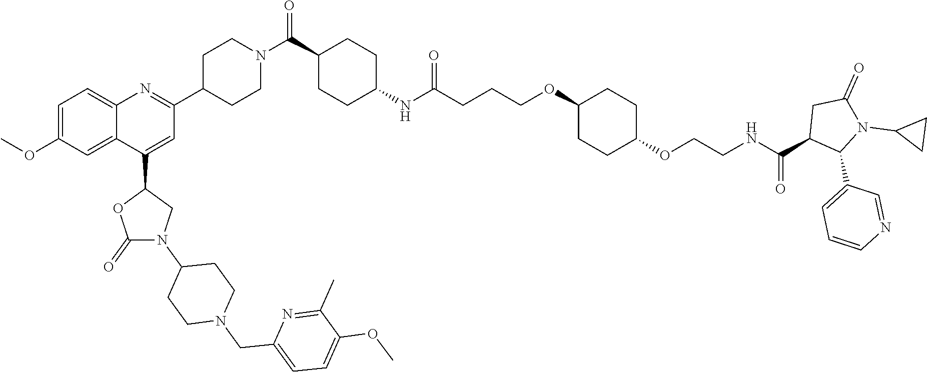 [SMILES: COc1ccc2nc(C3CCN(C(=O)[C@H]4CC[C@H](NC(=O)CCCO[C@H]5CC[C@H](OCCNC(=O)[C@H]6CC(=O)N(C7CC7)[C@@H]6c6cccnc6)CC5)CC4)CC3)cc([C@H]3CN(C4CCN(Cc5ccc(OC)c(C)n5)CC4)C(=O)O3)c2c1] |

### Targets and Target-Binding Moieties

The compounds of Formula (I) as disclosed herein are heterobifunctional synthetic agents designed such that one terminus interacts with a cell surface target, while the other terminus binds a specific antibody. More specifically, the ARM simultaneously binds the cell surface target as well as the specific antibody. This ternary complex directs immune surveillance to target expressing tissue/cells and unites the mechanisms of antibody function with the dose-control of small molecules. This mechanism may include increased cell killing of targeting-expressing cells, depletion of target-expressing cells, antibody dependent cellular cytotoxicity (ADCC), antibody dependent cellular phagocytosis (ADCP), or complement dependent cytotoxicity (CDC). The same Fc receptor expressing immune cells that initiate destruction of the ARM/antibody tagged cells also participate in presentation of endogenous antigens for the potential for long term cellular immunity.

The compounds of Formula (I) as disclosed herein include a target-binding moiety that is capable of binding a target protein (e.g., a receptor) present on the surface of a cell. A person skilled in the art can select molecules known to bind the target protein for use as the target-binding moiety in the ARM.

In one embodiment, the target of the target binding moiety is a cell surface protein. In a further embodiment, the target of the target binding moiety is a target protein expressed on a pathogenic cell.

In a further embodiment, the pathogenic cell is a pathogenic immune cell, a tumor cell or cancer cell, or a stromal cell (including stromal cells present in a tumor microenvironment).

In a further embodiment, the target of the target binding moiety is present on the surface of a pathogenic agent selected from a virus or a bacterial cell. Examples of a virus expressing cell surface targets include, but are not limited to, influenza. Examples of cell surface targets on influenza virus include, but are not limited to, neuraminidase.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or γδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), or exhausted T cells.

In a further embodiment, the tumor cells or cancer cells are solid tumor cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, brain cancer cells, endocrine cancer cells, testicular cancer cells, bladder cancer cells, bone cancer cells, esophageal cancer cells, gastric cancer cells, renal cell cancer cells, melanoma cancer cells, thyroid cancer cells, or breast cancer cells, preferably cells selected from mCRPC cells, breast cancer cells, lung cancer cells, colorectal cancer cells, or renal cell cancer cells.

In a further embodiment, the stromal cells are cancer associated fibroblasts (CAFs).

In one embodiment, the target of the target binding moiety is selected from a G protein-coupled receptor (GPCR), an enzyme (such as a dehydrogenase, an esterase, a phosphodiesterase, a hydrolase, a lipase, a phosphatase, a kinase, a reductase, or a transferase), a transporter (e.g., an ion channel), a protease, or a receptor. In a further embodiment, the target of the target binding moiety is selected from a GPCR, an enzyme (such as a dehydrogenase, an esterase, a phosphodiesterase, a hydrolase, a lipase, a phosphatase, a kinase, a reductase, or a transferase), a transporter (e.g, an ion channel), a protease, or a receptor, wherein the target is associated with and/or expressed on immune cells (including pathogenic immune cells), tumor cells or cancer cells, or stromal cells (including stromal cells present in a tumor microenvironment).

In a further embodiment, the target of the target binding moiety is selected from 15-hydroxyprostaglandin dehydrogenases, 5-hydroxytryptamine receptors, activated leukocyte cell adhesion molecules, ADAM metallopeptidases, adenosine receptors, adenosine deaminases, adrenoceptor beta, advanced glycosylation end-product specific receptors, membrane alanyl aminopeptidases, alkaline phosphatases, calcium voltage-gated channels, cannabinoid receptors, carcinoembryonic antigen related cell adhesion molecules, C-C motif chemokine receptors, CD14, CD19, CD200 receptors, CD22, CD274, CD276, CD33, CD37, CD38, CD3e, CD4, CD44, CD48, CD70, CD74, CD80, CD99, muscarinic cholinergic receptors, nicotinic cholinergic receptors, coagulation factor II thrombin receptors, colony stimulating factor 2 receptors, complement C5a receptors, C-type lectin domains, C—X—C motif chemokine receptors, cysteinyl leukotriene receptors, cytotoxic T-lymphocyte associated proteins, delta like canonical Notch ligands, dipeptidyl peptidases, ectonucleoside triphosphate diphosphohydrolases, erythropoietin receptors, F11 receptors, formyl peptide receptors, FXYD domain containing ion transport regulators, G protein-coupled bile acid receptors, G protein-coupled receptors, gamma-aminobutyric acid type A receptors, gastric inhibitory polypeptide receptors, glutamate metabotropic receptor, platelet glycoproteins, hepatitis A virus cellular receptors, histamine receptors, hydroxycarboxylic acid receptors, integrins, intercellular adhesion molecules, interleukin receptor accessory proteins, interleukin receptors, killer cell lectin like receptors, KISS1 receptors, leukotriene receptors, lymphocyte activating gene proteins, lymphocyte antigens, mannose receptors, membrane metalloendopeptidases, membrane spanning 4-domains, platelet activating factor receptors, potassium calcium-activated channels, potassium voltage-gated channels, programmed cell death proteins, prostaglandin receptors, prostaglandin synthases, protein tyrosine phosphatases, purinergic receptors, pyrimidinergic receptors, scavenger receptors, selectins, signaling lymphocytic activation molecule (SLAM) proteins, sodium voltage-gated channels, somatostatin receptors, sphingosine-1-phosphate receptors, suppression of tumorigenicity proteins, T cell immunoreceptors, thromboxane receptors, TNF receptors, toll like receptors, transient receptor potential cation channels, triggering receptors expressed on myeloid cells, or V-set immunoregulatory receptors.

In a further embodiment, the target of the target binding moiety is a target as listed in Table 4:

TABLE 4

| | | | | | Immune |
|---|---|---|---|---|---|
| | | | | | Class (based |
| Entrez | | | | | on GTEx, |
| Gene | | | Topology_{—} | TargetClass_{—} | GeneLogic, |
| ID | Symbol | Target | Type | Specific | Blueprint) |
| 1233 | CCR4 | C-C motif chemokine | MultiTM | GPCR | Immune |
| | | receptor 4 | | | |
| 1234 | CCR5 | C-C motif chemokine | MultiTM | GPCR | Immune |
| | | receptor 5 | | | |
| | | (gene/pseudogene) | | | |
| 1268 | CNR1 | cannabinoid receptor 1 | MultiTM | GPCR | Likely |
| | | | | | Immune |
| 7852 | CXCR4 | C-X-C motif chemokine | MultiTM | GPCR | Immune |
| | | receptor 4 | | | |
| 728 | C5AR1 | complement C5a | MultiTM | GPCR | Immune |
| | | receptor 1 | | | |
| 1909 | EDNRA | endothelin receptor | MultiTM | GPCR | Possible |
| | | type A | | | Immune |
| 3577 | CXCR1 | C-X-C motif chemokine | MultiTM | GPCR | Immune |
| | | receptor 1 | | | |
| 3579 | CXCR2 | C-X-C motif chemokine | MultiTM | GPCR | Immune |
| | | receptor 2 | | | |
| 8549 | LGR5 | leucine rich repeat | MultiTM | GPCR | Likely |
| | | containing G | | | Immune |
| | | protein-coupled | | | |
| | | receptor 5 | | | |
| 1232 | CCR3 | C-C motif chemokine | MultiTM | GPCR | Immune |
| | | receptor 3 | | | |
| 134391 | GPR151 | G protein-coupled | MultiTM | GPCR | Undetermined |
| | | receptor 151 | | | |
| 729230 | CCR2 | C-C motif chemokine | MultiTM | GPCR | Immune |
| | | receptor 2 | | | |
| 2843 | GPR20 | G protein-coupled | MultiTM | GPCR | Likely |
| | | receptor 20 | | | Immune |
| 4987 | OPRL1 | opioid related | MultiTM | GPCR | Likely |
| | | nociceptin | | | Immune |
| | | receptor 1 | | | |
| 5031 | P2RY6 | pyrimidinergic | MultiTM | GPCR | Immune |
| | | receptor P2Y6 | | | |
| 5732 | PTGER2 | prostaglandin E | MultiTM | GPCR | Immune |
| | | receptor 2 | | | |
| 5734 | PTGER4 | prostaglandin E | MultiTM | GPCR | Likely |
| | | receptor 4 | | | Immune |
| 10803 | CCR9 | C-C motif chemokine | MultiTM | GPCR | Immune |
| | | receptor 9 | | | |
| 1910 | EDNRB | endothelin receptor | MultiTM | GPCR | Non-Immune |
| | | type B | | | |
| 3356 | HTR2A | 5-hydroxytryptamine | MultiTM | GPCR | Possible |
| | | receptor 2A | | | Immune |
| 146 | ADRA1D | adrenoceptor alpha 1D | MultiTM | GPCR | Non-Immune |
| 887 | CCKBR | cholecystokinin B | MultiTM | GPCR | Possible |
| | | receptor | | | Immune |
| 2149 | F2R | coagulation factor II | MultiTM | GPCR | Immune |
| | | thrombin receptor | | | |
| 2492 | FSHR | follicle stimulating | MultiTM | GPCR | Undetermined |
| | | hormone receptor | | | |
| 2847 | MCHR1 | melanin concentrating | MultiTM | GPCR | Possible |
| | | hormone receptor 1 | | | Immune |
| 2862 | MLNR | motilin receptor | MultiTM | GPCR | Likely |
| | | | | | Immune |
| 3363 | HTR7 | 5-hydroxytryptamine | MultiTM | GPCR | Immune |
| | | receptor 7 | | | |
| 5733 | PTGER3 | prostaglandin E | MultiTM | GPCR | Possible |
| | | receptor 3 | | | Immune |
| 10886 | NPFFR2 | neuropeptide FF | MultiTM | GPCR | Likely |
| | | receptor 2 | | | Immune |
| 57105 | CYSLTR2 | cysteinyl leukotriene | MultiTM | GPCR | Immune |
| | | receptor 2 | | | |
| 350383 | GPR142 | G protein-coupled | MultiTM | GPCR | Immune |
| | | receptor 142 | | | |
| 4142 | MAS1 | MAS1 proto-oncogene, | MultiTM | GPCR | Non-Immune |
| | | G protein- | | | |
| | | coupled receptor | | | |
| 6752 | SSTR2 | somatostatin receptor 2 | MultiTM | GPCR | Immune |
| 134 | ADORA1 | adenosine A1 receptor | MultiTM | GPCR | Non-Immune |
| 150 | ADRA2A | adrenoceptor alpha 2A | MultiTM | GPCR | Likely |
| | | | | | Immune |
| 1230 | CCR1 | C-C motif chemokine | MultiTM | GPCR | Immune |
| | | receptor 1 | | | |
| 1235 | CCR6 | C-C motif chemokine | MultiTM | GPCR | Immune |
| | | receptor 6 | | | |
| 1813 | DRD2 | dopamine receptor D2 | MultiTM | GPCR | Non-Immune |
| 1814 | DRD3 | dopamine receptor D3 | MultiTM | GPCR | Undetermined |
| 2358 | FPR2 | formyl peptide receptor 2 | MultiTM | GPCR | Immune |
| 2925 | GRPR | gastrin releasing | MultiTM | GPCR | Likely |
| | | peptide receptor | | | Immune |
| 3269 | HRH1 | histamine receptor H1 | MultiTM | GPCR | Immune |
| 5724 | PTAFR | platelet activating | MultiTM | GPCR | Immune |
| | | factor receptor | | | |
| 9290 | GPR55 | G protein-coupled | MultiTM | GPCR | Immune |
| | | receptor 55 | | | |
| 151306 | GPBAR1 | G protein-coupled | MultiTM | GPCR | Immune |
| | | bile acid receptor 1 | | | |
| 338442 | HCAR2 | hydroxycarboxylic acid | MultiTM | GPCR | Immune |
| | | receptor 2 | | | |
| 136 | ADORA2B | adenosine A2b receptor | MultiTM | GPCR | Immune |
| 140 | ADORA3 | adenosine A3 receptor | MultiTM | GPCR | Undetermined |
| 151 | ADRA2B | adrenoceptor alpha 2B | MultiTM | GPCR | Likely |
| | | | | | Immune |
| 153 | ADRB1 | adrenoceptor beta 1 | MultiTM | GPCR | Likely |
| | | | | | Immune |
| 154 | ADRB2 | adrenoceptor beta 2 | MultiTM | GPCR | Immune |
| 155 | ADRB3 | adrenoceptor beta 3 | MultiTM | GPCR | Possible |
| | | | | | Immune |
| 185 | AGTR1 | angiotensin II receptor | MultiTM | GPCR | Likely |
| | | type 1 | | | Immune |
| 886 | CCKAR | cholecystokinin A | MultiTM | GPCR | Possible |
| | | receptor | | | Immune |
| 1128 | CHRM1 | cholinergic receptor | MultiTM | GPCR | Non-Immune |
| | | muscarinic 1 | | | |
| 1129 | CHRM2 | cholinergic receptor | MultiTM | GPCR | Non-Immune |
| | | muscarinic 2 | | | |
| 1131 | CHRM3 | cholinergic receptor | MultiTM | GPCR | Likely |
| | | muscarinic 3 | | | Immune |
| 1132 | CHRM4 | cholinergic receptor | MultiTM | GPCR | Immune |
| | | muscarinic 4 | | | |
| 1133 | CHRM5 | cholinergic receptor | MultiTM | GPCR | Possible |
| | | muscarinic 5 | | | Immune |
| 1237 | CCR8 | C-C motif chemokine | MultiTM | GPCR | Immune |
| | | receptor 8 | | | |
| 1241 | LTB4R | leukotriene B4 receptor | MultiTM | GPCR | Immune |
| 1269 | CNR2 | cannabinoid receptor 2 | MultiTM | GPCR | Immune |
| 1815 | DRD4 | dopamine receptor D4 | MultiTM | GPCR | Undetermined |
| 1816 | DRD5 | dopamine receptor D5 | MultiTM | GPCR | Non-Immune |
| 1901 | S1PR1 | sphingosine-1-phosphate | MultiTM | GPCR | Likely |
| | | receptor 1 | | | Immune |
| 1902 | LPAR1 | lysophosphatidic acid | MultiTM | GPCR | Possible |
| | | receptor 1 | | | Immune |
| 2693 | GHSR | growth hormone | MultiTM | GPCR | Possible |
| | | secretagogue receptor | | | Immune |
| 2798 | GNRHR | gonadotropin releasing | MultiTM | GPCR | Likely |
| | | hormone receptor | | | Immune |
| 2834 | PRLHR | prolactin releasing | MultiTM | GPCR | Non-Immune |
| | | hormone receptor | | | |
| 2864 | FFAR1 | free fatty acid | MultiTM | GPCR | Likely |
| | | receptor 1 | | | Immune |
| 3061 | HCRTR1 | hypocretin receptor 1 | MultiTM | GPCR | Possible |
| | | | | | Immune |
| 3062 | HCRTR2 | hypocretin receptor 2 | MultiTM | GPCR | Possible |
| | | | | | Immune |
| 3274 | HRH2 | histamine receptor H2 | MultiTM | GPCR | Likely |
| | | | | | Immune |
| 3350 | HTR1A | 5-hydroxytryptamine | MultiTM | GPCR | Non-Immune |
| | | receptor 1A | | | |
| 3351 | HTR1B | 5-hydroxytryptamine | MultiTM | GPCR | Possible |
| | | receptor 1B | | | Immune |
| 3352 | HTR1D | 5-hydroxytryptamine | MultiTM | GPCR | Possible |
| | | receptor 1D | | | Immune |
| 3354 | HTR1E | 5-hydroxytryptamine | MultiTM | GPCR | Non-Immune |
| | | receptor 1E | | | |
| 3355 | HTR1F | 5-hydroxytryptamine | MultiTM | GPCR | Likely |
| | | receptor 1F | | | Immune |
| 3357 | HTR2B | 5-hydroxytryptamine | MultiTM | GPCR | Possible |
| | | receptor 2B | | | Immune |
| 3358 | HTR2C | 5-hydroxytryptamine | MultiTM | GPCR | Possible |
| | | receptor 2C | | | Immune |
| 3361 | HTR5A | 5-hydroxytryptamine | MultiTM | GPCR | Possible |
| | | receptor 5A | | | Immune |
| 3362 | HTR6 | 5-hydroxytryptamine | MultiTM | GPCR | Possible |
| | | receptor 6 | | | Immune |
| 4157 | MC1R | melanocortin 1 receptor | MultiTM | GPCR | Non-Immune |
| 4543 | MTNR1A | melatonin receptor 1A | MultiTM | GPCR | Possible |
| | | | | | Immune |
| 4544 | MTNR1B | melatonin receptor 1B | MultiTM | GPCR | Undetermined |
| 4889 | NPY5R | neuropeptide Y | MultiTM | GPCR | Likely |
| | | receptor Y5 | | | Immune |
| 4985 | OPRD1 | opioid receptor delta 1 | MultiTM | GPCR | Possible |
| | | | | | Immune |
| 4986 | OPRK1 | opioid receptor kappa 1 | MultiTM | GPCR | Non-Immune |
| 5021 | OXTR | oxytocin receptor | MultiTM | GPCR | Likely |
| | | | | | Immune |
| 5029 | P2RY2 | purinergic receptor P2Y2 | MultiTM | GPCR | Immune |
| 5729 | PTGDR | prostaglandin D2 receptor | MultiTM | GPCR | Immune |
| 5731 | PTGER1 | prostaglandin E receptor 1 | MultiTM | GPCR | Likely |
| | | | | | Immune |
| 5737 | PTGFR | prostaglandin F receptor | MultiTM | GPCR | Likely |
| | | | | | Immune |
| 5739 | PTGIR | prostaglandin 12 receptor | MultiTM | GPCR | Immune |
| 6754 | SSTR4 | somatostatin receptor 4 | MultiTM | GPCR | Possible |
| | | | | | Immune |
| 6755 | SSTR5 | somatostatin receptor 5 | MultiTM | GPCR | Non-Immune |
| 6865 | TACR2 | tachykinin receptor 2 | MultiTM | GPCR | Non-Immune |
| 6869 | TACR1 | tachykinin receptor 1 | MultiTM | GPCR | Non-Immune |
| 6915 | TBXA2R | thromboxane A2 receptor | MultiTM | GPCR | Immune |
| 7201 | TRHR | thyrotropin releasing | MultiTM | GPCR | Likely |
| | | hormone receptor | | | Immune |
| 8477 | GPR65 | G protein-coupled | MultiTM | GPCR | Immune |
| | | receptor 65 | | | |
| 8698 | S1PR4 | sphingosine-1-phosphate | MultiTM | GPCR | Immune |
| | | receptor 4 | | | |
| 8811 | GALR2 | galanin receptor 2 | MultiTM | GPCR | Likely |
| | | | | | Immune |
| 8843 | HCAR3 | hydroxycarboxylic | MultiTM | GPCR | Immune |
| | | acid receptor 3 | | | |
| 9002 | F2RL3 | F2R like thrombin | MultiTM | GPCR | Likely |
| | | or trypsin receptor 3 | | | Immune |
| 10800 | CYSLTR1 | cysteinyl leukotriene | MultiTM | GPCR | Immune |
| | | receptor 1 | | | |
| 11251 | PTGDR2 | prostaglandin D2 | MultiTM | GPCR | Likely |
| | | receptor 2 | | | Immune |
| 11255 | HRH3 | histamine receptor H3 | MultiTM | GPCR | Possible |
| | | | | | Immune |
| 23432 | GPR161 | G protein-coupled | MultiTM | GPCR | Possible |
| | | receptor 161 | | | Immune |
| 29933 | GPR132 | G protein-coupled | MultiTM | GPCR | Immune |
| | | receptor 132 | | | |
| 53637 | S1PR5 | sphingosine-1-phosphate | MultiTM | GPCR | Immune |
| | | receptor 5 | | | |
| 53831 | GPR84 | G protein-coupled | MultiTM | GPCR | Immune |
| | | receptor 84 | | | |
| 59340 | HRH4 | histamine receptor H4 | MultiTM | GPCR | Likely |
| | | | | | Immune |
| 64805 | P2RY12 | purinergic receptor P2Y12 | MultiTM | GPCR | Immune |
| 84539 | MCHR2 | melanin concentrating | MultiTM | GPCR | Possible |
| | | hormone receptor 2 | | | Immune |
| 84634 | KISS1R | KISS1 receptor | MultiTM | GPCR | Immune |
| 84636 | GPR174 | G protein-coupled | MultiTM | GPCR | Immune |
| | | receptor 174 | | | |
| 124274 | GPR139 | G protein-coupled | MultiTM | GPCR | Undetermined |
| | | receptor 139 | | | |
| 139760 | GPR119 | G protein-coupled | MultiTM | GPCR | Undetermined |
| | | receptor 119 | | | |
| 338557 | FFAR4 | free fatty acid | MultiTM | GPCR | Likely |
| | | receptor 4 | | | Immune |
| 6870 | TACR3 | tachykinin receptor 3 | MultiTM | GPCR | Non-Immune |
| 10203 | CALCRL | calcitonin receptor | MultiTM | GPCR | Likely |
| | | like receptor | | | Immune |
| 2740 | GLP1R | glucagon like peptide | MultiTM | GPCR | Possible |
| | | 1 receptor | | | Immune |
| 2696 | GIPR | gastric inhibitory | MultiTM | GPCR | Immune |
| | | polypeptide receptor | | | |
| 5745 | PTH1R | parathyroid hormone | MultiTM | GPCR | Possible |
| | | 1 receptor | | | Immune |
| 1395 | CRHR2 | corticotropin releasing | MultiTM | GPCR | Non-Immune |
| | | hormone receptor 2 | | | |
| 2550 | GABBR1 | gamma-aminobutyric | MultiTM | GPCR | Non-Immune |
| | | acid type B | | | |
| | | receptor subunit 1 | | | |
| 2913 | GRM3 | glutamate metabotropic | MultiTM | GPCR | Likely |
| | | receptor 3 | | | Immune |
| 6608 | SMO | smoothened, frizzled | MultiTM | GPCR | Non-Immune |
| | | class receptor | | | |
| 799 | CALCR | calcitonin receptor | MultiTM | GPCR | Possible |
| | | | | | Immune |
| 846 | CASR | calcium sensing | MultiTM | GPCR | Likely |
| | | receptor | | | Immune |
| 1394 | CRHR1 | corticotropin releasing | MultiTM | GPCR | Non-Immune |
| | | hormone receptor 1 | | | |
| 2692 | GHRHR | growth hormone releasing | MultiTM | GPCR | Non-Immune |
| | | hormone receptor | | | |
| 2912 | GRM2 | glutamate metabotropic | MultiTM | GPCR | Immune |
| | | receptor 2 | | | |
| 2914 | GRM4 | glutamate metabotropic | MultiTM | GPCR | Non-Immune |
| | | receptor 4 | | | |
| 2915 | GRM5 | glutamate metabotropic | MultiTM | GPCR | Possible |
| | | receptor 5 | | | Immune |
| 2917 | GRM7 | glutamate metabotropic | MultiTM | GPCR | Undetermined |
| | | receptor 7 | | | |
| 9340 | GLP2R | glucagon like peptide | MultiTM | GPCR | Non-Immune |
| | | 2 receptor | | | |
| 3248 | HPGD | 15-hydroxyprostaglandin | Membrane | Enzyme_Dehydrogenase | Immune |
| | | dehydrogenase | | | |
| 43 | ACHE | acetylcholinesterase | Membrane | Enzyme_Esterase_{—} | Likely |
| | | (Cartwright blood group) | | Other | Immune |
| 5139 | PDE3A | phosphodiesterase 3A | MultiTM | Enzyme_Esterase_{—} | Possible |
| | | | | Phosphodiesterase | Immune |
| 953 | ENTPD1 | ectonucleoside triphosphate | MultiTM | Enzyme_Hydrolase | Immune |
| | | diphosphohydrolase 1 | | | |
| 4023 | LPL | lipoprotein lipase | Membrane | Enzyme_Lipase | Likely |
| | | | | | Immune |
| 4907 | NT5E | 5′-’ucleotidase ecto | Membrane | Enzyme_other | Likely |
| | | | | | Immune |
| 2984 | GUCY2C | guanylate cyclase 2C | SingleTM | Enzyme_other | Possible |
| | | | | | Immune |
| 760 | CA2 | carbonic anhydrase 2 | Membrane | Enzyme_other | Likely |
| | | | | | Immune |
| 7173 | TPO | thyroid peroxidase | SingleTM | Enzyme_other | Likely |
| | | | | | Immune |
| 50507 | NOX4 | NADPH oxidase 4 | MultiTM | Enzyme_other | Non-Immune |
| 3309 | HSPA5 | heat shock protein family A | Membrane | Enzyme_other | Likely |
| | | (Hsp70) member 5 | | | Immune |
| 100 | ADA | adenosine deaminase | Membrane | Enzyme_other | Immune |
| 2280 | FKBP1A | FK506 binding protein 1A | Membrane | Enzyme_other | Possible |
| | | | | | Immune |
| 9536 | PTGES | prostaglandin E synthase | MultiTM | Enzyme_other | Immune |
| 130013 | ACMSD | aminocarboxymuconate | SingleTM | Enzyme_other | Non-Immune |
| | | semialdehyde decarboxylase | | | |
| 5169 | ENPP3 | ectonucleotide | SingleTM | Enzyme_Phosphatase | Possible |
| | | pyrophosphatase/ | | | Immune |
| | | phosphodiesterase 3 | | | |
| 6647 | SOD1 | superoxide dismutase 1 | Membrane | Enzyme_Phosphatase | Possible |
| | | | | | Immune |
| 5167 | ENPP1 | ectonucleotide | SingleTM | Enzyme_Phosphatase | Non-Immune |
| | | pyrophosphatase/ | | | |
| | | phosphodiesterase 1 | | | |
| 249 | ALPL | alkaline phosphatase, | Membrane | Enzyme_Phosphatase | Immune |
| | | biomineralization associated | | | |
| 5788 | PTPRC | protein tyrosine | SingleTM | Enzyme_Phosphatase | Immune |
| | | phosphatase, receptor | | | |
| | | type C | | | |
| 8639 | AOC3 | amine oxidase, copper | SingleTM | Enzyme_Reductase | Non-Immune |
| | | containing 3 | | | |
| 6121 | RPE65 | RPE65, retinoid | Membrane | Enzyme_Reductase | Non-Immune |
| | | isomerohydrolase | | | |
| 952 | CD38 | CD38 molecule | SingleTM | Enzyme_Transferase | Immune |
| 7124 | TNF | tumor necrosis factor | SingleTM | Extracellular | Immune |
| | | | | MB_Bound | |
| | | | | Ligand | |
| 1435 | CSF1 | colony stimulating | SingleTM | Extracellular | Immune |
| | | factor 1 | | MB_Bound | |
| | | | | Ligand | |
| 6376 | CX3CL1 | C-X3-C motif chemokine | SingleTM | Extracellular | Non-Immune |
| | | ligand 1 | | MB_Bound | |
| | | | | Ligand | |
| 8743 | TNFSF10 | TNF superfamily member 10 | SingleTM | Extracellular | Likely |
| | | | | MB_Bound | Immune |
| | | | | Ligand | |
| 5027 | P2RX7 | purinergic receptor P2X 7 | MultiTM | Ion Channel | Likely |
| | | | | | Immune |
| 3738 | KCNA3 | potassium voltage-gated | MultiTM | Ion Channel | Immune |
| | | channel subfamily A | | | |
| | | member 3 | | | |
| 6335 | SCN9A | sodium voltage-gated | MultiTM | Ion Channel | Immune |
| | | channel alpha subunit 9 | | | |
| 6336 | SCN10A | sodium voltage-gated | MultiTM | Ion Channel | Non-Immune |
| | | channel alpha subunit 10 | | | |
| 7442 | TRPV1 | transient receptor | MultiTM | Ion Channel | Undetermined |
| | | potential cation | | | |
| | | channel subfamily | | | |
| | | V member 1 | | | |
| 8989 | TRPA1 | transient receptor | MultiTM | Ion Channel | Likely |
| | | potential cation | | | |
| | | channel subfamily | | | Immune |
| | | A member 1 | | | |
| 3783 | KCNN4 | potassium | MultiTM | Ion Channel | Immune |
| | | calcium-activated | | | |
| | | channel subfamily N | | | |
| | | member 4 | | | |
| 53827 | FXYD5 | FXYD domain containing | SingleTM | Ion Channel | Immune |
| | | ion transport | | | |
| | | regulator 5 | | | |
| 2554 | GABRA1 | gamma-aminobutyric | MultiTM | Ion Channel | Likely |
| | | acid type A | | | |
| | | receptor alpha1 | | | Immune |
| | | subunit | | | |
| 2890 | GRIA1 | glutamate ionotropic | MultiTM | Ion Channel | Possible |
| | | receptor AMPA type | | | |
| | | subunit 1 | | | Immune |
| 2891 | GRIA2 | glutamate ionotropic | MultiTM | Ion Channel | Non-Immune |
| | | receptor AMPA type | | | |
| | | subunit 2 | | | |
| 8913 | CACNA1G | calcium voltage-gated | MultiTM | Ion Channel | Non-Immune |
| | | channel subunit | | | |
| | | alpha1 G | | | |
| 22953 | P2RX2 | purinergic receptor | MultiTM | Ion Channel | Likely |
| | | P2X 2 | | | Immune |
| 774 | CACNA1B | calcium voltage-gated | MultiTM | Ion Channel | Possible |
| | | channel subunit | | | Immune |
| | | alpha1 B | | | |
| 778 | CACNA1F | calcium voltage-gated | MultiTM | Ion Channel | Immune |
| | | channel subunit | | | |
| | | alpha1 F | | | |
| 1136 | CHRNA3 | cholinergic receptor | MultiTM | Ion Channel | Likely |
| | | nicotinic alpha 3 | | | Immune |
| | | subunit | | | |
| 1139 | CHRNA7 | cholinergic receptor | MultiTM | Ion Channel | Undetermined |
| | | nicotinic alpha 7 | | | |
| | | subunit | | | |
| 1141 | CHRNB2 | cholinergic receptor | MultiTM | Ion Channel | Possible |
| | | nicotinic beta 2 | | | Immune |
| | | subunit | | | |
| 1143 | CHRNB4 | cholinergic receptor | MultiTM | Ion Channel | Possible |
| | | nicotinic beta 4 | | | Immune |
| | | subunit | | | |
| 2555 | GABRA2 | gamma-aminobutyric acid | MultiTM | Ion Channel | Non-Immune |
| | | type A receptor alpha2 | | | |
| | | subunit | | | |
| 2556 | GABRA3 | gamma-aminobutyric acid | MultiTM | Ion Channel | Possible |
| | | type A receptor alpha3 | | | Immune |
| | | subunit | | | |
| 2558 | GABRA5 | gamma-aminobutyric acid | MultiTM | Ion Channel | Possible |
| | | type A receptor alpha5 | | | Immune |
| | | subunit | | | |
| 2561 | GABRB2 | gamma-aminobutyric acid | MultiTM | Ion Channel | Immune |
| | | type A receptor beta2 | | | |
| | | subunit | | | |
| 2902 | GRIN1 | glutamate ionotropic | MultiTM | Ion Channel | Possible |
| | | receptor NMDA type | | | Immune |
| | | subunit 1 | | | |
| 2904 | GRIN2B | glutamate ionotropic | MultiTM | Ion Channel | Possible |
| | | receptor NMDA type | | | Immune |
| | | subunit 2B | | | |
| 3359 | HTR3A | 5-hydroxytryptamine | MultiTM | Ion Channel | Immune |
| | | receptor 3A | | | |
| 5025 | P2RX4 | purinergic receptor | MultiTM | Ion Channel | Likely |
| | | P2X 4 | | | Immune |
| 6337 | SCNN1A | sodium channel | MultiTM | Ion Channel | Possible |
| | | epithelial 1 alpha | | | Immune |
| | | subunit | | | |
| 7223 | TRPC4 | transient receptor | MultiTM | Ion Channel | Non-Immune |
| | | potential cation | | | |
| | | channel subfamily | | | |
| | | C member 4 | | | |
| 7225 | TRPC6 | transient receptor | MultiTM | Ion Channel | Likely |
| | | potential cation | | | Immune |
| | | channel subfamily | | | |
| | | C member 6 | | | |
| 8001 | GLRA3 | glycine receptor | MultiTM | Ion Channel | Possible |
| | | alpha 3 | | | Immune |
| 8973 | CHRNA6 | cholinergic receptor | MultiTM | Ion Channel | Immune |
| | | nicotinic alpha | | | |
| | | 6 subunit | | | |
| 54822 | TRPM7 | transient receptor | MultiTM | Ion Channel | Possible |
| | | potential cation | | | Immune |
| | | channel subfamily | | | |
| | | M member 7 | | | |
| 79054 | TRPM8 | transient receptor | MultiTM | Ion Channel | Possible |
| | | potential cation | | | Immune |
| | | channel subfamily | | | |
| | | M member 8 | | | |
| 84876 | ORAI1 | ORAI calcium | MultiTM | Ion Channel | Likely |
| | | release-activated | | | |
| | | calcium modulator 1 | | | Immune |
| 361 | AQP4 | aquaporin 4 | MultiTM | Ion Channel | Likely |
| | | | | | Immune |
| 3758 | KCNJ1 | potassium voltage-gated | MultiTM | Ion Channel | Likely |
| | | channel subfamily J | | | Immune |
| | | member 1 | | | |
| 3786 | KCNQ3 | potassium voltage-gated | MultiTM | Ion Channel | Likely |
| | | channel subfamily Q | | | Immune |
| | | member 3 | | | |
| 6323 | SCN1A | sodium voltage-gated | MultiTM | Ion Channel | Possible |
| | | channel alpha | | | Immune |
| | | subunit 1 | | | |
| 6326 | SCN2A | sodium voltage-gated | MultiTM | Ion Channel | Likely |
| | | channel alpha | | | Immune |
| | | subunit 2 | | | |
| 6328 | SCN3A | sodium voltage-gated | MultiTM | Ion Channel | Likely |
| | | channel alpha | | | Immune |
| | | subunit 3 | | | |
| 6334 | SCN8A | sodium voltage-gated | MultiTM | Ion Channel | Immune |
| | | channel alpha | | | |
| | | subunit 8 | | | |
| 57657 | HCN3 | hyperpolarization | MultiTM | Ion Channel | Non-Immune |
| | | activated cyclic | | | |
| | | nucleotide gated | | | |
| | | potassium | | | |
| | | channel 3 | | | |
| 59341 | TRPV4 | transient receptor | MultiTM | Ion Channel | Immune |
| | | potential cation | | | |
| | | channel subfamily | | | |
| | | V member 4 | | | |
| 359 | AQP2 | aquaporin 2 | MultiTM | Ion Channel | Possible |
| | | | | | Immune |
| 610 | HCN2 | hyperpolarization | MultiTM | Ion Channel | Possible |
| | | activated cyclic | | | Immune |
| | | nucleotide gated | | | |
| | | potassium and | | | |
| | | sodium channel 2 | | | |
| 775 | CACNA1C | calcium voltage-gated | MultiTM | Ion Channel | Non-Immune |
| | | channel subunit | | | |
| | | alpha1 C | | | |
| 779 | CACNA1S | calcium voltage-gated | MultiTM | Ion Channel | Non-Immune |
| | | channel subunit | | | |
| | | alpha1 S | | | |
| 1180 | CLCN1 | chloride voltage-gated | MultiTM | Ion Channel | Possible |
| | | channel 1 | | | Immune |
| 3751 | KCND2 | potassium voltage-gated | MultiTM | Ion Channel | Likely |
| | | channel subfamily D | | | Immune |
| | | member 2 | | | |
| 3757 | KCNH2 | potassium voltage-gated | MultiTM | Ion Channel | Likely |
| | | channel subfamily H | | | Immune |
| | | member 2 | | | |
| 3778 | KCNMA1 | potassium calcium-activated | MultiTM | Ion Channel | Possible |
| | | channel subfamily M alpha 1 | | | Immune |
| 3784 | KCNQ1 | potassium voltage-gated | MultiTM | Ion Channel | Immune |
| | | channel subfamily Q | | | |
| | | member 1 | | | |
| 3785 | KCNQ2 | potassium voltage-gated | MultiTM | Ion Channel | Likely |
| | | channel subfamily Q | | | Immune |
| | | member 2 | | | |
| 6329 | SCN4A | sodium voltage-gated | MultiTM | Ion Channel | Likely |
| | | channel alpha subunit 4 | | | Immune |
| 6331 | SCN5A | sodium voltage-gated | MultiTM | Ion Channel | Possible |
| | | channel alpha subunit 5 | | | Immune |
| 10021 | HCN4 | hyperpolarization | MultiTM | Ion Channel | Non-Immune |
| | | activated cyclic | | | |
| | | nucleotide gated | | | |
| | | potassium channel 4 | | | |
| 11280 | SCN11A | sodium voltage-gated | MultiTM | Ion Channel | Immune |
| | | channel alpha subunit 11 | | | |
| 55503 | TRPV6 | transient receptor | MultiTM | Ion Channel | Possible |
| | | potential cation | | | |
| | | channel subfamily | | | Immune |
| | | V member 6 | | | |
| 348980 | HCN1 | hyperpolarization | MultiTM | Ion Channel | Non-Immune |
| | | activated cyclic | | | |
| | | nucleotide gated | | | |
| | | potassium channel 1 | | | |
| 3760 | KCNJ3 | potassium voltage-gated | MultiTM | Ion Channel | Non-Immune |
| | | channel subfamily J | | | |
| | | member 3 | | | |
| 3762 | KCNJ5 | potassium voltage-gated | MultiTM | Ion Channel | Immune |
| | | channel subfamily J | | | |
| | | member 5 | | | |
| 23416 | KCNH3 | potassium voltage-gated | MultiTM | Ion Channel | Likely |
| | | channel subfamily H | | | Immune |
| | | member 3 | | | |
| 1137 | CHRNA4 | cholinergic receptor | MultiTM | Ion Channel | Likely |
| | | nicotinic alpha 4 | | | Immune |
| | | subunit | | | |
| 2563 | GABRD | gamma-aminobutyric | MultiTM | Ion Channel | Non-Immune |
| | | acid type A receptor | | | |
| | | delta subunit | | | |
| 2892 | GRIA3 | glutamate ionotropic | MultiTM | Ion Channel | Possible |
| | | receptor AMPA type | | | Immune |
| | | subunit 3 | | | |
| 2893 | GRIA4 | glutamate ionotropic | MultiTM | Ion Channel | Non-Immune |
| | | receptor AMPA type | | | |
| | | subunit 4 | | | |
| 776 | CACNA1D | calcium voltage-gated | MultiTM | Ion Channel | Likely |
| | | channel subunit | | | Immune |
| | | alpha1 D | | | |
| 1181 | CLCN2 | chloride voltage-gated | MultiTM | Ion Channel | Non-Immune |
| | | channel 2 | | | |
| 3746 | KCNC1 | potassium voltage-gated | MultiTM | Ion Channel | Non-Immune |
| | | channel subfamily C | | | |
| | | member 1 | | | |
| 3747 | KCNC2 | potassium voltage-gated | MultiTM | Ion Channel | Possible |
| | | channel subfamily C | | | Immune |
| | | member 2 | | | |
| 3776 | KCNK2 | potassium two pore | MultiTM | Ion Channel | Possible |
| | | domain channel | | | Immune |
| | | subfamily K member 2 | | | |
| 1956 | EGFR | epidermal growth | SingleTM | Kinase_Protein | Possible |
| | | factor receptor | | | Immune |
| 2064 | ERBB2 | erb-b2 receptor | SingleTM | Kinase_Protein | Non-Immune |
| | | tyrosine kinase 2 | | | |
| 3791 | KDR | kinase insert | SingleTM | Kinase_Protein | Non-Immune |
| | | domain receptor | | | |
| 94 | ACVRL1 | activin A receptor | SingleTM | Kinase_Protein | Possible |
| | | like type 1 | | | Immune |
| 1436 | CSF1R | colony stimulating | SingleTM | Kinase_Protein | Immune |
| | | factor 1 receptor | | | |
| 2065 | ERBB3 | erb-b2 receptor | SingleTM | Kinase_Protein | Possible |
| | | tyrosine kinase 3 | | | Immune |
| 2261 | FGFR3 | fibroblast growth | SingleTM | Kinase_Protein | Possible |
| | | factor receptor 3 | | | Immune |
| 3480 | IGF1R | insulin like growth | SingleTM | Kinase_Protein | Possible |
| | | factor 1 receptor | | | Immune |
| 4233 | MET | MET proto-oncogene, | SingleTM | Kinase_Protein | Possible |
| | | receptor tyrosine | | | Immune |
| | | kinase | | | |
| 90 | ACVR1 | activin A receptor | SingleTM | Kinase_Protein | Possible |
| | | type 1 | | | Immune |
| 558 | AXL | AXL receptor | SingleTM | Kinase_Protein | Likely |
| | | tyrosine kinase | | | Immune |
| 2263 | FGFR2 | fibroblast growth | SingleTM | Kinase_Protein | Possible |
| | | factor receptor 2 | | | Immune |
| 2322 | FLT3 | fms related tyrosine | SingleTM | Kinase_Protein | Immune |
| | | kinase 3 | | | |
| 4919 | ROR1 | receptor tyrosine | SingleTM | Kinase_Protein | Immune |
| | | kinase like | | | |
| | | orphan receptor 1 | | | |
| 2260 | FGFR1 | fibroblast growth | SingleTM | Kinase_Protein | Possible |
| | | factor receptor 1 | | | Immune |
| 3815 | KIT | KIT proto-oncogene | SingleTM | Kinase_Protein | Likely |
| | | receptor tyrosine | | | Immune |
| | | kinase | | | |
| 4914 | NTRK1 | neurotrophic receptor | SingleTM | Kinase_Protein | Likely |
| | | tyrosine kinase 1 | | | Immune |
| 2321 | FLT1 | fms related tyrosine | SingleTM | Kinase_Protein | Likely |
| | | kinase 1 | | | Immune |
| 2324 | FLT4 | fms related tyrosine | SingleTM | Kinase_Protein | Likely |
| | | kinase 4 | | | Immune |
| 7010 | TEK | TEK receptor tyrosine | SingleTM | Kinase_Protein | Likely |
| | | kinase | | | Immune |
| 238 | ALK | ALK receptor tyrosine | SingleTM | Kinase_Protein | Possible |
| | | kinase | | | Immune |
| 7046 | TGFBR1 | transforming growth | SingleTM | Kinase_Protein | Likely |
| | | factor beta receptor 1 | | | Immune |
| 10461 | MERTK | MER proto-oncogene, | SingleTM | Kinase_Protein | Likely |
| | | tyrosine kinase | | | Immune |
| 7301 | TYRO3 | TYRO3 protein tyrosine | SingleTM | Kinase_Protein | Possible |
| | | kinase | | | Immune |
| 2050 | EPHB4 | EPH receptor B4 | SingleTM | Kinase_Protein | Possible |
| | | | | | Immune |
| 1969 | EPHA2 | EPH receptor A2 | SingleTM | Kinase_Protein | Possible |
| | | | | | Immune |
| 2066 | ERBB4 | erb-b2 receptor | SingleTM | Kinase_Protein | Non-Immune |
| | | tyrosine kinase 4 | | | |
| 2264 | FGFR4 | fibroblast growth | SingleTM | Kinase_Protein | Likely |
| | | factor receptor 4 | | | Immune |
| 4915 | NTRK2 | neurotrophic receptor | SingleTM | Kinase_Protein | Non-Immune |
| | | tyrosine kinase 2 | | | |
| 5159 | PDGFRB | platelet derived | SingleTM | Kinase_Protein | Possible |
| | | growth factor | | | Immune |
| | | receptor beta | | | |
| 657 | BMPR1A | bone morphogenetic | SingleTM | Kinase_Protein | Non-Immune |
| | | protein receptor | | | |
| | | type 1A | | | |
| 780 | DDR1 | discoidin domain | SingleTM | Kinase_Protein | Undetermined |
| | | receptor tyrosine | | | |
| | | kinase 1 | | | |
| 2043 | EPHA4 | EPH receptor A4 | SingleTM | Kinase_Protein | Possible |
| | | | | | Immune |
| 4486 | MST1R | macrophage stimulating | SingleTM | Kinase_Protein | Non-Immune |
| | | 1 receptor | | | |
| 4916 | NTRK3 | neurotrophic receptor | SingleTM | Kinase_Protein | Non-Immune |
| | | tyrosine kinase 3 | | | |
| 4921 | DDR2 | discoidin domain | SingleTM | Kinase_Protein | Possible |
| | | receptor tyrosine | | | Immune |
| | | kinase 2 | | | |
| 5156 | PDGFRA | platelet derived | SingleTM | Kinase_Protein | Likely |
| | | growth factor | | | Immune |
| | | receptor alpha | | | |
| 5979 | RET | ret proto-oncogene | SingleTM | Kinase_Protein | Likely |
| | | | | | Immune |
| 7048 | TGFBR2 | transforming growth | SingleTM | Kinase_Protein | Immune |
| | | factor beta receptor 2 | | | |
| 6098 | ROS1 | ROS proto-oncogene 1, | SingleTM | Kinase_Protein | Possible |
| | | receptor tyrosine | | | Immune |
| | | kinase | | | |
| 2268 | FGR | FGR proto-oncogene, | Membrane | Kinase_Protein | Immune |
| | | Src family tyrosine | | | |
| | | kinase | | | |
| 5747 | PTK2 | protein tyrosine | Membrane | Kinase_Protein | Possible |
| | | kinase 2 | | | Immune |
| 23043 | TNIK | TRAF2 and NCK | SingleTM | Kinase_Protein | Immune |
| | | interacting kinase | | | |
| 4070 | TACSTD2 | tumor associated | SingleTM | Other | Likely |
| | | calcium signal | | | Immune |
| | | transducer 2 | | | |
| 4072 | EPCAM | epithelial cell | SingleTM | Other | Likely |
| | | adhesion molecule | | | Immune |
| 10232 | MSLN | mesothelin | Membrane | Other | Likely |
| | | | | | Immune |
| 94025 | MUC16 | mucin 16, cell | SingleTM | Other | Likely |
| | | surface associated | | | Immune |
| 3977 | LIFR | LIF receptor alpha | SingleTM | Other | Possible |
| | | | | | Immune |
| 4071 | TM4SF1 | transmembrane 4 L | MultiTM | Other | Possible |
| | | six family member 1 | | | Immune |
| 4851 | NOTCH1 | notch 1 | SingleTM | Other | Likely |
| | | | | | Immune |
| 7037 | TFRC | transferrin receptor | SingleTM | Other | Immune |
| 8788 | DLK1 | delta like non-canonical | SingleTM | Other | Likely |
| | | Notch ligand 1 | | | Immune |
| 29881 | NPC1L1 | NPC1 like intracellular | MultiTM | Other | Non-Immune |
| | | cholesterol | | | |
| | | transporter 1 | | | |
| 55107 | ANO1 | anoctamin 1 | MultiTM | Other | Non-Immune |
| 4853 | NOTCH2 | notch 2 | SingleTM | Other | Likely |
| | | | | | Immune |
| 10267 | RAMP1 | receptor activity | SingleTM | Other | Possible |
| | | modifying protein 1 | | | Immune |
| 781 | CACNA2D1 | calcium voltage-gated | SingleTM | Other | Possible |
| | | channel auxiliary | | | Immune |
| | | subunit alpha2delta 1 | | | |
| 9365 | KL | klotho | SingleTM | Other | Likely |
| | | | | | Immune |
| 59283 | CACNG8 | calcium voltage-gated | MultiTM | Other | Likely |
| | | channel auxiliary | | | Immune |
| | | subunit gamma 8 | | | |
| 3958 | LGALS3 | galectin 3 | Membrane | Other | Possible |
| | | | | | Immune |
| 1191 | CLU | clusterin | Membrane | Other | Likely |
| | | | | | Immune |
| 6280 | S100A9 | S100 calcium binding | Membrane | Other | Immune |
| | | protein A9 | | | |
| 10519 | CIB1 | calcium and integrin | Membrane | Other | Non-Immune |
| | | binding 1 | | | |
| 1803 | DPP4 | dipeptidyl peptidase 4 | SingleTM | Protease | Immune |
| 2346 | FOLH1 | folate hydrolase 1, | SingleTM | Protease | Undetermined |
| | | prostate specific | | | |
| | | membrane antigen, PSMA | | | |
| 8754 | ADAM9 | ADAM metallopeptidase | SingleTM | Protease | Immune |
| | | domain 9 | | | |
| 2028 | ENPEP | glutamyl aminopeptidase | SingleTM | Protease | Likely |
| | | | | | Immune |
| 23621 | BACE1 | beta-secretase 1 | SingleTM | Protease | Non-Immune |
| 2191 | FAP | fibroblast activation | SingleTM | Protease | Likely |
| | | protein alpha | | | Immune |
| 8751 | ADAM15 | ADAM metallopeptidase | SingleTM | Protease | Non-Immune |
| | | domain 15 | | | |
| 290 | ANPEP | alanyl aminopeptidase, | SingleTM | Protease | Immune |
| | | membrane | | | |
| 1636 | ACE | angiotensin I | SingleTM | Protease | Likely |
| | | converting enzyme | | | Immune |
| 4311 | MME | membrane | SingleTM | Protease | Immune |
| | | metalloendopeptidase | | | |
| 5651 | TMPRS | transmembrane serine | SingleTM | Protease | Possible |
| | S15 | protease 15 | | | Immune |
| 9097 | USP14 | ubiquitin specific | Membrane | Protease | Possible |
| | | peptidase 14 | | | Immune |
| 6768 | ST14 | suppression of | SingleTM | Protease | Immune |
| | | tumorigenicity 14 | | | |
| 931 | MS4A1 | membrane spanning | MultiTM | Receptor_all_{—} | Immune |
| | | 4-domains A1 | | others | |
| 916 | CD3E | CD3e molecule | SingleTM | Receptor_all_{—} | Immune |
| | | | | others | |
| 920 | CD4 | CD4 molecule | SingleTM | Receptor_all_{—} | Immune |
| | | | | others | |
| 930 | CD19 | CD19 molecule | SingleTM | Receptor_all_{—} | Immune |
| | | | | others | |
| 1493 | CTLA4 | cytotoxic T-lymphocyte | SingleTM | Receptor_all_{—} | Immune |
| | | associated protein 4 | | others | |
| 3559 | IL2RA | interleukin 2 receptor | SingleTM | Receptor_all_{—} | Immune |
| | | subunit alpha | | others | |
| 3570 | IL6R | interleukin 6 receptor | SingleTM | Receptor_all_{—} | Immune |
| | | | | others | |
| 3674 | ITGA2B | integrin subunit | SingleTM | Receptor_all_{—} | Immune |
| | | alpha 2b | | others | |
| 3676 | ITGA4 | integrin subunit | SingleTM | Receptor_all_{—} | Immune |
| | | alpha 4 | | others | |
| 3685 | ITGAV | integrin subunit | SingleTM | Receptor_all_{—} | Possible |
| | | alpha V | | others | Immune |
| 3688 | ITGB1 | integrin subunit | SingleTM | Receptor_all_{—} | Likely |
| | | beta 1 | | others | Immune |
| 3689 | ITGB2 | integrin subunit | SingleTM | Receptor_all_{—} | Immune |
| | | beta 2 | | others | |
| 3690 | ITGB3 | integrin subunit | SingleTM | Receptor_all_{—} | Immune |
| | | beta 3 | | others | |
| 3695 | TGB7 | integrin subunit | SingleTM | Receptor_all_{—} | Immune |
| | | beta 7 | | others | |
| 5133 | PDCD1 | programmed cell | SingleTM | Receptor_all_{—} | Immune |
| | | death 1 | | others | |
| 23765 | IL17RA | interleukin 17 | SingleTM | Receptor_all_{—} | Immune |
| | | receptor A | | others | |
| 29126 | CD274 | CD274 molecule | SingleTM | Receptor_all_{—} | Immune |
| | | | | others | |
| 57823 | SLAMF7 | SLAM family member 7 | SingleTM | Receptor_all_{—} | Immune |
| | | | | others | |
| 933 | CD22 | CD22 molecule | SingleTM | Receptor_all_{—} | Immune |
| | | | | others | |
| 941 | CD80 | CD80 molecule | SingleTM | Receptor_all_{—} | Immune |
| | | | | others | |
| 951 | CD37 | CD37 molecule | MultiTM | Receptor_all_{—} | Immune |
| | | | | others | |
| 351 | APP | amyloid beta | SingleTM | Receptor_all_{—} | Non-Immune |
| | | precursor protein | | others | |
| 970 | CD70 | CD70 molecule | SingleTM | Receptor_all_{—} | Immune |
| | | | | others | |
| 972 | CD74 | CD74 molecule | SingleTM | Receptor_all_{—} | Immune |
| | | | | others | |
| 1438 | CSF2RA | colony stimulating | SingleTM | Receptor_all_{—} | Immune |
| | | factor 2 receptor | | others | |
| | | alpha subunit | | | |
| 2152 | F3 | coagulation factor | SingleTM | Receptor_all_{—} | Non-Immune |
| | | III, tissue factor | | others | |
| 2348 | FOLR1 | folate receptor 1 | Membrane | Receptor_all_{—} | Possible |
| | | | | others | Immune |
| 2350 | FOLR2 | Folate receptor beta | Membrane | Receptor_all_{—} | Immune |
| | | | | others | |
| 3604 | TNFRSF9 | TNF receptor | SingleTM | Receptor_all_{—} | Immune |
| | | superfamily member 9 | | others | |
| 3693 | ITGB5 | integrin subunit beta 5 | SingleTM | Receptor_all_{—} | Likely |
| | | | | others | Immune |
| 3694 | ITGB6 | integrin subunit beta 6 | SingleTM | Receptor_all_{—} | Likely |
| | | | | others | Immune |
| 3821 | KLRC1 | killer cell lectin | SingleTM | Receptor_all_{—} | Immune |
| | | like receptor C1 | | others | |
| 3902 | LAG3 | lymphocyte activating 3 | SingleTM | Receptor_all_{—} | Immune |
| | | | | others | |
| 4345 | CD200 | CD200 molecule | SingleTM | Receptor_all_{—} | Likely |
| | | | | others | Immune |
| 4582 | MUC1 | mucin 1, cell surface | SingleTM | Receptor_all_{—} | Possible |
| | | associated | | others | Immune |
| 6403 | SELP | selectin P | SingleTM | Receptor_all_{—} | Possible |
| | | | | others | Immune |
| 7097 | TLR2 | toll like receptor 2 | SingleTM | Receptor_all_{—} | Immune |
| | | | | others | |
| 7293 | TNFRSF4 | TNF receptor superfamily | SingleTM | Receptor_all_{—} | Immune |
| | | member 4 | | others | |
| 8784 | TNFRSF18 | TNF receptor superfamily | SingleTM | Receptor_all_{—} | Immune |
| | | member 18 | | others | |
| 80381 | CD276 | CD276 molecule | SingleTM | Receptor_all_{—} | Immune |
| | | | | others | |
| 84868 | HAVCR2 | hepatitis A virus | SingleTM | Receptor_all_{—} | Immune |
| | | cellular receptor 2 | | others | |
| 201633 | TIGIT | T cell immunoreceptor | SingleTM | Receptor_all_{—} | Immune |
| | | with Ig and ITIM domains | | others | |
| 3556 | IL1RAP | interleukin 1 receptor | SingleTM | Receptor_all_{—} | Immune |
| | | accessory protein | | others | |
| 4680 | CEACAM6 | carcinoembryonic antigen | Membrane | Receptor_all_{—} | Immune |
| | | related cell adhesion | | others | |
| | | molecule 6 | | | |
| 7099 | TLR4 | toll like receptor 4 | SingleTM | Receptor_all_{—} | Immune |
| | | | | others | |
| 51206 | GP6 | glycoprotein VI | SingleTM | Receptor_all_{—} | Immune |
| | | platelet | | others | |
| 7162 | TPBG | trophoblast | SingleTM | Receptor_all_{—} | Possible |
| | | glycoprotein | | others | Immune |
| 81607 | NECTIN4 | nectin cell adhesion | SingleTM | Receptor_all_{—} | Likely |
| | | molecule 4 | | others | Immune |
| 943 | TNFRSF8 | TNF receptor | SingleTM | Receptor_all_{—} | Immune |
| | | superfamily member 8 | | others | |
| 945 | CD33 | CD33 molecule | SingleTM | Receptor_all_{—} | Immune |
| | | | | others | |
| 1048 | CEACAM5 | carcinoembryonic | Membrane | Receptor_all_{—} | Likely |
| | | antigen related cell | | others | Immune |
| | | adhesion molecule 5 | | | |
| 10683 | DLL3 | delta like canonical | SingleTM | Receptor_all_{—} | Immune |
| | | Notch ligand 3 | | others | |
| 3563 | IL3RA | interleukin 3 | SingleTM | Receptor_all_{—} | Immune |
| | | receptor subunit | | others | |
| | | alpha | | | |
| 160364 | CLEC12A | C-type lectin domain | SingleTM | Receptor_all_{—} | Immune |
| | | family 12 member A | | others | |
| 929 | CD14 | CD14 molecule | Membrane | Receptor_all_{—} | Immune |
| | | | | others | |
| 58985 | IL22RA1 | interleukin 22 receptor | SingleTM | Receptor_all_{—} | Non-Immune |
| | | subunit alpha 1 | | others | |
| 389400 | GFRAL | GDNF family receptor | SingleTM | Receptor_all_{—} | Undetermined |
| | | alpha like | | others | |
| 4179 | CD46 | CD46 molecule | SingleTM | Receptor_all_{—} | Possible |
| | | | | others | Immune |
| 131450 | CD200R1 | CD200 receptor 1 | SingleTM | Receptor_all_{—} | Immune |
| | | | | others | |
| 962 | CD48 | CD48 molecule | Membrane | Receptor_all_{—} | Immune |
| | | | | others | |
| 4065 | LY75 | lymphocyte antigen 75 | SingleTM | Receptor_all_{—} | Immune |
| | | | | others | |
| 131578 | LRRC15 | leucine rich repeat | SingleTM | Receptor_all_{—} | Possible |
| | | containing 15 | | others | Immune |
| 8718 | TNFRSF25 | TNF receptor | SingleTM | Receptor_all_{—} | Immune |
| | | superfamily member 25 | | others | |
| 64115 | VSIR | V-set immunoregulatory | SingleTM | Receptor_all_{—} | Immune |
| | | receptor | | others | |
| 2057 | EPOR | erythropoietin receptor | SingleTM | Receptor_all_{—} | Immune |
| | | | | others | |
| 3594 | IL12RB1 | interleukin 12 receptor | SingleTM | Receptor_all_{—} | Immune |
| | | subunit beta 1 | | others | |
| 3595 | IL12RB2 | interleukin 12 receptor | SingleTM | Receptor_all_{—} | Immune |
| | | subunit beta 2 | | others | |
| 3824 | KLRD1 | killer cell lectin | SingleTM | Receptor_all_{—} | Immune |
| | | like receptor D1 | | others | |
| 949 | SCARB1 | scavenger receptor | MultiTM | Receptor_all_{—} | Immune |
| | | class B member 1 | | others | |
| 3554 | IL1R1 | interleukin 1 | SingleTM | Receptor_all_{—} | Immune |
| | | receptor type 1 | | others | |
| 4352 | MPL | MPL proto-oncogene, | SingleTM | Receptor_all_{—} | Likely |
| | | thrombopoietin receptor | | others | Immune |
| 7850 | IL1R2 | interleukin 1 receptor | SingleTM | Receptor_all_{—} | Immune |
| | | type 2 | | others | |
| 177 | AGER | advanced glycosylation | SingleTM | Receptor_all_{—} | Immune |
| | | end-product specific | | others | |
| | | receptor | | | |
| 214 | ALCAM | activated leukocyte | SingleTM | Receptor_all_{—} | Immune |
| | | cell adhesion molecule | | others | |
| 3560 | IL2RB | interleukin 2 receptor | SingleTM | Receptor_all_{—} | Immune |
| | | subunit beta | | others | |
| 3683 | ITGAL | integrin subunit | SingleTM | Receptor_all_{—} | Immune |
| | | alpha L | | others | |
| 3696 | ITGB8 | integrin subunit | SingleTM | Receptor_all_{—} | Likely |
| | | beta 8 | | others | Immune |
| 4360 | MRC1 | mannose receptor | SingleTM | Receptor_all_{—} | Immune |
| | | C-type 1 | | others | |
| 6330 | SCN4B | sodium voltage-gated | SingleTM | Receptor_all_{—} | Possible |
| | | channel beta | | others | Immune |
| | | subunit 4 | | | |
| 7100 | TLR5 | toll like receptor 5 | SingleTM | Receptor_all_{—} | Immune |
| | | | | others | |
| 8764 | TNFRSF14 | TNF receptor superfamily | SingleTM | Receptor_all_{—} | Likely |
| | | member 14 | | others | Immune |
| 8792 | TNFRSF11A | TNF receptor superfamily | SingleTM | Receptor_all_{—} | Immune |
| | | member 11a | | others | |
| 50848 | F11R | F11 receptor | SingleTM | Receptor_all_{—} | Immune |
| | | | | others | |
| 8842 | PROM1 | prominin 1 | MultiTM | Receptor_all_{—} | Likely |
| | | | | others | Immune |
| 3678 | ITGA5 | integrin subunit | SingleTM | Receptor_all_{—} | Likely |
| | | alpha 5 | | others | Immune |
| 3105 | HLA-A | major histocompatibility | SingleTM | Receptor_all_{—} | Likely |
| | | complex, class I, A | | others | Immune |
| 3383 | ICAM1 | intercellular adhesion | SingleTM | Receptor_all_{—} | Immune |
| | | molecule 1 | | others | |
| 4804 | NGFR | nerve growth factor | SingleTM | Receptor_all_{—} | Possible |
| | | receptor | | others | Immune |
| 960 | CD44 | CD44 molecule (Indian | SingleTM | Receptor_all_{—} | Immune |
| | | blood group) | | others | |
| 1000 | CDH2 | cadherin 2 | SingleTM | Receptor_all_{—} | Possible |
| | | | | others | Immune |
| 1003 | CDH5 | cadherin 5 | SingleTM | Receptor_all_{—} | Possible |
| | | | | others | Immune |
| 4267 | CD99 | CD99 molecule (Xg | SingleTM | Receptor_all_{—} | Immune |
| | | blood group) | | others | |
| 6401 | SELE | selectin E | SingleTM | Receptor_all_{—} | Possible |
| | | | | others | Immune |
| 6402 | SELL | selectin L | SingleTM | Receptor_all_{—} | Immune |
| | | | | others | |
| 54106 | TLR9 | toll like receptor 9 | SingleTM | Receptor_all_{—} | Immune |
| | | | | others | |
| 54210 | TREM1 | triggering receptor | SingleTM | Receptor_all_{—} | Immune |
| | | expressed on myeloid | | others | |
| | | cells 1 | | | |
| 64581 | CLEC7A | C-type lectin domain | SingleTM | Receptor_all_{—} | Immune |
| | | containing 7A | | others | |
| 90952 | ESAM | endothelial cell | SingleTM | Receptor_all_{—} | Likely |
| | | adhesion molecule | | others | Immune |
| 3916 | LAMP1 | lysosomal associated | SingleTM | Receptor_all_{—} | Possible |
| | | membrane protein 1 | | others | Immune |
| 4155 | MBP | myelin basic protein | Membrane | Receptor_all_{—} | Likely |
| | | | | others | Immune |
| 476 | ATP1A1 | ATPase Na+/K+ transporting | MultiTM | Transporter | Non-Immune |
| | | subunit alpha 1 | | | |
| 6510 | SLC1A5 | solute carrier family | MultiTM | Transporter | Immune |
| | | 1 member 5 | | | |
| 10568 | SLC34A2 | solute carrier family | MultiTM | Transporter | Possible |
| | | 34 member 2 | | | Immune |
| 6517 | SLC2A4 | solute carrier family | MultiTM | Transporter | Possible |
| | | 2 member 4 | | | Immune |
| 116085 | SLC22A12 | solute carrier family | MultiTM | Transporter | Non-Immune |
| | | 22 member 12 | | | |
| 284111 | SLC13A5 | solute carrier family | MultiTM | Transporter | Likely |
| | | 13 member 5 | | | Immune |
| 8140 | SLC7A5 | solute carrier | MultiTM | Transporter | Immune |
| | | family 7 member 5 | | | |
| 19 | ABCA1 | ATP binding cassette | MultiTM | Transporter | Likely |
| | | subfamily A member 1 | | | Immune |
| 6531 | SLC6A3 | solute carrier family | MultiTM | Transporter | Non-Immune |
| | | 6 member 3 | | | |
| 6558 | SLC12A2 | solute carrier family | MultiTM | Transporter | Non-Immune |
| | | 12 member 2 | | | |
| 9152 | SLC6A5 | solute carrier | MultiTM | Transporter | Non-Immune |
| | | family 6 member 5 | | | |
| 9900 | SV2A | synaptic vesicle | MultiTM | Transporter | Possible |
| | | glycoprotein 2A | | | Immune |
| 30061 | SLC40A1 | solute carrier | MultiTM | Transporter | Immune |
| | | family 40 member 1 | | | |
| 496 | ATP4B | ATPase H+/K+ transporting | SingleTM | Transporter | Possible |
| | | subunit beta | | | Immune |
| 6554 | SLC10A1 | solute carrier family | MultiTM | Transporter | Possible |
| | | 10 member 1 | | | Immune |
| 6574 | SLC20A1 | solute carrier family | MultiTM | Transporter | Likely |
| | | 20 member 1 | | | Immune |
| 1080 | CFTR | cystic fibrosis | MultiTM | Transporter | Non-Immune |
| | | transmembrane | | | |
| | | conductance regulator | | | |
| 6530 | SLC6A2 | solute carrier family | MultiTM | Transporter | Non-Immune |
| | | 6 member 2 | | | |
| 6575 | SLC20A2 | solute carrier family | MultiTM | Transporter | Non-Immune |
| | | 20 member 2 | | | |
| 6833 | ABCC8 | ATP binding cassette | MultiTM | Transporter | Non-Immune |
| | | subfamily C member 8 | | | |
| 5243 | ABCB1 | ATP binding cassette | MultiTM | Transporter | Likely |
| | | subfamily B member 1 | | | Immune |
| 6532 | SLC6A4 | solute carrier family | MultiTM | Transporter | Immune |
| | | 6 member 4 | | | |
| 6546 | SLC8A1 | solute carrier family | MultiTM | Transporter | Possible |
| | | 8 member A1 | | | Immune |
| 6548 | SLC9A1 | solute carrier family | MultiTM | Transporter | Possible |
| | | 9 member A1 | | | Immune |
| 6566 | SLC16A1 | solute carrier family | MultiTM | Transporter | Immune |
| | | 16 member 1 | | | |
| 6573 | SLC19A1 | solute carrier family | MultiTM | Transporter | Immune |
| | | 19 member 1 | | | |
| 6535 | SLC6A8 | solute carrier family | MultiTM | Transporter | Likely |
| | | 6 member 8 | | | Immune |
| 6550 | SLC9A3 | solute carrier family | MultiTM | Transporter | Undetermined |
| | | 9 member A3 | | | |
| 6555 | SLC10A2 | solute carrier family | MultiTM | Transporter | Non-Immune |
| | | 10 member 2 | | | |
| 10060 | ABCC9 | ATP binding cassette | MultiTM | Transporter | Likely |
| | | subfamily C member 9 | | | Immune |
| 6529 | SLC6A1 | solute carrier family | MultiTM | Transporter | Possible |
| | | 6 member 1 | | | Immune |
| 6523 | SLC5A1 | solute carrier family | MultiTM | Transporter | Possible |
| | | 5 member 1 | | | Immune |
| 6524 | SLC5A2 | solute carrier family | MultiTM | Transporter | Non-Immune |
| | | 5 member 2 | | | |
| 6534 | SLC6A7 | solute carrier family | MultiTM | Transporter | Undetermined |
| | | 6 member 7 | | | |
| 6536 | SLC6A9 | solute carrier family | MultiTM | Transporter | Likely |
| | | 6 member 9 | | | Immune |
| 221074 | SLC39A12 | solute carrier family | MultiTM | Transporter | Possible |
| | | 39 member 12 | | | Immune |
| 6559 | SLC12A3 | solute carrier family | MultiTM | Transporter | Likely |
| | | 12 member 3 | | | Immune |
| 495 | ATP4A | ATPase H+/K+ transporting | MultiTM | Transporter | Likely |
| | | subunit alpha | | | Immune |
| 6557 | SLC12A1 | solute carrier family | MultiTM | Transporter | Possible |
| | | 12 member 1 | | | Immune |
| 1236 | CCR7 | C-C motif chemokine | MultiTM | GPCR | Immune |
| | | receptor 7 | | | |
| 2826 | CCR10 | C-C motif chemokine | MultiTM | GPCR | Immune |
| | | receptor 10 | | | |
| 2833 | CXCR3 | C-X-C motif chemokine | MultiTM | GPCR | Immune |
| | | receptor 3 | | | |
| 643 | CXCR5 | C-X-C motif chemokine | MultiTM | GPCR | Immune |
| | | receptor 5 | | | |
| 10663 | CXCR6 | C-X-C motif chemokine | MultiTM | GPCR | Immune |
| | | receptor 6 | | | |
| 57007 | ACKR3 | atypical chemokine | MultiTM | GPCR | Immune |
| | | receptor 3 | | | |
| 719 | C3AR1 | complement C3a | MultiTM | GPCR | Immune |
| | | receptor 1 | | | |
| 1880 | GPR183 | G-protein coupled | MultiTM | GPCR | Immune |
| | | receptor 183 | | | |
| 2859 | GPR35 | G protein-coupled | MultiTM | GPCR | Immune |
| | | receptor 35 | | | |
| 2866 | GPR42 | G protein-coupled | MultiTM | GPCR | Immune |
| | | receptor 42 | | | |
| 1524 | CX3CR1 | C-X3-C motif chemokine | MultiTM | GPCR | Immune |
| | | receptor 1 | | | |
| 4973 | OLR1 | oxidized low density | SingleTM | Receptor_all_{—} | Immune |
| | | lipoprotein | | others | |
| | | receptor 1 | | | |
| 5329 | PLAUR | plasminogen activator, | Membrane | Receptor_all_{—} | Immune |
| | | urokinase receptor | | others | |
| 768 | CA9 | Carbonic anhydrase 9 | SingleTM | Enzyme_other | Possible |
| | | | | | Immune |
| 771 | CA12 | carbonic anhydrase 12 | SingleTM | Enzyme_other | Possible |
| | | | | | Immune |
| 117194 | MRGPRX2 | Mas-related G-protein | MultiTM | Exposed | Possible |
| | | coupled receptor | | | Immune |
| | | member X2 | | | |
| 3320 | HSP90AA1 | heat shock protein | Unattached | Enzyme_other | Possible |
| | | 90 alpha family | | | |
| | | class A member 1 | | | Immune |
| 1803 | DPP4 | dipeptidyl peptidase 4 | SingleTM | Protease | Immune |
| 2358 | FPR2 | formyl peptide | MultiTM | GPCR | Immune |
| | | receptor 2 | | | |
| 56670 | SUCNR1 | succinate receptor 1 | MultiTM | GPCR | Immune |
| 6332 | SCN7A | sodium voltage-gated | MultiTM | Ion Channel | Possible |
| | | channel alpha | | | Immune |
| | | subunit 7 | | | |

In a further embodiment, the target of the target binding moiety is a chemokine receptor (OCR). In a further embodiment, the target of the target binding moiety is selected from CCR1, CCR2, CCR3, or CCR5.

In a further embodiment, the target of the target binding moiety is selected from C-C motif chemokine receptor (OCR) 2 (CCR2), CCR1, CCR3, CCR4, CCR5, CCR6, CCR7, CCR8, CCR9, CCR10, C—X—C motif chemokine receptor 1 (CXCR1), C—X—C motif chemokine receptor 2 (CXCR2), C—X—C motif chemokine receptor 3 (CXCR3), C—X—C motif chemokine receptor 4 (CXCR4), C—X—C motif chemokine receptor 5 (CXCR5), C—X—C motif chemokine receptor 6 (CXCR6), atypical chemokine receptor 3 (ACKR3), integrin αvβ36, fibroblast activation protein-alpha (FAPα), prostate specific membrane antigen (PSMA), folate receptor (folate receptor 1 or folate receptor beta), complement C3a receptor 1 (C3AR1), complement C5a receptor 1 (C₅AR1), G protein-coupled receptor (GPR) 65 (GPR65), GRP132, GPR84, GPR183, GPR35, GPR42, cholecystokinin A receptor (COKAR), leukotriene B4 receptor (LTB4R), somatostatin receptor 2 (SSTR2), free fatty acid receptor 1 (FFAR1), purinergic receptor P2Y2 (P2RY2), prostaglandin 02 receptor (PTGDR), calcitonin receptor (CALOR), 0038, purinergic receptor P2X 7 (P2RX7), integrin subunit alpha V (ITGAV), integrin subunit alpha 5 (ITGA5), integrin subunit beta 1 (ITGB1), integrin subunit beta 6 (ITGB6), integrin subunit beta 3 (ITGB3) prostaglandin 02 receptor 2 (PTGDR2), gastrin releasing peptide receptor (GRPR), MER proto-oncogene tyrosine kinase (MERTK), C-X3-C motif chemokine receptor 1 (CX3CR1), oxidized low density lipoprotein receptor 1 (OLR1), plasminogen activator urokinase receptor (PLAUR), carbonic anhydrase 9 (CA9), carbonic anhydrase 12 (CA12), mas-related G-protein coupled receptor member X2 (MRGPRX2), heat shock protein 90 alpha family class A member 1 (HSP90AA1), dipeptidyl peptidase 4 (DPP4), formyl peptide receptor 2 (FPR2), and succinate receptor 1 (SUCNR1).

In a further embodiment, the target-binding moiety T is a small molecule that binds a target as listed in Table 4. A person skilled in the art can select small molecules known to bind the target protein for use as the target-binding moiety in the ARM. In one embodiment, the target-binding small molecule is modified to include a functional group such as —NH₂ or —COOH to facilitate covalent coupling of the target-binding small molecule to the divalent linker moiety by amide bond formation.

The present disclosure also provides a combination comprising a heterobifunctional compound of Formula (I) as described herein and a modified immunomodulatory cell comprising a chimeric antigen receptor (CAR) comprising an extracellular ligand binding domain; a transmembrane domain; and an intracellular signalling domain; wherein the extracellular ligand binding domain comprises an anti-cotinine antibody or antigen-binding fragment thereof.

The present disclosure also provides a pharmaceutical composition comprising a compound of Formula (I) as disclosed herein, and a pharmaceutically acceptable excipient, carrier, or diluent.

### Statement of Use

The compounds of Formula (I) and pharmaceutically acceptable salts thereof are capable of simultaneously binding a cell surface-expressed target and a chimeric antigen receptor comprising an anti-cotinine antibody, or antigen binding fragment thereof to form a ternary complex for the treatment and/or prevention of diseases or disorders associated with target-expressing cells.

In one embodiment, the present disclosure provides a method of treating and/or preventing a disease or disorder in a patient in need thereof comprising administering to the patient a therapeutically effective amount of a combination comprising a modified immunomodulatory cell as disclosed herein and the compound of Formula (I), or a pharmaceutically acceptable salt thereof, wherein the disease or disorder is selected from a cancer, an inflammatory disease, an autoimmune disease, a viral infection, or a bacterial infection.

In a further embodiment, the compound and the modified immunomodulatory cell are administered simultaneously. In a further embodiment, the compound and the modified immunomodulatory cell are administered simultaneously from a single composition, including as a fixed-dose composition or by pre-mixing the compound and the modified immunomodulatory cell prior to administration. For example, the compound and the modified immunomodulatory cell can be pre-mixed about 2 seconds to about 30 seconds, about 30 seconds to about 2 minutes, about 2 minutes to about 10 minutes, about 10 minutes to about 30 minutes, or about 30 minutes to about 2 hours prior to administration. In a further embodiment, the compound and the modified immunomodulatory cell are administered simultaneously from two separate compositions.

In a further embodiment, the compound and the modified immunomodulatory cell are administered sequentially.

In certain embodiments, the compound and the modified immunomodulatory cell whether administered simultaneously or sequentially, may be administered by the same route or may be administered by different routes. In one embodiment, the compound and the modified immunomodulatory cell are both administered intravenously or subcutaneously, in the same composition or in separate compositions. In another embodiment, the compound is administered orally and the modified immunomodulatory cell is administered intravenously or subcutaneously.

In a further embodiment, the present disclosure provides a therapeutically effective amount of the compound of Formula (I), or a pharmaceutically acceptable salt thereof, and the modified immunomodulatory cell for use in therapy. The compound of Formula (I), or a pharmaceutically acceptable salt thereof, and the modified immunomodulatory cell can be used in treating or preventing a disease or disorder selected from a cancer, an inflammatory disease, an autoimmune disease, a viral infection, or a bacterial infection.

In a further embodiment, the present disclosure provides a therapeutically effective amount of the compound of Formula (I), or a pharmaceutically acceptable salt thereof, and the modified immunomodulatory cell for the manufacture of a medicament. The medicament can be used in treating or preventing a disease or disorder selected from a cancer, an inflammatory disease, an autoimmune disease, a viral infection, or a bacterial infection.

In a further embodiment, the disease or disorder is mediated by chemokine receptor 2 (CCR2) and/or is associated with CCR2-positive pathogenic cells. In a further embodiment, CCR2-positive cell types are identified by testing for expression of CCR2 such as by immunohistochemistry or flow cytometry.

In a further embodiment, the disease or disorder is mediated by C—X—C motif chemokine receptor 3 (CXCR3) and/or is associated with CXCR3-positive pathogenic cells. In a further embodiment, CXCR3-positive cell types are identified by testing for expression of CXCR3 such as by immunohistochemistry or flow cytometry.

In a further embodiment, the disease or disorder is mediated by PSMA and/or is associated with PSMA-positive pathogenic cells. In a further embodiment, PSMA-positive cell types are identified by testing for expression of PSMA such as by immunohistochemistry or flow cytometry.

In a further embodiment, the disease or disorder is mediated by integrin αVβ6 and/or is associated with integrin αVβ6-positive pathogenic cells. In a further embodiment, integrin αVβ6-positive cell types are identified by testing for expression of integrin αVβ6 such as by immunohistochemistry or flow cytometry.

In a further embodiment, the disease or disorder is mediated by folate receptor α (FRα) and/or folate receptor β (FRβ) and/or is associated with FRα- and/or FRβ-positive pathogenic cells. In a further embodiment, FRα- and/or FRβ-positive cell types are identified by testing for expression of FRα and/or FRβ such as by immunohistochemistry or flow cytometry.

In a further embodiment, the disease or disorder is mediated by fibroblast activation protein (FAP) and/or is associated with FAP-positive pathogenic cells. In a further embodiment, FAP-positive cell types are identified by testing for expression of FAP such as by immunohistochemistry or flow cytometry.

In a further embodiment, the disease or disorder is mediated by chemokine receptor 8 (CCR8) and/or is associated with CCR8-positive pathogenic cells. In a further embodiment, CCR8-positive cell types are identified by testing for expression of CCR8 such as by immunohistochemistry or flow cytometry.

In a further embodiment, the disease or disorder is a cancer selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, brain cancer, endocrine cancer, testicular cancer, bladder cancer, bone cancer, esophageal cancer, gastric cancer, renal cell cancer, melanoma cancer, thyroid cancer, or breast cancer, preferably a cancer selected from mCRPC, breast cancer, lung cancer, colorectal cancer, or renal cell cancer.

In a further embodiment, the disease or disorder is a solid tumor. In a further embodiment, the disease or disorder is a solid tumor selected from lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophageal cancer, gastric cancer, renal cell cancer, melanoma cancer, thyroid cancer, or breast cancer, preferably a solid tumor selected from mCRPC, breast cancer, lung cancer, colorectal cancer, or renal cell cancer.

In a further embodiment, the disease or disorder is a PD-1 relapsed or refractory cancer, such as a PD-1 relapsed or refractory lung cancer (e.g., NSCLC), HCC, CRC, cervical cancer (e.g., CESC), head and neck cancer (e.g., HNSC), pancreatic cancer, prostate cancer (e.g., mCRPC), ovarian cancer, endometrial cancer, brain cancer, endocrine cancer, testicular cancer, bladder cancer, bone cancer, esophageal cancer, gastric cancer, renal cell cancer, melanoma cancer, thyroid cancer, or breast cancer, preferably a PD-1 relapsed or refractory breast cancer, lung cancer, head and neck cancer, or cervical cancer.

In a further embodiment, the disease or disorder is a non-solid cancer. In a further embodiment, the disease or disorder is a leukemia, a lymphoma, or a myeloma.

In a further embodiment, the disease or disorder is a viral infection. In a further embodiment, the viral infection is caused by an influenza virus, a coronavirus (e.g., COVID-19), or a hepatitis B virus.

In a further embodiment, the disease or disorder is a bacterial infection. In a further embodiment, the bacterial infection is a chronic bacterial infection.

In a further embodiment, the disease is an autoimmune or inflammatory disease selected from vitiligo and type I diabetes.

In one embodiment, the present disclosure provides method of increasing cell killing of target-expressing cells comprising: contacting the cells with an effective amount of a combination comprising a modified immunomodulatory cell as disclosed herein and a heterobifunctional molecule as disclosed herein, wherein the target-binding moiety of the compound binds the target expressed on the cells.

In one embodiment, the present disclosure provides a method of depleting target-expressing cells comprising: contacting the cells with an effective amount of a combination comprising a modified immunomodulatory cell as disclosed herein and a heterobifunctional molecule as disclosed herein, wherein the target-binding moiety of the compound binds the target expressed on the cells.

In a further embodiment, the target-expressing cells are CCR2-expressing cells. In a further embodiment, the CCR2-expressing cells are myeloid-derived suppressor cells (MDSCs), T regulatory cells (Tregs), neutrophils, macrophages, B regulatory cells (Bregs), CD8 regulatory cells, (CD8regs), exhausted T cells, or cancer-associated fibroblasts (CAFs).

In a further embodiment, the target-expressing cells are CXCR3-expressing cells. In a further embodiment, the CXCR3-expressing cells are activated T cells, autoreactive T cells, T regulatory cells (Tregs), CD4 regulatory T cells (CD4regs), CD8 regulatory T cells, (CD8regs), T helper (Th) T cells, Th1 T cells, natural killer T (NKT) cells, natural killer (NK) cells, dendritic cells, B cells, γδT cells, or tumor cells.

In a further embodiment, the target-expressing cells are PSMA-expressing cells. In a further embodiment, the PSMA-expressing cells are tumor cells.

In a further embodiment, the target-expressing cells are integrin αVβ6-expressing cells. In a further embodiment, the integrin αVβ6-expressing cells are tumor cells.

In a further embodiment, the target-expressing cells are FRα- and/or FRβ-expressing cells. In a further embodiment, the FRα- and/or FRβ-expressing cells are myeloid derived suppressor cells (MDSCs), macrophages, B cells, or tumor cells.

In a further embodiment, the target-expressing cells are FAP-expressing cells. In a further embodiment, the FAP-expressing cells are cancer-associated fibroblasts (CAFs), macrophages, or tumor cells.

In a further embodiment, the target-expressing cells are CCR8-expressing cells. In a further embodiment, the CCR8-expressing cells are T regulatory cells (Tregs) or tumor cells.

In a further embodiment, the target-expressing cells are pathogenic cells.

In a further embodiment, the pathogenic cell is a pathogenic immune cell, a tumor cell or cancer cell, or a stromal cell.

In a further embodiment, the pathogenic immune cells are monocytes, myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs, memory B cells), plasma cells, CD8 cells (e.g., CD8 regulatory cells (CD8regs), memory CD8 cells, effector CD8 cells, naïve CD8 Tcells, TEMRA), exhausted T cells, eosinophils, basophils, mast cells, dendritic cells, natural killer (NK cells), innate lymphoid cells, NK T cells (NKT), or γδT cells.

In a further embodiment, the pathogenic immune cells are myeloid derived suppressor cells (MDSC), such as monocytic MDSCs (mMDSCs) and polymorphonuclear MDSCs (PMN_MDSCs), T regulatory cells (Tregs), neutrophils (e.g., N2 neutrophils), macrophages (e.g., M2 macrophages), B regulatory cells (Bregs), CD8 regulatory cells (CD8regs), exhausted T cells.

In a further embodiment, the tumor cells or cancer cells are lung cancer cells (e.g., non-small cell lung cancer (NSCLC) cells), hepatocellular carcinoma (HCC) cells, colorectal cancer (CRC) cells, cervical cancer cells (e.g., cervical squamous cell carcinoma (CESC) cells), head and neck cancer cells (e.g., head and neck squamous cell carcinoma (HNSC) cells), pancreatic cancer cells, prostate cancer cells (e.g., metastatic castration-resistant prostate cancer (mCRPC) cells), ovarian cancer cells, endometrial cancer cells, brain cancer cells, endocrine cancer cells, testicular cancer cells, bladder cancer cells, bone cancer cells, esophageal cancer cells, gastric cancer cells, renal cell cancer cells, melanoma cancer cells, thyroid cancer cells, or breast cancer cells, preferably cells selected from mCRPC cells, breast cancer cells, lung cancer cells, colorectal cancer cells, or renal cell cancer cells.

In a further embodiment, the stromal cells are cancer associated fibroblasts (CAFs).

### Combination Therapies

The compounds of the invention may be employed alone or in combination with other therapeutic agents. Combination therapies according to the present invention thus comprise the administration of at least one compound of Formula (I) or a pharmaceutically acceptable salt thereof, and the use of at least one other pharmaceutically active agent. The compounds of the invention and the other pharmaceutically active agents may be administered together in a single pharmaceutical composition or separately and, when administered separately this may occur simultaneously or sequentially in any order. The amounts of the compounds of the invention and the other pharmaceutically active agents and the relative timings of administration will be selected in order to achieve the desired combined therapeutic effect.

It will be appreciated that when the compound of the present invention is administered in combination with one or more other therapeutically active agents normally administered by the inhaled, intravenous, oral, intranasal, ocular topical or other route, that the resultant pharmaceutical composition may be administered by the same route. Alternatively, the individual components of the composition may be administered by different routes.

In one embodiment, the compounds and pharmaceutical composition disclosed herein are used in combination with, or include, one or more additional therapeutic agents. In a further embodiment, the additional therapeutic agent is a checkpoint inhibitor or an immune modulator.

In a further embodiment, the checkpoint inhibitor is selected from a PD-1 inhibitor (e.g., an anti-PD-1 antibody including, but not limited to, pembrolizumab, nivolumab, cemiplimab, or dostarlimab), a PD-L1 inhibitor (e.g., an anti-PD-L1 antibody including, but not limited to, atezolizumab, avelumab, or durvalumab), or a CTLA-4 inhibitor (e.g., an anti-CTLA-4 antibody including, but not limited to, ipilimumab or tremilumumab).

In a further embodiment, the checkpoint inhibitor is selected from a CD226 axis inhibitor, including but not limited to a TIGIT inhibitor (e.g., an anti-TIGIT antibody), a CD96 inhibitor (e.g., an anti-CD96 antibody), and/or a PVRIG inhibitor (e.g., an anti-PVRIG antibody).

In a further embodiment, the immune modulator is an ICOS agonist (e.g., an anti-ICOS antibody including, but not limited to feladilimab), a PARP inhibitor (e.g., niraparib, olaparib or a STING agonist.

### Pharmaceutical Compositions, Dosages, and Dosage Forms

For the purposes of administration, in certain embodiments, the ARMs described herein are administered as a raw chemical or are formulated as pharmaceutical compositions. Pharmaceutical compositions disclosed herein include an ARM and one or more of: a pharmaceutically acceptable carrier, diluent or excipient. An ARM is present in the composition in an amount which is effective to treat a particular disease, disorder or condition of interest. The activity of the ARM can be determined by one skilled in the art, for example, as described in the biological assays described below. Appropriate concentrations and dosages can be readily determined by one skilled in the art. In certain embodiments, the ARM is present in the pharmaceutical composition in an amount from about 25 mg to about 500 mg. In certain embodiments, the ARM is present in the pharmaceutical composition in an amount of about 0.01 mg to about 300 mg. In certain embodiments, the ARM is present in the pharmaceutical composition in an amount of about 0.01 mg, 0.1 mg, 1 mg, 5 mg, 10 mg, 25 mg, 50 mg, 100 mg, 200 mg, 300 mg, 400 mg or about 500 mg.

Administration of the compounds of the invention, or their pharmaceutically acceptable salts, in pure form or in an appropriate pharmaceutical composition, is carried out via any of the accepted modes of administration of agents for serving similar utilities. The pharmaceutical compositions of the invention are prepared by combining a compound of the invention with an appropriate pharmaceutically acceptable carrier, diluent or excipient, and in specific embodiments are formulated into preparations in solid, semi-solid, liquid or gaseous forms, such as tablets, capsules, powders, granules, ointments, solutions, suppositories, injections, inhalants, gels, microspheres, and aerosols. Exemplary routes of administering such pharmaceutical compositions include, without limitation, oral, topical, transdermal, inhalation, parenteral (e.g., intramuscular, subcutaneous, intravenous, or intradermal), sublingual, buccal, rectal, vaginal, and intranasal. Pharmaceutical compositions of the invention are formulated so as to allow the active ingredients contained therein to be bioavailable upon administration of the composition to a patient.

Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see Remington: The Science and Practice of Pharmacy, 20th ^{E}dition (Philadelphia. College of Pharmacy and Science, 2000). The composition to be administered will, in any event, contain a therapeutically effective amount of a compound of the invention, or a pharmaceutically acceptable salt thereof, for treatment of a disease or condition of interest in accordance with the teachings described herein.

The pharmaceutical compositions disclosed herein are prepared by methodologies well known in the pharmaceutical art. For example, in certain embodiments, a pharmaceutical composition intended to be administered by injection is prepared by combining a compound of the invention with sterile, distilled water so as to form a solution. In some embodiments, a surfactant is added to facilitate the formation of a homogeneous solution or suspension. Surfactants are compounds that non-covalently interact with the compound of the invention so as to facilitate dissolution or homogeneous suspension of the compound in the aqueous delivery system.

### EXAMPLES

The following examples illustrate the invention. These Examples are not intended to limit the scope of the invention, but rather to provide guidance to the skilled artisan to prepare and use the compounds, compositions, and methods of the invention. While particular embodiments of the invention are described, the skilled artisan will appreciate that various changes and modifications can be made.

### Compound Synthesis

The compounds according to Formula (I) were prepared using conventional organic synthetic methods as described, for example, in International Patent Application No. PCT/IB2022/057562, which is hereby incorporated by reference in its entirety.

### Biological Assays

Compounds 1-7 which are compounds of Formula (I) having a PSMA-, integrin αVβ6-FRα- and/or FRβ-, or FAP-binding moiety were tested in various biological assays as described in more detail below.

### Example 1: Generation of Anti-Cotinine CAR Constructs and Vector Production

### Vector Constructs and Vector Production

A vector encoding an anti-cotinine single chain variable fragment (scFv) was prepared and used to produce lentiviral vector for transduction of CAR-T cells and retroviral vector for transduction of NK cells.

For production of lentiviral vector, the anti-cotinine scFv of SEQ ID NO: 16 was inserted into a second-generation CAR cassette including a CD8 leader, CD8 hinge domain, CD8 transmembrane region, a 41BB costimulatory domain, and a CD3z activation domain. A FLAG tag (DYKDDDDK [SEQ ID NO: 33]) was appended to the CAR construct after the CD3z activation domain. Lentiviral supernatants were produced via co-transfection of Lenti-X 293T cells (Takara) with the lentiviral vector plasmid encoding the anti-cotinine scFv CAR, Lipofectamine 2000 Reagent (Invitrogen), and a Ready-to-Use Lentiviral Packaging Plasmid Mix (psPAX2 and pMD.2 4:1 w:w ratio, Cellecta). After 48-72 hours of transfection, supernatants containing the lentivirus with the full length CAR gene of SEQ ID NO: 19 were harvested and filtered through a 0.45 μm filter. The resultant lentivirus was stored on ice in a 4° C. refrigerator until used for transduction with human T-cells, as described below in Example 2.

For production of retroviral vector, the anti-cotinine scFv of SEQ ID NO: 18 was inserted into a retroviral cassette including a CMV promoter, CD8α signal peptide and hinge region, CD28 costimulatory domain, CD3z activation domain, and woodchuck hepatitis virus posttranslational regulatory element (WPRE) within the retroviral vector pQCXIP (Clontech Cat. No. 631516). mCherry and blasticidin resistant genes were incorporated into the construct to assist in expression confirmation via imaging and antibiotic enrichment of the transduced population. Recombinant retrovirus supernatants with the full length CAR gene of SEQ ID NO: 28 were produced by the protocol described by Miah and Campbell Methods Mol Biol. 2010; 612: 199-208 and used for transduction with the NK92 cell line as described below in Example 3.

### Example 2: Transduction of Human T Cells

### Lentiviral Transduction of T Cells

CD8 T cells (obtained commercially from AllCells and/or isolated from fresh blood according to standard protocols) were activated with human T Cell TransAct reagent (Miltenyi Biotec) for 18-24 hours in the presence of human IL-2 (200 IU/mL). Lentivirus encoding an anti-cotinine CAR gene prepared as described above in Example 1 was added to the wells along with a transduction enhancer (TransDux™ Max). The cells were centrifuged at 800×g for 90 minutes at 32° C., then incubated at 37° C. for 24 h. Additional lentivirus encoding an anti-cotinine CAR gene was added to the wells, and the cells were again centrifuged at 800×g for 90 minutes at 32° C. The cells were incubated at 37° C. for a total of 10-12 days, during which the media was partially changed every 2 to 3 days. The cells were harvested after 10-12 days and their functional activity was confirmed using a pro-inflammatory cytokine (e.g. IFN-Gamma) production assay described below in Example 4.

### Example 3: Transduction of NK Cells

### Retroviral Transduction of NK Cells

Amphotropic retroviral particles were prepared using the Retro-X Universal packaging system (cat. 631530, Clontech) that includes GP2-293 cells and Amphotropic (4070A) envelope protein as indicated in the manufacturing protocol. Supernatants containing retroviruses containing CAR-cotinine were concentrated 20× using Retro-X concentrator (PT5063-2, Clontech). and aliquoted for further use. NK cells were transduced according to the protocol described by Miah and Campbell Methods Mol Biol. 2010; 612: 199-208 including Lipofectamine and Plus reagent. The plate was centrifuged for 2 hours at 1100×g at RT, followed by an overnight incubation at 37° C. On the following day, cells were centrifuged briefly (5 min at 360×g) and transduction mixtures were removed by aspiration and replaced with 1 ml of fresh media per well. After 24 hr, another round of transduction was performed using the same conditions as described above. After a second round was completed, NK cells were transferred into a T25 flask and cultured for 5 days. Expression was confirmed via to imaging/flow cytometry using mCherry tag. Note: 10 μg/mL of Blasticidin was added for selection but later reduced to 1 μg/mL.

### Example 4: Immune Effector Cell (i.e., Anti-Cotinine CAR-T Cell, Anti-Cotinine CAR-NK92 Cell) Functional Study: Pro-Inflammatory Cytokine (e.g., IFN-Gamma) Production Assay

An ELISA assay was performed to quantify the level of IFN-gamma production by anti-cotinine CAR-T cells in the presence of CyTaC using a Meso Scale Discovery (MSD) T-Plex Multispot Cytokine Assay System. To perform the ELISA assay, each sample was obtained from the co-incubation of anti-cotinine immune effector cells (i.e. CAR-T, CAR-NK92), antigen-expressing target cells, and respective antigen-targeting CyTaCs. CAR-T and CAR-NK cells were prepared as described above in Examples 2 and 3, respectively.

Antigen-expressing target cells were pre-seeded at a density 1.5×10⁴ cells per 100 μL of media in each well of a 96-well plate and grown overnight. For functional confirmation of immune effector cell (i.e. anti-cotinine CAR-T cells, anti-cotinine CAR-NK92 cells) activation, 100 nM or 10 nM of CyTaC was introduced into each well where the target cells were seeded to activate the anti-cotinine immune effector cell. For dose response determinations, 10 μM to 0.01 nM of CyTaC was added to the wells. The anti-cotinine immune effector cells (i.e. CAR-T, CAR-NK92) were next added to the wells at a fixed ratio (between 1:1 and 10:1) of effector cells to antigen-expressing After co-incubation, the supernatants were removed from the wells and analyzed to determine IFNγ release using a pro-inflammatory cytokine production assay (Meso Scale Diagnostics V-PLEX Human IFNγ Kit) according to manufacturer's protocol.

For dose response determinations, EC₅₀ calculations were done using Graphpad Prism Software, specifically a nonlinear regression curve fit (Y=Bottom+(Top−Bottom)/(1+10\hat{ }((Log EC50−X)*HillSlope))).

CyTaC compounds of Formula (I) were tested for anti-cotinine immune cell (i.e. CAR-T, CAR-NK92) activation as measured by IFN-Gamma production in the above assay in one or more experimental runs and the results are shown in Table 5. Potency of the compounds of Formula (I) is reported as a pEC₅₀ value. The pEC₅₀ value is the negative log of the EC₅₀ value, wherein the EC₅₀ value is half maximal effective concentration measured in molar (M). For compounds tested in more than one experimental run, the pEC₅₀ value is reported as an average.

TABLE 5
Results for PSMA-Targeting CyTaCs for CAR-T Cell Activation
Measured by IFN-Gamma Production (Example 4)

| Compound No. | IFNγ Production pEC50, LNCap |
|---|---|
| 1 | 9.8 |
| 2 | 7.3 |
| 3 | 7.0 |
| 4 | 7.3 |

### Example 5: In Vitro Cell Killing Assay of Antigen-Expressing Target Cells with Anti-Cotinine Effector Cells (i.e., CAR-T Cells and CAR-NK Cells) in the Presence of Antigen Matched CyTaCs

In vitro cell killing of antigen expressing target cells in the presence of anti-cotinine CAR-T or CAR-NK cells and antigen matched CyTaCs was evaluated by three different methods: Cell Titler Glo assay (method 1); Real-Time Cell Analysis (RTCA) Impedance Assay (method 2); and live cell analysis assay (method 3), as described below.

### Method 1: Cell Titer Glo

A luminescent assay was performed to quantify the number of viable cells remaining following co-incubation of antigen-expressing adherent target cells, anti-cotinine CAR-T cells, and antigen matched CyTaCs using a CellTiter-Glo Luminescent Cell Viability Assay (Promega). To perform the luminescent assay, each sample was obtained from the co-incubation of anti-cotinine effector cells (i.e. CAR-T Cells and CAR-NK Cells), antigen-expressing target cells, and respective antigen-targeting CyTaCs. Antigen-expressing target cells were pre-seeded at a density 1.5×10⁴ cells per 100 μL of media in each well of a 96-well plate and grown overnight. The following day, anti-cotinine effector cells (i.e. CAR-T Cells and CAR-NK Cells), were introduced into each well where the target cells were seeded. For functional confirmation of CAR-T cell activation, 100 nM or 10 nM of CyTaC was introduced to activate the anti-cotinine CAR-T cells. For dose response determinations, 10 μM to 0.01 nM of CyTaC was added to the wells. The cells were then cultured for 24 hours. After co-incubation, the supernatants were removed from the wells, the residual adherent target cells in the wells were then analyzed for cell viability using CellTiter-Glo® Luminescent Cell Viability Assay (Promega) according to manufacturer's instructions, as described below in Example 5.

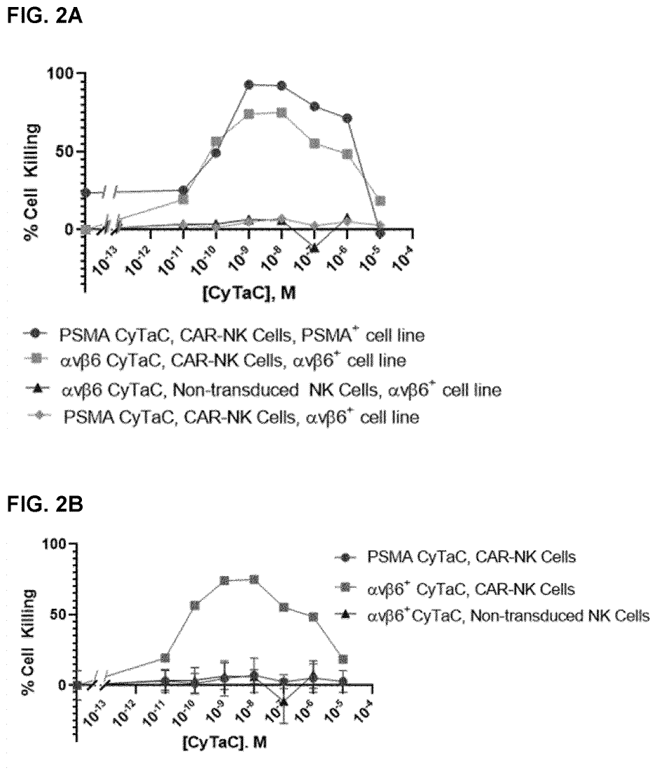

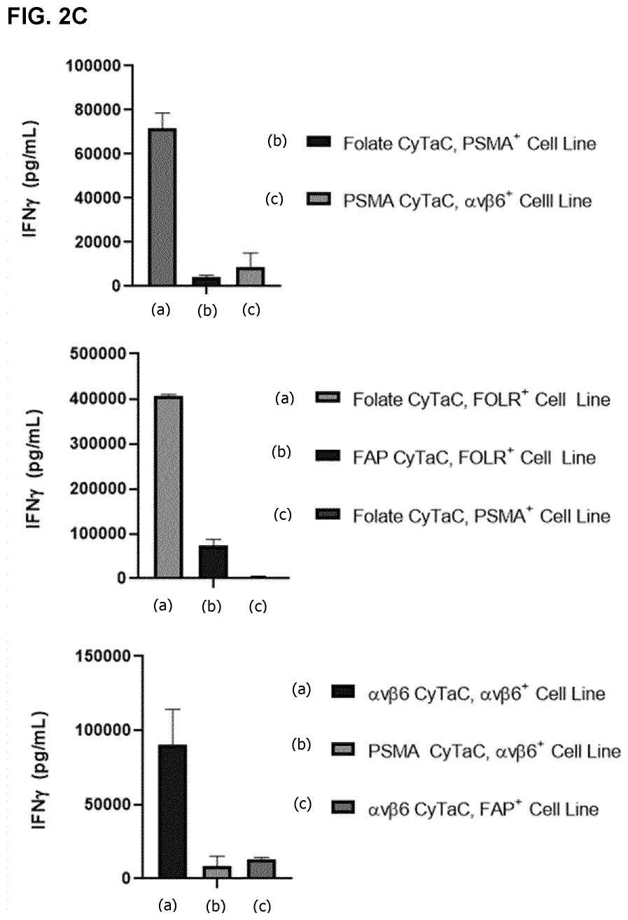

CyTaC compounds of Formula (I) were evaluated for anti-cotinine immune cell mediated cell cytotoxicity in the above assay in one or more experimental runs and the results are shown in Table 6 below and FIGS. 2A, 2B, and 2C. Potency of the compounds of Formula (I) is reported as a pEC₅₀ value. The pEC₅₀ value is the negative log of the EC₅₀ value, wherein the EC₅₀ value is half maximal effective concentration measured in molar (M). For compounds tested in more than one experimental run, the pEC₅₀ value is reported as an average.

TABLE 6
Results for IFNγ Production and Cell Killing as Measured
by Live Cell Analysis (Cell Titer Glo) using PSMA-, αVβ6-,
FOLR-, or FAP-Expressing Target Cells (Example 5)

| | Cell | | | |
|---|---|---|---|---|
| | Titler | IFNγ | Cell | IFNγ |
| | Glo | Production | Titler Glo | Production |
| Compound No. | pEC50 | pEC50 | pEC50 | pEC50 |
| (Target) | (CAR-T) | (CAR-T) | (CAR-NK) | (CAR-NK) |
| 1 (PSMA) | 9.8 | 9.8 | 10.4 | 10.8 |
| 5 (αVβ6) | 9.6 | 9.2 | 11.2 | 9.3 |
| 6 (FOLR) | Inactive | 7.8 | 9.0 | 11.6 |
| 7 (FAP) | 9.7 | 9.0 | 9.3 | 10.3 |

Method 2: Real-Time Cell Analysis (RTCA) Impedance Assay (xCELLigence®)

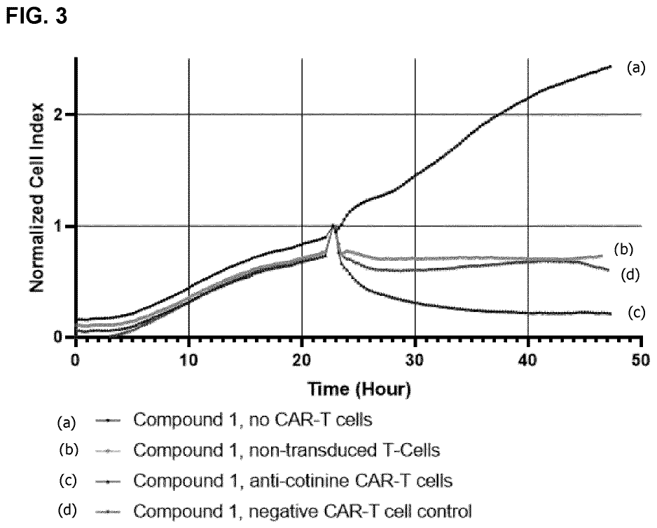

A Real-Time Cell Analysis (RTCA) Impedance Assay (xCELLigence®) was performed to assess in vitro cell killing of antigen-expressing adherent target cells co-incubated with anti-cotinine CAR-T cells and antigen matched CyTaCs. To perform the RTCA assay, 50 μl of medium was added to each well of the E-plate, and a background reading was conducted. Antigen-expressing target cells were then seeded at a density of 3×10⁴ to 5×10⁴ cells per 100 μL of media, and grown overnight in the RTCA machine. During this time, the impedance of the wells was recorded every 5 minutes. The next day, the medium was removed from the wells and replaced with 75 μL of antigen-matched CyTaC solution (200 nM) and 75 μL of anti-cotinine CAR-T cells at a ratio of 1:1 to 10:1 CAR-T cells to antigen-presenting target cells. The plate was returned to the RTCA machine, and the impedance of the wells was recorded every 5 minutes for an additional 24 hours. Representative data for cell killing of antigen expressing cells by anti-cotinine CAR-T cells in the presence of PSMA targeting CyTaC (Compound No. 1) is shown in FIG. 3.

### Method 3: Live-Cell Analysis Assay (Incucyte®)

A Live-Cell Analysis assay (Incucyte®) was performed to assess in vitro cell killing of of fluorescently-labelled antigen-expressing adherent target cells co-incubated with anti-cotinine immune effector cells (i.e. CAR-T cells, CAR-NK92 cells) and antigen matched CyTaCs. To perform the live-cell analysis assay, fluorescently-labelled antigen-expressing target cells were seeded at a density of 1.0×10⁴ to 3×10⁴ cells per 100 μL of media, and grown overnight in a 37° C. incubator. The next day, 50 μL of antigen-matched CyTaC solution was added at a concentration to result in 10 μM to 0.01 nM final assay concentration, followed by the addition of 50 μL anti-cotinine immune effector cells (i.e. CAR-T cells, CAR-NK92 cells) at a ratio of 5:1 effector cells to antigen-presenting target cells. The samples were placed in Live-Cell Analysis (Incucyte®) instrument, and the fluorescence of the wells was recorded every 2 hours for an additional 24 to 48 hours.

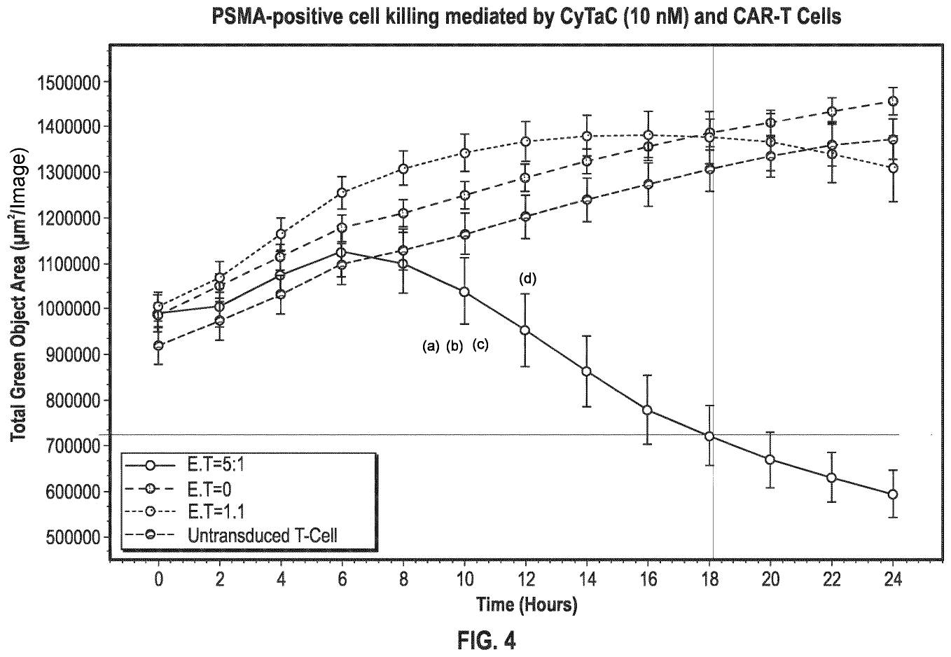

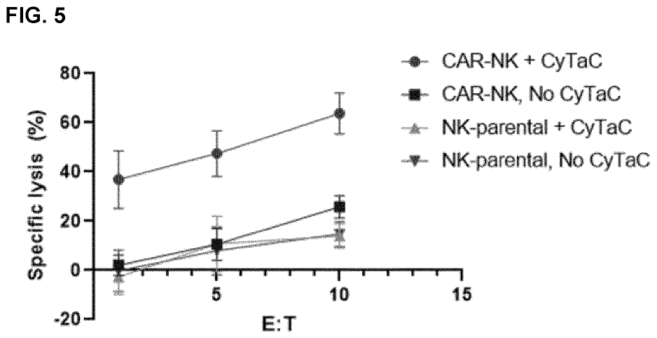

CyTaC compounds of Formula (I) were evaluated for anti-cotinine immune cell (i.e. CAR-T, CAR-NK92) mediated cell cytotoxicity as measured by fluorescence of viable cells in the above assay in one or more experimental runs and the results are shown in Table 7 and FIG. 4. Potency of the compounds of Formula (I) is reported as a pEC₅₀ value at the 24 hour timepoint. The pEC₅₀ value is the negative log of the EC₅₀ value, wherein the EC₅₀ value is half maximal effective concentration measured in molar (M). For compounds tested in more than one experimental run, the pEC₅₀ value is reported as an average.

TABLE 7
Results for Cell Killing as Measured by Live
Cell Analysis (Incucyte ®) using
PSMA-Expressing Target Cells (Example 5)

| Compound No. | Incucyte ® pEC50, LNCap |
|---|---|
| 1 | 9.6 |
| 2 | 8.4 |
| 3 | 7.9 |
| 4 | 8.0 |

### Example 6: Calcein-AM Release Assay

Cell killing of CCR2-expressing cells by anti-cotinine CAR-NK cells in the presence of CCR2 targeting CyTaC molecule was evaluated.

CCR2-expressing target cells were pelleted and resuspended in a 50 μg/μL Calcein AM working solution containing sulfinpyrazone (0.1-0.25 mM). The cells were incubated in a 37° C. incubator with 5% CO₂ for 30 minutes with gentle mixing every 10 minutes. The cells were pelleted, washed, then resuspended and plated at a density of 1.0×10⁴ cells per 100 μL of media in each well of a 96-well plate. CCR2 targeting CyTaC solutions (Compound No. 8) were added at a final well concentration range of 100 nM and the cells were incubated in a 37° C. incubator with 5% CO₂ for 30 minutes. Anti-cotinine CAR-NK effector cells were introduced into each well at a ratios of 10:1, 5:1, and 1:1. The plates were incubated in a 37° C. incubator with 5% CO₂ for 3 hours. The plates were centrifuged at 400 g for 5 minutes, and the supernatants were removed from the wells and analyzed for calcein-AM release via fluorescence imaging. Target cell lysis was calculated using the following equation: % Target lysis=((Test Average−spontaneous)/(max−spontaneous))*100 and the results are shown in FIG. 5.

SEQUENCE LISTINGS
Heavy chain variable region CDR1 amino acid sequence
SEQ ID NO: 1
NYWMS
Heavy chain variable region CDR2 amino acid sequence
SEQ ID NO: 2
DIHGNRGFNYHASWAKG
Heavy chain variable region CDR3 amino acid sequence
SEQ ID NO: 3
ADDSGSHDI
Light chain variable region CDR1 amino acid sequence
SEQ ID NO: 4
QSSQSVYSAKLS
Light chain variable region CDR2 amino acid sequence
SEQ ID NO: 5
YGSTLAS
Light chain variable region CDR3 amino acid sequence
SEQ ID NO: 6
QGTFYGPDWYFA
Heavy chain variable region amino acid sequence
SEQ ID NO: 7
EVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIW
GQGTLVTVSS
Light chain variable region amino acid sequence
SEQ ID NO: 8
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIK
Heavy chain amino acid sequence
SEQ ID NO: 9
EVQLVESGGGLVQPGGSLRLSCAASGFTFSNYWMSWVRQAPGKGLEWVGDIHGNRGFN
YHASWAKGRFTVSRSKNTLYLQMNSLRAEDTAVYYCAKADDSGSHDIWGQGTLVTVSSAS
TKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPDVF
LFPPKPKDTLMISRTPEVTCVVVDVSHEDPEVKFNWYVDGVEVHNAKTKPREEQYNSTYR
VVSVLTVLHQDWLNGKEYKCKVSNKALPAPEEKTISKAKGQPREPQVYTLPPSRDELTKNQ
VSLTCLVKGFYPSDIAVEWESNGQPENNYKTTPPVLDSDGSFFLYSKLTVDKSRWQQGNV
FSCSVMHEALHNHYTQKSLSLSPGK
Light chain amino acid sequence
SEQ ID NO: 10
DIQMTQSPSSLSASVGDRVTITCQSSQSVYSAKLSWYQQKPGKAPKLLIYYGST
LASGVPSRFSGSGSGTQFTLTISSLQPEDFATYYCQGTFYGPDWYFAFGGGTKV
EIKRTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRG
EC
Heavy chain amino acid sequence
SEQ ID NO: 11
QQQLVESGGR LVTPGGSLTL TCTASGFSLN NYWMSWVRQA PGKGLEWIGD
IHGNRGENYH ASWAKGRFTV SRTSTTVDLR MTSLTTEDTA IYFCARADDS
GSHDIWGPGT LVTVSSASTK GPSVFPLAPS SKSTSGGTAA LGCLVKDYFP
EPVTVSWNSG ALTSGVHTFP AVLQSSGLYS LSSVVTVPSS SLGTQTYICN
VNHKPSNTKV DKKVEPKSCD KTHTCPPCPA PELLGGPDVF LFPPKPKDTL
MISRTPEVTC VVVDVSHEDP EVKFNWYVDG VEVHNAKTKP REEQYNSTYR
VVSVLTVLHQ DWLNGKEYKC KVSNKALPAP EEKTISKAKG QPREPQVYTL
PPSRDELTKN QVSLTCLVKG FYPSDIAVEW ESNGQPENNY KTTPPVLDSD
GSFFLYSKLT VDKSRWQQGN VFSCSVMHEA LHNHYTQKSL SLSPGK
Light chain amino acid sequence
SEQ ID NO: 12
ELDLTQTPSPVSAAVGDTVTINCQSSQSVYSAKLSWYQQKPGQPPKLLIYYGSTLASGVPS
RFKGSGSGTQFSLTISDVQCADAATYYCQGTYYGPDWYFAFGGGTEVVVKRTVAAPSVFI
FPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
Heavy chain amino acid sequence
SEQ ID NO: 13
QQQLVESGGRLVTPGGSLTLTCTASGFSLNNYWMSWVRQAPGKGLEWIGDIHGNRGFNY
HASWAKGRFTVSRTSTTVDLRMTSLTTEDTAIYFCARADDSGSHDIWGPGTLVTVSSAKTT
APSVYPLAPVCGDTTGSSVTLGCLVKGYFPEPVTLTWNSGSLSSGVHTFPAVLQSDLYTLS
SSVTVTSSTWPSQSITCNVAHPASSTKVDKKIEPRGPTIKPCPPCKCPAPNLLGGPSVFIFPP
KIKDVLMISLSPMVTCVVVDVSEDDPDVQISWFVNNVEVLTAQTQTHREDYNSTLRVVSALP
IQHQDWMSGKEFKCKVNNKALPAPIERTISKPKGSVRAPQVYVLPPPEEEMTKKQVTLTCM
VTDFMPEDIYVEWTNNGKTELNYKNTEPVLDSDGSYFMYSKLRVEKKNWVERNSYSCSVV
HEGLHNHHTTKSFSRTPGK
Light chain amino acid sequence
SEQ ID NO: 14
ELDLTQTPSPVSAAVGDTVTINCQSSQSVYSAKLSWYQQKPGQPPKLLIYYGSTLASGVPS
RFKGSGSGTQFSLTISDVQCADAATYYCQGTYYGPDWYFAFGGGTEVVVKRADAAPTVSI
FPPSSEQLTSGGASVVCFLNNFYPKDINVKWKIDGSERQNGVLNSWTDQDSKDSTYSMSS
TLTLTKDEYERHNSYTCEATHKTSTSPIVKSFNRNEC
scFv-1
SEQ ID NO: 15
ELDLTQTPASVSAAVGGTVTINCQSSQSPYSNEWLSWYQQKPGQAPKVLISRISTLASGVS
SRFKGSGSGTQFTLTISDLECGDAATYFCAGGYNFGLFPFGGGTELEILSSGGGGSGGGG
GGSSRSSQSVKESEGRLVTPGGSLTLTCTVSGIDLSRDWMNWVRQAPGEGLEWIGAIGRS
GDTYYATWAKGRFTISKTSSRTVTLTVTDLQRSDTATYFCARIPYFGWNNGDIWGPGTLVTI
SS
scFv-2
SEQ ID NO: 16
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSGGGGSEVQLVESGGGLVQPGGSLRLSCAASGFTFSNYWMSWVRQAPGKGLEW
VGDIHGNRGFNYHASWAKGRFTVSRSKNTLYLQMNSLRAEDTAVYYCAKADDSGSHDIW
GQGTLVTVSS
scFv-3
SEQ ID NO: 17
EVOLVESGGGLVQPGGSLRLSCAASGFTFSNYWMSWVRQAPGKGLEWVGDIHGNRGFN
YHASWAKGRFTVSRSKNTLYLQMNSLRAEDTAVYYCAKADDSGSHDIWGQGTLVTVSSG
GGGSGGGGSGGGGSGGGGSDIQMTQSPSSLSASVGDRVTITCQSSQSVYSAKLSWYQQ
KPGKAPKLLIYYGSTLASGVPSRFSGSGSGTQFTLTISSLQPEDFATYYCQGTFYGPDWYF
AFGGGTKVEIK
scFv-4
SEQ ID NO: 18
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGGGGGS
GGGGSEVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIWGQGTLV
TVSS
Full-length CAR
SEQ ID NO: 19
MALPVTALLLPLALLLHAARPDIQMTQSPSSLSASVGDRVTITCQSSQSVYSAKLSWYQQK
PGKAPKLLIYYGSTLASGVPSRFSGSGSGTQFTLTISSLQPEDFATYYCQGTFYGPDWYFA
FGGGTKVEIKGGGGSGGGGSGGGGSGGGGSEVQLVESGGGLVQPGGSLRLSCAASGFT
FSNYWMSWVRQAPGKGLEWVGDIHGNRGFNYHASWAKGRFTVSRSKNTLYLQMNSLRA
EDTAVYYCAKADDSGSHDIWGQGTLVTVSSKPTTTPAPRPPTPAPTIASQPLSLRPEACRP
AAGGAVHTRGLDFACDIYIWAPLAGTCGVLLLSLVITLYCKRGRKKLLYIFKQPEMRPVQTT
QEEDGCSCREPEEEEGGCELRVKESRSADAPAYKOGQNQLYNELNLGRREEYDVLDKR
RGRDPEMGGKPRRKNPQEGLYNELQKDKMAEAYSEIGMKGERRRGKGHDGLYQGLST
ATKDTYDALHMQALPPRDYKDDDDK
Signal peptide; VH/VL sequences; linker sequence; CD8 hinge; CD8
transmembrane domain; 4-1BB-CD3-ζ intracellular domain; FLAG tag
CD8α SP
SEQ ID NO: 20
MALPVTALLLPLALLLHAARP
(G₄S)₄ Linker
SEQ ID NO: 21
GGGGSGGGGSGGGGS
SEQ ID NO: 22
GGGGSGGGGSGGGGSGGGGS
CD8a hinge modified (C164S)
SEQ ID NO: 23
ALSNSIMYFSHFVPVFLPAKPTTTPAPRPPTPAPTIASQPLSLRPEASRPAAGGAVHTRGLD
CD28 transmembrane domain and intracellular domains
SEQ ID NO: 24
KPFWVLVVVGGVLACYSLLVTVAFIIFWVRSKRSRLLHSDYMNMTPRRPGPTRKHYQPYAP
PRDFAAYRS
CD3-ζ intracellular domain
SEQ ID NO: 25
RVKFSRSADAPAYQQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
T2A
SEQ ID NO: 26
GSGEGRGSLLTCGDVEENPGP
mCherry-BSD fusion
SEQ ID NO: 27
MVSKGEEDNMAIIKEFMRFKVHMEGSVNGHEFEIEGEGEGRPYEGTQTAKLKVTKGGPLP
FAWDILSPQFMYGSKAYVKHPADIPDYLKLSFPEGFKWERVMNFEDGGVVTVTQDSSLQD
GEFIYKVKLRGTNFPSDGPVMQKKTMGWEASSERMYPEDGALKGEIKQRLKLKDGGHYDA
EVKTTYKAKKPVQLPGAYNVNIKLDITSHNEDYTIVEQYERAEGRHSTGGMDELYKAKPLSQ
EESTLIERATATINSIPISEDYSVASAALSSDGRIFTGVNVYHFTGGPCAELVVLGTAAAAAAG
NLTCIVAIGNENRGILSPCGRCRQVLLDLHPGIKAIVKDSDGQPTAVGIRELLPSGYVWEG
Full-length CAR
SEQ ID NO: 28
MALPVTALLLPLALLLHAARPDIQMTQSPSSLSASVGDRVTITCQSSQSVYSAKLSWYQQK
PGKAPKLLIYYGSTLASGVPSRFSGSGSGTQFTLTISSLQPEDFATYYCQGTFYGPDWYFA
FGGGTKVEIKGGGGSGGGGSGGGGSEVQLVESGGGLVQPGGSLRLSCAASGFTFSNYW
MSWVRQAPGKGLEWVGDIHGNRGFNYHASWAKGRFTVSRSKNTLYLQMNSLRAEDTAVY
YCAKADDSGSHDIWGQGTLVTVSSALSNSIMYFSHFVPVFLPAKPTTTPAPRPPTPAPTIAS
QPLSLRPEASRPAAGGAVHTRGLDKPFWVLVVVGGVLACYSLLVTVAFIIFWVRSKRSRLL
HSDYMNMTPRRPGPTRKHYQPYAPPRDFAAYRSRVKESRSADAPAYQQGONOLYNELN
LGRREEYDVLDKRRGRDPEMGGKPRRKNPQEGLYNELOKDKMAEAYSEIGMKGERRRG

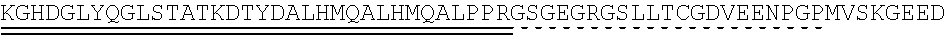

NMAIIKEFMRFKVHMEGSVNGHEFEIEGEGEGRPYEGTQTAKLKVTKGGPLPFAWDILSPQ
FMYGSKAYVKHPADIPDYLKLSFPEGFKWERVMNFEDGGVVTVTQDSSLQDGEFIYKVKLR
GTNFPSDGPVMQKKTMGWEASSERMYPEDGALKGEIKQRLKLKDGGHYDAEVKTTYKAK
KPVQLPGAYNVNIKLDITSHNEDYTIVEQYERAEGRHSTGGMDELYKAKPLSQEESTLIERA
TATINSIPISEDYSVASAALSSDGRIFTGVNVYHFTGGPCAELVVLGTAAAAAAGNLTCIVAIG
NENRGILSPCGRCRQVLLDLHPGIKAIVKDSDGQPTAVGIRELLPSGYVWEG
Signal peptide; VH/VL sequences; linker sequence; CD8alpha hinge modified
(C164S); CD28 transmembrane and intracellular domains;
CD3-7 intracellular domain;

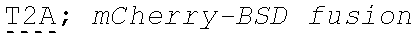

Whitlow Linker
SEQ ID NO: 29
GSTGSGSKPGSGEGSTKG
CD8 Hinge
SEQ ID NO: 30
KPTTTPAPRPPTPAPTIASQPLSLRPEASRPAAGGAVHTRGLDFASDKP
CD8 transmembrane domain
SEQ ID NO: 31
IYIWAPLAGTCGVLLLSLVITLYC
4-1BB-CD3ζ signalling domain
SEQ ID NO: 32
KRGRKKLLYIFKQPFMRPVQTTQEEDGCSCRFPEEEEGGCELRVKFSRSADAPAYQQGQN
QLYNELNLGRREEYDVLDKRRGRDPEMGGKPRRKNPQEGLYNELQKDKMAEAYSEIGMK
GERRRGKGHDGLYQGLSTATKDTYDALHMQALPPR
FLAG tag
SEQ ID NO: 33
DYKDDDDK
Full length CAR (without FLAG tag)
SEQ ID NO: 34
MALPVTALLLPLALLLHAARPDIQMTQSPSSLSASVGDRVTITCQSSQSVYSAKLSWYQQK
PGKAPKLLIYYGSTLASGVPSRFSGSGSGTQFTLTISSLQPEDFATYYCQGTFYGPDWYFA
FGGGTKVEIKGGGGSGGGGSGGGGSGGGGSEVQLVESGGGLVQPGGSLRLSCAASGFT
FSNYWMSWVRQAPGKGLEWVGDIHGNRGFNYHASWAKGRFTVSRSKNTLYLQMNSLRA
EDTAVYYCAKADDSGSHDIWGQGTLVTVSSKPTTTPAPRPPTPAPTIASQPLSLRPEACRP
AAGGAVHTRGLDFACDIYIWAPLAGTCGVLLLSLVITLYCKRGRKKLLYIFKQPFMRPVQTT
QEEDGCSCREPEEEEGGCELRVKESRSADAPAYKOGQNQLYNELNLGRREEYDVLDKR
RGRDPEMGGKPRRKNPQEGLYNELQKDKMAEAYSEIGMKGERRRGKGHDGLYQGLSI
ATKDTYDALHMQALPPR
Signal peptide; VH/VL sequences; linker sequence; CD8 hinge; CD8
transmembrane domain; 4-1BB-CD3-ζ intracellular domain
Full length CAR (without FLAG tag and signal peptide)
SEQ ID NO: 35
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSGGGGSEVQLVESGGGLVQPGGSLRLSCAASGFTFSNYWMSWVRQAPGKGLEW
VGDIHGNRGFNYHASWAKGRFTVSRSKNTLYLQMNSLRAEDTAVYYCAKADDSGSHDIW
GQGTLVTVSSKPTTTPAPRPPTPAPTIASQPLSLRPEACRPAAGGAVHTRGLDFACDIYIWA
PLAGTCGVLLLSLVITLYCKRGRKKLLYIFKOPFMRPVOTTQEEDGCSCREPEEEEGGCEL
RVKESRSADAPAYKQGQNQLYNELNLGRREEYDVLDKRRGRDPEMGGKPRRKNPQEGL
YNELQKDKMAEAYSEIGMKGERRRGKGHDGLYQGLSTATKDTYDALHMQALPPR
VH/VL sequences; linker sequence; CD8 hinge; CD8 transmembrane domain; 4-
1BB-CD3-7 intracellular domain
Full length CAR
SEQ ID NO: 36
MALPVTALLLPLALLLHAARPDIQMTQSPSSLSASVGDRVTITCQSSQSVYSAKLSWYQQK
PGKAPKLLIYYGSTLASGVPSRFSGSGSGTQFTLTISSLQPEDFATYYCQGTFYGPDWYFA
FGGGTKVEIKGGGGSGGGGSGGGGSGGGGSEVQLVESGGGLVQPGGSLRLSCAASGFT
FSNYWMSWVRQAPGKGLEWVGDIHGNRGFNYHASWAKGRFTVSRSKNTLYLQMNSLRA
EDTAVYYCAKADDSGSHDIWGQGTLVTVSSFVPVFLPAKPTTTPAPRPPTPAPTIASQPLSL
RPEACRPAAGGAVHTRGLDFACDIYIWAPLAGTCGVLLLSLVITLYCNHRNKRGRKKLLYIE
KQPFMRPVOTTQEEDGCSCREPEEEEGGCELRVKESRSADAPAYKOGONOLYNELNLG
RREEYDVLDKRRGRDPEMGGKPRRKNPQEGLYNELQKDKMAEAYSEIGMKGERRRGK
GHDGLYQGLSTATKDTYDALHMQALPPR
Signal peptide; VH/VL sequences; linker sequence; CD8 hinge; CD8
transmembrane domain; 4-1BB-CD3- intracellular domain
Full length CAR (without signal peptide)
SEQ ID NO: 37
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSGGGGSEVQLVESGGGLVQPGGSLRLSCAASGFTFSNYWMSWVRQAPGKGLEW
VGDIHGNRGFNYHASWAKGRFTVSRSKNTLYLQMNSLRAEDTAVYYCAKADDSGSHDIW
GQGTLVTVSSFVPVFLPAKPTTTPAPRPPTPAPTIASQPLSLRPEACRPAAGGAVHTRGLDF
ACDIYIWAPLAGTCGVLLLSLVITLYCNHRNKRGRKKLLYIFKQPEMRPVOTTQEEDGCSCR
EPEEEEGGCELRVKESRSADAPAYKOGQNQLYNELNLGRREEYDVLDKRRGRDPEMGG
KPRRKNPQEGLYNELQKDKMAEAYSEIGMKGERRRGKGHDGLYQGLSTATKDTYDALH
MQALPPR
Signal peptide; VH/VL sequences; linker sequence; CD8 hinge; CD8
transmembrane domain; 4-1BB-CD3-ζ intracellular domain
Full length CAR (without mCherry-BSD fusion)
SEQ ID NO: 38
MALPVTALLLPLALLLHAARPDIQMTQSPSSLSASVGDRVTITCQSSQSVYSAKLSWYQQK
PGKAPKLLIYYGSTLASGVPSRFSGSGSGTQFTLTISSLQPEDFATYYCQGTFYGPDWYFA
FGGGTKVEIKGGGGSGGGGSGGGGSEVQLVESGGGLVQPGGSLRLSCAASGFTFSNYW
MSWVRQAPGKGLEWVGDIHGNRGFNYHASWAKGRFTVSRSKNTLYLQMNSLRAEDTAVY
YCAKADDSGSHDIWGQGTLVTVSSALSNSIMYFSHFVPVFLPAKPTTTPAPRPPTPAPTIAS
QPLSLRPEASRPAAGGAVHTRGLDKPFWVLVVVGGVLACYSLLVTVAFIIFWVRSKRSRLL
HSDYMNMTPRRPGPTRKHYQPYAPPRDFAAYRSRVKESRSADAPAYQQGQNQLYNELN
LGRREEYDVLDKRRGRDPEMGGKPRRKNPQEGLYNELQKDKMAEAYSEIGMKGERRRG
KGHDGLYQGLSTATKDTYDALHMQALPPR
Signal peptide; VH/VL sequences; linker sequence; CD8alpha hinge modified
(C164S); CD28 transmembrane and intracellular domains; ;
CD3-7 intracellular domain
Full length CAR (without mCherry-BSD fusion and signal peptide)
SEQ ID NO: 39
DIQMTQSPSSLSASVGDRVTITCQSSQSVYSAKLSWYQQKPGKAPKLLIYYGSTLASGVPS
RFSGSGSGTQFTLTISSLQPEDFATYYCQGTFYGPDWYFAFGGGTKVEIKGGGGSGGGGS
GGGGSEVQLVESGGGLVQPGGSLRLSCAASGFTFSNYWMSWVRQAPGKGLEWVGDIHG
NRGFNYHASWAKGRFTVSRSKNTLYLQMNSLRAEDTAVYYCAKADDSGSHDIWGQGTLV
TVSSALSNSIMYFSHFVPVFLPAKPTTTPAPRPPTPAPTIASQPLSLRPEASRPAAGGAVHT
RGLDKPFWVLVVVGGVLACYSLLVTVAFIIFWVRSKRSRLLHSDYMNMTPRRPGPTRKHY
QPYAPPRDFAAYRSRVKESRSADAPAYQQGQNOLYNELNLGRREEYDVLDKRRGRDPE
MGGKPRRKNPQEGLYNELQKDKMAEAYSEIGMKGERRRGKGHDGLYQGLSTATKDTYD
ALHMQALPPR
VH/VL sequences; linker sequence; CD8alpha hinge modified (C164S); CD28
transmembrane and intracellular domains; CD3-7 intracellular domain;

1. A chimeric antigen receptor comprising: an extracellular ligand binding domain; a transmembrane domain; and an intracellular signalling domain; wherein the extracellular ligand binding domain comprises an anti-cotinine antibody or antigen-binding fragment thereof comprising a heavy chain variable region CDR1 having SEQ ID NO: 1, a heavy chain variable region CDR2 having SEQ ID NO: 2, a heavy chain variable region CDR3 having SEQ ID NO: 3, a light chain variable region CDR1 having SEQ ID NO: 4, a light chain variable region CDR2 having SEQ ID NO: 5, and a light chain variable region CDR3 having SEQ ID NO: 6.

2. The chimeric antigen receptor of claim 1, wherein the antigen-binding fragment is a single chain variable fragment (scFv).

3. The chimeric antigen receptor of claim 2, wherein the scFv comprises a heavy chain variable region (VH) and a light chain variable region (VL) joined by a linker.

4. The chimeric antigen receptor of claim 3, wherein the VH comprises the sequence set forth in SEQ ID NO: 7 and the VL comprises the sequence set forth in SEQ ID NO: 8.

5. The chimeric antigen receptor of claim 4, wherein the scFv comprises the sequence set forth in SEQ ID NO: 15, SEQ ID NO: 16, SEQ ID NO: 17, or SEQ ID NO: 18.

6. The chimeric antigen receptor of any one of the preceding claims, wherein the chimeric antigen receptor comprises the sequence set forth in SEQ ID NO: 19, SEQ ID NO: 28, SEQ ID NO: 34, SEQ ID NO: 35, SEQ ID NO: 36, SEQ ID NO: 37, SEQ ID NO: 38 or SEQ ID NO: 39.

7. A polynucleotide encoding the chimeric antigen receptor of any one of claims 1 to 6.

8. An expression vector comprising the polynucleotide of claim 7.

9. A cell comprising the polynucleotide of claim 7 or expression vector of claim 8.

10. The cell of claim 9, wherein the cell is an immunomodulatory cell.

11. The cell of claim 10, wherein the immunomodulatory cell is a T-cell.

12. The cell of claim 10, wherein the immunomodulatory cell is a natural killer (NK) cell.

13. A modified immunomodulatory cell, wherein the modified immunomodulatory cell expresses the chimeric antigen receptor of any one of claims 1 to 6.

14. The modified immunomodulatory cell of claim 13, wherein the modified immunomodulatory cell is a T cell.

15. The modified immunomodulatory cell of claim 13, wherein the modified immunomodulatory cell is an NK cell.

16. A combination comprising the modified immunomodulatory cell of any one of claims 13 to 15 and a heterobifunctional molecule comprising a moiety that binds a target cell-surface protein covalently linked to a cotinine moiety or a cotinine derivative moiety.

17. The combination of claim 16, wherein the heterobifunctional molecule is a compound of Formula (I): 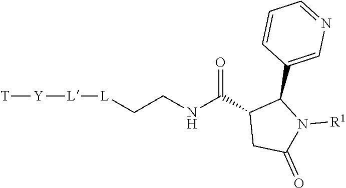
or a pharmaceutically acceptable salt thereof,
wherein:
T is a target binding moiety;
R¹ is C₁₋₄ alkyl or C₃₋₆ cycloalkyl;
L′ is a bond, 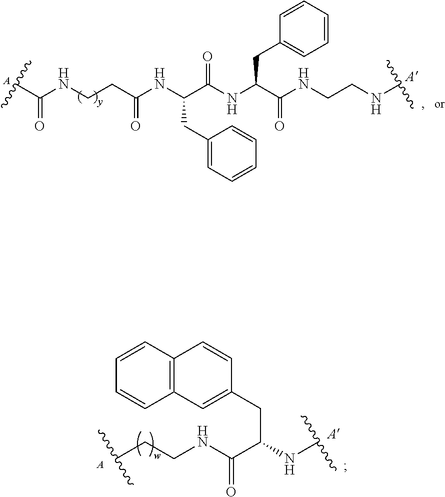
y is an integer of 1 to 9;
w is an integer of 0 to 5;
L is a divalent linker of Formula (L-a), (L-b), (L-c), (L-d), (L-e), (L-f), (L-g), (L-h), (L-i), (L-j), (L-k), (L-m), (L-n-i), (L-n-ii), (L-n-iii), (L-n-iv), (L-p), (L-q), (L-r), or (L-s): 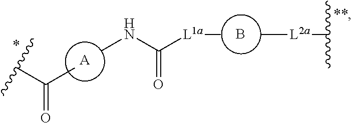
or a stereoisomer thereof,
wherein:
Ring A and Ring B are each independently C₄₋₆ cycloalkylene;
L^{1a} is C₃₋₅ linear alkylene, wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—; 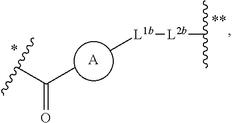
or a stereoisomer thereof,
wherein:
Ring A is C₄₋₆ cycloalkylene or C₇-9 bridged bicyclic cycloalkylene;
L^{1b} is —CH₂—NH—C(O)—, —NHC(O)—, or —C(O)NH—;
L^{2b} is C₆₋₁₂ linear alkylene, wherein 1, 2, 3, or 4 methylene units are replaced with —O—, —NR^{1b}—, —C(O)NR^{1b}—, or —NR^{1b}C(O)—; or
L^{2b} is 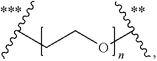
wherein n is 1, 2, 3, or 4, and 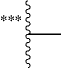
represents a covalent bond to L^{1b}; and
each R^{1b} is independently hydrogen or C₁₋₃ alkyl; 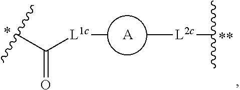
or a stereoisomer thereof,
wherein:
L^{1c} is C₂₋₁₀ linear alkylene, wherein 1, 2, or 3 methylene units are replaced with —O—, —NH—, —NHC(O)—, or —C(O)NH—;
Ring A is C₄₋₆ cycloalkylene or C₇-9 bridged bicyclic cycloalkylene; and
L^{2c} is —O— or a saturated C₂₋₁₀ linear alkylene, wherein 1, 2, or 3 methylene units are replaced with —O—, —NH—, —NHC(O)—, or —C(O)NH—; 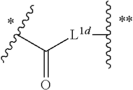
wherein:
L^{1d} is C₁₂₋₂₂ linear alkylene, wherein 1, 2, 3, 4, 5, 6, 7, 8, 9, 10, or 11 methylene units are replaced with —NH—, —O—, —C(O)NH—, —NHC(O)—, or —NHC(O)—NH—; 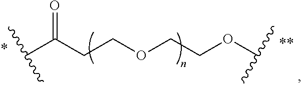
wherein n is an integer of 3 to 50; 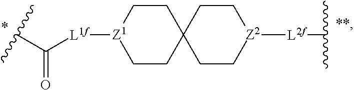
or a stereoisomer thereof,
wherein:
L^{1f} is a bond; C₁₋₆ linear alkylene, wherein 0, 1, or 2 methylene units are replaced with —O—, —NH—, or —C(O)—; or -(−₃₋₆ cycloalkylene)-NHC(O)—;
L^{2f} is a bond, —NHC(O)—, —C(O)NH—, or a C₁₋₆ linear alkylene, wherein 0, 1, or 2 methylene units are replaced with —O—; and
each of Z¹ and Z² is independently N or CH; 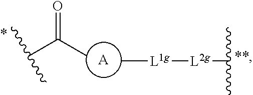
wherein:
Ring A is a 5 to 6 membered heteroarylene having 1 or 2 nitrogen ring atoms;
L^{1g} is a bond, —CH₂—, —NH—, or —O—; and
L^{2g} is 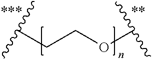
wherein n is 1, 2, 3, 4, or 5, and 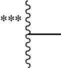
represents a covalent bond to L^{1g}; 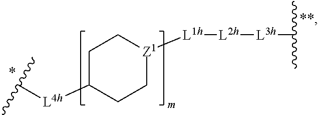
or a stereoisomer thereof,
wherein:
each Z¹ is independently N or CH;
L^{1h} is a bond, —C(O)—, —C(O)—NH—, or —NHC(O)—;
L^{2h} is C₂₋₁₀ linear alkylene or 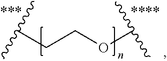
wherein n is 1, 2, 3, or 4, and 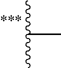
represents a covalent bond to L^{1h} and 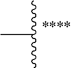
represents a covalent bond to L^{3h};
L^{3h} is a bond, —C(O)CH₂—, —O—(C₃₋₆ cycloalkylene)-O—, or —C(O)NH(CH₂)₃OCH₂—;
L^{4h} is a bond, —C(O)—, —CH₂C(O)—, or —C(O)CH₂—; and
m is 1, 2, or 3; 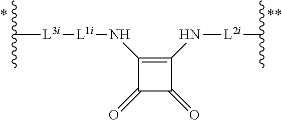
wherein:
L¹ⁱ is a bond, C₁₋₁₂ linear alkylene, or 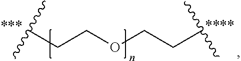
wherein n is 1, 2, 3, 4, or 5, and 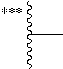
represents a covalent bond to L³ⁱ and 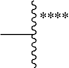
represents a covalent bond to NH;
L²ⁱ is a bond, C₁₋₁₂ linear alkylene, or 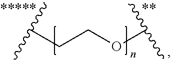
wherein n is 1, 2, 3, 4, or 5, and 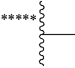
represents a covalent bond to HN; and
L³ⁱ is a bond or —C(O)—; 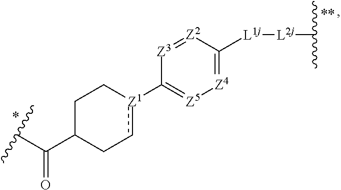
or a stereoisomer thereof,
wherein:
Z¹ is C, CH, or N;
each of Z², Z³, Z⁴ and Z⁵ is independently CH or N, provided that no more than two of Z², Z³, Z⁴ and Z⁵ are N;
L^{1j} is-NH—, —C(O) NH—, —NHC(O)—, or —O—;
L^{2j} is C₁₋₆ linear alkylene or 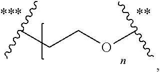
wherein n is 1 or 2, and 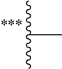
represents a covalent bond to L^{1j}; and [symbol]
represents a single bond or a double bond; 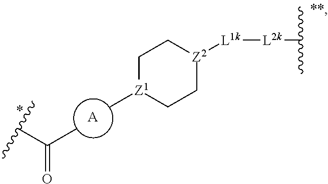
or a stereoisomer thereof,
wherein:
Ring A is phenyl or a 5 or 6 membered heteroarylene having 1 or 2 nitrogen ring atoms;
each of Z¹ and Z² is independently CH or N;
L^{1k} is a bond, —C(O)—, —C(O)NH— or —NHC(O)—; and
L^{2k} is a C₃₋₈ straight chain alkylene or 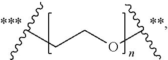
wherein n is 1, 2, or 3, and 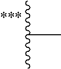
represents a covalent bond to L^{1k}; 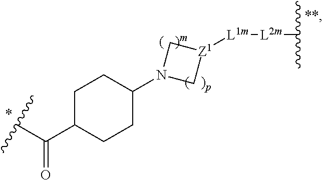
or a stereoisomer thereof,
wherein:
Z¹ is CH or N;
m is 1 or 2;
p is 1 or 2;
0, 1, or 2 hydrogen atoms of 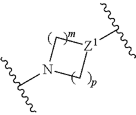
are replaced with F;
L^{1m} is a bond, —C(O)—, —C(O) NH—, —NHC(O)—, —S(O)₂NH— or —NHS(O)₂—; and
L^{2m} is C₃₋₆ linear alkylene, C₃₋₆ cycloalkylene, or 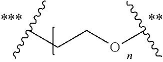
wherein n is 1 or 2, and 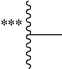
represents a covalent bond to L^{1m}; 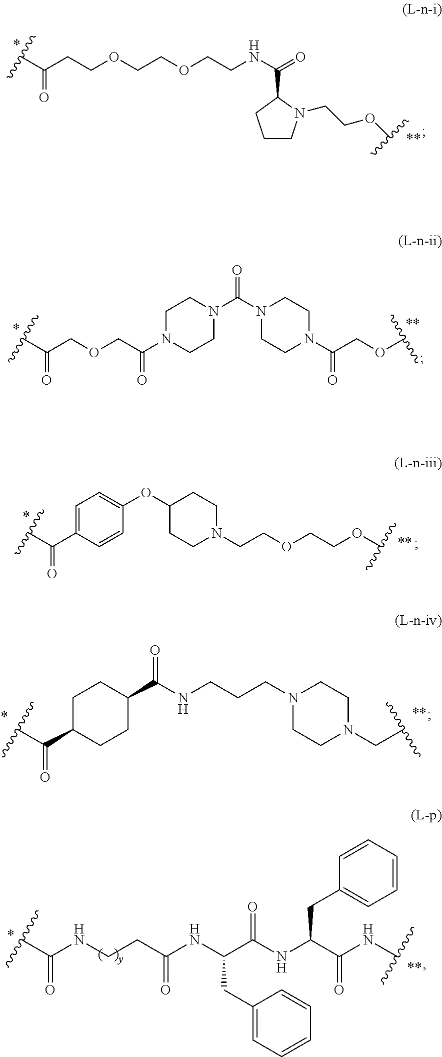
or a stereoisomer thereof, wherein y is an integer of 1 to 9; 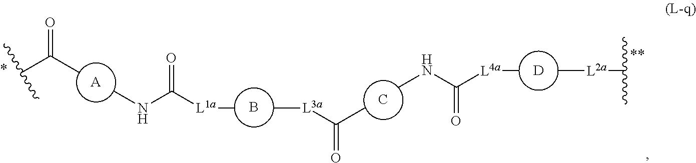
or a stereoisomer thereof,
wherein:
Ring A, Ring B, Ring C, and Ring D are each independently C₄₋₆ cycloalkylene;
L^{1a}, L^{3a}, and L^{4a} are each independently C₃₋₅ linear alkylene,
wherein 1 or 2 methylene units are replaced with —O— or —NR^{a}—;
each R^{a} is independently hydrogen or C₁₋₃ alkyl; and
L^{2a} is —O—, —NHC(O)—, or —CH₂—O—; 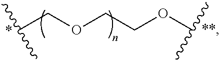
wherein n is an integer of 10 to 30; or 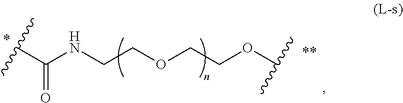
wherein n is an integer of 10 to 30, wherein each 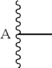
represents a covalent bond to the Y group of Formula (I), or when Y is a bond, a covalent bond to the T group of Formula (I), and each 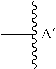
represents a covalent bond to the L group Formula (I), and each of Formula (I); and
wherein each 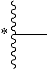
represents a covalent bond to the L′ group of Formula (I), or when L′ is a bond, a covalent bond to the Y group of Formula (I), or when both L′ and Y are a bond, a covalent bond to the T group of Formula (I), and each 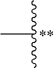
represents a covalent bond to the methylene group of Formula (I); and
Y is a bond or a divalent spacer moiety of one to twelve atoms in length.

18. The combination of claim 17, wherein R¹ is —CH₃.

19. The combination of claim 17 or 18, wherein L′ is a bond.

20. The combination of any one of claims 17 to 19, wherein L is a divalent linker of Formula (L-a-i): 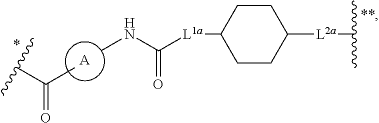
or a stereoisomer thereof,
wherein Ring A, L^{1a}, L^{2a}, 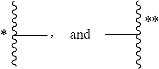
are as defined for Formula (L-a).

21. The combination of any one of claims 17 to 20, wherein L is a divalent linker of Formula (L-a-ii): 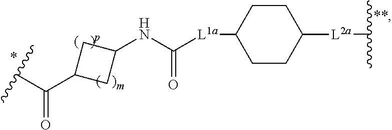
or a stereoisomer thereof,
wherein L^{1a}, L^{2a}, 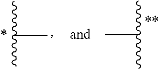
are as defined for Formula (L-a); p is 1 or 2; and m is 1 or 2.

22. The combination of any one of claims 17 to 21, wherein L is a divalent linker of Formula (L-a-iii): 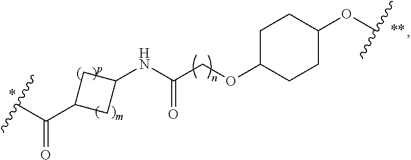
or a stereoisomer thereof,
wherein p is 1 or 2; m is 1 or 2; n is 1, 2, or 3; and 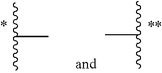
are as defined for Formula (L-a).

23. The combination of any one of claims 17 to 21, wherein L is a divalent linker of Formula (L-a) selected from the group consisting of:

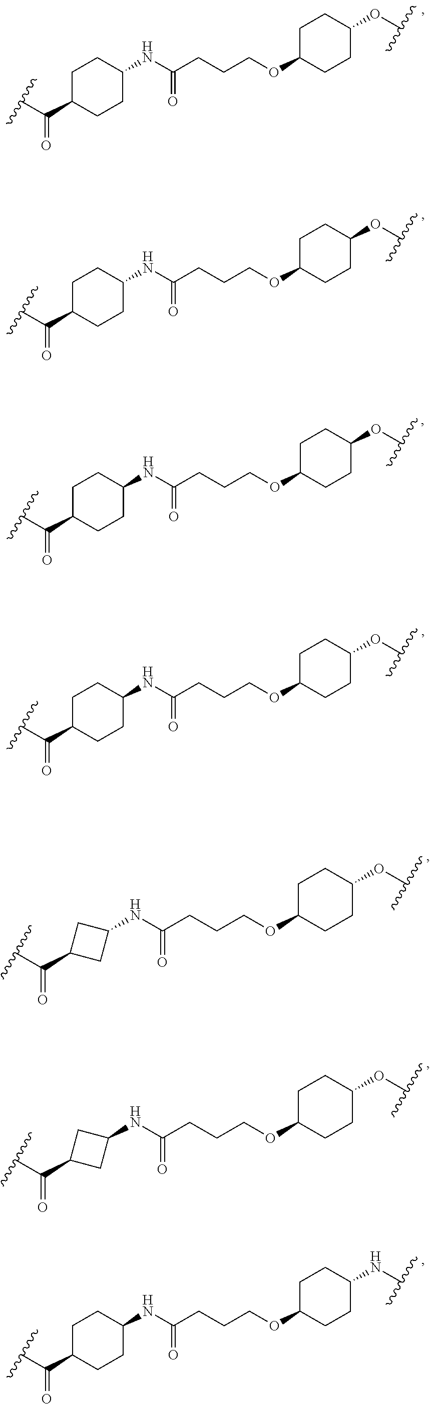

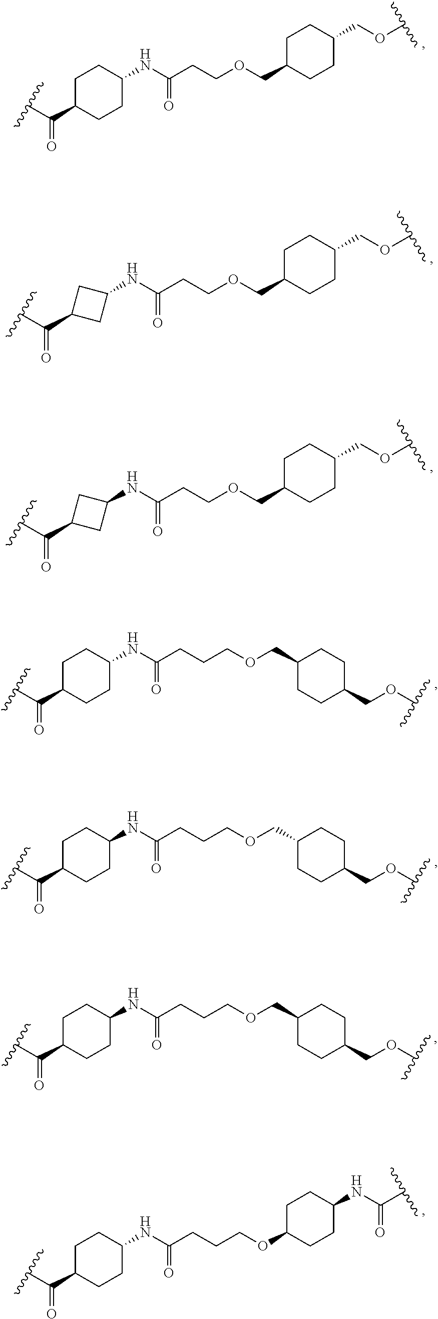

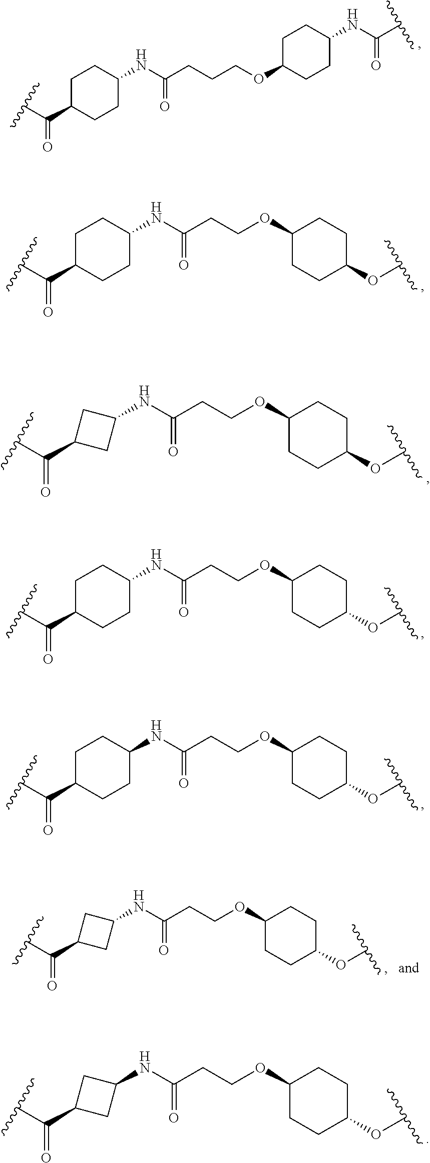

24. The combination of any one of claims 17 to 19, wherein L is a divalent linker of Formula (L-b-i): 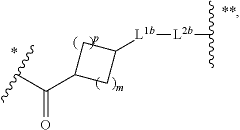
or a stereoisomer thereof,
wherein L^{1b}, L^{2b}, 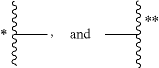
are as defined for Formula (L-b); p is 1 or 2; and m is 1 or 2.

25. The combination of any one of claim 17, 18, 19, or 24, wherein L is a divalent linker of Formula (L-b) selected from the group consisting of:

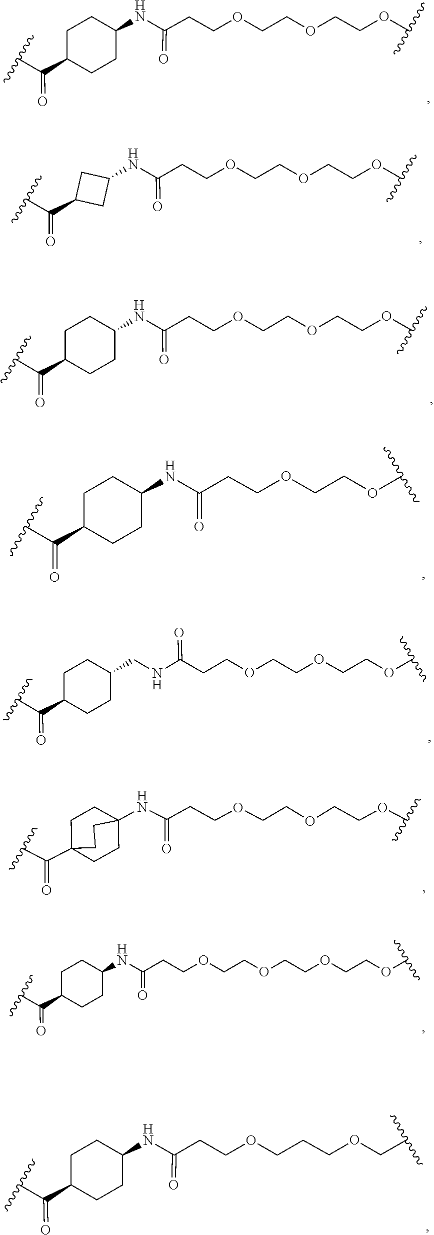

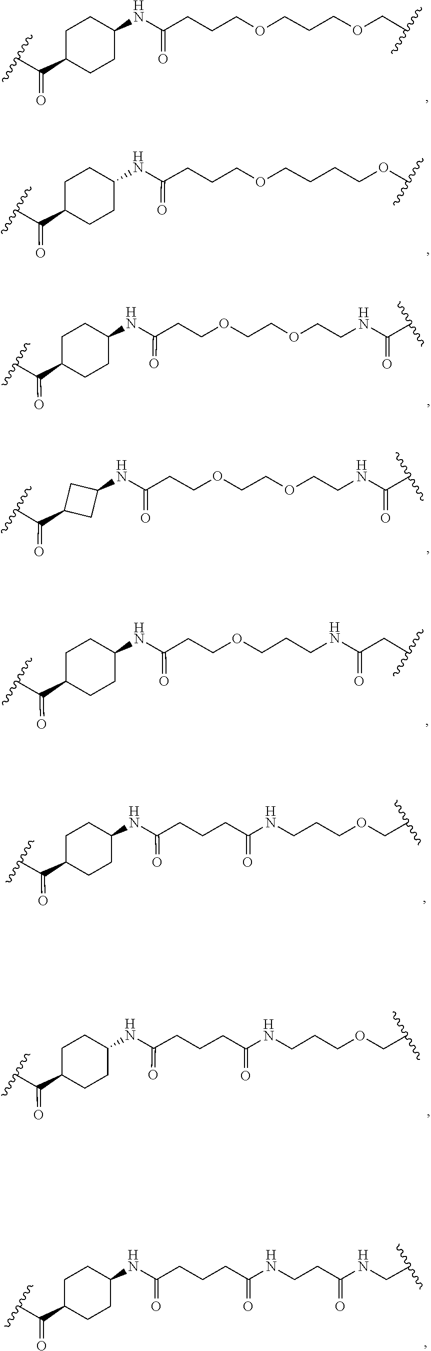

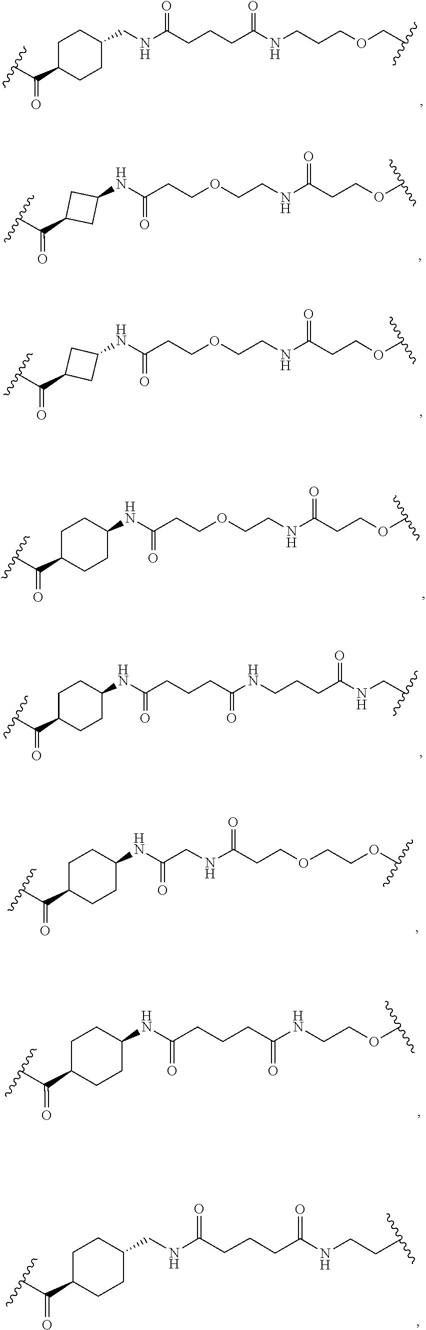

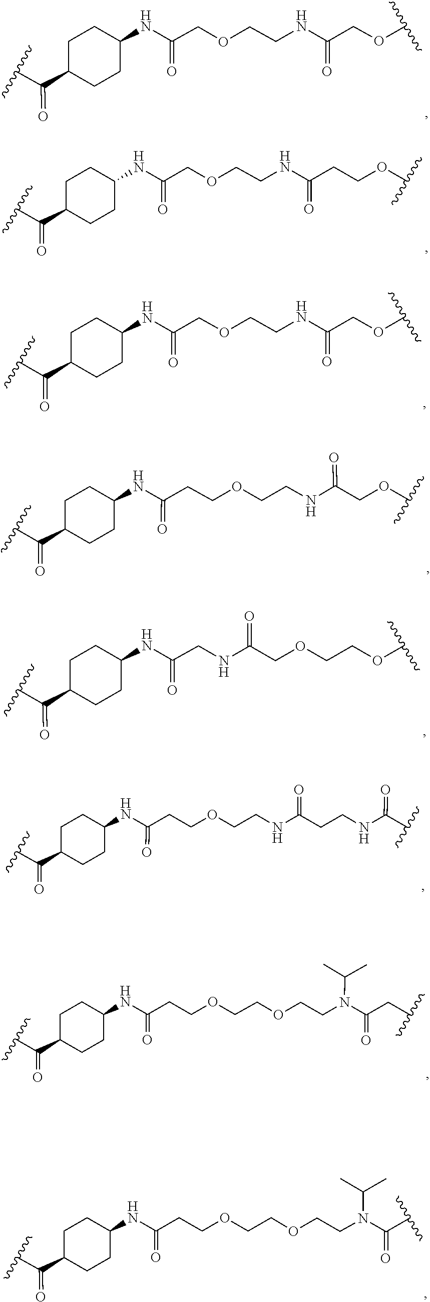

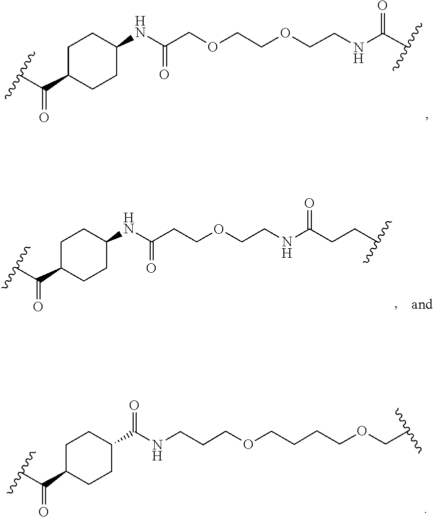 [SMILES: *C(=O)NCCOCCOCC(=O)N[C@H]1CC[C@@H](C(*)=O)CC1 ; *CCC(=O)NCCOCCC(=O)N[C@H]1CC[C@@H](C(*)=O)CC1 ; *COCCCCOCCCNC(=O)[C@H]1CC[C@H](C(*)=O)CC1]

26. The combination of any one of claim 17, 18, or 19, wherein L is a divalent linker of Formula (L-c-i): 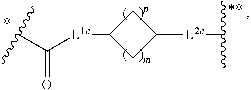
or a stereoisomer thereof,
wherein L^{1c}, L^{2c}, 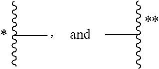
are as defined for Formula (L-c); p is 1 or 2; and m is 1 or 2.

27. The combination of any one of claim 17, 18, or 19, wherein L is a divalent linker of Formula (L-c) selected from the group consisting of:

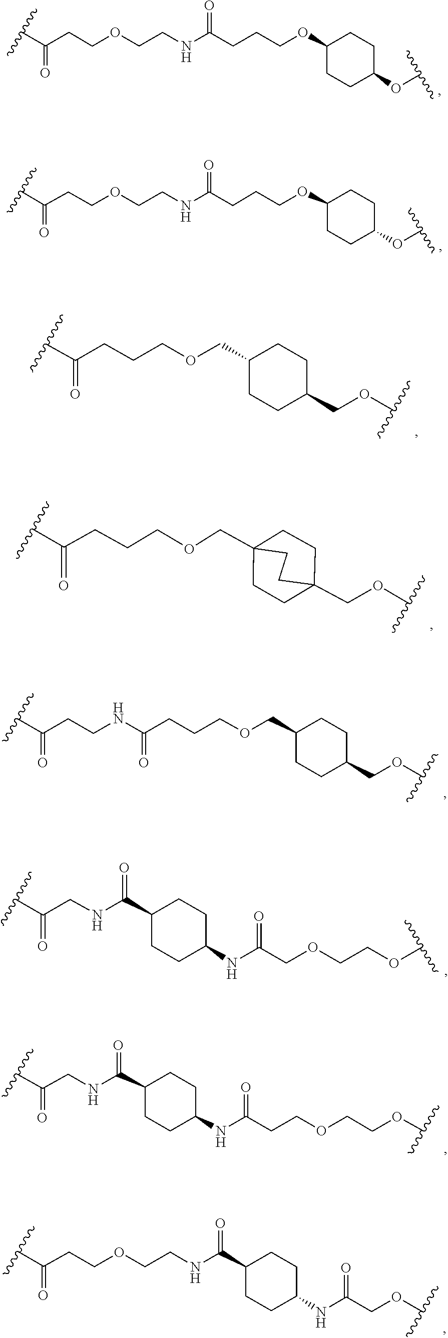

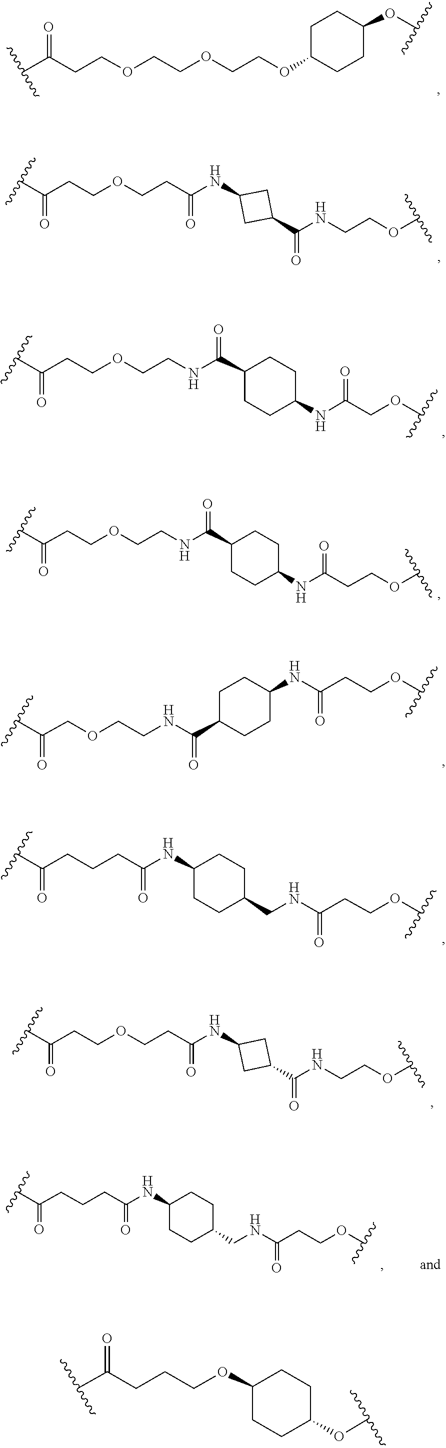

28. The combination of any one of claim 17, 18, or 19, wherein L is a divalent linker of Formula (L-d) selected from the group consisting of:

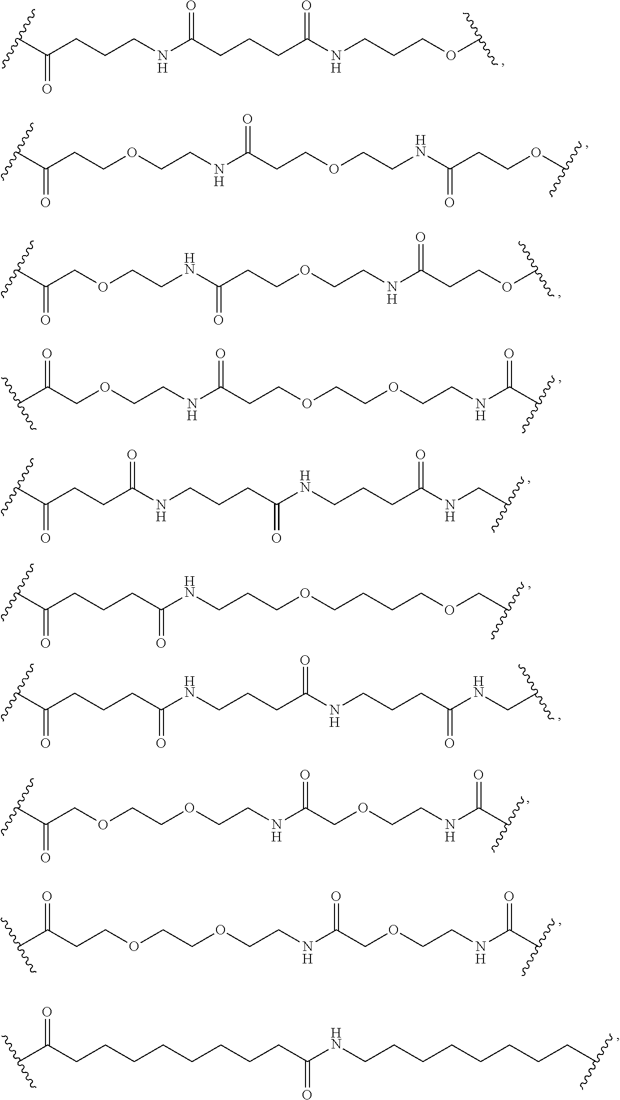

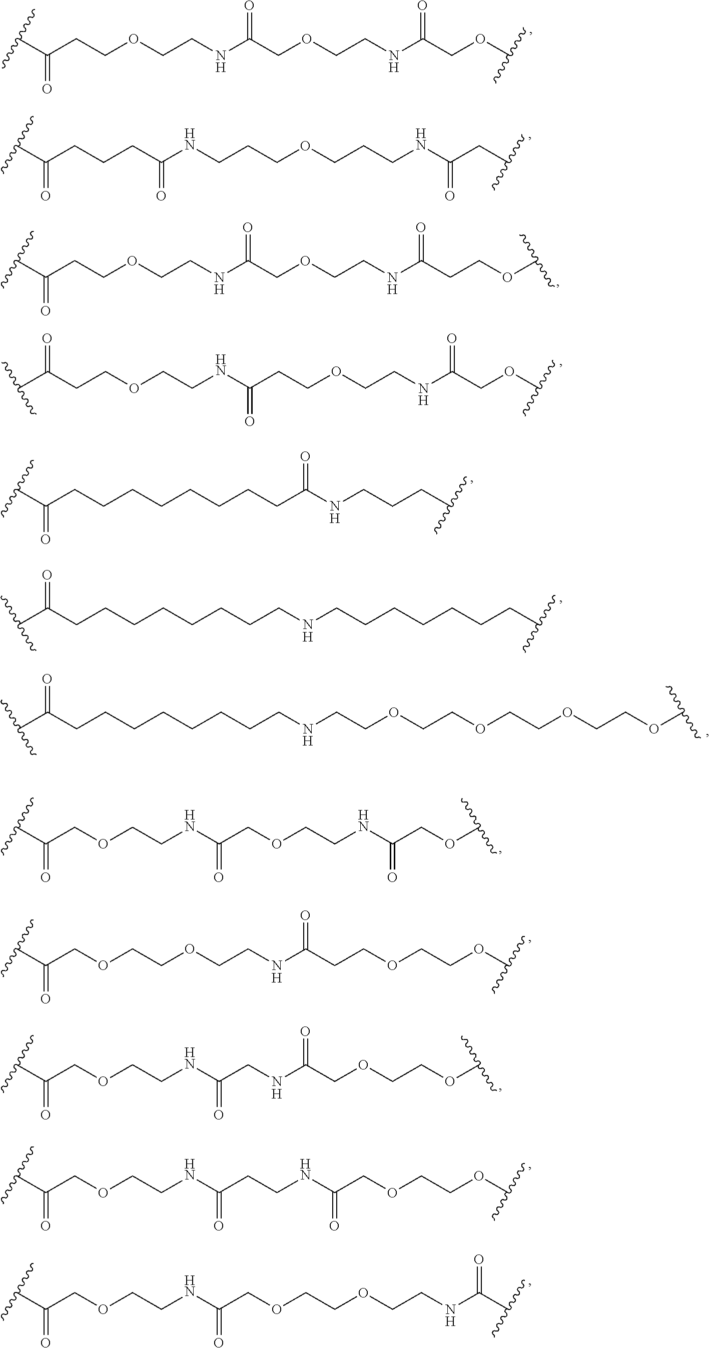

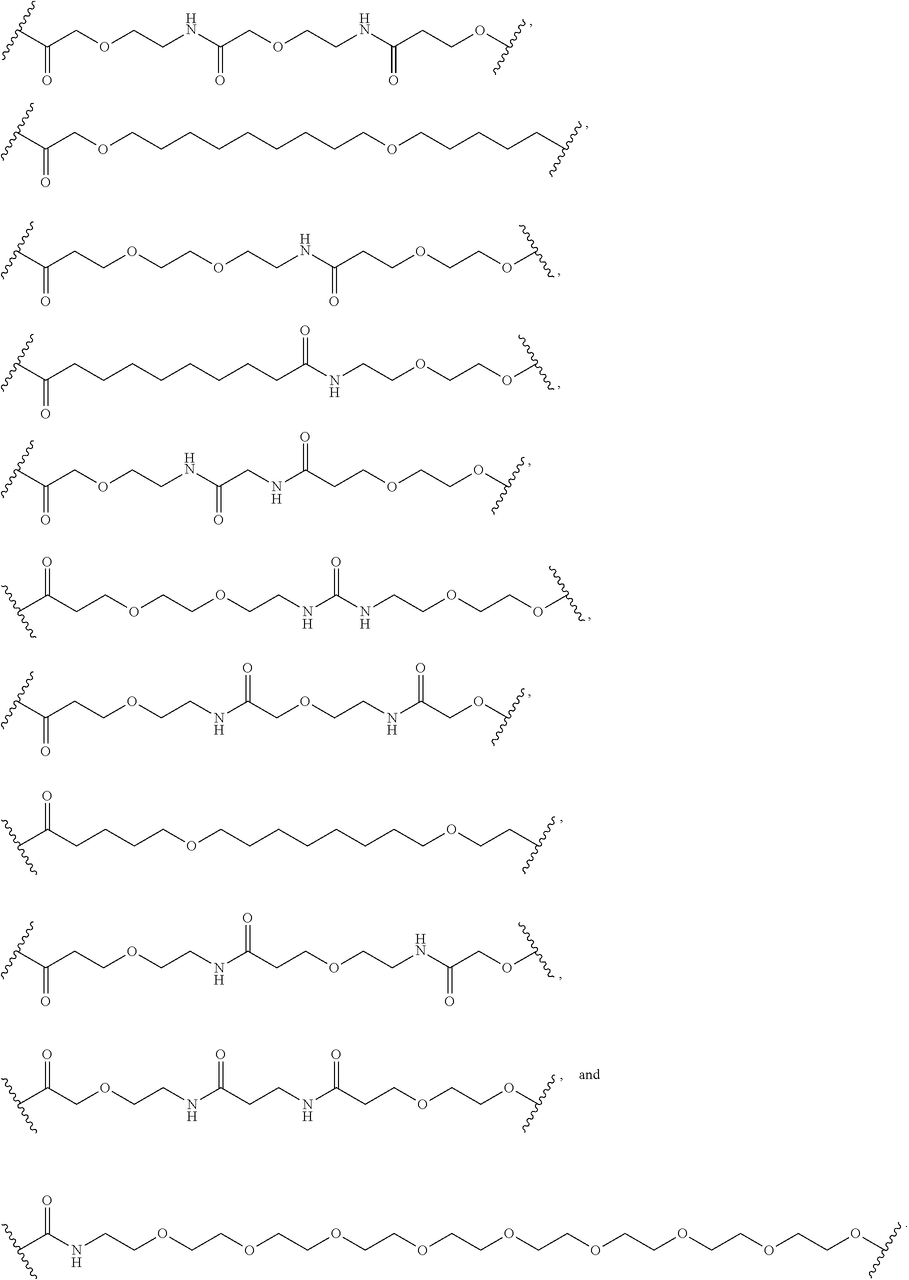

29. The combination of any one of claim 17, 18, or 19, wherein L is a divalent linker of Formula (L-f) selected from the group consisting of:

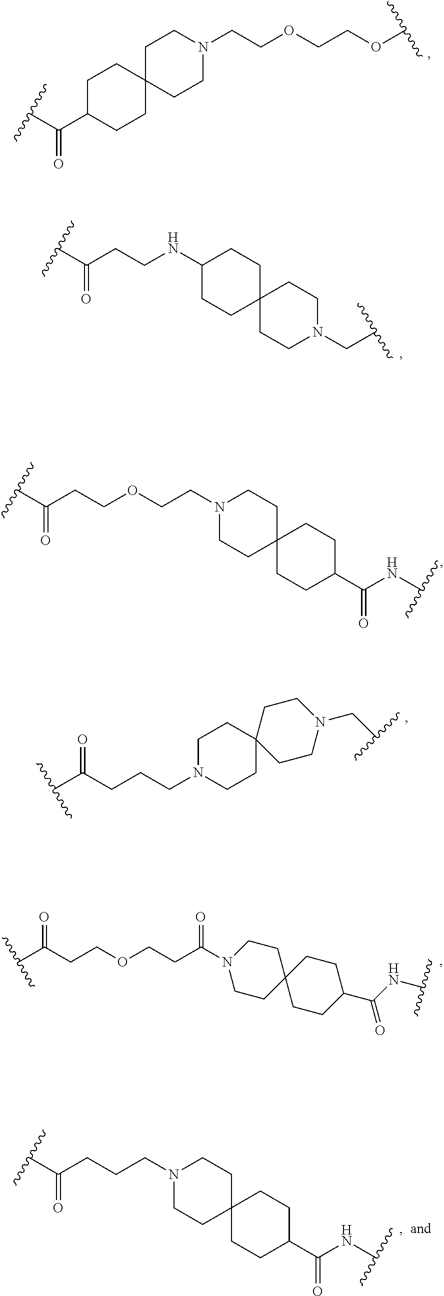 [SMILES: *OCCOCCN1CCC2(CCC(C(*)=O)CC2)CC1 ; *CN1CCC2(CCC(NCCC(*)=O)CC2)CC1 ; *NC(=O)C1CCC2(CC1)CCN(CCOCCC(*)=O)CC2 ; *CN1CCC2(CC1)CCN(CCCC(*)=O)CC2 ; *NC(=O)C1CCC2(CC1)CCN(C(=O)CCOCCC(*)=O)CC2 ; *NC(=O)C1CCC2(CC1)CCN(CCCC(*)=O)CC2]

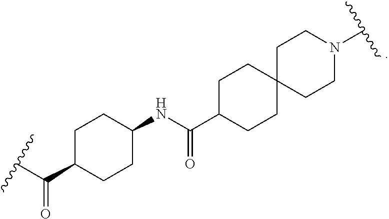 [SMILES: *C(=O)[C@H]1CC[C@@H](NC(=O)C2CCC3(CC2)CCN(*)CC3)CC1]

30. The combination of any one of claim 17, 18, or 19, wherein L is a divalent linker of Formula (L-g-i): 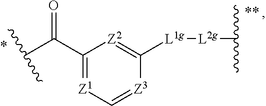
wherein L^{1g}, L^{2g}, 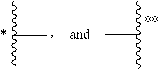
are as defined for Formula (L-g); Z¹, Z², and Z³ are each independently selected from N or CH, provided that one or two of Z¹, Z², and Z³ is N.

31. The combination of any one of claim 17, 18, 19, or 30, wherein L is a divalent linker of Formula (L-g) selected from the group consisting of:

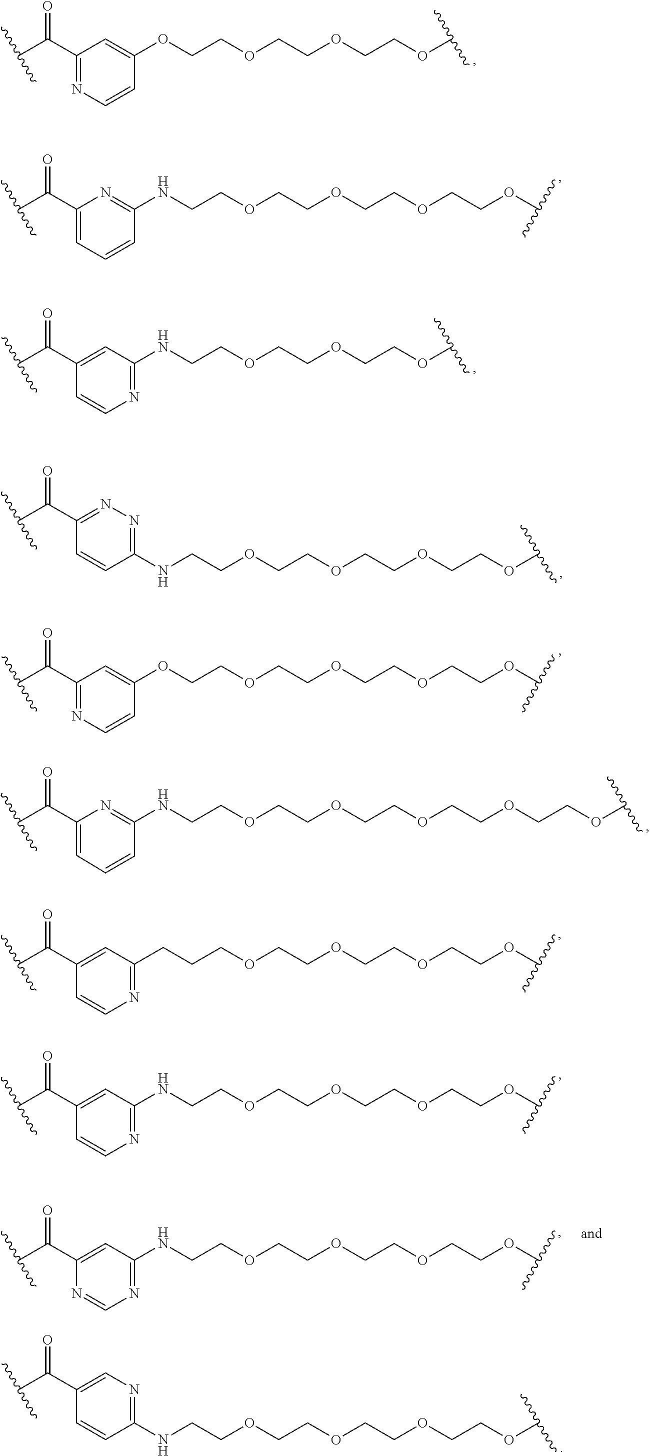

32. The combination of any one of claim 17, 18, or 19, wherein L is a divalent linker of Formula (L-h) selected from the group consisting of:

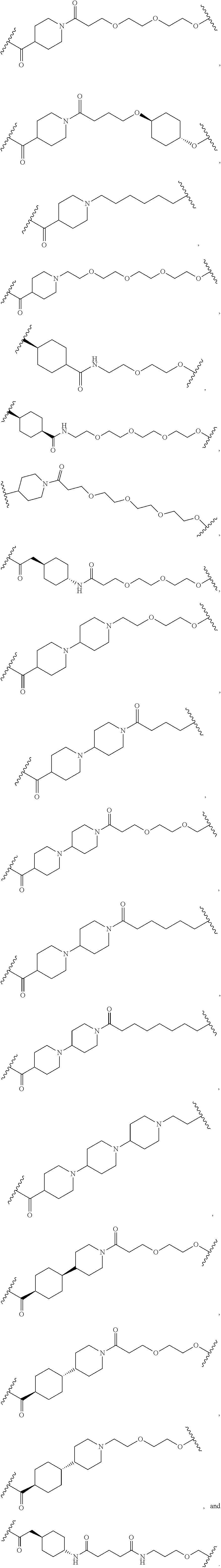

33. The combination of any one of claim 17, 18, or 19, wherein L is a divalent linker of Formula (L-i) selected from the group consisting of:

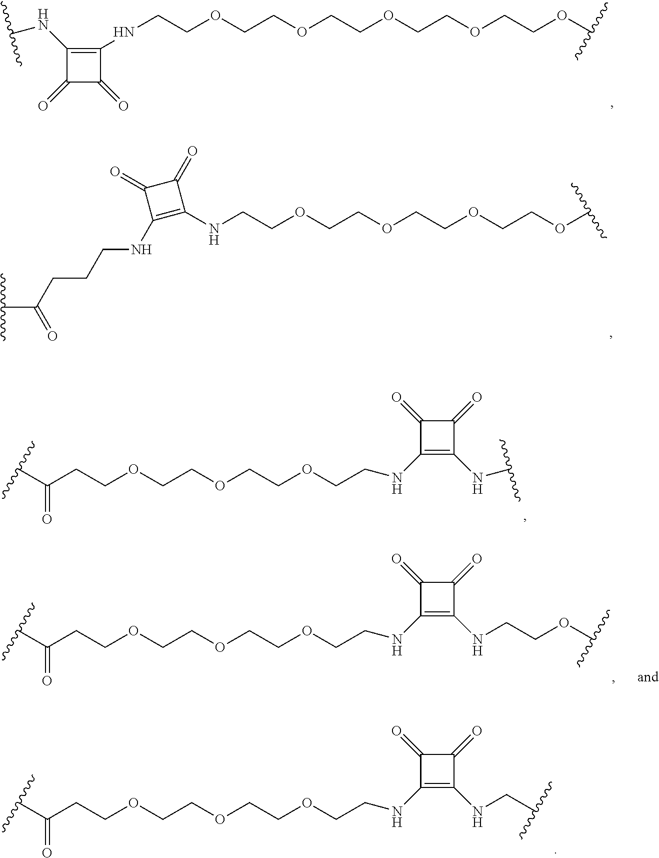 [SMILES: *Nc1c(NCCOCCOCCOCCOCCO*)c(=O)c1=O ; *OCCOCCOCCOCCNc1c(NCCCC(*)=O)c(=O)c1=O ; *Nc1c(NCCOCCOCCOCCC(*)=O)c(=O)c1=O ; *OCCNc1c(NCCOCCOCCOCCC(*)=O)c(=O)c1=O ; *CNc1c(NCCOCCOCCOCCC(*)=O)c(=O)c1=O]

34. The combination of any one of claim 17, 18, or 19, wherein L is a divalent linker of Formula (L-j) selected from the group consisting of:

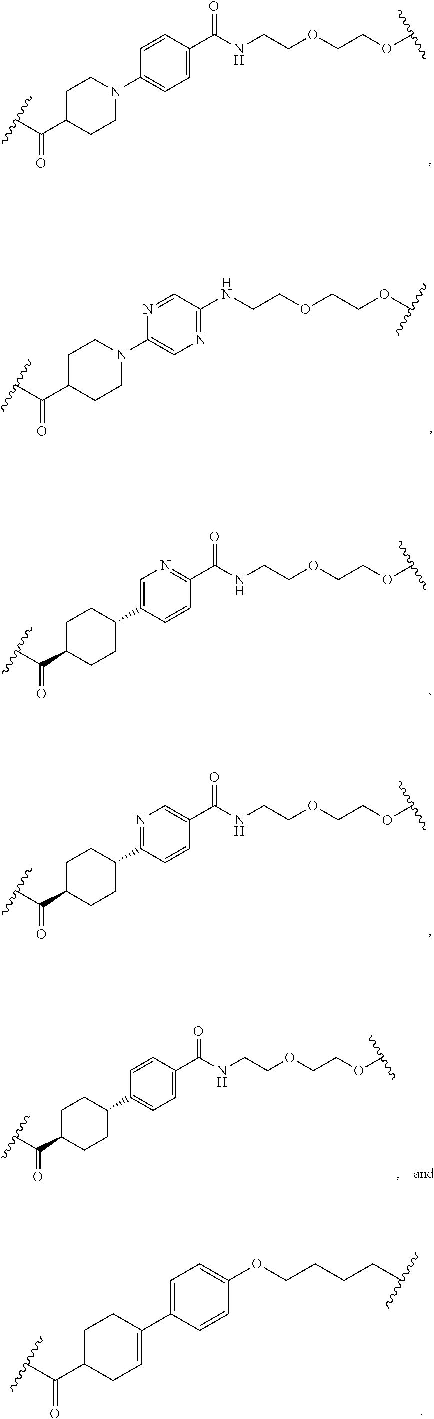 [SMILES: *OCCOCCNC(=O)c1ccc(N2CCC(C(*)=O)CC2)cc1 ; *OCCOCCNc1cnc(N2CCC(C(*)=O)CC2)cn1 ; *OCCOCCNC(=O)c1ccc([C@H]2CC[C@H](C(*)=O)CC2)cn1 ; *OCCOCCNC(=O)c1ccc([C@H]2CC[C@H](C(*)=O)CC2)nc1 ; *OCCOCCNC(=O)c1ccc([C@H]2CC[C@H](C(*)=O)CC2)cc1 ; *CCCCOc1ccc(C2=CCC(C(*)=O)CC2)cc1]

35. The combination of any one of claim 17, 18, or 19, wherein L is a divalent linker of Formula (L-k) selected from the group consisting of:

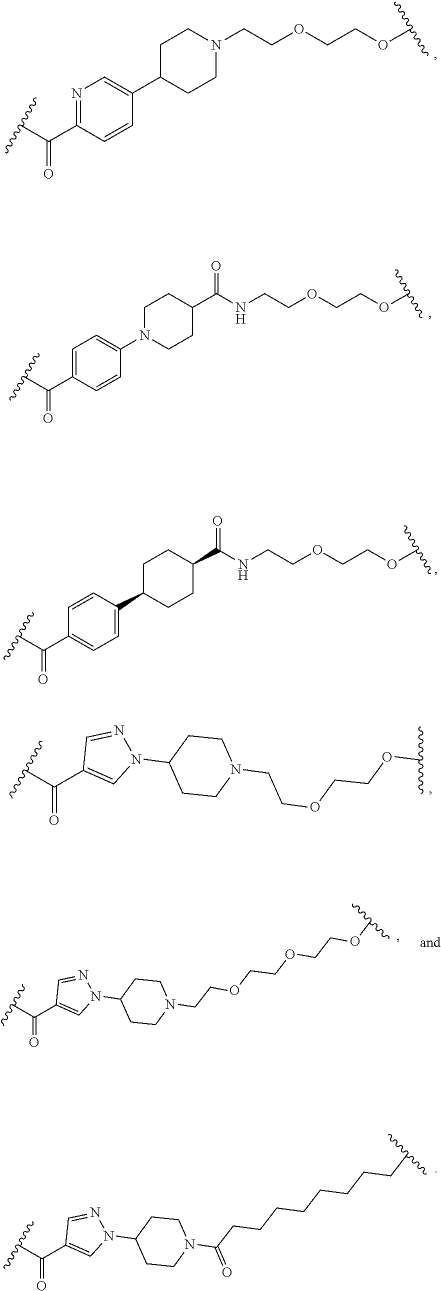 [SMILES: *OCCOCCN1CCC(c2ccc(C(*)=O)nc2)CC1 ; *OCCOCCNC(=O)C1CCN(c2ccc(C(*)=O)cc2)CC1 ; *OCCOCCNC(=O)[C@H]1CC[C@@H](c2ccc(C(*)=O)cc2)CC1 ; *OCCOCCN1CCC(n2cc(C(*)=O)cn2)CC1 ; *OCCOCCOCCN1CCC(n2cc(C(*)=O)cn2)CC1 ; *CCCCCCCCC(=O)N1CCC(n2cc(C(*)=O)cn2)CC1]

36. The combination of any one of claim 17, 18, or 19, wherein L is a divalent linker of Formula (L-m) selected from the group consisting of:

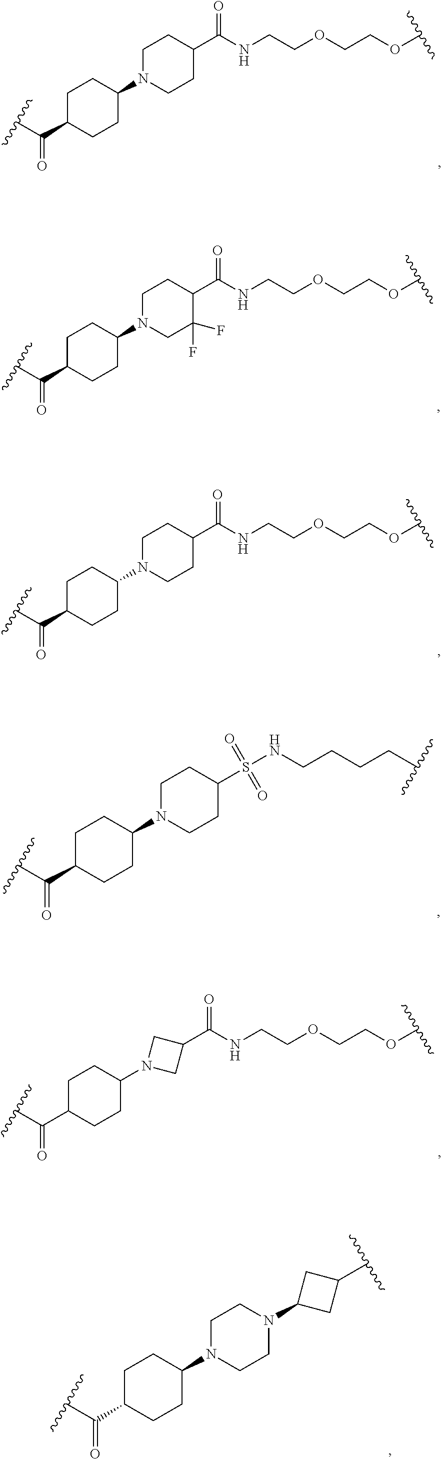

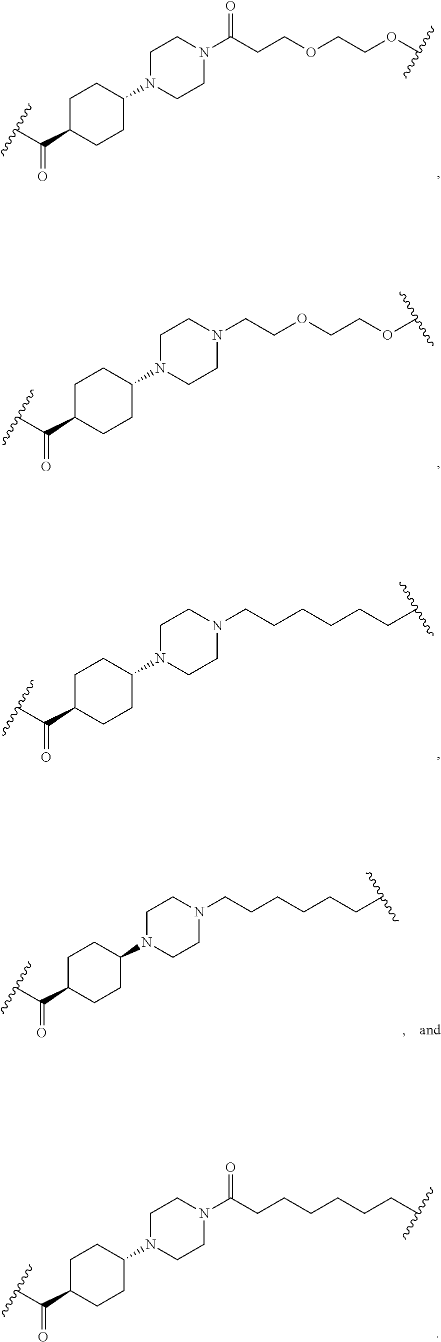 [SMILES: *OCCOCCC(=O)N1CCN([C@H]2CC[C@H](C(*)=O)CC2)CC1 ; *OCCOCCN1CCN([C@H]2CC[C@H](C(*)=O)CC2)CC1 ; *CCCCCCN1CCN([C@H]2CC[C@H](C(*)=O)CC2)CC1 ; *CCCCCCN1CCN([C@H]2CC[C@@H](C(*)=O)CC2)CC1 ; *CCCCCCC(=O)N1CCN([C@H]2CC[C@H](C(*)=O)CC2)CC1]

37. The combination of any one of claim 17, 18, or 19, wherein L is a divalent linker of Formula (L-q-i): 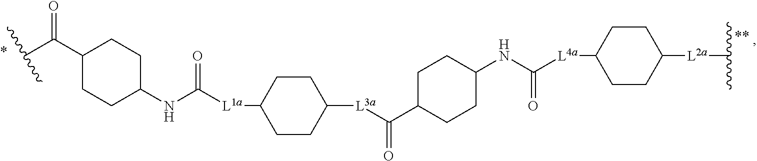
or a stereoisomer thereof,
wherein L^{1a}, L^{3a}, L^{4a}, L^{2a}, 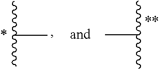
are as defined for Formula (L-q).

38. The combination of any one of claim 17, 18, 19, or 37, wherein L is a divalent linker of Formula (L-q-ii): 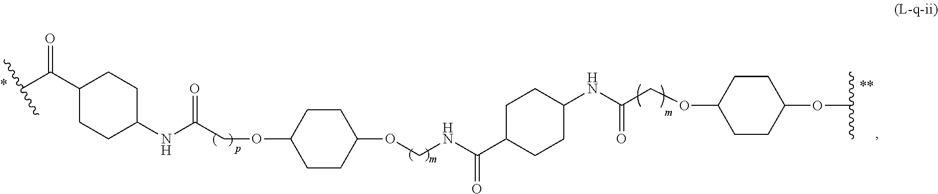
or a stereoisomer thereof,
wherein p is 1, 2, or 3; m is 1, 2, or 3; n is 1, 2, or 3; and 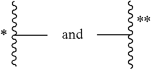
are as defined for Formula (L-q).

39. The combination of any one of claims 17 to 38, wherein Y is selected from a bond; —NH—; —(C₁₋₁₂ alkylene)-, wherein 1, 2, or 3 methylene units are replaced with —O—, —NH—, N(CH₃)—, —C(O)—, NHC(O)—, —C(O) NH—, —(C₃₋₆ cycloalkylene)-, —(C₃₋₆ cycloalkenylene)-, 3- to 10-membered heterocycloalkylene, arylene, or heteroarylene; or -(−₂₋₁₂ alkenylene)-, wherein 1, 2, or 3 methylene units are replaced with —O—, —NH—, N(CH₃)—, —C(O)—, NHC(O)—, —C(O) NH—, —(C₃₋₆ cycloalkylene)-, —(C₃₋₆ cycloalkenylene)-, 3- to 10-membered heterocycloalkylene, arylene, or heteroarylene.

40. The combination of any one of claims 17 to 39, wherein Y is selected from a bond; —NH—; —(C₁₋₆ alkylene)-O—; —O—(C₁₋₆ alkylene)-; —(C₂₋₆ alkenylene)-O—; —(C₁₋₆ alkylene)-C(O)—; —(C₂₋₆ alkenylene)-C(O)—; phenylene; piperidinylene; hydroxypiperidinylene; fluoropiperidinylene; azetidinylene; —C(O)-piperazinylene-; —(C₁₋₆ alkylene)-oxopiperazinylene-; pyrrolidinylene; 7- to 9-membered bridged bicyclic heterocycloalkylene; —(C₁₋₆ alkylene)-O-phenylene-; —(C₂₋₆ alkenylene)-O-piperidinylene; —(C₁₅ alkylene)-NH—, wherein 0, 1, or 2 methylene units are replaced with —O—; —NH—(C₁₋₅ alkylene)-NH—; —N(CH₃)—(C₁₋₅ alkylene)-NH—; NH—(C₁₋₅ alkylene)-N(CH₃)—; —N(CH₃)—(C₁₋₅ alkylene)-N(CH₃)—; —(C₃₋₆ cycloalkylene)-NH—; —C(O)NH—(C₁₋₅ alkylene)-NH—; —C(O) NH—(C₃₋₆ cycloalkylene)-NH—; —(C₁₋₅ alkylene)-O—(C₃₋₆ cycloalkylene)-NH—; —(C₃₋₆ cycloalkenylene)-NH—; or 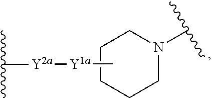
wherein Y^{1a} is a bond, —O—, —NH—, —NHC(O)—, —C(O) NH—, or C₁₋₃ alkylene; and Y^{2a} is a bond, —O—, —NH—, —NHC(O)—, —C(O) NH—, or C₁₋₃ alkylene.

41. The combination of any one of claims 17 to 40, wherein Y is selected from the group consisting of:

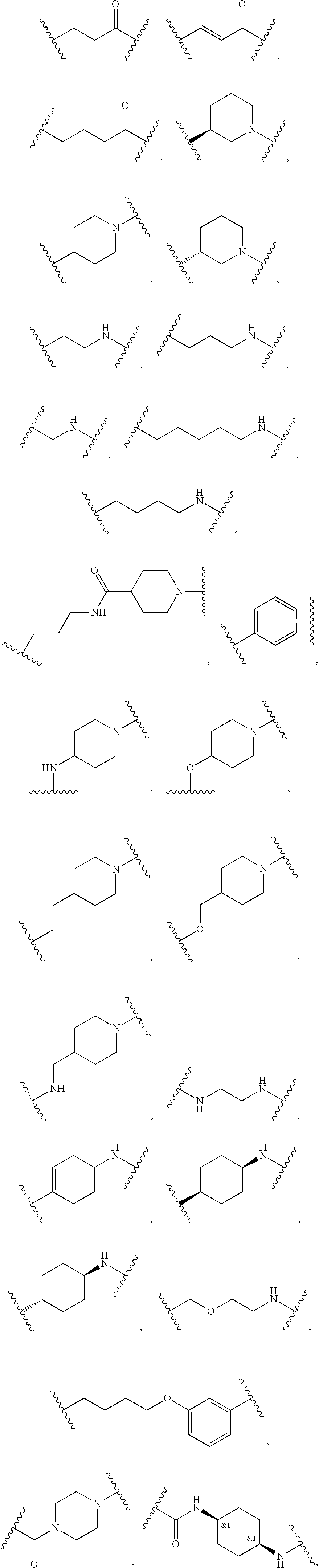

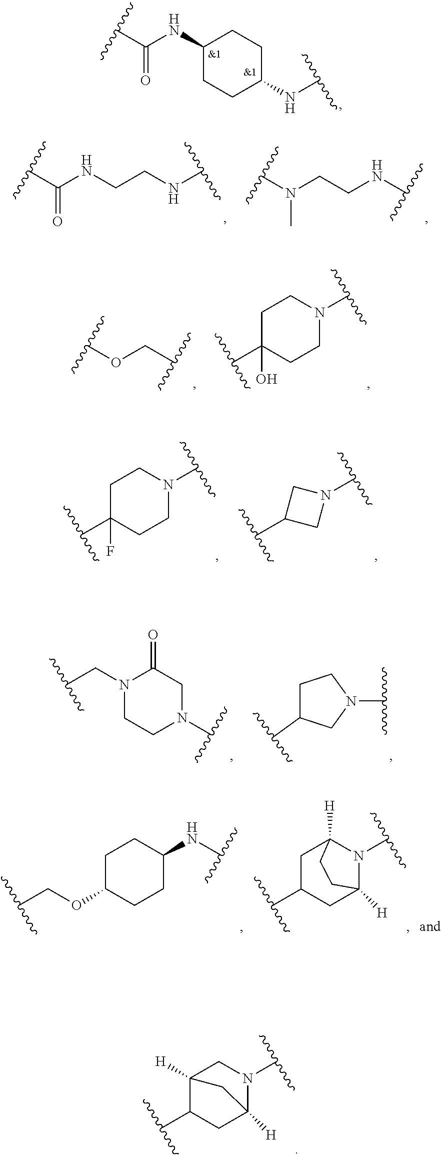

42. The combination of any one of claims 17 to 40, wherein Y is a bond or

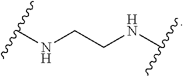 [SMILES: *NCCN*]

43. The combination of any one of claims 17 to 41, wherein Y is selected from the group consisting of:

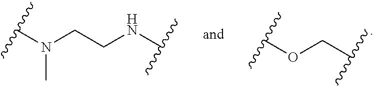

44. The combination of any one of claims 17 to 43, wherein T is: 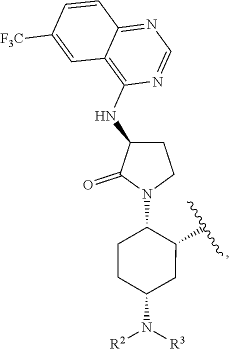
wherein R² is hydrogen or C₁₋₄ alkyl; and R³ is hydrogen or C₁₋₄ alkyl.

45. The combination of claim 44, wherein T is:

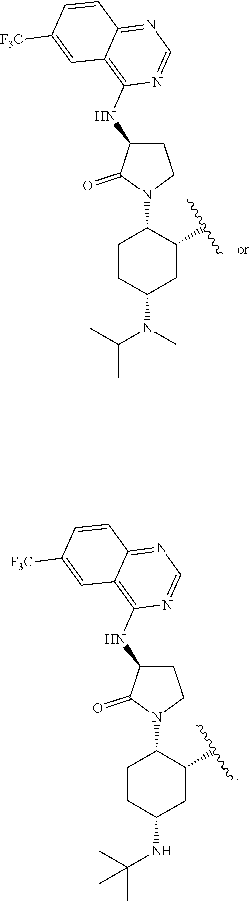 [SMILES: *[C@@H]1C[C@H](N(C)C(C)C)CC[C@@H]1N1CC[C@H](Nc2ncnc3ccc(C(F)(F)F)cc23)C1=O ; *[C@@H]1C[C@H](NC(C)(C)C)CC[C@@H]1N1CC[C@H](Nc2ncnc3ccc(C(F)(F)F)cc23)C1=O]

46. The combination of any one of claims 17 to 43, wherein T is selected from the group consisting of:

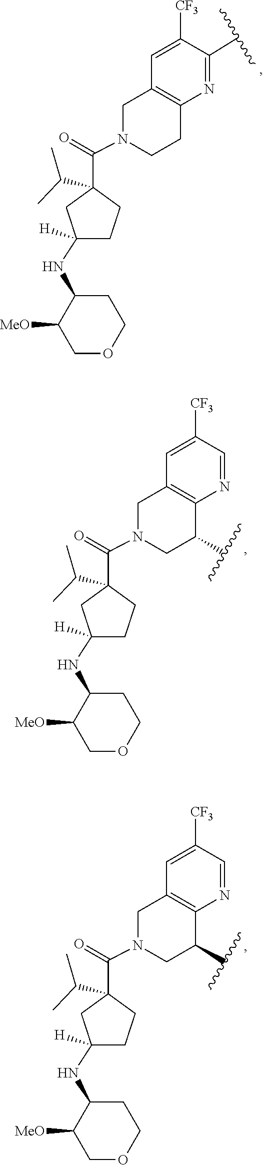 [SMILES: *c1nc2c(cc1C(F)(F)F)CN(C(=O)[C@@]1(C(C)C)CC[C@@H](N[C@H]3CCOC[C@H]3OC)C1)CC2 ; *[C@@H]1CN(C(=O)[C@@]2(C(C)C)CC[C@@H](N[C@H]3CCOC[C@H]3OC)C2)Cc2cc(C(F)(F)F)cnc21 ; *[C@H]1CN(C(=O)[C@@]2(C(C)C)CC[C@@H](N[C@H]3CCOC[C@H]3OC)C2)Cc2cc(C(F)(F)F)cnc21]

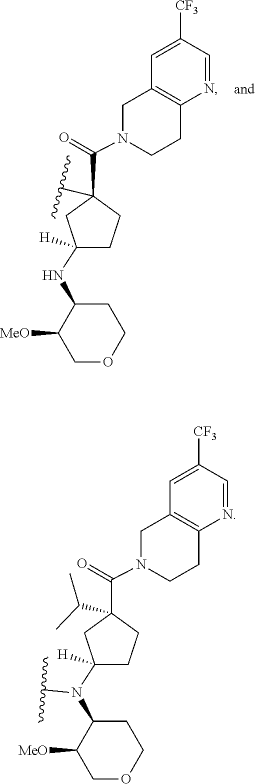 [SMILES: *[C@]1(C(=O)N2CCc3ncc(C(F)(F)F)cc3C2)CC[C@@H](N[C@H]2CCOC[C@H]2OC)C1 ; "and" ; *N([C@@H]1CC[C@@](C(=O)N2CCc3ncc(C(F)(F)F)cc3C2)(C(C)C)C1)[C@H]1CCOC[C@H]1OC]

47. The combination of any one of claims 17 to 43, wherein T is:

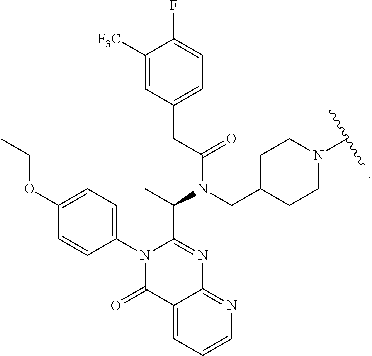 [SMILES: *N1CCC(CN(C(=O)Cc2ccc(F)c(C(F)(F)F)c2)[C@H](C)c2nc3ncccc3c(=O)n2-c2ccc(OCC)cc2)CC1]

48. The combination of any one of claims 17 to 43, wherein T is:

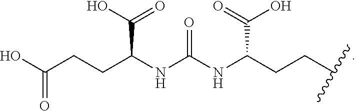 [SMILES: *CC[C@H](NC(=O)N[C@@H](CCC(=O)O)C(=O)O)C(=O)O]

49. The combination of any one of claims 17 to 43, wherein T is:

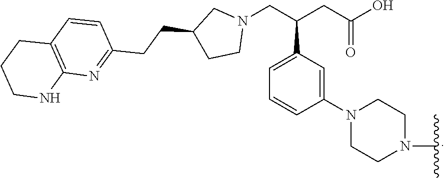 [SMILES: *N1CCN(c2cccc([C@H](CC(=O)O)CN3CC[C@@H](CCc4ccc5c(n4)NCCC5)C3)c2)CC1]

50. The combination of any one of claims 17 to 43, wherein T is:

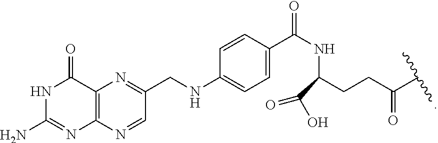 [SMILES: *C(=O)CC[C@H](NC(=O)c1ccc(NCc2cnc3nc(N)[nH]c(=O)c3n2)cc1)C(=O)O]

51. The combination of any one of claims 17 to 43, wherein T is selected from the group consisting of: 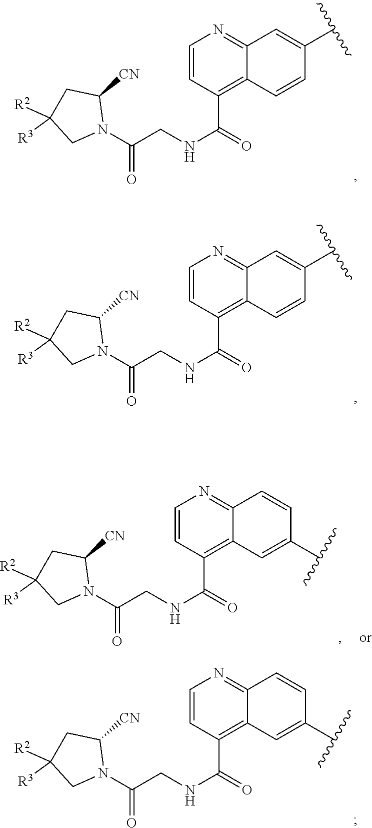
and
R² and R³ are each independently F or H.

52. The combination of any one of claims 17 to 43, wherein T is: 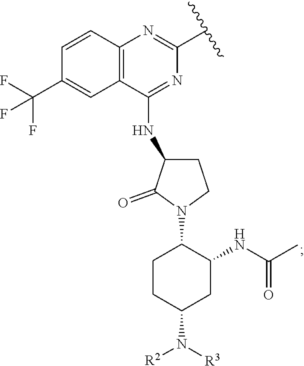
R² is hydrogen or C₁₋₄ alkyl; and
R³ is hydrogen or C₁₋₄ alkyl.

53. The combination of any one of claims 17 to 43, wherein T is: 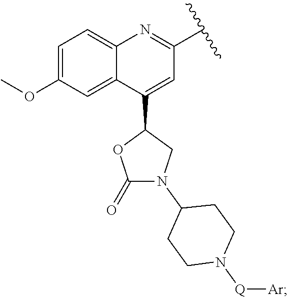
Q is C₁₋₅ alkylene, wherein 0, 1, or 2 methylene units are replaced with —O—; and
Ar is an optionally substituted 5- to 10-membered aromatic ring or 9- or 10-membered unsaturated fused bicyclic ring.

54. The combination of any one of claims 17 to 43, wherein the target of the target binding moiety T is selected from G protein-coupled receptor (GPCRs), enzymes, ion channels, proteases, and receptors.

55. The combination of any one claims 17 to 43, wherein the target of the target binding moiety T is present on a surface of a pathogenic immune cell, a tumor cell or cancer cell, or a stromal cell.

56. The combination of any one of claims 17 to 43, wherein the target of the target binding moiety T is present on the surface of a pathogenic agent selected from a virus or a bacterial cell.

57. The combination of any one claims 17 to 43, wherein the target of the target binding moiety T is present on a surface of monocytic myeloid-derived suppressor cells (mMDSCs), T regulatory cells (Tregs), neutrophils, macrophages, B regulatory cells (Bregs), CD8 regulatory cells (CD8regs), exhausted T cells, polymorphonuclear myeloid derived suppressor cells (PMN-MDSCs), or cancer-associated fibroblasts (CAFs).

58. The combination of any one of claims 17 to 43, wherein the target of the target binding moiety T is a chemokine receptor (CCR).

59. The combination of any one of claims 17 to 43, wherein the target of the target binding moiety T is selected from CCR1, CCR2, CCR3, CCR5, or CCR8.

60. The combination of any one of claims 17 to 43, wherein the target of the target binding moiety T is selected from C-C motif chemokine receptor (CCR) 2 (CCR2), CCR1, CCR3, CCR4, CCR5, CCR6, CCR7, CCR8, CCR9, CCR10, C—X—C motif chemokine receptor 1 (CXCR1), C—X—C motif chemokine receptor 2 (CXCR2), C—X—C motif chemokine receptor 3 (CXCR3), C—X—C motif chemokine receptor 4 (CXCR4), C—X—C motif chemokine receptor 5 (CXCR5), C—X—C motif chemokine receptor 6 (CXCR6), atypical chemokine receptor 3 (ACKR3), integrin αvβ6, fibroblast activation protein-alpha (FAPa), prostate specific membrane antigen (PSMA), folate receptor (folate receptor 1 or folate receptor beta), complement C3a receptor 1 (C3AR1), complement C5a receptor 1 (C5AR1), G protein-coupled receptor (GPR) 65 (GPR65), GRP132, GPR84, GPR183, GPR35, GPR42, cholecystokinin A receptor (CCKAR), leukotriene B4 receptor (LTB4R), somatostatin receptor 2 (SSTR2), free fatty acid receptor 1 (FFAR1), purinergic receptor P2Y2 (P2RY2), prostaglandin D2 receptor (PTGDR), calcitonin receptor (CALCR), CD38, purinergic receptor P2X 7 (P2RX7), integrin subunit alpha V (ITGAV), integrin subunit alpha 5 (ITGA5), integrin subunit beta 1 (ITGB1), integrin subunit beta 6 (ITGB6), integrin subunit beta 3 (ITGB3) prostaglandin D2 receptor 2 (PTGDR2), gastrin releasing peptide receptor (GRPR), MER proto-oncogene tyrosine kinase (MERTK), C-X3-C motif chemokine receptor 1 (CX3CR1), oxidized low density lipoprotein receptor 1 (OLR1), plasminogen activator urokinase receptor (PLAUR), carbonic anhydrase 9 (CA9), carbonic anhydrase 12 (CA12), mas-related G-protein coupled receptor member X2 (MRGPRX2), heat shock protein 90 alpha family class A member 1 (HSP90AA1), dipeptidyl peptidase 4 (DPP4), formyl peptide receptor 2 (FPR2), and succinate receptor 1 (SUCNR1).

61. A method of treating and/or preventing a disease or disorder in a patient in need thereof, the method comprising: administering to the patient a therapeutically effective amount of the combination of any one of claims 16 to 60, wherein the disease or disorder is selected from a cancer, an inflammatory disease, an autoimmune disease, a viral infection, or a bacterial infection.

62. The method of claim 61, wherein the disease or disorder is mediated by chemokine receptor 2 (CCR2) and/or is associated with CCR2-positive pathogenic cells.

63. The method of claim 61, wherein the disease or disorder is mediated by C—X—C motif chemokine receptor 3 (CXCR3) and/or is associated with CXCR3-positive pathogenic cells.

64. The method of claim 61, wherein the disease or disorder is mediated by PSMA and/or is associated with PSMA-positive pathogenic cells.

65. The method of claim 61, wherein the disease or disorder is mediated by integrin αVβ6 and/or is associated with integrin αVβ6-positive pathogenic cells.

66. The method of claim 61, wherein the disease or disorder is mediated by folate receptor α (FRα) and/or folate receptor β (FRB) and/or is associated with FRα- and/or FRβ-positive pathogenic cells.

67. The method of claim 61, wherein the disease or disorder is mediated by fibroblast activation protein (FAP) and/or is associated with FAP-positive pathogenic cells.

68. The method of claim 61, wherein the disease or disorder is mediated by chemokine receptor 8 (CCR8) and/or is associated with CCR8-positive pathogenic cells.

69. The method of any one of claims 61 to 68, wherein the disease is a cancer that is a solid tumor.

70. The method of any one of claims 61 to 69, wherein the cancer is selected from lung cancer (e.g., non-small cell lung cancer (NSCLC)), hepatocellular carcinoma (HCC), colorectal cancer (CRC), cervical cancer (e.g., cervical squamous cell carcinoma (CESC)), head and neck cancer (e.g., head and neck squamous cell carcinoma (HNSC)), pancreatic cancer, prostate cancer (e.g., metastatic castration-resistant prostate cancer (mCRPC)), ovarian cancer, endometrial cancer, brain cancer, endocrine cancer, testicular cancer, bladder cancer, bone cancer, esophageal cancer, gastric cancer, renal cell cancer, melanoma cancer, thyroid cancer, or breast cancer.

71. The method of claim 61 or 63, wherein the disease is an autoimmune or inflammatory disease selected from vitiligo and type I diabetes.

72. The method of any one of claims 61 to 71, wherein the compound and the modified immunomodulatory cell are administered simultaneously.

73. The method of any one of claims 61 to 71, wherein the compound and the modified immunomodulatory cell are administered sequentially.

74. A method of increasing cell killing of target-expressing cells, the method comprising: contacting the cells with an effective amount of the combination of any one of claims 16 to 60, wherein the target-binding moiety of the compound binds the target expressed on the cells.

75. A method of depleting target-expressing cells, the method comprising: contacting the cells with an effective amount of the combination of any one of claims 16 to 60, wherein the target-binding moiety of the compound binds the target expressed on the cells.

76. The method of claim 74 or 75, wherein the target-expressing cells are myeloid-derived suppressor cells (MDSCs), T regulatory cells (Tregs), neutrophils, macrophages, B regulatory cells (Bregs), CD8 regulatory cells (CD8regs), exhausted T cells, or cancer-associated fibroblasts (CAFs).

77. The method of any one of claims 74 to 76, wherein the target-expressing cells are CCR2-expressing cells.

78. The method of claim 74 or 75, wherein the target-expressing cells are CXCR3-expressing cells.

79. The method of claim 74 or 75, wherein the target-expressing cells are PSMA-expressing cells.

80. The method of claim 74 or 75, wherein the target-expressing cells are integrin αVβ6-expressing cells.

81. The method of claim 74 or 75, wherein the target-expressing cells are FRα- and/or FRβ-expressing cells.

82. The method of claim 74 or 75, wherein the target-expressing cells are FAP-expressing cells.

83. The method of claim 74 or 75, wherein the target-expressing cells are CCR8-expressing cells.
